SEQUENCE LISTING (257 sequences)

SEQ ID NO: 1 - protein, 16 aa, Artificial Sequence
  Chemically Synthesized
NDECELCVNVACTGCL

SEQ ID NO: 2 - protein, 15 aa, Artificial Sequence
  Chemically Synthesized
DECELCVNVACTGCL

SEQ ID NO: 3 - protein, 14 aa, Artificial Sequence
  Chemically Synthesized
DECELCVNVACTGC

SEQ ID NO: 4 - protein, 14 aa, Artificial Sequence
  Chemcially Synthesized
ECELCVNVACTGCL

SEQ ID NO: 5 - protein, 13 aa, Artificial Sequence
  Chemically Synthesized
ECELCVNVACTGC

SEQ ID NO: 6 - protein, 13 aa, Artificial Sequence
  Chemically Synthesized
CELCVNVACTGCL

SEQ ID NO: 7 - protein, 12 aa, Artificial Sequence
  Chemically Synthesized
CELCVNVACTGC

SEQ ID NO: 8 - protein, 16 aa, Artificial Sequence
  Chemically Synthesized
  MISC_FEATURE (16)..(16): wherein LEU is a D amino acid
NDECELCVNVACTGCL

SEQ ID NO: 9 - protein, 16 aa, Artificial Sequence
  Chemically Synthesized
  MISC_FEATURE (1)..(1): wherein ASN is a D amino acid
  MISC_FEATURE (16)..(16): wherein LEU is a D amino acid
NDECELCVNVACTGCL

SEQ ID NO: 10 - protein, 16 aa, Artificial Sequence
  Chemically Synthesized
  MISC_FEATURE (1)..(1): wherein ASN is a D amino acid
  MISC_FEATURE (2)..(2): wherein ASP is a D amino acid
  MISC_FEATURE (16)..(16): wherein LEU is a D amino acid
NDECELCVNVACTGCL

SEQ ID NO: 11 - protein, 16 aa, Artificial Sequence
  Chemically Synthesized
  MISC_FEATURE (1)..(1): wherein ASN is a D amino acid
  MISC_FEATURE (2)..(2): wherein ASP is a D amino acid
  MISC_FEATURE (3)..(3): wherein GLU is a D amino acid
  MISC_FEATURE (16)..(16): wherein LEU is a D amino acid
NDECELCVNVACTGCL

SEQ ID NO: 12 - protein, 16 aa, Artificial Sequence
  Chemically Synthesized
  MISC_FEATURE (1)..(1): wherein ASN is a D amino acid
NDECELCVNVACTGCL

SEQ ID NO: 13 - protein, 16 aa, Artificial Sequence
  Chemically Synthesized
  MISC_FEATURE (1)..(1): wherein ASN is a D amino acid
  MISC_FEATURE (6)..(6): wherein LEU is a D amino acid
  MISC_FEATURE (16)..(16): wherein LEU is a D amino acid
NDECELCVNVACTGCL

SEQ ID NO: 14 - protein, 15 aa, Artificial Sequence
  Chemically Synthesized
NDECELCVNVACTGC

SEQ ID NO: 15 - protein, 16 aa, Artificial Sequence
  Chemically Synthesized
  MISC_FEATURE (1)..(1): wherein ASN at position 1 is attached to polyethylene glycol
  MISC_FEATURE (16)..(16): wherein LEU is a D amino acid
  MISC_FEATURE (16)..(16): wherein LEU at position 16 is attached to polyethylene glycol
NDECELCVNVACTGCL

SEQ ID NO: 16 - protein, 16 aa, Artificial Sequence
  Chemically Synthesized
  MISC_FEATURE (1)..(1): wherein ASN at position 1 is attached to polyethylene glycol
  MISC_FEATURE (1)..(1): wherein ASN is a D amino acid
  MISC_FEATURE (16)..(16): wherein LEU is a D amino acid
  MISC_FEATURE (16)..(16): wherein LEU at position 16 is attached to polyethylene glycol
NDECELCVNVACTGCL

SEQ ID NO: 17 - protein, 16 aa, Artificial Sequence
  Chemically Synthesized
  MISC_FEATURE (1)..(1): wherein ASN at position 1 is attached to polyethylene glycol
  MISC_FEATURE (1)..(1): wherein ASN is a D amino acid
  MISC_FEATURE (2)..(2): wherein ASP is a D amino acid
  MISC_FEATURE (16)..(16): wherein LEU is a D amino acid
  MISC_FEATURE (16)..(16): wherein LEU at position 16 is attached to polyethylene glycol
NDECELCVNVACTGCL

SEQ ID NO: 18 - protein, 16 aa, Artificial Sequence
  Chemically Synthesized
  MISC_FEATURE (1)..(1): wherein ASN is a D amino acid
  MISC_FEATURE (16)..(16): wherein LEU at position 16 is attached to polyethylene glycol
  MISC_FEATURE (16)..(16): wherein LEU is a D amino acid
NDECELCVNVACTGCL

SEQ ID NO: 19 - protein, 16 aa, Artificial Sequence
  Chemically Synthesized
  MISC_FEATURE (1)..(1): wherein ASN at position 1 is attached to polyethylene glycol
  MISC_FEATURE (16)..(16): wherein LEU is a D amino acid
NDECELCVNVACTGCL

SEQ ID NO: 20 - protein, 16 aa, Artificial Sequence
  Chemically Synthesized
  MISC_FEATURE (1)..(1): wherein ASN at position 1 is attached to polyethylene glycol
  MISC_FEATURE (1)..(1): wherein ASN is a D amino acid
  MISC_FEATURE (16)..(16): wherein LEU is a D amino acid
NDECELCVNVACTGCL

SEQ ID NO: 21 - protein, 16 aa, Artificial Sequence
  Chemically Synthesized
  MISC_FEATURE (16)..(16): wherein LEU at position 16 is attached to polyethylene glycol
  MISC_FEATURE (16)..(16): wherein LEU is a D amino acid
NDECELCVNVACTGCL

SEQ ID NO: 22 - protein, 16 aa, Artificial Sequence
  Chemically Synthesized
  MISC_FEATURE (1)..(1): wherein ASN at position 1 is attached to polyethylene glycol
  MISC_FEATURE (1)..(1): wherein ASN is a D amino acid
  MISC_FEATURE (2)..(2): wherein ASP is a D amino acid
  MISC_FEATURE (3)..(3): wherein GLU is a D amino acid
  MISC_FEATURE (16)..(16): wherein LEU is a D amino acid
  MISC_FEATURE (16)..(16): wherein LEU at position 16 is attached to polyethylene glycol
NDECELCVNVACTGCL

SEQ ID NO: 23 - protein, 16 aa, Artificial Sequence
  Chemically Synthesized
  MISC_FEATURE (1)..(1): wherein ASN at position 1 is attached to polyethylene glycol
  MISC_FEATURE (1)..(1): wherein ASN is a D amino acid
  MISC_FEATURE (2)..(2): wherein ASP is a D amino acid
  MISC_FEATURE (3)..(3): wherein GLU is a D amino acid
  MISC_FEATURE (16)..(16): wherein LEU is a D amino acid
NDECELCVNVACTGCL

SEQ ID NO: 24 - protein, 16 aa, Artificial Sequence
  Chemically Synthesized
  MISC_FEATURE (1)..(1): wherein ASN is a D amino acid
  MISC_FEATURE (2)..(2): wherein ASP is a D amino acid
  MISC_FEATURE (3)..(3): wherein GLU is a D amino acid
  MISC_FEATURE (16)..(16): wherein LEU at position 16 is attached to polyethylene glycol
  MISC_FEATURE (16)..(16): wherein LEU is a D amino acid
NDECELCVNVACTGCL

SEQ ID NO: 25 - protein, 16 aa, Artificial Sequence
  Chemically Synthesized
  MISC_FEATURE (1)..(1): wherein ASN is a D amino acid
  MISC_FEATURE (2)..(2): wherein ASP is a D amino acid
  MISC_FEATURE (16)..(16): wherein LEU at position 16 is attached to polyethylene glycol
  MISC_FEATURE (16)..(16): wherein LEU is a D amino acid
NDECELCVNVACTGCL

SEQ ID NO: 26 - protein, 16 aa, Artificial Sequence
  Chemically Synthesized
  MISC_FEATURE (1)..(1): wherein ASN at position 1 is attached to polyethylene glycol
  MISC_FEATURE (1)..(1): wherein ASN is a D amino acid
  MISC_FEATURE (2)..(2): wherein ASP is a D amino acid
  MISC_FEATURE (16)..(16): wherein LEU is a D amino acid
NDECELCVNVACTGCL

SEQ ID NO: 27 - protein, 16 aa, Artificial Sequence
  Chemically Synthesized
  MISC_FEATURE (1)..(1): wherein ASN is a D amino acid
  MISC_FEATURE (16)..(16): wherein x is 3-(2-naphthyl)alanine
  MISC_FEATURE (16)..(16): wherein x is a D amino acid
NDECELCVNVACTGCX

SEQ ID NO: 28 - protein, 16 aa, Artificial Sequence
  Chemically Synthesized
  MISC_FEATURE (1)..(1): wherein ASN is a D amino acid
  MISC_FEATURE (8)..(8): wherein x at position 8 is alpha-amino isobutyric acid
  MISC_FEATURE (10)..(10): wherein x at position 10 is alpha-amino isobutyric acid
  MISC_FEATURE (16)..(16): wherein LEU is a D amino acid
NDECELCXNXACTGCL

SEQ ID NO: 29 - protein, 16 aa, Artificial Sequence
  Chemically Synthesized
  MISC_FEATURE (1)..(1): wherein ASN is a D amino acid
  MISC_FEATURE (7)..(7): wherein ASP at position 7 is attached to a Lactam bridge
  MISC_FEATURE (15)..(15): wherein x at position 15 is ornithine
  MISC_FEATURE (16)..(16): wherein LEU is a D amino acid
NDECELDVNVACTGXL

SEQ ID NO: 30 - protein, 16 aa, Artificial Sequence
  Chemically Synthesized
  MISC_FEATURE (1)..(1): wherein ASN is a D amino acid
  MISC_FEATURE (16)..(16): wherein LEU is a D amino acid
NDECEYCVNVACTGCL

SEQ ID NO: 31 - protein, 16 aa, Artificial Sequence
  Chemically Synthesized
  MISC_FEATURE (1)..(1): wherein ASN is a D amino acid
  MISC_FEATURE (16)..(16): wherein LEU is a D amino acid
NDECESCVNVACTGCL

SEQ ID NO: 32 - protein, 16 aa, Artificial Sequence
  Chemically Synthesized
  MISC_FEATURE (1)..(1): wherein ASN at position 1 is attached to polyethylene glycol
  MISC_FEATURE (1)..(1): wherein ASN is a D amino acid
  MISC_FEATURE (16)..(16): wherein LEU is a D amino acid
  MISC_FEATURE (16)..(16): wherein LEU at position 16 is attached to polyethylene glycol
NDECEYCVNVACTGCL

SEQ ID NO: 33 - protein, 16 aa, Artificial Sequence
  Chemically Synthesized
  MISC_FEATURE (1)..(1): wherein ASN at position 1 is attached to polyethylene glycol
  MISC_FEATURE (1)..(1): wherein ASN is a D amino acid
  MISC_FEATURE (16)..(16): wherein LEU is a D amino acid
NDECEYCVNVACTGCL

SEQ ID NO: 34 - protein, 16 aa, Artificial Sequence
  Chemically Synthesized
  MISC_FEATURE (1)..(1): wherein ASN is a D amino acid
  MISC_FEATURE (16)..(16): wherein LEU at position 16 is attached to polyethylene glycol
  MISC_FEATURE (16)..(16): wherein LEU is a D amino acid
NDECEYCVNVACTGCL

SEQ ID NO: 35 - protein, 16 aa, Artificial Sequence
  Chemically Synthesized
  MISC_FEATURE (1)..(1): wherein ASN at position 1 is attached to polyethylene glycol
  MISC_FEATURE (1)..(1): wherein ASN is a D amino acid
  MISC_FEATURE (16)..(16): wherein LEU is a D amino acid
  MISC_FEATURE (16)..(16): wherein LEU at position 16 is attached to polyethylene glycol
NDECESCVNVACTGCL

SEQ ID NO: 36 - protein, 16 aa, Artificial Sequence
  Chemically Synthesized
  MISC_FEATURE (1)..(1): wherein ASN at position 1 is attached to polyethylene glycol
  MISC_FEATURE (1)..(1): wherein ASN is a D amino acid
  MISC_FEATURE (16)..(16): wherein LEU is a D amino acid
NDECESCVNVACTGCL

SEQ ID NO: 37 - protein, 16 aa, Artificial Sequence
  Chemically Synthesized
  MISC_FEATURE (1)..(1): wherein ASN is a D amino acid
  MISC_FEATURE (16)..(16): wherein LEU at position 16 is attached to polyethylene glycol
  MISC_FEATURE (16)..(16): wherein LEU is a D amino acid
NDECESCVNVACTGCL

SEQ ID NO: 38 - protein, 16 aa, Artificial Sequence
  Chemically Synthesized
NDECELCVNVACTGCS

SEQ ID NO: 39 - protein, 16 aa, Artificial Sequence
  Chemically Synthesized
  MISC_FEATURE (1)..(1): wherein ASN at position 1 is attached to polyethylene glycol
  MISC_FEATURE (16)..(16): wherein SER at position 16 is attached to polyethylene glycol
NDECELCVNVACTGCS

SEQ ID NO: 40 - protein, 16 aa, Artificial Sequence
  Chemically Synthesized
  MISC_FEATURE (1)..(1): wherein ASN at position 1 is attached to polyethylene glycol
NDECELCVNVACTGCS

SEQ ID NO: 41 - protein, 16 aa, Artificial Sequence
  Chemically Synthesized
  MISC_FEATURE (16)..(16): wherein SER at position 16 is attached to polyethylene glycol
NDECELCVNVACTGCS

SEQ ID NO: 42 - protein, 16 aa, Artificial Sequence
  Chemically Synthesized
  MISC_FEATURE (1)..(1): wherein ASN at position 1 is attached to polyethylene glycol
  MISC_FEATURE (16)..(16): wherein SER is a D amino acid
  MISC_FEATURE (16)..(16): wherein SER at position 16 is attached to polyethylene glycol
NDECELCVNVACTGCS

SEQ ID NO: 43 - protein, 16 aa, Artificial Sequence
  Chemically Synthesized
  MISC_FEATURE (1)..(1): wherein ASN at position 1 is attached to polyethylene glycol
  MISC_FEATURE (16)..(16): wherein SER is a D amino acid
NDECELCVNVACTGCS

SEQ ID NO: 44 - protein, 16 aa, Artificial Sequence
  Chemically Synthesized
  MISC_FEATURE (16)..(16): wherein SER at position 16 is attached to polyethylene glycol
  MISC_FEATURE (16)..(16): wherein SER is a D amino acid
NDECELCVNVACTGCS

SEQ ID NO: 45 - protein, 16 aa, Artificial Sequence
  Chemically Synthesized
  MISC_FEATURE (5)..(6): x is natural, unnatural, an analogue, L, D, or methylated amino acid or any combination
  MISC_FEATURE (8)..(11): x is natural, unnatural, an analogue, L, D, or methylated amino acid or any combination
  MISC_FEATURE (13)..(14): x is natural, unnatural, an analogue, L, D, or methylated amino acid or any combination
  MISC_FEATURE (16)..(16): x is natural, unnatural, an analogue, L, D, or methylated amino acid or any combination
NDECXXCXXXXCXXCX

SEQ ID NO: 46 - protein, 16 aa, Artificial Sequence
  Chemically Synthesized
  MISC_FEATURE (1)..(1): x is none or a natural, unnatural or an analogue amino acid, may be L, D, or a methylated amino acid, or any combination.
  MISC_FEATURE (2)..(2): x is none or a natural, unnatural or an analogue amino acid, may be L, D, or a methylated amino acid, or any combination.
  MISC_FEATURE (3)..(3): x is any natural, unnatural or an analogue amino acid, may be L, D, or a methylated amino acid, or any combination.
  MISC_FEATURE (5)..(6): x is natural, unnatural, analogue, L, D, or methylated amino acid or any combination
  MISC_FEATURE (8)..(11): x is natural, unnatural, analogue, L, D, or methylated amino acid or any combination
  MISC_FEATURE (13)..(14): x is natural, unnatural, analogue, L, D, or methylated amino acid or any combination
  MISC_FEATURE (16)..(16): x is none or one natural, unnatural or an analogue amino acid, may be L, D, or a methylated amino acid, or any combination
XXXCXXCXXXXCXXCX

SEQ ID NO: 47 - protein, 16 aa, Artificial Sequence
  Chemically Synthesized
  MISC_FEATURE (1)..(1): x is none or a natural, unnatural or an analogue amino acid,  may be L, D, or a methylated amino acid or any combination thereof
  MISC_FEATURE (2)..(2): x is none or a natural, unnatural or an analogue amino acid,  may be L, D, or a methylated amino acid or any combination thereof
  MISC_FEATURE (3)..(3): x is any natural, unnatural or an analogue amino acid,  may be L, D, or a methylated amino acid or any combination thereof
  MISC_FEATURE (4)..(4): wherein x is a cysteine, penicillamine homocysteine, or 3-mercaptoproline
  MISC_FEATURE (6)..(6): x is natural, unnatural, analogue, L, D, or methylated amino acid or any combination
  MISC_FEATURE (7)..(7): wherein x is a cysteine, penicillamine homocysteine, or 3-mercaptoproline
  MISC_FEATURE (12)..(12): wherein x is a cysteine, penicillamine homocysteine, or 3-mercaptoproline
  MISC_FEATURE (15)..(15): wherein x is a cysteine, penicillamine homocysteine, or 3-mercaptoproline
  MISC_FEATURE (16)..(16): x is natural, unnatural or an analogue, zero or one residue in length, may be L, D, or a methylated amino acid or any combination thereof
XXXXEXXVNVAXTGXX

SEQ ID NO: 48 - protein, 16 aa, Artificial Sequence
  Chemically Synthesized
  MISC_FEATURE (1)..(1): x is none or a natural, unnatural or an analogue amino acid, may be L, D, or a methylated amino acid or any combination thereof
  MISC_FEATURE (2)..(2): x is none or a natural, unnatural or an analogue amino acid, may be L, D, or a methylated amino acid or any combination thereof
  MISC_FEATURE (3)..(3): x is any natural, unnatural or an analogue amino acid, may be L, D, or a methylated amino acid or any combination thereof
  MISC_FEATURE (4)..(4): wherein x is a cysteine, penicillamine homocysteine, or 3-mercaptoproline
  MISC_FEATURE (5)..(6): x is any natural, unnatural or an analogue amino acid, may be L, D, or a methylated amino acid or any combination thereof
  MISC_FEATURE (7)..(7): wherein x is a cysteine, penicillamine homocysteine, or 3-mercaptoproline
  MISC_FEATURE (8)..(11): x is natural, unnatural, analogue, L, D, or methylated amino acid or any combination
  MISC_FEATURE (12)..(12): wherein x is a cysteine, penicillamine homocysteine, or 3-mercaptoproline
  MISC_FEATURE (13)..(14): x is natural, unnatural, analogue, L, D, or methylated amino acid or any combination
  MISC_FEATURE (15)..(15): wherein x is a cysteine, penicillamine homocysteine, or 3-mercaptoproline
  MISC_FEATURE (16)..(16): x is natural, unnatural or an analogue, zero or one residue in length, may be L, D, or a methylated amino acid or any combination thereof
XXXXXXXXXXXXXXXX

SEQ ID NO: 49 - protein, 16 aa, Artificial Sequence
  Chemically Synthesized
  MISC_FEATURE (5)..(6): x is natural, unnatural, analogue, L, D, or methylated amino acid or any combination
  MISC_FEATURE (8)..(8): x is natural, unnatural, analogue, L, D, or methylated amino acid or any combination
  MISC_FEATURE (10)..(11): x is natural, unnatural, analogue, L, D, or methylated amino acid or any combination
  MISC_FEATURE (13)..(14): x is natural, unnatural, analogue, L, D, or methylated amino acid or any combination
  MISC_FEATURE (16)..(16): x is natural, unnatural, analogue, L, D, or methylated amino acid or any combination
NDDCXXCXNXXCXXCX

SEQ ID NO: 50 - protein, 16 aa, Artificial Sequence
  Chemically Synthesized
  MISC_FEATURE (1)..(1): wherein ASN is a D amino acid
  MISC_FEATURE (5)..(6): x is natural, unnatural, analogue, L, D, or methylated amino acid or any combination
  MISC_FEATURE (8)..(8): x is any natural, unnatural, analogue, L, D, or methylated amino acid or any combination
  MISC_FEATURE (10)..(11): x is natural, unnatural, analogue, L, D, or methylated amino acid or any combination
  MISC_FEATURE (13)..(14): x is natural, unnatural, analogue, L, D, or methylated amino acid or any combination
  MISC_FEATURE (16)..(16): x is natural, unnatural, analogue, L, D, or methylated amino acid or any combination
  MISC_FEATURE (16)..(16): wherein x is a D amino acid
NEECXXCXNXXCXXCX

SEQ ID NO: 51 - protein, 16 aa, Artificial Sequence
  Chemically Synthesized
  MISC_FEATURE (1)..(1): wherein ASN is a D amino acid
  MISC_FEATURE (2)..(2): wherein GLU is a D amino acid
  MISC_FEATURE (5)..(6): x is natural, unnatural, analogue, L, D, or methylated amino acid or any combination
  MISC_FEATURE (8)..(8): x is natural, unnatural, analogue, L, D, or methylated amino acid or any combination
  MISC_FEATURE (10)..(11): x is natural, unnatural, analogue, L, D, or methylated amino acid or any combination
  MISC_FEATURE (13)..(14): x is natural, unnatural, analogue, L, D, or methylated amino acid or any combination
  MISC_FEATURE (16)..(16): wherein x is a D amino acid
  MISC_FEATURE (16)..(16): x is natural, unnatural, analogue, L, D, or methylated amino acid or any combination
NEDCXXCXNXXCXXCX

SEQ ID NO: 52 - protein, 16 aa, Artificial Sequence
  Chemically Synthesized
  MISC_FEATURE (1)..(1): wherein ASN is a D amino acid
  MISC_FEATURE (2)..(2): wherein ASP is a D amino acid
  MISC_FEATURE (5)..(6): x is natural, unnatural, analogue, L, D, or methylated amino acid or any combination
  MISC_FEATURE (8)..(8): x is natural, unnatural, analogue, L, D, or methylated amino acid or any combination
  MISC_FEATURE (10)..(11): x is natural, unnatural, analogue, L, D, or methylated amino acid or any combination
  MISC_FEATURE (13)..(14): x is natural, unnatural, analogue, L, D, or methylated amino acid or any combination
  MISC_FEATURE (16)..(16): wherein x is a D amino acid
  MISC_FEATURE (16)..(16): x is natural, unnatural, analogue, L, D, or methylated amino acid or any combination
NDECXXCXNXXCXXCX

SEQ ID NO: 53 - protein, 16 aa, Artificial Sequence
  Chemically Synthesized
  MISC_FEATURE (1)..(1): wherein ASN is a D amino acid
  MISC_FEATURE (2)..(2): wherein ASP is a D amino acid
  MISC_FEATURE (3)..(3): wherein GLU is a D amino acid
  MISC_FEATURE (5)..(6): x is natural, unnatural, analogue, L, D, or methylated amino acid or any combination
  MISC_FEATURE (8)..(8): x is natural, unnatural, analogue, L, D, or methylated amino acid or any combination
  MISC_FEATURE (10)..(11): x is natural, unnatural, analogue, L, D, or methylated amino acid or any combination
  MISC_FEATURE (13)..(14): x is natural, unnatural, analogue, L, D, or methylated amino acid or any combination
  MISC_FEATURE (16)..(16): wherein x is a D amino acid
  MISC_FEATURE (16)..(16): x is natural, unnatural, analogue, L, D, or methylated amino acid or any combination
NDECXXCXYXXCXXCX

SEQ ID NO: 54 - protein, 16 aa, Artificial Sequence
  Chemically Synthesized
  MISC_FEATURE (1)..(1): wherein ASN is a D amino acid
  MISC_FEATURE (2)..(2): wherein GLU is a D amino acid
  MISC_FEATURE (3)..(3): wherein GLU is a D amino acid
  MISC_FEATURE (5)..(6): x is natural, unnatural, analogue, L, D, or methylated amino acid or any combination
  MISC_FEATURE (8)..(8): x is natural, unnatural, analogue, L, D, or methylated amino acid or any combination
  MISC_FEATURE (10)..(11): x is natural, unnatural, analogue, L, D, or methylated amino acid or any combination
  MISC_FEATURE (13)..(14): x is natural, unnatural, analogue, L, D, or methylated amino acid or any combination
  MISC_FEATURE (16)..(16): wherein x is a D amino acid
  MISC_FEATURE (16)..(16): x is natural, unnatural, analogue, L, D, or methylated amino acid or any combination
NEECXXCXYXXCXXCX

SEQ ID NO: 55 - protein, 14 aa, Artificial Sequence
  Chemically Synthesized
CCEYCCNPACTGCY

SEQ ID NO: 56 - protein, 13 aa, Artificial Sequence
  Chemically Synthesized
CCEYCCNPACTGC

SEQ ID NO: 57 - protein, 14 aa, Artificial Sequence
  Chemically Synthesized
  MISC_FEATURE (1)..(1): wherein CYS at position 1 is attached to polyethylene glycol
  MISC_FEATURE (14)..(14): wherein TYR at position 14 is attached to polyethylene glycol
CCEYCCNPACTGCY

SEQ ID NO: 58 - protein, 16 aa, Artificial Sequence
  Chemically Synthesized
NFCCESCCNPACTGCY

SEQ ID NO: 59 - protein, 16 aa, Artificial Sequence
  Chemically Synthesized
NFCCEFCCNPACTGCY

SEQ ID NO: 60 - protein, 14 aa, Artificial Sequence
  Chemically Synthesized
  MISC_FEATURE (14)..(14): wherein TYR is a D amino acid
CCEYCCNPACTGCY

SEQ ID NO: 61 - protein, 14 aa, Artificial Sequence
  Chemically Synthesized
  MISC_FEATURE (1)..(1): wherein CYS at position 1 is attached to polyethylene glycol
CCEYCCNPACTGCY

SEQ ID NO: 62 - protein, 16 aa, Artificial Sequence
  Chemically Synthesized
NFCCETCCNPACTGCY

SEQ ID NO: 63 - protein, 16 aa, Artificial Sequence
  Chemically Synthesized
  MISC_FEATURE (16)..(16): wherein TYR is a D amino acid
NFCCESCCNPACTGCY

SEQ ID NO: 64 - protein, 16 aa, Artificial Sequence
  Chemically Synthesized
  MISC_FEATURE (1)..(1): wherein ASN is a D amino acid
NFCCESCCNPACTGCY

SEQ ID NO: 65 - protein, 16 aa, Artificial Sequence
  Chemically Synthesized
  MISC_FEATURE (1)..(1): wherein ASN is a D amino acid
  MISC_FEATURE (16)..(16): wherein TYR is a D amino acid
NFCCESCCNPACTGCY

SEQ ID NO: 66 - protein, 16 aa, Artificial Sequence
  Chemically Synthesized
  MISC_FEATURE (16)..(16): wherein TYR is a D amino acid
NFCCETCCNPACTGCY

SEQ ID NO: 67 - protein, 16 aa, Artificial Sequence
  Chemically Synthesized
  MISC_FEATURE (1)..(1): wherein ASN is a D amino acid
NFCCETCCNPACTGCY

SEQ ID NO: 68 - protein, 16 aa, Artificial Sequence
  Chemically Synthesized
  MISC_FEATURE (1)..(1): wherein ASN is a D amino acid
  MISC_FEATURE (16)..(16): wherein TYR is a D amino acid
NFCCETCCNPACTGCY

SEQ ID NO: 69 - protein, 16 aa, Artificial Sequence
  Chemically Synthesized
  MISC_FEATURE (16)..(16): wherein TYR is a D amino acid
NFCCEFCCNPACTGCY

SEQ ID NO: 70 - protein, 16 aa, Artificial Sequence
  Chemically Synthesized
  MISC_FEATURE (1)..(1): wherein ASN is a D amino acid
NFCCEFCCNPACTGCY

SEQ ID NO: 71 - protein, 16 aa, Artificial Sequence
  Chemically Synthesized
  MISC_FEATURE (1)..(1): wherein ASN is a D amino acid
  MISC_FEATURE (16)..(16): wherein TYR is a D amino acid
NFCCEFCCNPACTGCY

SEQ ID NO: 72 - protein, 14 aa, Artificial Sequence
  Chemically Synthesized
  MISC_FEATURE (14)..(14): wherein TYR at position 14 is attached to polyethylene glycol
CCEYCCNPACTGCY

SEQ ID NO: 73 - protein, 13 aa, Artificial Sequence
  Chemically Synthesized
  MISC_FEATURE (1)..(1): wherein CYS at position 1 is attached to polyethylene glycol
  MISC_FEATURE (13)..(13): wherein CYS at position 13 is attached to polyethylene glycol
CCEYCCNPACTGC

SEQ ID NO: 74 - protein, 13 aa, Artificial Sequence
  Chemically Synthesized
  MISC_FEATURE (1)..(1): wherein CYS at position 1 is attached to polyethylene glycol
CCEYCCNPACTGC

SEQ ID NO: 75 - protein, 13 aa, Artificial Sequence
  Chemically Synthesized
  MISC_FEATURE (13)..(13): wherein CYS at position 13 is attached to polyethylene glycol
CCEYCCNPACTGC

SEQ ID NO: 76 - protein, 16 aa, Artificial Sequence
  Chemically Synthesized
  MISC_FEATURE (1)..(1): wherein ASN at position 1 is attached to polyethylene glycol
  MISC_FEATURE (16)..(16): wherein TYR at position 16 is attached to polyethylene glycol
NFCCESCCNPACTGCY

SEQ ID NO: 77 - protein, 16 aa, Artificial Sequence
  Chemically Synthesized
  MISC_FEATURE (1)..(1): wherein ASN at position 1 is attached to polyethylene glycol
NFCCESCCNPACTGCY

SEQ ID NO: 78 - protein, 16 aa, Artificial Sequence
  Chemically Synthesized
  MISC_FEATURE (16)..(16): wherein TYR at position 16 is attached to polyethylene glycol
NFCCESCCNPACTGCY

SEQ ID NO: 79 - protein, 16 aa, Artificial Sequence
  Chemically Synthesized
  MISC_FEATURE (1)..(1): wherein ASN at position 1 is attached to polyethylene glycol
  MISC_FEATURE (16)..(16): wherein TYR at position 16 is attached to polyethylene glycol
NFCCEFCCNPACTGCY

SEQ ID NO: 80 - protein, 16 aa, Artificial Sequence
  Chemically Synthesized
  MISC_FEATURE (1)..(1): wherein ASN at position 1 is attached to polyethylene glycol
NFCCEFCCNPACTGCY

SEQ ID NO: 81 - protein, 16 aa, Artificial Sequence
  Chemically Synthesized
  MISC_FEATURE (16)..(16): wherein TYR at position 16 is attached to polyethylene glycol
NFCCEFCCNPACTGCY

SEQ ID NO: 82 - protein, 16 aa, Artificial Sequence
  Chemically Synthesized
  MISC_FEATURE (1)..(1): wherein ASN at position 1 is attached to polyethylene glycol
  MISC_FEATURE (16)..(16): wherein TYR at position 16 is attached to polyethylene glycol
NFCCEYCCNPACTGCY

SEQ ID NO: 83 - protein, 16 aa, Artificial Sequence
  Chemically Synthesized
  MISC_FEATURE (1)..(1): wherein ASN at position 1 is attached to polyethylene glycol
NFCCEYCCNPACTGCY

SEQ ID NO: 84 - protein, 16 aa, Artificial Sequence
  Chemically Synthesized
  MISC_FEATURE (16)..(16): wherein TYR at position 16 is attached to polyethylene glycol
NFCCEYCCNPACTGCY

SEQ ID NO: 85 - protein, 14 aa, Artificial Sequence
  Chemically Synthesized
CCESCCNPACTGCY

SEQ ID NO: 86 - protein, 14 aa, Artificial Sequence
  Chemically Synthesized
CCEFCCNPACTGCY

SEQ ID NO: 87 - protein, 13 aa, Artificial Sequence
  Chemically Synthesized
CCESCCNPACTGC

SEQ ID NO: 88 - protein, 13 aa, Artificial Sequence
  Chemically Synthesized
CCEFCCNPACTGC

SEQ ID NO: 89 - protein, 14 aa, Artificial Sequence
  Chemically Synthesized
  MISC_FEATURE (1)..(2): wherein x is penicillamine
  MISC_FEATURE (5)..(6): wherein x is penicillamine
  MISC_FEATURE (10)..(10): wherein x is penicillamine
  MISC_FEATURE (13)..(13): wherein x is penicillamine
XXEYXXNPAXTGXY

SEQ ID NO: 90 - protein, 13 aa, Artificial Sequence
  Chemically Synthesized
  MISC_FEATURE (1)..(2): wherein x is penicillamine
  MISC_FEATURE (5)..(6): wherein x is penicillamine
  MISC_FEATURE (10)..(10): wherein x is penicillamine
  MISC_FEATURE (13)..(13): wherein x is penicillamine
XXEYXXNPAXTGX

SEQ ID NO: 91 - protein, 22 aa, Artificial Sequence
  Chemically Synthesized
  MISC_FEATURE (1)..(6): x is natural, unnatural, analogue, L, D, or methylated amino acid or any combination
  MISC_FEATURE (11)..(11): x is natural, unnatural, analogue, L, D, or methylated amino acid or any combination
  MISC_FEATURE (15)..(17): x is natural, unnatural, analogue, L, D, or methylated amino acid or any combination
  MISC_FEATURE (19)..(20): x is natural, unnatural, analogue, L, D, or methylated amino acid or any combination
  MISC_FEATURE (22)..(22): x is natural, unnatural, analogue, L, D, or methylated amino acid or any combination
XXXXXXNYCCXYCCXXXCXXCX

SEQ ID NO: 92 - protein, 22 aa, Artificial Sequence
  Chemically Synthesized
  MISC_FEATURE (1)..(6): x is natural, unnatural, analogue, L, D, or methylated amino acid or any combination
  MISC_FEATURE (11)..(11): x is natural, unnatural, analogue, L, D, or methylated amino acid or any combination
  MISC_FEATURE (15)..(17): x is natural, unnatural, analogue, L, D, or methylated amino acid or any combination
  MISC_FEATURE (19)..(20): x is natural, unnatural, analogue, L, D, or methylated amino acid or any combination
  MISC_FEATURE (22)..(22): x is natural, unnatural, analogue, L, D, or methylated amino acid or any combination
XXXXXXNFCCXFCCXXXCXXCX

SEQ ID NO: 93 - protein, 16 aa, Artificial Sequence
  Chemically Synthesized
  MISC_FEATURE (5)..(5): x is natural, unnatural, analogue, L, D, or methylated amino acid or any combination
  MISC_FEATURE (9)..(11): x is natural, unnatural, analogue, L, D, or methylated amino acid or any combination
  MISC_FEATURE (13)..(14): x is natural, unnatural, analogue, L, D, or methylated amino acid or any combination
  MISC_FEATURE (16)..(16): x is natural, unnatural, analogue, L, D, or methylated amino acid or any combination
NFCCXFCCXXXCXXCX

SEQ ID NO: 94 - protein, 16 aa, Artificial Sequence
  Chemically Synthesized
  MISC_FEATURE (3)..(3): wherein x is penicillamine
  MISC_FEATURE (5)..(5): x is natural, unnatural, analogue, L, D, or methylated amino acid or any combination
  MISC_FEATURE (8)..(8): wherein x is penicillamine
  MISC_FEATURE (9)..(11): x is natural, unnatural, analogue, L, D, or methylated amino acid or any combination
  MISC_FEATURE (13)..(14): x is natural, unnatural, analogue, L, D, or methylated amino acid or any combination
  MISC_FEATURE (16)..(16): x is natural, unnatural, analogue, L, D, or methylated amino acid or any combination
NFXCXFCXXXXCXXCX

SEQ ID NO: 95 - protein, 16 aa, Artificial Sequence
  Chemically Synthesized
  MISC_FEATURE (3)..(4): wherein x is a cysteine, penicillamine homocysteine, or 3-mercaptoproline
  MISC_FEATURE (5)..(6): x is natural, unnatural, analogue, L, D, or methylated amino acid or any combination
  MISC_FEATURE (7)..(8): wherein x is a cysteine, penicillamine homocysteine, or 3-mercaptoproline
  MISC_FEATURE (9)..(11): x is natural, unnatural, analogue, L, D, or methylated amino acid or any combination
  MISC_FEATURE (12)..(12): wherein x is a cysteine, penicillamine homocysteine, or 3-mercaptoproline
  MISC_FEATURE (13)..(14): x is natural, unnatural, analogue, L, D, or methylated amino acid or any combination
  MISC_FEATURE (15)..(15): wherein x is a cysteine, penicillamine homocysteine, or 3-mercaptoproline
  MISC_FEATURE (16)..(16): x is natural, unnatural, analogue, L, D, or methylated amino acid or any combination
NFXXXXXXXXXXXXXX

SEQ ID NO: 96 - protein, 14 aa, Artificial Sequence
  Chemically Synthesized
  MISC_FEATURE (1)..(2): wherein x is a cysteine, penicillamine homocysteine, or 3-mercaptoproline
  MISC_FEATURE (4)..(4): x is natural, unnatural, analogue, L, D, or methylated amino acid or any combination
  MISC_FEATURE (5)..(6): wherein x is a cysteine, penicillamine homocysteine, or 3-mercaptoproline
  MISC_FEATURE (10)..(10): wherein x is a cysteine, penicillamine homocysteine, or 3-mercaptoproline
  MISC_FEATURE (13)..(13): wherein x is a cysteine, penicillamine homocysteine, or 3-mercaptoproline
XXEXXXNPAXTGXY

SEQ ID NO: 97 - protein, 13 aa, Artificial Sequence
  Chemically Synthesized
  MISC_FEATURE (1)..(2): wherein x is a cysteine, penicillamine homocysteine, or 3-mercaptoproline
  MISC_FEATURE (4)..(4): x is natural, unnatural, analogue, L, D, or methylated amino acid or any combination
  MISC_FEATURE (5)..(6): wherein x is a cysteine, penicillamine homocysteine, or 3-mercaptoproline
  MISC_FEATURE (10)..(10): wherein x is a cysteine, penicillamine homocysteine, or 3-mercaptoproline
  MISC_FEATURE (13)..(13): wherein x is a cysteine, penicillamine homocysteine, or 3-mercaptoproline
XXEXXXNPAXTGX

SEQ ID NO: 98 - protein, 20 aa, Artificial Sequence
  Chemically Synthesized
  MISC_FEATURE (1)..(1): x is none or a natural, unnatural, analogue, L, D, or methylated amino acid or any combination
  MISC_FEATURE (2)..(2): x is none or a natural, unnatural, analogue, L, D, or methylated amino acid or any combination
  MISC_FEATURE (3)..(3): x is none or a natural, unnatural, analogue, L, D, or methylated amino acid or any combination
  MISC_FEATURE (4)..(4): x is none or a natural, unnatural, analogue, L, D, or methylated amino acid or any combination
  MISC_FEATURE (5)..(5): x is none or a natural, unnatural, analogue, L, D, or methylated amino acid or any combination
  MISC_FEATURE (6)..(6): x is none or a natural, unnatural, analogue, L, D, or methylated amino acid or any combination
  MISC_FEATURE (7)..(8): wherein x is a cysteine, penicillamine homocysteine, or 3-mercaptoproline
  MISC_FEATURE (9)..(10): x is natural, unnatural, analogue, L, D, or methylated amino acid or any combination
  MISC_FEATURE (11)..(12): wherein x is a cysteine, penicillamine homocysteine, or 3-mercaptoproline
  MISC_FEATURE (13)..(15): x is natural, unnatural, analogue, L, D, or methylated amino acid or any combination
  MISC_FEATURE (16)..(16): wherein x is a cysteine, penicillamine homocysteine, or 3-mercaptoproline
  MISC_FEATURE (17)..(18): x is natural, unnatural, analogue, L, D, or methylated amino acid or any combination
  MISC_FEATURE (19)..(19): wherein x is a cysteine, penicillamine homocysteine, or 3-mercaptoproline
  MISC_FEATURE (20)..(20): x is none or a natural, unnatural, analogue, L, D, or methylated amino acid or any combination
XXXXXXXXXXXXXXXXXXXX

SEQ ID NO: 99 - protein, 16 aa, Artificial Sequence
  Chemically Synthesized
  MISC_FEATURE (1)..(1): wherein ASN is a D amino acid
  MISC_FEATURE (16)..(16): wherein LEU is a D amino acid
  MISC_FEATURE (16)..(16): wherein LEU is conjugated to an AMIDE
NDECELCVNVACTGCL

SEQ ID NO: 100 - protein, 16 aa, Artificial Sequence
  Chemically Synthesized
  MISC_FEATURE (1)..(1): wherein ASN is a D amino acid
  MISC_FEATURE (16)..(16): wherein SER is a D amino acid
NDECELCVNVACTGCS

SEQ ID NO: 101 - protein, 16 aa, Artificial Sequence
  Chemically Synthesized
  MISC_FEATURE (1)..(1): wherein ASN is a D amino acid
  MISC_FEATURE (16)..(16): wherein SER is a D amino acid
  MISC_FEATURE (16)..(16): wherein SER is conjugated to an AMIDE
NDECELCVNVACTGCS

SEQ ID NO: 102 - protein, 16 aa, Artificial Sequence
  Chemically Synthesized
  MISC_FEATURE (1)..(1): wherein ASN is a D amino acid
  MISC_FEATURE (16)..(16): wherein TYR is a D amino acid
NDECELCVNVACTGCY

SEQ ID NO: 103 - protein, 16 aa, Artificial Sequence
  Chemically Synthesized
  MISC_FEATURE (1)..(1): wherein ASN is a D amino acid
  MISC_FEATURE (16)..(16): wherein TYR is a D amino acid
  MISC_FEATURE (16)..(16): wherein TYR is conjugated to an AMIDE
NDECELCVNVACTGCY

SEQ ID NO: 104 - protein, 16 aa, Artificial Sequence
  Chemically Synthesized
  MISC_FEATURE (1)..(1): wherein GLU is conjugated to Py
  MISC_FEATURE (16)..(16): wherein LEU is conjugated to an AMIDE
  MISC_FEATURE (16)..(16): wherein LEU is a D amino acid
EDECELCVNVACTGCL

SEQ ID NO: 105 - protein, 16 aa, Artificial Sequence
  Chemically Synthesized
  MISC_FEATURE (1)..(1): wherein ASN is attached to polyethylene glycol
  MISC_FEATURE (16)..(16): wherein LEU is attached to polyethylene glycol
NDECELCVNVACTGCL

SEQ ID NO: 106 - protein, 16 aa, Artificial Sequence
  Chemically Synthesized
  MISC_FEATURE (1)..(1): wherein ASN is attached to polyethylene glycol
NDECELCVNVACTGCL

SEQ ID NO: 107 - protein, 16 aa, Artificial Sequence
  Chemically Synthesized
  MISC_FEATURE (16)..(16): wherein LEU is attached to polyethylene glycol
NDECELCVNVACTGCL

SEQ ID NO: 108 - protein, 16 aa, Artificial Sequence
  Chemically Synthesized
  MISC_FEATURE (1)..(3): x is natural, unnatural, analogue, L, D, or methylated amino acid or any combination
  MISC_FEATURE (4)..(4): wherein x is a cysteine, penicillamine homocysteine, or 3-mercaptoproline
  MISC_FEATURE (5)..(6): x is natural, unnatural, analogue, L, D, or methylated amino acid or any combination
  MISC_FEATURE (7)..(7): wherein x is a cysteine, penicillamine homocysteine, or 3-mercaptoproline
  MISC_FEATURE (8)..(11): x is natural, unnatural, analogue, L, D, or methylated amino acid or any combination
  MISC_FEATURE (12)..(12): wherein x is a cysteine, penicillamine homocysteine, or 3-mercaptoproline
  MISC_FEATURE (13)..(14): x is natural, unnatural, analogue, L, D, or methylated amino acid or any combination
  MISC_FEATURE (15)..(15): wherein x is a cysteine, penicillamine homocysteine, or 3-mercaptoproline
  MISC_FEATURE (16)..(16): x is natural, unnatural, analogue, L, D, or methylated amino acid or any combination
XXXXXXXXXXXXXXXX

SEQ ID NO: 109 - protein, 16 aa, Artificial Sequence
  Chemically Synthesized
NDDCELCVNVACTGCL

SEQ ID NO: 110 - protein, 16 aa, Artificial Sequence
  Chemically Synthesized
EDDCELCVNVACTGCL

SEQ ID NO: 111 - protein, 16 aa, Artificial Sequence
  Chemically Synthesized
EDECELCVNVACTGCL

SEQ ID NO: 112 - protein, 16 aa, Artificial Sequence
  Chemically Synthesized
EEDCELCVNVACTGCL

SEQ ID NO: 113 - protein, 16 aa, Artificial Sequence
  Chemically Synthesized
EEECELCVNVACTGCL

SEQ ID NO: 114 - protein, 16 aa, Artificial Sequence
  Chemically Synthesized
DDDCELCVNVACTGCL

SEQ ID NO: 115 - protein, 16 aa, Artificial Sequence
  Chemically Synthesized
DDECELCVNVACTGCL

SEQ ID NO: 116 - protein, 16 aa, Artificial Sequence
  Chemically Synthesized
DEDCELCVNVACTGCL

SEQ ID NO: 117 - protein, 16 aa, Artificial Sequence
  Chemically Synthesized
DEECELCVNVACTGCL

SEQ ID NO: 118 - protein, 16 aa, Artificial Sequence
  Chemically Synthesized
QDDCELCVNVACTGCL

SEQ ID NO: 119 - protein, 16 aa, Artificial Sequence
  Chemically Synthesized
QDECELCVNVACTGCL

SEQ ID NO: 120 - protein, 16 aa, Artificial Sequence
  Chemically Synthesized
QEDCELCVNVACTGCL

SEQ ID NO: 121 - protein, 16 aa, Artificial Sequence
  Chemically Synthesized
QEECELCVNVACTGCL

SEQ ID NO: 122 - protein, 16 aa, Artificial Sequence
  Chemically Synthesized
KDDCELCVNVACTGCL

SEQ ID NO: 123 - protein, 16 aa, Artificial Sequence
  Chemically Synthesized
KDECELCVNVACTGCL

SEQ ID NO: 124 - protein, 16 aa, Artificial Sequence
  Chemically Synthesized
KEDCELCVNVACTGCL

SEQ ID NO: 125 - protein, 16 aa, Artificial Sequence
  Chemically Synthesized
KEECELCVNVACTGCL

SEQ ID NO: 126 - protein, 16 aa, Artificial Sequence
  Chemically Synthesized
EDDCELCVNVACTGCS

SEQ ID NO: 127 - protein, 16 aa, Artificial Sequence
  Chemically Synthesized
EDECELCVNVACTGCS

SEQ ID NO: 128 - protein, 16 aa, Artificial Sequence
  Chemically Synthesized
EEDCELCVNVACTGCS

SEQ ID NO: 129 - protein, 16 aa, Artificial Sequence
  Chemically Synthesized
EEECELCVNVACTGCS

SEQ ID NO: 130 - protein, 16 aa, Artificial Sequence
  Chemically Synthesized
DDDCELCVNVACTGCS

SEQ ID NO: 131 - protein, 16 aa, Artificial Sequence
  Chemically Synthesized
DDECELCVNVACTGCS

SEQ ID NO: 132 - protein, 16 aa, Artificial Sequence
  Chemically Synthesized
DEDCELCVNVACTGCS

SEQ ID NO: 133 - protein, 16 aa, Artificial Sequence
  Chemically Synthesized
DEECELCVNVACTGCS

SEQ ID NO: 134 - protein, 16 aa, Artificial Sequence
  Chemically Synthesized
QDDCELCVNVACTGCS

SEQ ID NO: 135 - protein, 16 aa, Artificial Sequence
  Chemically Synthesized
QDECELCVNVACTGCS

SEQ ID NO: 136 - protein, 16 aa, Artificial Sequence
  Chemically Synthesized
QEDCELCVNVACTGCS

SEQ ID NO: 137 - protein, 16 aa, Artificial Sequence
  Chemically Synthesized
QEECELCVNVACTGCS

SEQ ID NO: 138 - protein, 16 aa, Artificial Sequence
  Chemically Synthesized
KDDCELCVNVACTGCS

SEQ ID NO: 139 - protein, 16 aa, Artificial Sequence
  Chemically Synthesized
KDECELCVNVACTGCS

SEQ ID NO: 140 - protein, 16 aa, Artificial Sequence
  Chemically Synthesized
KEDCELCVNVACTGCS

SEQ ID NO: 141 - protein, 16 aa, Artificial Sequence
  Chemically Synthesized
KEECELCVNVACTGCS

SEQ ID NO: 142 - protein, 16 aa, Artificial Sequence
  Chemically Synthesized
EDDCELCINMACTGCL

SEQ ID NO: 143 - protein, 16 aa, Artificial Sequence
  Chemically Synthesized
EDECELCINMACTGCL

SEQ ID NO: 144 - protein, 16 aa, Artificial Sequence
  Chemically Synthesized
EEDCELCINMACTGCL

SEQ ID NO: 145 - protein, 16 aa, Artificial Sequence
  Chemically Synthesized
EEECELCINMACTGCL

SEQ ID NO: 146 - protein, 16 aa, Artificial Sequence
  Chemically Synthesized
DDDCELCINMACTGCL

SEQ ID NO: 147 - protein, 16 aa, Artificial Sequence
  Chemically Synthesized
DDECELCINMACTGCL

SEQ ID NO: 148 - protein, 16 aa, Artificial Sequence
  Chemically Synthesized
DEDCELCINMACTGCL

SEQ ID NO: 149 - protein, 16 aa, Artificial Sequence
  Chemically Synthesized
DEECELCINMACTGCL

SEQ ID NO: 150 - protein, 16 aa, Artificial Sequence
  Chemically Synthesized
QDDCELCINMACTGCL

SEQ ID NO: 151 - protein, 16 aa, Artificial Sequence
  Chemically Synthesized
QDECELCINMACTGCL

SEQ ID NO: 152 - protein, 16 aa, Artificial Sequence
  Chemically Synthesized
QEDCELCINMACTGCL

SEQ ID NO: 153 - protein, 16 aa, Artificial Sequence
  Chemically Synthesized
QEECELCINMACTGCL

SEQ ID NO: 154 - protein, 16 aa, Artificial Sequence
  Chemically Synthesized
KDDCELCINMACTGCL

SEQ ID NO: 155 - protein, 16 aa, Artificial Sequence
  Chemically Synthesized
KDECELCINMACTGCL

SEQ ID NO: 156 - protein, 16 aa, Artificial Sequence
  Chemically Synthesized
KEDCELCINMACTGCL

SEQ ID NO: 157 - protein, 16 aa, Artificial Sequence
  Chemically Synthesized
KEECELCINMACTGCL

SEQ ID NO: 158 - protein, 16 aa, Artificial Sequence
  Chemically Synthesized
EDDCELCINMACTGCS

SEQ ID NO: 159 - protein, 16 aa, Artificial Sequence
  Chemically Synthesized
EDECELCINMACTGCS

SEQ ID NO: 160 - protein, 16 aa, Artificial Sequence
  Chemically Synthesized
EEDCELCINMACTGCS

SEQ ID NO: 161 - protein, 16 aa, Artificial Sequence
  Chemically Synthesized
EEECELCINMACTGCS

SEQ ID NO: 162 - protein, 16 aa, Artificial Sequence
  Chemically Synthesized
DDDCELCINMACTGCS

SEQ ID NO: 163 - protein, 16 aa, Artificial Sequence
  Chemically Synthesized
DDECELCINMACTGCS

SEQ ID NO: 164 - protein, 16 aa, Artificial Sequence
  Chemically Synthesized
DEDCELCINMACTGCS

SEQ ID NO: 165 - protein, 16 aa, Artificial Sequence
  Chemically Synthesized
DEECELCINMACTGCS

SEQ ID NO: 166 - protein, 16 aa, Artificial Sequence
  Chemically Synthesized
QDDCELCINMACTGCS

SEQ ID NO: 167 - protein, 16 aa, Artificial Sequence
  Chemically Synthesized
QDECELCINMACTGCS

SEQ ID NO: 168 - protein, 16 aa, Artificial Sequence
  Chemically Synthesized
QEDCELCINMACTGCS

SEQ ID NO: 169 - protein, 16 aa, Artificial Sequence
  Chemically Synthesized
QEECELCINMACTGCS

SEQ ID NO: 170 - protein, 16 aa, Artificial Sequence
  Chemically Synthesized
KDDCELCINMACTGCS

SEQ ID NO: 171 - protein, 16 aa, Artificial Sequence
  Chemically Synthesized
KDECELCINMACTGCS

SEQ ID NO: 172 - protein, 16 aa, Artificial Sequence
  Chemically Synthesized
KEDCELCINMACTGCS

SEQ ID NO: 173 - protein, 16 aa, Artificial Sequence
  Chemically Synthesized
KEECELCINMACTGCS

SEQ ID NO: 174 - protein, 15 aa, Artificial Sequence
  Chemically Synthesized
  MISC_FEATURE (1)..(3): x is natural, unnatural, analogue, L, D, or methylated amino acid or any combination
  MISC_FEATURE (4)..(4): wherein x is a cysteine, penicillamine homocysteine, or 3-mercaptoproline
  MISC_FEATURE (5)..(6): x is natural, unnatural, analogue, L, D, or methylated amino acid or any combination
  MISC_FEATURE (7)..(7): wherein x is a cysteine, penicillamine homocysteine, or 3-mercaptoproline
  MISC_FEATURE (8)..(11): x is natural, unnatural, analogue, L, D, or methylated amino acid or any combination
  MISC_FEATURE (12)..(12): wherein x is a cysteine, penicillamine homocysteine, or 3-mercaptoproline
  MISC_FEATURE (13)..(14): x is natural, unnatural, analogue, L, D, or methylated amino acid or any combination
  MISC_FEATURE (15)..(15): wherein x is a cysteine, penicillamine homocysteine, or 3-mercaptoproline
XXXXXXXXXXXXXXX

SEQ ID NO: 175 - protein, 15 aa, Artificial Sequence
  Chemically Synthesized
SHTCEICAFAACAGC

SEQ ID NO: 176 - protein, 15 aa, Artificial Sequence
  Chemically Synthesized
SHTCEICANAACAGC

SEQ ID NO: 177 - protein, 15 aa, Artificial Sequence
  Chemically Synthesized
SHTCELCANAACAGC

SEQ ID NO: 178 - protein, 15 aa, Artificial Sequence
  Chemically Synthesized
SHTCEVCANAACAGC

SEQ ID NO: 179 - protein, 15 aa, Artificial Sequence
  Chemically Synthesized
SHTCEYCANAACAGC

SEQ ID NO: 180 - protein, 15 aa, Artificial Sequence
  Chemically Synthesized
SHTCEICANAACAGC

SEQ ID NO: 181 - protein, 15 aa, Artificial Sequence
  Chemically Synthesized
SHTCELCANAACAGC

SEQ ID NO: 182 - protein, 15 aa, Artificial Sequence
  Chemically Synthesized
SHTCEVCANAACAGC

SEQ ID NO: 183 - protein, 15 aa, Artificial Sequence
  Chemically Synthesized
SHTCEYCANAACAGC

SEQ ID NO: 184 - protein, 15 aa, Artificial Sequence
  Chemically Synthesized
SHTCEICANAACAGC

SEQ ID NO: 185 - protein, 15 aa, Artificial Sequence
  Chemically Synthesized
SHTCELCANAACAGC

SEQ ID NO: 186 - protein, 15 aa, Artificial Sequence
  Chemically Synthesized
SHTCEVCANAACAGC

SEQ ID NO: 187 - protein, 15 aa, Artificial Sequence
  Chemically Synthesized
SHTCEYCANAACAGC

SEQ ID NO: 188 - protein, 15 aa, Artificial Sequence
  Chemically Synthesized
SHTCEICANAACAGC

SEQ ID NO: 189 - protein, 15 aa, Artificial Sequence
  Chemically Synthesized
SHTCELCANAACAGC

SEQ ID NO: 190 - protein, 15 aa, Artificial Sequence
  Chemically Synthesized
SHTCEVCANAACAGC

SEQ ID NO: 191 - protein, 15 aa, Artificial Sequence
  Chemically Synthesized
SHTCEYCANAACAGC

SEQ ID NO: 192 - protein, 15 aa, Artificial Sequence
  Chemically Synthesized
NDECEICANAACAGC

SEQ ID NO: 193 - protein, 15 aa, Artificial Sequence
  Chemically Synthesized
NDECELCANAACAGC

SEQ ID NO: 194 - protein, 15 aa, Artificial Sequence
  Chemically Synthesized
NDECEVCANAACAGC

SEQ ID NO: 195 - protein, 15 aa, Artificial Sequence
  Chemically Synthesized
NDECEYCANAACAGC

SEQ ID NO: 196 - protein, 15 aa, Artificial Sequence
  Chemically Synthesized
NDECEICANAACAGC

SEQ ID NO: 197 - protein, 15 aa, Artificial Sequence
  Chemically Synthesized
NDECELCANAACAGC

SEQ ID NO: 198 - protein, 15 aa, Artificial Sequence
  Chemically Synthesized
NDECEVCANAACAGC

SEQ ID NO: 199 - protein, 15 aa, Artificial Sequence
  Chemically Synthesized
NDECEYCANAACAGC

SEQ ID NO: 200 - protein, 15 aa, Artificial Sequence
  Chemically Synthesized
NDECEICANAACAGC

SEQ ID NO: 201 - protein, 15 aa, Artificial Sequence
  Chemically Synthesized
NDECELCANAACAGC

SEQ ID NO: 202 - protein, 15 aa, Artificial Sequence
  Chemically Synthesized
NDECEVCANAACAGC

SEQ ID NO: 203 - protein, 15 aa, Artificial Sequence
  Chemically Synthesized
NDECEYCANAACAGC

SEQ ID NO: 204 - protein, 15 aa, Artificial Sequence
  Chemically Synthesized
NDECEICANAACAGC

SEQ ID NO: 205 - protein, 15 aa, Artificial Sequence
  Chemically Synthesized
NDECELCANAACAGC

SEQ ID NO: 206 - protein, 15 aa, Artificial Sequence
  Chemically Synthesized
NDECEVCANAACAGC

SEQ ID NO: 207 - protein, 15 aa, Artificial Sequence
  Chemically Synthesized
NDECEYCANAACAGC

SEQ ID NO: 208 - protein, 17 aa, Artificial Sequence
  Chemically Synthesized
  MISC_FEATURE (1)..(3): x is natural, unnatural, analogue, L, D, or methylated amino acid or any combination
  MISC_FEATURE (4)..(4): wherein x is a cysteine, penicillamine homocysteine, or 3-mercaptoproline
  MISC_FEATURE (5)..(6): x is natural, unnatural, analogue, L, D, or methylated amino acid or any combination
  MISC_FEATURE (7)..(7): wherein x is a cysteine, penicillamine homocysteine, or 3-mercaptoproline
  MISC_FEATURE (8)..(11): x is natural, unnatural, analogue, L, D, or methylated amino acid or any combination
  MISC_FEATURE (12)..(12): wherein x is a cysteine, penicillamine homocysteine, or 3-mercaptoproline
  MISC_FEATURE (13)..(14): x is natural, unnatural, analogue, L, D, or methylated amino acid or any combination
  MISC_FEATURE (15)..(15): x is none or a natural, unnatural, analogue, L, D, or methylated amino acid or any combination
  MISC_FEATURE (16)..(16): x is none or a natural, unnatural, analogue, L, D, or methylated amino acid or any combination
  MISC_FEATURE (17)..(17): x is any natural, unnatural, analogue, L, D, or methylated amino acid or any combination
XXXXXXXXXXXXXXXXX

SEQ ID NO: 209 - protein, 15 aa, Artificial Sequence
  Chemically Synthesized
QEECELCINMACTGY

SEQ ID NO: 210 - protein, 15 aa, Artificial Sequence
  Chemically Synthesized
QEECETCINMACTGY

SEQ ID NO: 211 - protein, 15 aa, Artificial Sequence
  Chemically Synthesized
QDECETCINMACTGY

SEQ ID NO: 212 - protein, 15 aa, Artificial Sequence
  Chemically Synthesized
QDDCETCINMACTGY

SEQ ID NO: 213 - protein, 15 aa, Artificial Sequence
  Chemically Synthesized
QEDCETCINMACTGY

SEQ ID NO: 214 - protein, 15 aa, Artificial Sequence
  Chemically Synthesized
QEECEECINMACTGY

SEQ ID NO: 215 - protein, 15 aa, Artificial Sequence
  Chemically Synthesized
QDECEECINMACTGY

SEQ ID NO: 216 - protein, 15 aa, Artificial Sequence
  Chemically Synthesized
QDDCEECINMACTGY

SEQ ID NO: 217 - protein, 15 aa, Artificial Sequence
  Chemically Synthesized
QEDCEECINMACTGY

SEQ ID NO: 218 - protein, 15 aa, Artificial Sequence
  Chemically Synthesized
QEECEYCINMACTGY

SEQ ID NO: 219 - protein, 15 aa, Artificial Sequence
  Chemically Synthesized
QDECEYCINMACTGY

SEQ ID NO: 220 - protein, 15 aa, Artificial Sequence
  Chemically Synthesized
QDDCEYCINMACTGY

SEQ ID NO: 221 - protein, 15 aa, Artificial Sequence
  Chemically Synthesized
QEDCEYCINMACTGY

SEQ ID NO: 222 - protein, 15 aa, Artificial Sequence
  Chemically Synthesized
QEECEICINMACTGY

SEQ ID NO: 223 - protein, 15 aa, Artificial Sequence
  Chemically Synthesized
QDECEICINMACTGY

SEQ ID NO: 224 - protein, 15 aa, Artificial Sequence
  Chemically Synthesized
QDDCEICINMACTGY

SEQ ID NO: 225 - protein, 15 aa, Artificial Sequence
  Chemically Synthesized
QEDCEICINMACTGY

SEQ ID NO: 226 - protein, 16 aa, Artificial Sequence
  Chemically Synthesized
QEECETCINMACTGCS

SEQ ID NO: 227 - protein, 16 aa, Artificial Sequence
  Chemically Synthesized
QDECETCINMACTGCS

SEQ ID NO: 228 - protein, 16 aa, Artificial Sequence
  Chemically Synthesized
QDDCETCINMACTGCS

SEQ ID NO: 229 - protein, 16 aa, Artificial Sequence
  Chemically Synthesized
QEDCETCINMACTGCS

SEQ ID NO: 230 - protein, 16 aa, Artificial Sequence
  Chemically Synthesized
QEECEECINMACTGCS

SEQ ID NO: 231 - protein, 16 aa, Artificial Sequence
  Chemically Synthesized
QDECEECINMACTGCS

SEQ ID NO: 232 - protein, 16 aa, Artificial Sequence
  Chemically Synthesized
QDDCEECINMACTGCS

SEQ ID NO: 233 - protein, 16 aa, Artificial Sequence
  Chemically Synthesized
QEDCEECINMACTGCS

SEQ ID NO: 234 - protein, 16 aa, Artificial Sequence
  Chemically Synthesized
QEECEYCINMACTGCS

SEQ ID NO: 235 - protein, 16 aa, Artificial Sequence
  Chemically Synthesized
QDECEYCINMACTGCS

SEQ ID NO: 236 - protein, 16 aa, Artificial Sequence
  Chemically Synthesized
QDDCEYCINMACTGCS

SEQ ID NO: 237 - protein, 16 aa, Artificial Sequence
  Chemically Synthesized
QEDCEYCINMACTGCS

SEQ ID NO: 238 - protein, 16 aa, Artificial Sequence
  Chemically Synthesized
QEECEICINMACTGCS

SEQ ID NO: 239 - protein, 16 aa, Artificial Sequence
  Chemically Synthesized
QDECEICINMACTGCS

SEQ ID NO: 240 - protein, 16 aa, Artificial Sequence
  Chemically Synthesized
QDDCEICINMACTGCS

SEQ ID NO: 241 - protein, 16 aa, Artificial Sequence
  Chemically Synthesized
QEDCEICINMACTGCS

SEQ ID NO: 242 - protein, 22 aa, Artificial Sequence
  Chemically Synthesized
NSSNSSNYCCEKCCNPACTGCY

SEQ ID NO: 243 - protein, 16 aa, Artificial Sequence
  Chemically Synthesized
  MISC_FEATURE (1)..(1): wherein ASN is attached to polyethylene glycol
  MISC_FEATURE (16)..(16): wherein TYR is attached to polyethylene glycol
NFCCETCCNPACTGCY

SEQ ID NO: 244 - protein, 16 aa, Artificial Sequence
  Chemically Synthesized
  MISC_FEATURE (1)..(1): wherein ASN is attached to polyethylene glycol
NFCCETCCNPACTGCY

SEQ ID NO: 245 - protein, 16 aa, Artificial Sequence
  Chemically Synthesized
  MISC_FEATURE (16)..(16): wherein TYR is attached to polyethylene glycol
NFCCETCCNPACTGCY

SEQ ID NO: 246 - protein, 16 aa, Artificial Sequence
  Chemically Synthesized
NFCCEYCCNPACTGCY

SEQ ID NO: 247 - protein, 16 aa, Artificial Sequence
  Chemically Synthesized
  MISC_FEATURE (1)..(1): wherein ASN is a D amino acid
  MISC_FEATURE (16)..(16): wherein TYR is a D amino acid
NFCCEYCCNPACTGCY

SEQ ID NO: 248 - protein, 16 aa, Artificial Sequence
  Chemically Synthesized
  MISC_FEATURE (16)..(16): wherein TYR is a D amino acid
NFCCEYCCNPACTGCY

SEQ ID NO: 249 - protein, 16 aa, Artificial Sequence
  Chemically Synthesized
  MISC_FEATURE (1)..(1): wherein ASN is a D amino acid
NFCCEYCCNPACTGCY

SEQ ID NO: 250 - protein, 16 aa, Artificial Sequence
  Synthesized peptide
  MISC_FEATURE (1)..(2): wherein Xaa is absent or any natural, unnatural amino acid or analogue thereof
  MISC_FEATURE (3)..(3): wherein Xaa is any natural, unnatural amino acid or analogue thereof
  MISC_FEATURE (5)..(11): wherein Xaa is any natural, unnatural amino acid or analogue thereof
  MISC_FEATURE (13)..(15): wherein Xaa is any natural, unnatural amino acid or analogue thereof
  MISC_FEATURE (16)..(16): wherein Xaa is absent or any natural, unnatural amino acid or analogue thereof
XXXCXXXXXXXCXXXX

SEQ ID NO: 251 - protein, 16 aa, Artificial Sequence
  Synthesized peptide
  MISC_FEATURE (1)..(1): wherein Xaa is a pyroglutamic acid
XDECELCVNVACTGCL

SEQ ID NO: 252 - protein, 15 aa, Artificial Sequence
  Synthesized peptide
PGTCEICAYAACTGC

SEQ ID NO: 253 - protein, 8 aa, Artificial Sequence
  Synthesized peptide
VRGEHNPR

SEQ ID NO: 254 - protein, 9 aa, Artificial Sequence
  Synthesized peptide
VRGPEHNPR

SEQ ID NO: 255 - protein, 10 aa, Artificial Sequence
  Synthesized peptide
VRGPREHNPR

SEQ ID NO: 256 - protein, 11 aa, Artificial Sequence
  Synthesized peptide
VRGPRREHNPR

SEQ ID NO: 257 - protein, 6 aa, Artificial Sequence
  Synthesized peptide
REHNPR

(19) United States
(12) Patent Application Publication
(10) Pub. No.: US 2020/0113968 A1
(43) Pub. Date: Apr. 16, 2020

(54) AGONISTS OF GUANYLATE CYCLASE USEFUL FOR DOWNREGULATION OF PRO-INFLAMMATORY CYTOKINES

(71) Applicant: Bausch Health Ireland Limited, Dublin (IE)

(72) Inventor: Kunwar Shailubhai, Audubon, PA (US)

(21) Appl. No.: 16/661,979

(22) Filed: Oct. 23, 2019

Related U.S. Application Data
(63) Continuation of application No. 15/026,560, filed on Mar. 31, 2016.
(60) Provisional application No. 61/888,744, filed on Oct. 9, 2013.

(51) Int. Cl. A61K 38/10; A61K 31/5377; A61K 31/40; A61K 9/00; A61K 45/06; A61K 31/519; C07K 7/06; A61K 31/655; C07K 7/08; A61K 38/08

(52) U.S. Cl. CPC A61K 38/10; A61K 31/5377; A61K 31/40; A61K 9/0053; A61K 38/08; A61K 31/519; C07K 7/06; A61K 31/655; C07K 7/08; A61K 45/06

(57) ABSTRACT

This invention provides a method to prevent, control, and/or treat an inflammatory disease or disorder by administering at least one agonist of guanalyte cyclase receptor, or pharmaceutical compositions thereof, either alone or either concurrently or sequentially with another compound or an active agent used to treat the disease or disorder, and/or with an inhibitor of cGMP-dependent phosphodieasterases.

### CROSS REFERENCE TO RELATED APPLICATIONS

This application is a continuation of, and claims priority to, U.S. application Ser. No. 15/026,560, filed Mar. 31, 2016, which is a 35 U.S.C. § 371 national stage application of, and claims priority to, International Application No. PCT/US2014/059914, filed Oct. 9, 2014, which claims priority under 35 U.S.C. § 119(e) to U.S. Provisional Application No. 61/888,744, filed Oct. 9, 2013, all of which are herein incorporated by reference in their entireties.

### INCORPORATION-BY-REFERENCE OF SEQUENCE LISTING

The contents of the text file named 376464-2400US2 Sequence Listing.txt, which was created on Oct. 23, 2019 and is 102 KB in size, are hereby incorporated by reference in their entireties.

### FIELD OF THE INVENTION

The present invention relates to the therapeutic use of guanylate cyclase C (GC-C) agonists for downregulation of pro-inflammatory cytokines. The agonists may be used either alone or either concurrently or sequentially with additional active agents to prevent or downregulate NF-κB activation and pro-inflammatory cytokines in the human body. The GC-C agonists may be used to prevent or treat colitis, including dextran sulphate sodium (DSS) induced colitis, ulcerative colitis, Crohn's disease, colon cancer, and/or any swelling or inflammation of the large intestine.

### BACKGROUND OF THE INVENTION

The human chronic inflammatory bowel diseases (IBD), Crohn's disease (CD) and ulcerative colitis (UC), affect over one million Americans. While the etiology of these disorders remains unknown, contributing factors include a poorly regulated immune response against the enteric microbiota in a genetically predisposed individual. There is currently no cure for IBD and existing therapies such as corticosteroids, 5-aminosalicylates, and immunomodulatory agents (6-mercaptopurine, methotrexate) are of limited effectiveness, have the potential for side effects, and/or are designed to non-specifically reduce intestinal inflammation. As a result of the burden of their clinical symptoms and side effects of medications, most patients with IBD have a significantly impaired quality of life. Due to complications of the disease, surgical intervention occurs in a significant proportion of patients over their lifetime. Increased understanding of specific immune pathways that modulate inflammation in IBD has led to the FDA approval of anti-TNF antibodies in CD and UC. Although a significant therapeutic advance, these agents are effective in less than 40% of patients long term, there is the potential for short- and long-term systemic side effects, high costs, and the need to deliver the drug on a repeated maintenance basis by injection. Therefore, there are urgent needs to identify targets that provide safer and effective means of therapeutic intervention.

### SUMMARY OF THE INVENTION

The invention provides a composition that includes a guanylate cyclase receptor agonist (GCRA). In some embodiments, the invention provides a composition that includes a guanylate cyclase receptor agonist (GCRA) and another therapeutic compound. In one embodiment, the additional therapeutic compound is a NF-κB inhibitor, a c-Src inhibitor, or 5-ASA. In another embodiment, the guanylate cyclase receptor agonist is a CGRA peptide. In another embodiment, the guanylate is plecanatide (SP-304) or SP-333. The composition of the invention may further include a pharmaceutical carrier, excipient or diluent. In a further embodiment, the NF-κB inhibitor is pyrrolidine dithiocarbamate (PTDC). In a further embodiment, the c-Src tyrosine kinase inhibitor is KX2-391.

The invention provides a method for preventing or treating a condition by administering to a subject in need of a therapeutically effective amount of the composition of the invention. For example, the condition is colitis, ulcerative colitis, Crohn's disease, irritable bowel syndrome (IBS), non-ulcer dyspepsia, chronic intestinal pseudo-obstruction, functional dyspepsia, colonic pseudo-obstruction, duodenogastric reflux, constipation, constipation associated with use of opiate pain killers, post-surgical constipation, IBS-associated constipation, constipation associated with neuropathic disorders, gastroesophageal reflux disease (GERD), Celiac disease, gastroparesis, heartburn, poor gastrointestinal motility, congestive heart failure, hypertension, benign prostatic hyperplasia (BPH), gastrointestinal cancer, lung cancer, bladder cancer, liver cancer, salivary gland cancer, skin cancer, colon cancer, bronchitis, tissue inflammation, organ inflammation, respiratory inflammation, asthma, COPD, lipid metabolism disorder, biliary disorder, cardiovascular disease, obesity or an endocrine disorder.

The invention provides a method of treating or alleviating a symptom of a NF-κB mediated inflammation by administering to a subject in need thereof an effective amount of a GCRA peptide or pharmaceutical composition thereof. The invention provides that the amount is effective to inhibit NF-κB activation, thus treating or alleviating a symptom of an inflammatory disorder or a NF-κB mediated inflammation. For example, the inflammatory disorder or a NF-κB mediated inflammation is colitis, ulcerative colitis, Crohn's disease, irritable bowel syndrome (IBS), tissue inflammation, organ inflammation, kidney inflammation, gastrointestinal system inflammation, necrotizing enterocolitis, pancreatic inflammation, lung inflammation, respiratory inflammation, asthma, COPD or skin inflammation.

The invention further provides a method of modulating NF-κB induction in a cell by contacting the cell with an effective amount of a GCRA peptide or pharmaceutical composition thereof, where the GCRA peptide inhibits NF-κB activation.

The invention also provides a method of modulating NF-κB-dependent target gene expression in a cell by contacting the cell with an effective amount of a GCRA peptide or pharmaceutical composition thereof. For example, the NF-κB-dependent target gene is IL-1, IL-2, TNF, IL-12p40, IL-17, IL-23, IL-8, RANTES, MIP-1α or IL-10.

In some embodiments, an effective amount of a GCRA peptide is sufficient to inhibit NF-κB activation.

In some embodiments, an effective dose of a cGMP-dependent phosphodiesterase inhibitor is also administered to a subject in need thereof. In some embodiments of the current invention, the cGMP-dependent phosphodiesterase inhibitor may be administered either concurrently or sequentially with a GCRA peptide, or pharmaceutical composition thereof. In some embodiments, the cGMP-dependent phosphodiesterase inhibitor is sulindac sulfone, zaprinast, motapizone, vardenafil, or sildenafil.

In some embodiments, the invention provides concurrent or sequential administration of an anti-inflammatory agent with a GCRA peptide or a pharmaceutical composition thereof, to a subject in need thereof. The anti-inflammatory agent is a steroid or nonsteroid anti-inflammatory drug (NSAIDs).

The GCRA peptide may be any one of Tables 1-7. Preferably, the GCRA peptide is Plecanatide (SP304), SP333 or SP373.

Other features and advantages of the invention will be apparent from and are encompassed by the following detailed description and claims.

### BRIEF DESCRIPTION OF DRAWINGS

FIG. 1 is a graph showing amino acid sequences of human uroguanylin and its synthetic analogs Plecanatide and SP-333. Single letter abbreviations for amino acids are depicted. In designing synthetic hexadecapeptides, aspartic acid (D) at position 3 from the NH2-terminus of UG is substituted with glutamic acid (E). This substitution stabilizes the peptides in an active conformation in aqueous media. In addition, SP-333 has D-stereoisomers of asparagine (N) and leucine (L) instead of the L-forms at position 1 and 16, respectively. It is designed to be particularly stable against proteolytic degradation that normally occurs in intestinal fluid as part of the normal digestive process. Uroguanylin as well as its analogs have four cysteine (C) residues enabling the formation of two intramolecular disulfide bonds. Substituted amino acids in plecanatide and SP-333 are shown in bold type.

FIG. 2 is a graph showing stimulation of cGMP accumulation in T84 cells by plecanatide and SP-333. Synthetically generated peptides are able to activate GC-C receptors and stimulate accumulation of cGMP in T84 cells. The activity was assayed as described earlier¹⁴. Results are expressed as an average of three determinations±SD.

FIG. 3A-D is a series of panels showing that SP-333 inhibits LPS induced activation of NF-κB in T84 cells by a cGMP mediated mechanism. T84 cells were stimulated with LPS (10 μg/ml) for 4 h and then treated either with 8-Br-cGMP (A) or SP-333 (B) in the presence of 500 μM Zaprinast for 16 h. Subsequent to the treatment, nuclear and cytosolic extracts were prepared. Nuclear extract was used to measure phosphorylated p65 levels and cytosolic extract was used to examine levels of IκB, phosphorylated IκB and IKK-β by Western blot. IKK-α/β phosphorylation was examined by stimulating T84 cells by LPS (10 μg/ml, 4 h) followed by 2 h treatment with SP-333 in the presence of 500 μM Zaprinast (C). Cytosolic extracts were prepared and levels of activated IKK-α/β, total IKK-β and actin were detected by Western using appropriate antibodies. Representative immunoblot depicting the levels of phosphorylated p65, IκB, p-IκB and total IKK-β, phosphorylated IKK-α/β and actin are shown. Relative transcript levels normalized to GAPDH levels in the sample from three independent determinations±SD are depicted in panel (D).

FIGS. 4A-B is a series of graphs showing that SP-333 inhibits LPS-induced secretion of pro-inflammatory cytokines by T84 cells. IL-8 (A) and TNF (B) levels were estimated by ELISA in supernatants of LPS stimulated T84 cells treated with 0.1, 1 and 10 μM of SP-333 as described. Each ELISA was performed in triplicate with cell-free supernatants from two independent experiments. The protein concentration of each well was assessed by Bio-Rad protein dye detection kit. The cytokine/protein ratio was employed to express the cytokine production and each result is expressed as the mean value of independent experiments±SD. Statistical significance calculated by comparing cytokine secretion from T84 cells incubated with SP-333 subsequent to LPS treatment versus corresponding secretion observed in cells treated with LPS alone.

FIG. 5A-B is a series of graphs showing that Plecanatide treatment ameliorates GI inflammation in chemically induced colitis in BDF1 mice. Efficacy of plecanatide in DSS (A) and TNBS (B) induced colitis was examined as described. Plecanatide formulated in 0.1M phosphate buffer (pH 7) was administered by oral gavage, once a day at indicated dosage. Sulfasalazine (80 mg/kg) was used as reference compound. At the end of the study, mice were sacrificed and distal section of the large intestine was fixed and embedded in paraffin. Colitis severity scores were assigned after visualization of H&E stained sections. All slides were scored in a blinded manner. Mean severity score±SEM plotted for indicated treatment groups. A significantly lower DSS induced colitis score was observed in animals that were administered 0.005 mg/kg of plecanatide compared to untreated controls (p=0.02) (A). In TNBS induced colitis model, animals that received plecanatide at 0.05 mg/kg and higher, exhibited significantly lower colitis scores as compared to their respective untreated controls. In both models, plecanatide treatment was superior to treatment with reference compound. Statistical significance calculated by comparing severity score observed for plecanatide or sulfasalazine treated group versus corresponding score for vehicle treated group. All slides were scored in a blinded manner. Mean histological severity of colitis score±SEM plotted for the indicated treatment groups. Statistical significance was calculated by comparing severity score observed for plecanatide or sulfasalazine treated group versus corresponding values in the vehicle treated group.

FIG. 6A-B is a series of graphs showing Effects of plecanatide treatment on GI Inflammation (A) and secretion of the pro-Inflammatory cytokines IL-12 p40, IL-23 and TNF in intestinal explants derived from TNBS-induced colitis in BALB/C mice (B). Colitis was induced in Balb/c mice via rectal instillation of TNBS. Mice were administered an oral gavage of vehicle or plecanatide (0.5 and 2.5 mg/kg) on day 0 and animals were euthanized on day 7 and colitis scores were determined (A). Colon tissues from the study were harvested for explant culture. Cells from explants were cultured for 24 h in the presence and absence of plecanatide. At the end of the incubation, culture media was snap frozen until cytokine analysis was performed. Average levels±SD of IL-12 p40, IL-23 and TNF in treated explant culture are depicted in B. Mice administered plecanatide exhibited significantly lower colitis scores (p<0.05) and correspondingly lower secretion of pro-inflammatory cytokines IL-12 p40, IL-23 and TNF as compared to untreated controls. Plotted values represent mean±SEM.

FIG. 7A-C is a series of graphs showing that plecanatide abrogates colitis in TCRα_{-/-}mice. Oral administration of plecanatide (0.5 and 2.5 mg/kg) for 14 days reduced the colitis score (A). At the end of the study, colon tissues were harvested and a portion was used for histopathological colitis scoring, and the remainder was immediately processed for explant culture for 24 hours. The culture media was snap frozen until analysis of IL-17, RANTES, MIP-1α, and IL-10. Intestinal explant cultures derived from plecanatide treated and untreated mice exhibit reduced secretion of pro-inflammatory cytokine (IL-17) and chemokines (RANTES and MIP-1α) and increased production of anti-inflammatory cytokine IL-10 as compared to vehicle treated samples (Figure B and C). Plotted values represent mean±SD.

FIGS. 8A-F is a series of graphs showing stability and biological activity of plecanatide and SP-333 in SIF. HPLC chromatographic analyses of plecanatide and SP-333 after digestion with heat inactivated SIF for 300 min (B and E) or SIF for 120 min. (C and F) respectively. SIF incubation completely converts plecanatide into a shorter peptide eluting at 9.4 min (indicated by * in C). Arrows indicate the position of plecanatide. As expected, SP-333 is resistant to digestion by SIF (F). Cyclic GMP synthesis by T84 cells in response to treatment with control or SIF digested plecanatide and SP-333 is shown in A and D. cGMP stimulation at 0 min is taken as 100%. The activities in samples at indicated incubation time is calculated as percent of that observed at 0 min. The data is average of triplicates±SD. After 6 h of incubation in SIF, SP-333 retains most of its biological activity while plecanatide retains ˜70% of its activity after 2.5-5 h incubation in SIF.

FIG. 9A-B is a series of graphs showing that oral administration of SP-333 abrogates DSS-induced colitis in BDF-1 mice. The figure depicts results for colitis severity (A), and disease activity index (B). BDF1 mice (n=12) were administered with 5% DSS in drinking water to induce colitis on day 1. Oral gavage with 5-ASA and vehicle (phosphate buffer) served as positive and negative control, respectively. SP-333 was administered by oral gavage from day 1 through day 7. DAI calculated (A) as per the described criteria²⁴. Scatter plot depicting DAI values for individual mice, together with mean values (horizontal bar) for each group. Oral administration of SP-333 at 0.05 mg/kg exhibits significant reduction (p=0.041) in DAI as compared to DSS vehicle control. Colitis severity (B) was calculated according to outlined criteria. Data shown are Mean scores±SEM for each group on day 7, determined from the observation of up to 5 mid-colon cross-sections. Oral dose of SP-333 (0.005 mg/kg) was as effective as 5-ASA (100 mg/kg) in ameliorating colitis in mice. Statistical significance calculated by comparing DAI or colitis severity score observed for SP-333 or 5ASA treated group versus corresponding score for vehicle treated group.

FIG. 10A-F is a series of tissue staining showing efficacy of SP-333 in DSS-induced colitis mouse model. Colitis was induced by providing 5% DSS in the drinking water. Control group received normal drinking water. SP-333 was administered by oral gavage, once a day at 0.005, 0.05, 0.5 and 5 mg/kg, from study day −1 (i.e. prior to initiation of DSS treatment) until study day 6. Reference compound, 5-ASA (100 mg/kg) was administered in a similar manner. All mice were euthanized on day 7, large bowel processed for histopathological analyses. Representative images of the histopathological evaluation of the large bowels from the DSS-induced colitis study (described in FIG. 9) are shown. A) untreated naïve mice, histopathology score=0; B) DSS-treated, histopathology score=4; C) DSS+SP-333 (0.005 mg/kg), histopathology score=1; D) DSS+SP-333 (0.05 mg/kg), histopathology score=2; E) DSS+SP-333 (0.5 mg/kg, histopathology score=2; and F) DSS+5-ASA (100 mg/kg), histopathology score=2.

FIG. 11A-B is a series of graphs showing DSS-induced changes in the Ki-67 labeling of crypt epithelial cells (A) and MPO activity in lysates prepared from mid-colon samples (B). Samples from 40 mice in the SP-333-administered groups, together with the vehicle control and 5-ASA treated groups were randomly selected for analyses. Values plotted represent Mean±SEM for each group. Administration of SP-333 improved symptoms of DSS-induced colitis in BDF1 mice. Samples derived from mice administered 0.005 mg/kg SP-333 exhibit the greatest percentage of crypts with normal Ki-67 labeling (A). Myeloperoxidase activity is shown as average increase in absorbance. Values are normalized to a sample protein concentration of 10 mg/ml. DSS/vehicle group demonstrated the largest increase in absorbance. With the exception of mice administered 0.5 mg/kg, absorbance values in samples from SP-333 treatment groups are significantly lower than DSS vehicle group (p<0.05) (B). Statistical significance calculated by comparing % crypt labeling or MPO activity observed for SP-333 or 5ASA treated group versus corresponding score for vehicle treated group. MPO activity is significantly lower in colon tissues from animals dosed with 0.05 mg/kg of SP-333.

FIG. 12 is a schematic illustration of the proposed mechanism for the anti-inflammatory effect of synthetic UG analogs. Binding of plecanatide and/or SP-333 to GC-C receptor located on the apical surface of intestinal epithelial cells results in receptor activation and increased intracellular cGMP production, leading to the activation of PKGII. Enhanced cGMP levels down regulate NF-κB signaling by blocking activation of IKK kinases necessary for phosphorylation and subsequent degradation of IκB inhibitor. Increased level of unphosphorylated cytosolic IκB binds p65 and p50 subunits and prevents their activation and subsequent translocation into the nucleus to initiate pro-inflammatory cascade.

FIG. 13 is a graph showing that activation of Src by PV and HgCl2 inhibited GCRA-mediated cGMP production by T84 cells.

FIG. 14 is a graph showing that SP-333 is a biologically active agonist of GC-C Receptor. SP-333 treatment stimulated cGMP synthesis in a dose-dependent manner in T84 cells, and approached a plateau at a concentration of 1 μM.

FIG. 15A-C is a series of graphs showing treatment with SP-333 enhanced cGMP production and Expression of Protein Kinase G I and II Transcripts.

FIG. 16A-B is a series of graphs demonstrating compared to vehicle, treatment with SP-333 downregulated NF-κB subunits, IKK-β, c-Src, and p65 as judged by reduction in their transcript and protein levels. After treatment with SP-333, a 59% decrease in IKKβ expression, a 55% decrease in p65 expression, and a 52% decrease in c-Src expression compared to untreated cells.

FIG. 17A-D is series of graphs demonstrating SP-333 downregulates c-Myc and transcripts of genes related to cell-cycle in T84 cells. Treatment with SP-333 results in a 92% decrease in c-Myc expression a 58% decrease in Cyclin D1 expression, and a 50% decrease in Survivin expression. Treatment with SP-333 appears to have no effect on the expression of β-Catenin.

FIG. 18A-B is a series of graphs showing SP-333 treatment modulates miRNAs known to be dysregulated in inflammation and cancer. In IBD and colon cancer, treatment with SP-333 upregulates miR-21 and MiR-155 levels, while treatment with SP-333 downregulates levels of miR-126 and miR-101 in colon cancer.

FIG. 19A-C is a series of graphs demonstrating that SP-333 upregulates expression of miRNAs that are known to be expressed following NF-κB activation. NF-κB activation down-regulates miR-29 family and let-7i. Treatment with SP-333 upregulated miRs such as miR-15a (p<0.05), miR-16 (p<0.01), let-7i (p<0.005), miR-125b (p<0.001) and the family of miR-29 (p<0.05), all of which are negative regulators of NF-κB signaling, which is known to augment production of pro-inflammatory cytokines during GI inflammation.

FIG. 20 is a schematic representing the mechanism by which SP-333 modulates expression of genes and miRNAs implicated in inflammation and cancer. These data will facilitate evaluation of the select miRNAs and corresponding target genes in IBD tissues.

### DETAILED DESCRIPTION

It should be understood that singular forms such as “a,” “an,” and “the” are used throughout this application for convenience, however, except where context or an explicit statement indicates otherwise, the singular forms are intended to include the plural. Further, it should be understood that every journal article, patent, patent application, publication, and the like that is mentioned herein is hereby incorporated by reference in its entirety and for all purposes. All numerical ranges should be understood to include each and every numerical point within the numerical range, and should be interpreted as reciting each and every numerical point individually. The endpoints of all ranges directed to the same component or property are inclusive, and intended to be independently combinable.

“About” includes all values having substantially the same effect, or providing substantially the same result, as the reference value. Thus, the range encompassed by the term “about” will vary depending on context in which the term is used, for instance the parameter that the reference value is associated with. Thus, depending on context, “about” can mean, for example, ±15%, ±10%, ±5%, ±4%, ±3%, ±2%, ±1%, or ±less than 1%. Importantly, all recitations of a reference value preceded by the term “about” are intended to also be a recitation of the reference value alone.

The present invention is based upon the development of agonists of guanylate cyclase-C (GC-C) for the treatment of inflammatory disorders and cancer. Exemplary GC-C agonists are analogs of plecanatide, uroguanylin, guanylin, lymphoguanylin and E.coli ST peptide. The invention relates to a composition including at least one GC-C peptide (i.e., GCRA peptide)

The invention is based upon the surprising discovery that CG-C agonists can inhibit NF-κB signaling, thereby exerting anti-inflammatory effects. Plecanatide (SP-304) and SP-333, structural analogs of uroguanylin, an endogenous natriuretic peptide that activates guanylate cyclase-C (CG-C), ameliorates DSS- and TNBS-induced acute colitis in murine models. Plecanatide treatment also ameliorated spontaneous colitis in T-cell receptor alpha knockout mice. Consistent with its in vivo anti-inflammatory activity, plecanatide treatment suppressed production of inflammatory cytokines and chemokines such as IL-12p40, IL-23, TNF, MIP-1α, IL-17 and RANTES in colon explants. Similarly, SP-333 also ameliorated DSS-induced colitis in mice.SP-333 treatment inhibited lipopolysaccharide-mediated activation of nuclear factor-κB (NF-κB) in T84 cells.

The present invention is based upon several concepts. The first is that there is a cGMP-dependent mechanism which regulates the balance between cellular proliferation and apoptosis and that a reduction in cGMP levels, due to a deficiency of uroguanylin/guanylin and/or due to the activation of cGMP-dependent phosphodiesterases, is an early and critical step in neoplastic transformation. A second concept is that the release of arachidonic acid from membrane phospholipids, which leads to the activation of cytoplasmic phospholipase A2 (cPLA2), cyclooxygenase-2 (COX-2) and possibly 5-lipoxygenase (5-LO) during the process of inflammation, is down-regulated by a cGMP-dependent mechanism, leading to reduced levels of prostaglandins and leukotrienes, and that increasing intracellular levels of cGMP may therefore produce an anti-inflammatory response. In addition, a cGMP-dependent mechanism, is thought to be involved in the control of pro-inflammatory processes. Therefore, elevating intracellular levels of cGMP may be used as a means of treating and controlling lipid metabolism disorders, biliary disorders, gastrointestinal disorders, inflammatory disorders, lung disorders, cancer, cardiac disorders including cardiovascular disorders, eye disorders, oral disorders, blood disorders, liver disorders, skin disorders, prostate disorders, endocrine disorders, increasing gastrointestinal motility and obesity. Lipid metabolism disorders include, but not limited to, dyslipidemia, hyperlipidemia, hypercholesterolemia, hypertriglyceridemia, sitosterolemia, familial hypercholesterolemia, xanthoma, combined hyperlipidemia, lecithin cholesterol acyltransferase deficiency, tangier disease, abetalipoproteinemia, erectile dysfunction, fatty liver disease, and hepatitis. Billary disorders include gallbladder disorders such as for example, gallstones, gall bladder cancer cholangitis, or primary sclerosing cholangitis; or bile duct disorders such as for example, cholecystitis, bile duct cancer or fascioliasis. Gastrointestinal disorders include for example, irritable bowel syndrome (IBS), non-ulcer dyspepsia, chronic intestinal pseudo-obstruction, functional dyspepsia, colonic pseudo-obstruction, duodenogastric reflux, gastroesophageal reflux disease (GERD), ileus inflammation (e.g., post-operative ileus), gastroparesis, heartburn (high acidity in the GI tract), constipation (e.g., IBS-associated constipation, constipation associated with use of medications such as opioids, osteoarthritis drugs, osteoporosis drugs; post surgical constipation, constipation associated with neuropathic disorders. Inflammatory disorders include tissue and organ inflammation such as kidney inflammation (e.g., nephritis), gastrointestinal system inflammation (e.g., Crohn's disease and ulcerative colitis); necrotizing enterocolitis (NEC); pancreatic inflammation (e.g., pancreatis), lung inflammation (e.g., bronchitis or asthma) or skin inflammation (e.g., psoriasis, eczema). Lung Disorders include for example chronic obstructive pulmonary disease (COPD), and fibrosis. Cancer includes tissue and organ carcinogenesis including metastases such as for example gastrointestinal cancer (e.g., gastric cancer, esophageal cancer, pancreatic cancer colorectal cancer including colorectal metastasis, intestinal cancer, anal cancer, liver cancer, gallbladder cancer, or colon cancer); lung cancer; thyroid cancer; skin cancer (e.g., melanoma); oral cancer; urinary tract cancer (e.g. bladder cancer or kidney cancer); blood cancer (e.g. myeloma or leukemia) or prostate cancer. Cardiac disorders include for example, congestive heart failure, trachea cardia hypertension, high cholesterol, or high triglycerides. Cardiovascular disorders include for example aneurysm, angina, atherosclerosis, cerebrovascular accident (stroke), cerebrovasculardisease, congestive heart failure, coronary artery disease, myocardial infarction (heart attack), or peripheral vascular disease. Liver disorders include for example cirrhosis and fibrosis. In addition, GC-C agonist may also be useful to facilitate liver regeneration in liver transplant patients. Eye disorders include for example increased intra-ocular pressure, glaucoma, dry eyes retinal degeneration, disorders of tear glands or eye inflammation. Skin disorders include for example xerosis. Oral disorders include for example dry mouth (xerostomia), Sjogren's syndrome, gum diseases (e.g., periodontal disease), or salivary gland duct blockage or malfunction. Prostate disorders include for example benign prostatic hyperplasia (BPH). Endocrine disorders include for example diabetes mellitus, hyperthyroidism, hypothyroidism, and cystic fibrosis.

Uroguanylin is a circulating peptide hormone with natriuretic activity and has been found to stimulate fluid and electrolyte transport in a manner similar to another family of heat stable enterotoxins (ST peptides) secreted by pathogenic strains of E. coli and other enteric bacteria that activate guanylate cyclase receptor and cause secretory diarrhea. Unlike bacterial ST peptides, the binding of uroguanylin to guanylate cyclase receptor is dependent on the physiological pH of the gut. Therefore, uroguanylin is expected to regulate fluid and electrolyte transport in a pH dependent manner and without causing severe diarrhea.

The invention also provides a method for preventing or treating a condition by administering to a subject in need of a therapeutically effective amount of the composition of the present invention. The condition that can be treated by this composition includes colitis, ulcerative colitis, Crohn's disease, irritable bowel syndrome (IBS), non-ulcer dyspepsia, chronic intestinal pseudo-obstruction, functional dyspepsia, colonic pseudo-obstruction, duodenogastric reflux, constipation, IBS-associated constipation, constipation associated with use of opiate pain killers, post-surgical constipation, constipation associated with neuropathic disorders, gastroesophageal reflux disease (GERD), Celiac disease, gastroparesis, heartburn, poor gastrointestinal motility, congestive heart failure, hypertension, benign prostatic hyperplasia (BPH), gastrointestinal cancer, lung cancer, bladder cancer, liver cancer, salivary gland cancer, skin cancer, colon cancer, bronchitis, tissue inflammation, organ inflammation, respiratory inflammation, asthma, COPD, lipid metabolism disorder, biliary disorder, cardiovascular disease, obesity and an endocrine disorder. Preferably, the condition is a gastrointestinal inflammatory disease (for example IBS-associated constipation, ulcerative colitis and Crohn's disease), a gastrointestinal cancer, or colorectal metastasis.

The invention also relates in part to the use of GC-C agonists to inhibit activation of NF-κB, to reduce production of pro-inflammatory cytokines/chemokines and to increase secretion of anti-inflammatory cytokines to ameliorate colitis. Thus, GC-C agonists and compositions described herein may be used either alone or in combination with other anti-inflammatory drugs, for example, Sulfasalazine (Azulfidine) Mesalamine (Asacol, Lialda), balsalazide (Colazal), olsalazine (Dipentum), Corticosteroids, immune system suppressors, for example, Azathioprine (Azasan, Imuran) and mercaptopurine (Purinethol), Cyclosporine (Gengraf, Neoral, Sandimmune), Infliximab (Remicade), and/or immunomodulatory agents (such as 6-mercaptopurine and methotrexate).

NF-κB may include one or more transcription factor of the NF-κB family, for example without being limited to the list herein, NF-κB1 (p50), NF-κB2 (p52), p65 (RelA), c-Rel, and RelB, or any protein that share a common structural motif called the Rel homology domain.

Pro-inflammatory cytokines include, but are not limited to, IL-1, IL-2, TNF, IL-12p40, IL-17, and IL-23. Chemokines include, but are not limited to, IL-8, RANTES and MIP-1α. Anti-inflammatory cytokines include, but are not limited to, IL-10.

In some embodiments, GC-C agonists inhibit the nuclear localization of NF-κB.

In some embodiments, GC-C agonists mediate inhibition of NF-κB activating factors. NF-κB activating factors are, without being limited to the examples herein, are cytokines such as tumor necrosis factor (TNF) and interleukin (IL)-1, lipopolysaccharides, bacterial and viral infections, activators of protein kinase C, and oxidants. The inhibition of NF-κB may result in reduced production of cytokines (such as, without being limited to the examples herein, TNF, IL-1, IL-2, IL-6, IL-8, IL-12p40, IL-17, and IL-23), adhesion molecules (such as ICAM-1, VCAM-1, E-selectin, and MAdCAM-1), and enzymes that are involved in inflammation, such as inducible nitric oxide synthase and cyclooxygenase-2. In some embodiments, the invention may provide inhibition of proteins whose genes are switched on by NF-κB, such as, without being limited to the examples herein, TNF and IL-1.

Some embodiments provide dysregulation of the expression of NF-κB target genes, such as TNF, implicated in the pathogenesis of inflammatory disorders or diseases, such as inflammatory bowel diseases. In some embodiments, the inhibition of NF-κB activation with GC-C agonists may prevent IkB degradation and may attenuate chronic inflammation such as that associated with Crohn's disease. In another embodiment, inhibition of p65 subunit of NF-κB may effectively abrogate colonic inflammation such as that associated with colitis.

In another embodiment, inhibition of NF-κB with GC-C agonists may prevent mucosal NF-κB activation. In some embodiments, inhibition of NF-κB with GC-C agonists may prevent mucosal NF-κB activation in ulcerative colitis patients. In particular the GC-C agonists of the current invention may inhibit NF-κB activation in macrophages.

The current invention also provides GC-C agonists mediated enhancement or stimulation of cGMP signaling pathway results in the inhibition of NF-κB. According to some embodiments, the GC-C agonist mediated enhancement or stimulation of cGMP may result in the activation of the cyclic dependent protein kinase (PKG). The cyclic GMP-dependent kinase (PKG) is an important mediator of signal transduction that may induce gene expression through cAMP response element binding protein (CREB).

In a merely illustrative embodiment, Plecanatide (SP304), SP333 or SP373 increases cGMP production, leading to the activation of PKG, which is a key regulator that turns on downstream signaling to activate ion channels, cyclic nucleotide gated channels and fluid homeostasis ENREF 37. Subsequent downstream signaling leads to inhibition of NF-κB activation, resulting in reduced production of pro-inflammatory cytokines TNF, IL-12p40, IL-17, IL-23; chemokines IL8, RANTES and MIP-1α and in increased secretion of anti-inflammatory cytokine IL-10, contributing to amelioration of colitis in chemically induced and genetically altered mouse models of colitis.

The present invention provides a method of preventing a subject at risk of, treating a subject suffering from, or ameliorating a symptom of a NF-κB mediated inflammatory disorder by administering to the subject an effective amount of a GC-C agonist or pharmaceutical composition thereof, or a composition described herein. The invention provides that the effective amount is sufficient to inhibit NF-κB activation, thus preventing, treating a subject at risk or suffering from or ameliorating a symptom of a NF-κB mediated inflammatory disorder. In some embodiments, the invention provides a method of preventing a subject at risk of, treating a subject suffering from, or ameliorating a symptom of gastrointestinal inflammation comprising administering to the subject an effective amount of a GC-C agonist or pharmaceutical composition thereof, or a composition described herein. In some embodiments, the invention provides a method of preventing a subject at risk of, treating a subject suffering from, or ameliorating a symptom of colitis comprising administering to the subject an effective amount of a GC-C agonist or pharmaceutical composition thereof, or a composition described herein. In some embodiments, the invention provides a method of preventing a subject at risk of, treating a subject suffering from, or ameliorating a symptom of cancer comprising administering to the subject an effective amount of a GC-C agonist or pharmaceutical composition thereof, or a composition described herein.

In some embodiments, the GC-C agonist is administered to the subject concurrently or sequentially with lipopolysaccharide (LPS).

The invention also relates to a method of modulating NF-κB induction in a cell by contacting the cell with an effective amount of a GC-C agonist or pharmaceutical composition thereof or a composition described herein.

The present invention also provides a method for modulating NF-κB-dependent target gene expression in a cell by contacting the cell with an effective amount of a GC-C agonist, where the GC-C agonist inhibits NF-κB activation, thereby modulating NF-κB-dependent target gene expression in a cell. Exemplary NF-κB-dependent target genes include, but are not limited to, IL-1, IL-2, TNF, IL-12p40, IL-17, IL-23, IL-8, RANTES, MIP-1α, and IL-10.

Any methods of the present invention may further include administering to the subject or contacting the cell with one or more other agents. The one or more other agents include, for example, inhibitor of a NF-κB, inhibitor of c-Src, inhibitor of cGMP-dependent phosphodiesterase, anti-colitis agent, anti-inflammatory drugs, for example, Sulfasalazine (Azulfidine), Mesalamine (Asacol, Lialda), balsalazide (Colazal), olsalazine (Dipentum), Corticosteroids, immune system suppressors, for example, Azathioprine (Azasan, Imuran) and mercaptopurine (Purinethol), Cyclosporine (Gengraf, Neoral, Sandimmune), Infliximab (Remicade) or immunomodulatory agents (such as 6-mercaptopurine and methotrexate). In some embodiment of the current invention, the one or more other agents may be administered either concurrently or sequentially with a GC-C agonist or pharmaceutical composition thereof.

The GC-C agonists according to the invention include amino acid sequences represented by Formulas I-XX as well as those amino acid sequence summarized below in Tables 1-7. The GC-C agonists according to the invention are collectively referred to herein as “GCRA peptides”. In some embodiments, the GC-C agonist has the sequence of SEQ ID NO: 1 (SP-304), SEQ ID NO.:9 (SP-333), or SEQ ID NO: 250 (SP-373).

The GCRA peptides described herein bind the guanylate cyclase C (GC-C) and stimulate intracellular production of cyclic guanosine monophosphate (cGMP). Optionally, the GCRA peptides induce apoptosis. In some aspects, the GCRA peptides stimulate intracellular cGMP production at higher levels than naturally occurring GC-C agonists (e.g., uroguanylin, guanylin, lymphoguanylin and E.coli ST peptides).

For example, the GCRA peptides of the invention stimulate 5%, 10%, 20%, 30%, 40%, 50%, 75%, 90% or more intracellular cGMP compared to naturally occurring GC-C agonists. The terms induced and stimulated are used interchangeably throughout the specification. The GCRA peptides described herein are more stable than naturally occurring GC-C agonists.

The GCRA peptides described herein have therapeutic value in the treatment of a wide variety of disorders and conditions including for example lipid metabolism disorders, biliary disorders, gastrointestinal disorders, inflammatory disorders, lung disorders, cancer, cardiac disorders including cardiovascular disorders, eye disorders, oral disorders, blood disorders, liver disorders, skin disorders, prostate disorders, endocrine disorders, increasing gastrointestinal motility and obesity. Lipid metabolism disorders include, but not limited to, dyslipidemia, hyperlipidemia, hypercholesterolemia, hypertriglyceridemia, sitosterolemia, familial hypercholesterolemia, xanthoma, combined hyperlipidemia, lecithin cholesterol acyltransferase deficiency, tangier disease, abetalipoproteinemia, erectile dysfunction, fatty liver disease, and hepatitis. Binary disorders include gallbladder disorders such as for example, gallstones, gall bladder cancer cholangitis, or primary sclerosing cholangitis; or bile duct disorders such as for example, cholecystitis, bile duct cancer or fascioliasis. Gastrointestinal disorders include for example, irritable bowel syndrome (IBS), non-ulcer dyspepsia, chronic intestinal pseudo-obstruction, functional dyspepsia, colonic pseudo-obstruction, duodenogastric reflux, gastroesophageal reflux disease (GERD), ileus inflammation (e.g., post-operative ileus), gastroparesis, heartburn (high acidity in the GI tract), constipation (e.g., IBS-associated constipation, constipation associated with use of medications such as opioids, osteoarthritis drugs, osteoporosis drugs; post surgical constipation, constipation associated with neuropathic disorders). Inflammatory disorders include tissue and organ inflammation such as kidney inflammation (e.g., nephritis), gastrointestinal system inflammation (e.g., chronic inflammatory bowel disease, Crohn's disease, colitis, and ulcerative colitis); necrotizing enterocolitis (NEC); pancreatic inflammation (e.g., pancreatis), lung inflammation (e.g., bronchitis or asthma) or skin inflammation (e.g., psoriasis, eczema). Lung Disorders include for example chronic obstructive pulmonary disease (COPD), and fibrosis. Cancer includes tissue and organ carcinogenesis including metatases such as for example gastrointestinal cancer (e.g., gastric cancer, esophageal cancer, pancreatic cancer, colorectal cancer including colorectal metastasis, intestinal cancer, anal cancer, liver cancer, gallbladder cancer, or colon cancer); lung cancer; thyroid cancer; skin cancer (e.g., melanoma); oral cancer; urinary tract cancer (e.g. bladder cancer or kidney cancer); blood cancer (e.g. myeloma or leukemia) or prostate cancer. Cardiac disorders include for example, congestive heart failure, trachea cardia hypertension, high cholesterol, or high tryglycerides. Cardiovascular disorders include for example aneurysm, angina, atherosclerosis, cerebrovascular accident (stroke), cerebrovasculardisease, congestive heart failure, coronary artery disease, myocardial infarction (heart attack), or peripheral vascular disease. Liver disorders include for example cirrhosis and fibrosis. In addition, GC-C agonist may also be useful to facilitate liver regeneration in liver transplant patients. Eye disorders include for example increased intra-ocular pressure, glaucoma, dry eyes retinal degeneration, disorders of tear glands or eye inflammation. Skin disorders include for example xerosis. Oral disorders include for example dry mouth (xerostomia), Sjogren's syndrome, gum diseases (e.g., periodontal disease), or salivary gland duct blockage or malfunction. Prostate disorders include for example benign prostatic hyperplasia (BPH). Endocrine disorders include for example diabetes mellitus, hyperthyroidism, hypothyroidism, and cystic fibrosis.

As used herein, the term “guanylate cyclase receptor (GCR)” refers to the class of guanylate cyclase C receptor on any cell type to which the inventive agonist peptides or natural agonists described herein bind. As used herein, “intestinal guanylate cyclase receptor” is found exclusively on epithelial cells lining the GI mucosa. Uroguanylin, guanylin, and ST peptides are expected to bind to these receptors and may induce apoptosis. The possibility that there may be different receptors for each agonist peptide is not excluded. Hence, the term refers to the class of guanylate cyclase receptors on epithelial cells.

As used herein, the term “GCR agonist” is meant to refer to peptides and/or other compounds that bind to an intestinal guanylate cyclase receptor and stimulate fluid and electrolyte transport. This term also covers fragments and pro-peptides that bind to GCR and stimulate fluid and water secretion.

As used herein, the term “substantially equivalent” is meant to refer to a peptide that has an amino acid sequence equivalent to that of the binding domain where certain residues may be deleted or replaced with other amino acids without impairing the peptide's ability to bind to an intestinal guanylate cyclase receptor and stimulate fluid and electrolyte transport.

Addition of carriers (e.g., phosphate-buffered saline or PBS) and other components to the composition of the present invention is well within the level of skill in this art. In addition to the compound, such compositions may contain pharmaceutically acceptable carriers and other ingredients known to facilitate administration and/or enhance uptake. Other formulations, such as microspheres, nanoparticles, liposomes, and immunologically-based systems may also be used in accordance with the present invention. Other examples include formulations with polymers (e.g., 20% w/v polyethylene glycol) or cellulose, or enteric formulations.

GCRA Peptides

The GCRA peptides of the present invention are analogues of plecanatide, uroguanylin, guanylin, lymphoguanylin and ST peptides. No particular length is implied by the term “peptide”. In some embodiments, the GCRA peptide is less than 25 amino acids in length, e.g., less than or equal to 20, 15, 14, 13, 12, 11, 10, or 5 amino acid in length.

The GCRA peptides can be polymers of L-amino acids, D-amino acids, or a combination of both. For example, in various embodiments, the peptides are D retro-inverso peptides. The term “retro-inverso isomer” refers to an isomer of a linear peptide in which the direction of the sequence is reversed and the chirality of each amino acid residue is inverted. See, e.g., Jameson et al., Nature, 368, 744-746 (1994); Brady et al., Nature, 368, 692-693 (1994). The net result of combining D-enantiomers and reverse synthesis is that the positions of carbonyl and amino groups in each amide bond are exchanged, while the position of the side-chain groups at each alpha carbon is preserved. Unless specifically stated otherwise, it is presumed that any given L-amino acid sequence of the invention may be made into a D retro-inverso peptide by synthesizing a reverse of the sequence for the corresponding native L-amino acid sequence. For example a GCRA peptide includes the sequence defined by Formulas I-XX and those listed on Tables 1-7.

By inducing cGMP production is meant that the GCRA peptide induces the production of intracellular cGMP. Intracellular cGMP is measured by methods known in the art. For example, the GCRA peptide of the invention stimulate 5%, 10%, 20%, 30%, 40%, 50%, 75%, 90% or more intracellular cGMP compared to naturally occurring GC-C agonists. In further embodiments, the GCRA peptide stimulates apoptosis, e.g., programmed cell death or activates the cystic fibrosis transmembrane conductance regulator (CFTR). In further embodiments, the GCRA peptide modulates NF-κB expression. In further embodiments, the GCRA peptide modulates NF-κB signaling. In yet a further embodiment, the NF-κB expression and/or signaling is inhibited.

As used herein PEG3, 3 PEG, is meant to denote polyethylene glycol such as include aminoethyloxy-ethyloxy-acetic acid (AeeA).

As used herein, the term “AMIDE” is meant to denote that the terminal carboxylic acid is replaced with an amide group, i.e., the terminal COOH is replaced with CONH_{2.}

As used herein, the term “pyGlu” refers to pyroglutamic acid.

As used herein, (e.g., in Formulas I-XX) Xₐₐ is any natural, unnatural amino acid or amino acid analogue; Mₐₐ is a Cysteine (Cys), Penicillamine (Pen) homocysteine, or 3-mercaptoproline. Xaaₙ₁ is meant to denote an amino acid sequence of any natural, unnatural amino acid or amino acid analogue that is one, two or three residues in length; Xaaₙ₂ is meant to denote an amino acid sequence of any natural, unnatural amino acid or amino acid analogue that is zero or one residue in length; and Xaaₙ₃ is meant to denote an amino acid sequence of any natural, unnatural amino acid or amino acid analogue that is zero, one, two, three, four, five or six residues in length. Additionally, any amino acid represented by Xaa, may be an L-amino acid, a D-amino acid, a methylated amino acid, a florinated amino acid or any combination of thereof. Preferably the amino acids at the N-terminus, C-terminus or both are D-amino acids. Optionally, any GCRA peptide represented by Formulas I-XX may contain on or more polyethylene glycol residues at the N-terminus, C-terminus or both. An exemplary polyethylene glycol includes aminoethyloxy-ethyloxy-acetic acid and polymers thereof.

Specific examples of GCC agonist peptides that can be used in the methods and formulations of the invention include a peptide selected from Tables 1-7.

In some embodiments, GCC agonist peptides include peptides having the amino acid sequence of Formula I, wherein at least one amino acid of Formula I is a D-amino acid or a methylated amino acid and/or the amino acid at position 16 is a serine. Preferably, the amino acid at position 16 of Formula I is a D-amino acid or a methylated amino acid. For example, the amino acid at position 16 of Formula I is a d-leucine or a d-serine. Optionally, one or more of the amino acids at positions 1-3 of Formula I are D-amino acids or methylated amino acids or a combination of D-amino acids or methylated amino acids. For example, Asn¹, Asp² or Glu³ (or a combination thereof) of Formula I is a D-amino acid or a methylated amino acid. Preferably, the amino acid at position Xaa⁶ of Formula I is a leucine, serine or tyrosine.

In alternative embodiments, GCC agonist peptides include peptides having the amino acid sequence of Formula II, wherein at least one amino acid of Formula II is a D-amino acid or a methylated amino acid. Preferably, the amino acid denoted by Xaaₙ₂ of Formula II is a D-amino acid or a methylated amino acid. In some embodiments, the amino acid denoted by Xaaₙ₂ of Formula II is a leucine, a d-leucine, a serine, or a d-serine. Preferably, the one or more amino acids denoted by Xaaₙ₁ of Formula II are D-amino acids or methylated amino acids. Preferably, the amino acid at position Xaa⁶ of Formula II is a leucine, a serine, or a tyrosine.

In some embodiments, GCC agonist peptides include peptides having the amino acid sequence of Formula III, wherein at least one amino acid of Formula III is a D-amino acid or a methylated amino acid and/or Maa is not a cysteine. Preferably, the amino acid denoted by Xaaₙ₂ of Formula III is a D-amino acid or a methylated amino acid. In some embodiments the amino acid denoted by Xaaₙ₂ of Formula III is a leucine, a d-leucine, a serine, or a d-serine. Preferably, the one or more amino acids denoted by Xaaₙ₁ of Formula III are D-amino acids or methylated amino acids. Preferably, the amino acid at position Xaa⁶ of Formula III is a leucine, a serine, or a tyrosine.

In other embodiments, GCC agonist peptides include peptides having the amino acid sequence of Formula IV, wherein at least one amino acid of Formula IV is a D-amino acid or a methylated amino acid, and/or Maa is not a cysteine. Preferably, the Xaaₙ₂ of Formula IV is a D-amino acid or a methylated amino acid. In some embodiments, the amino acid denoted by Xaaₙ₂ of Formula IV is a leucine, a d-leucine, a serine, or a d-serine. Preferably, the one or more of the amino acids denoted by Xaaₙ₁ of Formula IV are D-amino acids or methylated amino acids. Preferably, the amino acid denoted Xaa⁶ of Formula IV is a leucine, a serine, or a tyrosine.

In further embodiments, GCC agonist peptides include peptides having the amino acid sequence of Formula V, wherein at least one amino acid of Formula V is a D-amino acid or a methylated amino acid. Preferably, the amino acid at position 16 of Formula V is a D-amino acid or a methylated amino acid. For example, the amino acid at position 16 (i.e., Xaa¹⁶) of Formula V is a d-leucine or a d-serine. Optionally, one or more of the amino acids at position 1-3 of Formula V are D-amino acids or methylated amino acids or a combination of D-amino acids or methylated amino acids. For example, Asn¹, Asp² or Glu³ (or a combination thereof) of Formula V is a D-amino acids or a methylated amino acid. Preferably, the amino acid denoted at Xaa⁶ of Formula V is a leucine, a serine, or a tyrosine.

In additional embodiments, GCRA peptides include peptides having the amino acid sequence of Formula VI, VII-a, VII-b, VIII, or IX. Preferably, the amino acid at position 6 of Formula VI, VII-a, VII-b, VIII, or IX is a leucine, a serine or a tyrosine. In some aspects the amino acid at position 16 of Formula VI, VII-a, VII-b, VIII or IX is a leucine or a serine. Preferably, the amino acid at position 16 of Formula VI, VII-a, VII-b, VIII or IX is a D-amino acid or a methylated amino acid.

In additional embodiments, GCRA peptides include peptides having the amino acid sequence of Formula X, XI, XII, XIII, XIV, XV, XVI or XVII. Optionally, one or more amino acids of Formulas X, XI, XII, XIII, XIV, XV, XVI or XVII are D-amino acids or methylated amino acids. Preferably, the amino acid at the carboxyl terminus of the peptides according to Formulas X, XI, XII, XIII, XIV, XV, XVI or XVII is a D-amino acid or a methylated amino acid. For example the amino acid at the carboxyl terminus of the peptides according to Formulas X, XI, XII, XIII, XIV, XV, XVI or XVII is a D-tyrosine.

Preferably, the amino acid denoted by Xaa⁶ of Formula XIV is a tyrosine, phenylalanine or a serine. Most preferably the amino acid denoted by Xaa⁶ of Formula XIV is a phenylalanine or a serine. Preferably, the amino acid denoted by Xaa⁴ of Formula XV, XVI or XVII is a tyrosine, a phenylalanine, or a serine. Most preferably, the amino acid position Xaa⁴ of Formula XV, XVI or XVII is a phenylalanine or a serine.

In some embodiments, GCRA peptides include peptides containing the amino acid sequence of Formula XVIII. Preferably, the amino acid at position 1 of Formula XVIII is a glutamic acid, aspartic acid, glutamine or lysine. Preferably, the amino acid at position 2 and 3 of Formula XVIII is a glutamic acid, or an aspartic acid. Preferably, the amino acid at position 5 is a glutamic acid. Preferably, the amino acid at position 6 of Formula XVIII is an isoleucine, valine, serine, threonine or tyrosine. Preferably, the amino acid at position 8 of Formula XVIII is a valine or isoleucine. Preferably, the amino acid at position 9 of Formula XVIII is an asparagine. Preferably, the amino acid at position 10 of Formula XVIII is a valine or a methionine. Preferably, the amino acid at position 11 of Formula XVIII is an alanine. Preferably, the amino acid at position 13 of Formula XVIII is a threonine. Preferably, the amino acid at position 14 of Formula XVIII is a glycine. Preferably, the amino acid at position 16 of Formula XVIII is a leucine, serine or threonine

In alternative embodiments, GCRA peptides include peptides containing the amino acid sequence of Formula XIX. Preferably, the amino acid at position 1 of Formula XIX is a serine or asparagine. Preferably, the amino acid at position 2 of Formula XIX is a histidine or an aspartic acid. Preferably, the amino acid at position 3 of Formula XIX is a threonine or a glutamic acid. Preferably, the amino acid at position 5 of Formula XIX is a glutamic acid. Preferably, the amino acid at position 6 of Formula XIX is an isoleucine, leucine, valine or tyrosine. Preferably, the amino acid at position 8, 10, 11, or 13 of Formula XIX is an alanine. Preferably, the amino acid at position 9 of Formula XIX is an asparagine or a phenylalanine. Preferably, the amino acid at position 14 of Formula XIX is a glycine.

In further embodiments, GCRA peptides include peptides containing the amino acid sequence of Formula XX. Preferably, the amino acid at position 1 of Formula XX is a glutamine. Preferably, the amino acid at position 2 or 3 of Formula XX is a glutamic acid or an aspartic acid. Preferably, the amino acid at position 5 of Formula XX is a glutamic acid. Preferably, the amino acid at position 6 of Formula XX is threonine, glutamine, tyrosine, isoleucine, or leucine. Preferably, the amino acid at position 8 of Formula XX is isoleucine or valine. Preferably, the amino acid at position 9 of Formula XX is asparagine. Preferably, the amino acid at position 10 of Formula XX is methionine or valine. Preferably, the amino acid at position 11 of Formula XX is alanine. Preferably, the amino acid at position 13 of Formula XX is a threonine. Preferably, the amino acid at position 1 of Formula XX is a glycine. Preferably, the amino acid at position 15 of Formula XX is a tyrosine. Optionally, the amino acid at position 15 of Formula XX is two-amino acid in length and is Cysteine (Cys), Penicillamine (Pen) homocysteine, or 3-mercaptoproline and serine, leucine or threonine.

In certain embodiments, one or more amino acids of the GCRA peptides can be replaced by a non-naturally occurring amino acid or a naturally or non-naturally occurring amino acid analog. There are many amino acids beyond the standard 20 (Ala, Arg, Asn, Asp, Cys, Gln, Glu, Gly, His, Ile, Leu, Lys, Met, Phe, Pro, Ser, Thr, Trp, Tyr, and Val). Some are naturally-occurring others are not. (See, for example, Hunt, The Non-Protein Amino Acids: In Chemistry and Biochemistry of the Amino Acids, Barrett, Chapman and Hall, 1985). For example, an aromatic amino acid can be replaced by 3,4-dihydroxy-L-phenylalanine, 3-iodo-L-tyrosine, triiodothyronine, L-thyroxine, phenylglycine (Phg) or nor-tyrosine (norTyr). Phg and norTyr and other amino acids including Phe and Tyr can be substituted by, e.g., a halogen, —CH3, —OH, —CH2NH3, —C(O)H, —CH2CH3, —CN, —CH2CH2CH3, —SH, or another group. Any amino acid can be substituted by the D-form of the amino acid.

With regard to non-naturally occurring amino acids or naturally and non-naturally occurring amino acid analogs, a number of substitutions in the polypeptide and agonists described herein are possible alone or in combination.

For example, glutamine residues can be substituted with gamma-Hydroxy-Glu or gamma-Carboxy-Glu. Tyrosine residues can be substituted with an alpha substituted amino acid such as L-alpha-methylphenylalanine or by analogues such as: 3-Amino-Tyr; Tyr(CH3); Tyr(PO3(CH3)2); Tyr(SO3H); beta-Cyclohexyl-Ala; beta-(1-Cyclopentenyl)-Ala; beta-Cyclopentyl-Ala; beta-Cyclopropyl-Ala; beta-Quinolyl-Ala; beta-(2-Thiazolyl)-Ala; beta-(Triazole-1-yl)-Ala; beta-(2-Pyridyl)-Ala; beta-(3-Pyridyl)-Ala; Amino-Phe; Fluoro-Phe; Cyclohexyl-Gly; tBu-Gly; beta-(3-benzothienyl)-Ala; beta-(2-thienyl)-Ala; 5-Methyl-Trp; and A-Methyl-Trp. Proline residues can be substituted with homopro (L-pipecolic acid); hydroxy-Pro; 3,4-Dehydro-Pro; 4-fluoro-Pro; or alpha-methyl-Pro or an N(alpha)-C(alpha) cyclized amino acid analogues with the structure: n=0, 1, 2, 3 Alanine residues can be substituted with alpha-substitued or N-methylated amino acid such as alpha-amino isobutyric acid (aib), L/D-alpha-ethylalanine (L/D-isovaline), L/D-methylvaline, or L/D-alpha-methylleucine or a non-natural amino acid such as beta-fluoro-Ala. Alanine can also be substituted with: n=0, 1, 2, 3 Glycine residues can be substituted with alpha-amino isobutyric acid (aib) or L/D-alpha-ethylalanine (L/D-isovaline).

Further examples of unnatural amino acids include: an unnatural analog of alanine (e.g., L-1-Nal or L-2-Nal); an unnatural analog of tyrosine; an unnatural analogue of glutamine; an unnatural analogue of phenylalanine; an unnatural analogue of serine; an unnatural analogue of threonine; an alkyl, aryl, acyl, azido, cyano, halo, hydrazine, hydrazide, hydroxyl, alkenyl, alkynl, ether, thiol, sulfonyl, seleno, ester, thioacid, borate, boronate, phospho, phosphono, phosphine, heterocyclic, enone, imine, aldehyde, hydroxylamine, keto, or amino substituted amino acid, or any combination thereof; an amino acid with a photoactivatable cross-linker; a spin-labeled amino acid; a fluorescent amino acid; an amino acid with a novel functional group; an amino acid that covalently or noncovalently interacts with another molecule; a metal binding amino acid; an amino acid that is amidated at a site that is not naturally amidated, a metal-containing amino acid; a radioactive amino acid; a photocaged and/or photoisomerizable amino acid; a biotin or biotin-analogue containing amino acid; a glycosylated or carbohydrate modified amino acid; a keto containing amino acid; amino acids comprising polyethylene glycol or polyether; a heavy atom substituted amino acid (e.g., an amino acid containing deuterium, tritium, ¹³C, ¹⁵N, or ¹⁸O); a chemically cleavable or photocleavable amino acid; an amino acid with an elongated side chain; an amino acid containing a toxic group; a sugar substituted amino acid, e.g., a sugar substituted serine or the like; a carbon-linked sugar-containing amino acid; a redox-active amino acid; an α-hydroxy containing acid; an amino thio acid containing amino acid; an α, α disubstituted amino acid; a β-amino acid; a cyclic amino acid other than proline; an O-methyl-L-tyrosine; an L-3-(2-naphthyl)alanine; a 3-methyl-phenylalanine; a σ-acetyl-L-phenylalanine; an O-4-allyl-L-tyrosine; a 4-propyl-L-tyrosine; a tri-O-acetyl-GlcNAc β-serine; an L-Dopa; a fluorinated phenylalanine; an isopropyl-L-phenylalanine; a p-azido-L-phenylalanine; a p-acyl-L-phenylalanine; a p-benzoyl-L-phenylalanine; an L-phosphoserine; a phosphonoserine; a phosphonotyrosine; a p-iodo-phenylalanine; a 4-fluorophenylglycine; a p-bromophenylalanine; a p-amino-L-phenylalanine; an isopropyl-L-phenylalanine; L-3-(2-naphthyl)alanine; D-3-(2-naphthyl)alanine (dNal); an amino-, isopropyl-, or O-allyl-containing phenylalanine analogue; a dopa, 0-methyl-L-tyrosine; a glycosylated amino acid; a p-(propargyloxy)phenylalanine; dimethyl-Lysine; hydroxy-proline; mercaptopropionic acid; methyl-lysine; 3-nitro-tyrosine; norleucine; pyro-glutamic acid; Z (Carbobenzoxyl); ε-Acetyl-Lysine; β-alanine; β-aspartic acid; β-cyclohexylalanine; aminobenzoyl derivative; aminobutyric acid (Abu); citrulline; aminohexanoic acid (Ahx); aminoisobutyric acid (AIB); cyclohexylalanine; d-cyclohexylalanine; cyclohexylglycine; hydroxyproline; nitro-arginine; nitro-phenylalanine; nitro-tyrosine; norvaline; octahydroindole carboxyl ate; ornithine (Orn); penicillamine (PEN); tetrahydroisoquinoline; diaminobutyric acid; diaminopimelic acid; pyroglutamic acid; homocysteine; homoserine; N-ε-dinitrophenyl-lysine; N-ε-methyl-lysine; N-ε-dimethyl-lysine; N,N,N-ε-trimethyl-lysine; acetamidomethyl protected amino acids and pegylated amino acids. Further examples of unnatural amino acids and amino acid analogs can be found in U.S. 20030108885, U.S. 20030082575, US20060019347 (paragraphs 410-418) and the references cited therein. The polypeptides of the invention can include further modifications including those described in US20060019347, paragraph 589.

“Nal” used herein refers to both L-1-naphthylalanine (L-1-Nal) and L-2-naphthylalanine (L-2-Nal).

In some embodiments, an amino acid can be replaced by a naturally-occurring, non-essential amino acid, e.g., taurine.

Alternatively, the GCRA peptides are cyclic peptides. GCRA cyclic peptides are prepared by methods known in the art. For example, macrocyclization is often accomplished by forming an amide bond between the peptide N- and C-termini, between a side chain and the N- or C-terminus [e.g., with K₃Fe(CN)₆ at pH 8.5] (Samson et al., Endocrinology, 137: 5182-5185 (1996)), or between two amino acid side chains, such as cysteine. See, e.g., DeGrado, Adv Protein Chem, 39: 51-124 (1988). In some embodiments, the GCRA peptides of the present invention are bicyclic peptides. In various aspects the GCRA peptides are [4,12; 7,15] bicycles.

In some GCRA peptides one or both members of one or both pairs of Cys residues which normally form a disulfide bond can be replaced by homocysteine, penicillamine, 3-mercaptoproline (Kolodziej et al. 1996 Int J Pept Protein Res 48:274); β,β dimethylcysteine (Hunt et al. 1993 Int JPept Protein Res 42:249) or diaminopropionic acid (Smith et al. 1978 J Med Chem 2 1:117) to form alternative internal cross-links at the positions of the normal disulfide bonds.

In addition, one or more disulfide bonds can be replaced by alternative covalent cross-links, e.g., an amide linkage (—CH2CH(O)NHCH 2- or —CH2NHCH(O)CH 2-), an ester linkage, a thioester linkage, a lactam bridge, a carbamoyl linkage, a urea linkage, a thiourea linkage, a phosphonate ester linkage, an alkyl linkage (—CH2CH2CH2CH2-), an alkenyl linkage (—CH 2CH═CHCH 2-), an ether linkage (—CH2CH2OCH2- or —CH2OCH2CH2-), a thioether linkage (—CH2CH2SCH2- or —CH2SCH2CH2-), an amine linkage (—CH2CH2NHCH2- or —CH2NHCH 2CH2-) or a thioamide linkage (—CH2CH(S)HNHCH 2- or —CH2NHCH(S)CH 2-). For example, Ledu et al. (Proc Nat'l Acad. Sci. 100:11263-78, 2003) describe methods for preparing lactam and amide cross-links. Exemplary GCRA peptides which include a lactam bridge include for example SP-370.

The GCRA peptides can have one or more conventional polypeptide bonds replaced by an alternative bond. Such replacements can increase the stability of the polypeptide. For example, replacement of the polypeptide bond between a residue amino terminal to an aromatic residue (e.g. Tyr, Phe, Trp) with an alternative bond can reduce cleavage by carboxy peptidases and may increase half-life in the digestive tract. Bonds that can replace polypeptide bonds include: a retro-inverso bond (C(O)—NH instead of NH—C(O); a reduced amide bond (NH—CH2); a thiomethylene bond (S—CH2 or CH2-S); an oxomethylene bond (0-CH 2 or CH2-O); an ethylene bond (CH2-CH2); a thioamide bond (C(S)—NH); a trans-olefine bond (CH═CH); a fluoro substituted trans-olefme bond (CF═CH); a ketomethylene bond (C(O)—CHR or CHR—C(O) wherein R is H or CH3; and a fluoro-ketomethylene bond (C(O)—CFR or CFR—C(O) wherein R is H or F or CH3.

The GCRA peptides can be modified using standard modifications. Modifications may occur at the amino (N—), carboxy (C—) terminus, internally or a combination of any of the preceding. In one aspect described herein, there may be more than one type of modification on the polypeptide. Modifications include but are not limited to: acetylation, amidation, biotinylation, cinnamoylation, farnesylation, formylation, myristoylation, palmitoylation, phosphorylation (Ser, Tyr or Thr), stearoylation, succinylation, sulfurylation and cyclisation (via disulfide bridges or amide cyclisation), and modification by Cys3 or Cys5. The GCRA peptides described herein may also be modified by 2,4-dinitrophenyl (DNP), DNP-lysine, modification by 7-Amino-4-methyl-coumarin (AMC), flourescein, NBD (7-Nitrobenz-2-Oxa-1,3-Diazole), p-nitro-anilide, rhodamine B, EDANS (5((2-aminoethyl)amino)naphthalene-1-sulfonic acid), dabcyl, dabsyl, dansyl, texas red, FMOC, and Tamra (Tetramethylrhodamine). The GCRA peptides described herein may also be conjugated to, for example, polyethylene glycol (PEG); alkyl groups (e.g., C1-C20 straight or branched alkyl groups); fatty acid radicals; combinations of PEG, alkyl groups and fatty acid radicals (See, U.S. Pat. No. 6,309,633; Soltero et al., 2001 Innovations in Pharmaceutical Technology 106-110); BSA and KLH (Keyhole Limpet Hemocyanin). The addition of PEG and other polymers which can be used to modify polypeptides of the invention is described in US2006019347 section IX.

Also included in the invention are peptides that biologically or functional equivalent to the peptides described herein. The term “biologically equivalent” or functional equivalent” is intended to mean that the compositions of the present invention are capable of demonstrating some or all of the cGMP production modulatory effects.

GCRA peptides can also include derivatives of GCRA peptides which are intended to include hybrid and modified forms of GCRA peptides in which certain amino acids have been deleted or replaced and modifications such as where one or more amino acids have been changed to a modified amino acid or unusual amino acid and modifications such as glycosylation so long the modified form retains the biological activity of GCRA peptides. By retaining the biological activity, it is meant that cGMP and or apoptosis is induced by the GCRA peptide, although not necessarily at the same level of potency as that of a naturally-occurring GCRA peptide identified.

Preferred variants are those that have conservative amino acid substitutions made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a GCRA polypeptide is replaced with another amino acid residue from the same side chain family. Alternatively, in another embodiment, mutations can be introduced randomly along all or part of a GCRA coding sequence, such as by saturation mutagenesis, and the resultant mutants can be screened to identify mutants that retain activity.

The GCRA peptides of the invention also include analogs that contain an α-aminoadipic acid (Aad), preferably at the 3rd position from the N-terminus of each peptide or at the position to the N-terminal side next to the first cysteine (“Cys”) residue. See, for example, US 2014/0256762 filed Mar. 13, 2014 which is hereby incorporated by reference in its entirety for all purposes.

The GCRA peptides of the invention also include anoalogs, where 5-aminosalicylic acid (“5-ASA”; also called mesalamine or mesalazine) or its derivative or pharmaceutically acceptable salt thereof is covalently linked to the N terminus and/or the C terminus of a GCRA peptide (referred herein “5-ASA GCRA analog peptide”) (see, US 20140256762, filed Mar. 13, 2014 which is hereby incorporated in its entirety for all purposes). These peptides are biologically inactive or biologically less active than a GCRA peptide alone. However, upon cleavage of the glycosidic bond between peptide and sugar residues of the 5-ASA molecule or the PEG molecule by sugar hydrolases produced by colon bacteria, released GCRA peptide and 5-ASA molecule then produce a colon-specific synergistic effect to stimulate cGMP production, to induce apoptosis, and/or to enhance anti-inflammation. Such 5-ASA GCRA analog peptides also prevent or reduce the potential side effect of a GCRA peptide before reaching to colon. In some embodiments, 5-ASA or its derivative or pharmaceutically acceptable salt thereof is covalently linked to the N terminus and/or the C terminus of a GCRA peptide (referred herein “5-ASA GCRA analog peptide”). Preferably, the derivative is sulfasalazine.

In some embodiments, the 5-ASA GCRA analog peptide includes:

[5-ASA]-GCRA (formula A),

GCRA-[5-ASA] (formula B) or

[5-ASA]-GCRA-[5-ASA] (formula C).

A skilled artisan would readily recognize that the N-terminus of the peptide is on the left side and the C-terminus of the peptide is on the right side in these formulas.

In a merely illustrative embodiment, a 5-ASA GCRA analog peptide of the invention has the following formula:

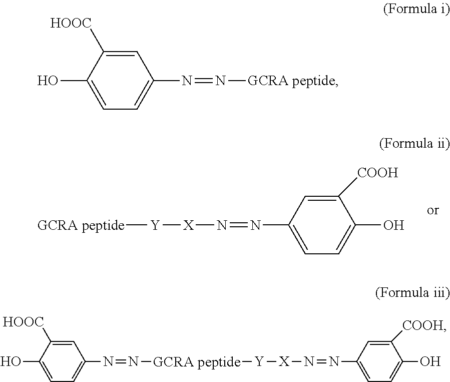

wherein X is absent, aryl or alkyl and Y is absent or any function group that reacts with the carboxyl group of the GCRA peptide. A skilled artisan could readlily determine the function groups that can react with the carboxyl group of the GCRA peptide. In certain embodiments, when the last amino acid (i.e., the amino acid at the most c-terminus end) in the GCRA peptide contains a free NH₂ group in its side chain (for example, lysine), X and Y can be absent. 5-ASA GCRA analog peptides described herein are biologically inactive or biologically less active than a GCRA peptide alone. However, upon cleavage of the glycosidic bond between peptide and sugar residues of the 5-ASA molecule or the PEG molecule by sugar hydrolases produced by colon bacteria, released GCRA peptide and 5-ASA molecule then produce a colon-specific synergistic effect to stimulate cGMP production, to induce apoptosis, and/or to enhance anti-inflammation. Such 5-ASA GCRA analog peptides also prevent or reduce the potential side effect of a GCRA peptide before reaching to colon.

In some embodiments, the 5-ASA GCRA analog peptides described herein are formulated in a pH dependent release form. Alternatively, such analog peptides are formulated in a form that releases the peptides at a specific region of the gastrointestinal (GI) tract (e.g., duodenum, jejunum, ileum, terminal ileum, or ascending colon). The formulation may contain an inert carrier coated with 5-ASA GCRA analog peptides and an enteric coating which releases the peptides at a specific pH (such as pH5 or pH7). Preferred pH for duodenum or jejunum release is pH 4.5-5.5 or pH 5.5-6.5. Preferred pH for ileum, terminal ileum, or ascending colon release is pH 5.5-6.5 or pH 6.5-7.5. Preferably, the inert carrier is a selected from mannitol, lactose, a microcrystalline cellulose, or starch.

The term “consisting essentially of” includes peptides that are identical to a recited sequence (any one from Tables 1-7) and other sequences that do not differ substantially in terms of either structure or function. For the purpose of the present application, a peptide differs substantially if its structure varies by more than three amino acids from a peptide of any one from Tables 1-7 or if its activation of cellular cGMP production is reduced or enhanced by more than 50%. Preferably, substantially similar peptides should differ by no more than two amino acids and not differ by more than about 25% with respect to activating cGMP production.

Also included within the meaning of substantially homologous is any GCRA peptide which may be isolated by virtue of cross-reactivity with antibodies to the GCRA peptide.

TABLE 1
GCRA Peptides (SP-304 and Derivatives)

| | | | SEQ |
|---|---|---|---|
| | Position of | | ID |
| Name | Disulfide bonds | Structure | NO |
| SP-304 | C4:C12, C7:C15 | Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Leu¹⁶ | 1 |
| SP-326 | C3:C11, C6:C14 | Asp¹-Glu²-Cys³-Glu⁴-Leu⁵-Cys⁶-Val⁷-Asn⁸-Val⁹-Ala¹⁰-Cys¹¹-Thr¹²-Gly¹³-Cys¹⁴-Leu¹⁵ | 2 |
| SP-327 | C3:C11, C6:C14 | Asp¹-Glu²-Cys³-Glu⁴-Leu⁵-Cys⁶-Val⁷-Asn⁸-Val⁹-Ala¹⁰-Cys¹¹-Thr¹²-Gly¹³-Cys¹⁴ | 3 |
| SP-328 | C2:C10, C5:C13 | Glu¹-Cys²-Glu³-Leu⁴-Cys⁵-Val⁶-Asn⁷-Val⁸-Ala⁹-Cys¹⁰-Thr¹¹-Gly¹²-Cys¹³-Leu¹⁴ | 4 |
| SP-329 | C2:C10, C5:C13 | Glu¹-Cys²-Glu³-Leu⁴-Cys⁵-Val⁶-Asn⁷-Val⁸-Ala⁹-Cys¹⁰-Thr¹¹-Gly¹²-Cys¹³ | 5 |
| SP-330 | C1:C9, C4:C12 | Cys¹-Glu²-Leu³-Cys⁴-Val⁵-Asn⁶-Val⁷-Ala⁸-Cys⁹-Thr¹⁰-Gly¹¹-Cys¹²-Leu¹³ | 6 |
| SP-331 | C1:C9, C4:C12 | Cys¹-Glu²-Leu³-Cys⁴-Val⁵-Asn⁶-Val⁷-Ala⁸-Cys⁹-Thr¹⁰-Gly¹¹-Cys¹² | 7 |
| SP332 | C4:C12, C7:C15 | Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶ | 8 |
| SP-333 | C4:C12, C7:C15 | dAsn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶ | 9 |
| SP-334 | C4:C12, C7:C15 | dAsn¹-dAsp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶ | 10 |
| SP-335 | C4:C12, C7:C15 | dAsn¹-dAsp²-dGlu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶ | 11 |
| SP-336 | C4:C12, C7:C15 | dAsn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Leu¹⁶ | 12 |
| SP-337 | C4:C12, C7:C15 | dAsn¹-Asp²-Glu³-Cys⁴-Glu⁵-dLeu⁶-Cys⁷-Val⁸-Asn⁹-Val¹⁰-Ala¹¹-Cys¹²-Thr¹³-Gly¹⁴-Cys¹⁵-dLeu¹⁶ | 13 |
| SP-338 | C4:C12, C7:C15 | Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵ | 14 |
| SP-342 | C4:C12, C7:C15 | PEG3-Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶-PEG3 | 15 |
| SP-343 | C4:C12, C7:C15 | PEG3-dAsn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶-PEG3 | 16 |
| SP-344 | C4:C12, C7:C15 | PEG3-dAsn¹-dAsp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶-PEG3 | 17 |
| SP-347 | C4:C12, C7:C15 | dAsn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶-PEG3 | 18 |
| SP-348 | C4:C12, C7:C15 | PEG3-Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶ | 19 |
| SP-350 | C4:C12, C7:C15 | PEG3-dAsn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶ | 20 |
| SP-352 | C4:C12, C7:C15 | Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶-PEG3 | 21 |
| SP-358 | C4:C12, C7:C15 | PEG3-dAsn¹-dAsp²-dGlu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶- | 22 |
| | | PEG3 | |
| SP-359 | C4:C12, C7:C15 | PEG3-dAsn¹-dAsp²-dGlu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶ | 23 |
| SP-360 | C4:C12, C7:C15 | dAsn¹-dAsp²-dGlu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶-PEG3 | 24 |
| SP-361 | C4:C12, C7:C15 | dAsn¹-dAsp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶-PEG3 | 25 |
| SP-362 | C4:C12, C7:C15 | PEG3-dAsn¹-dAsp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶ | 26 |
| SP-368 | C4:C12, C7:C15 | dAsn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dNal¹⁶ | 27 |
| SP-369 | C4:C12, C7:C15 | dAsn¹-Asp²-Glu³-Cys⁴-Glu⁵-Leu⁶-Cys⁷-AIB⁸-Asn⁹-AIB¹⁰-Ala¹¹-Cys¹²-Thr¹³-Gly¹⁴-Cys¹⁵-dLeu¹⁶ | 28 |
| SP-370 | C4:C12, 7:15 | dAsn¹-Asp²-Glu³-Cys⁴-Glu⁵-Leu⁶-Asp[Lactam]⁷-Val⁸-Asn⁹-Val¹⁰-Ala¹¹-Cys¹²-Thr¹³-Gly¹⁴-Orn¹⁵-dLeu¹ | 29 |
| SP-371 | C4:C12, C7:C15 | dAsn¹-Asp²-Glu³-Cys⁴-Glu⁵-Tyr⁶-Cys⁷-Val⁸-Asn⁹-Val¹⁰-Ala¹¹-Cys¹²-Thr¹³-Gly¹⁴-Cys¹⁵-dLeu¹⁶ | 30 |
| SP-372 | C4:C12, C7:C15 | dAsn¹-Asp²-Glu³-Cys⁴-Glu⁵-Ser⁶-Cys⁷-Val⁸-Asn⁹-Val¹⁰-Ala¹¹-Cys¹²-Thr¹³-Gly¹⁴-Cys¹⁵-dLeu¹⁶ | 31 |
| N1 | C4:C12, C7:C15 | PEG3-dAsn¹-Asp²-Glu³-Cys⁴-Glu⁵-Tyr⁶-Cys⁷-Val⁸-Asn⁹-Val¹⁰-Ala¹¹-Cys¹²-Thr¹³-Gly¹⁴-Cys¹⁵-dLeu¹⁶-PEG3 | 32 |
| N2 | C4:C12, C7:C15 | PEG3-dAsn¹-Asp²-Glu³-Cys⁴-Glu⁵-Tyr⁶-Cys⁷-Val⁸-Asn⁹-Val¹⁰-Ala¹¹-Cys¹²-Thr¹³-Gly¹⁴-Cys¹⁵-dLeu¹⁶ | 33 |
| N3 | C4:C12, C7:C15 | dAsn¹-Asp²-Glu³-Cys⁴-Glu⁵-Tyr⁶-Cys⁷-Val⁸-Asn⁹-Val¹⁰-Ala¹¹-Cys¹²-Thr¹³-Gly¹⁴-Cys¹⁵-dLeu¹⁶-PEG3 | 34 |
| N4 | C4:C12, C7:C15 | PEG3-dAsn¹-Asp²-Glu³-Cys⁴-Glu⁵-Ser⁶-Cys⁷-Val⁸-Asn⁹-Val¹⁰-Ala¹¹-Cys¹²-Thr¹³-Gly¹⁴-Cys¹⁵-dLeu¹⁶-PEG3 | 35 |
| N5 | C4:C12, C7:C15 | PEG3-dAsn¹-Asp²-Glu³-Cys⁴-Glu⁵-Ser⁶-Cys⁷-Val⁸-Asn⁹-Val¹⁰-Ala¹¹-Cys¹²-Thr¹³-Gly¹⁴-Cys¹⁵-dLeu¹⁶ | 36 |
| N6 | C4:C12, C7:C15 | dAsn¹-Asp²-Glu³-Cys⁴-Glu⁵-Ser⁶-Cys⁷-Val⁸-Asn⁹-Val¹⁰-Ala¹¹-Cys¹²-Thr¹³-Gly¹⁴-Cys¹⁵-dLeu¹⁶-PEG3 | 37 |
| N7 | C4:C12, C7:C15 | Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Ser¹⁶ | 38 |
| N8 | C4:C12, C7:C15 | PEG3-Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Ser¹⁶-PEG3 | 39 |
| N9 | C4:C12, C7:C15 | PEG3-Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Ser¹⁶ | 40 |
| N10 | C4:C12, C7:C15 | Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Ser¹⁶-PEG3 | 41 |
| N11 | C4:C12, C7:C15 | PEG3-Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dSer¹⁶-PEG3 | 42 |
| N12 | C4:C12, C7:C15 | PEG3-Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dSer¹⁶ | 43 |
| N13 | C4:C12, C7:C15 | Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dSer¹⁶-PEG3 | 44 |
| Formula I | C4:C12, C7:C15 | Asn¹-Asp²-Glu³-Cys⁴-Xaa⁵-Xaa⁶-Cys⁷-Xaa⁸-Xaa⁹-Xaa¹⁰-Aaa¹¹-Cys¹²-Aaa¹³-Xaa¹⁴-Cys¹⁵-Xaa¹⁶ | 45 |
| Formula II | C4:C12, C7:C15 | Xaaₙ₁-Cys⁴-Xaa⁵-Xaa⁶-Cys⁷-Xaa⁸-Xaa⁹-Xaa¹⁰-Xaa¹¹-Cys¹²-Xaa¹³-Xaa¹⁴-Cys¹⁵-Xaaₙ₂ | 46 |
| Formula | 4:12, 7:15 | Xaaₙ₁-Maa⁴-Glu⁵-Xaa⁶-Maa⁷-Val⁸-Asn⁹-Val¹⁰-Ala¹¹-Maa¹²-Thr¹³-Gly¹⁴-Maa¹⁵-Xaaₙ₂ | 47 |
| III | | | |
| Formula | 4:12, 7:15 | Xaaₙ₁-Maa⁴-Xaa⁵-Xaa⁶-Maa⁷-Xaa⁸-Xaa⁹-Xaa¹⁰-Xaa¹¹-Maa¹²-Xaa¹³-Xaa¹⁴-Maa¹⁵-Xaaₙ₂ | 48 |
| IV | | | |
| Formula V | C4:C12, C7:C15 | Asn¹-Asp²-Asp³-Cys⁴-Xaa⁵-Xaa⁶-Cys⁷-Xaa⁸-Asn⁹-Xaa¹⁰-Xaa¹¹-Cys¹²-Xaa¹³-Xaa¹⁴-Cys¹⁵-Xaa¹⁶ | 49 |
| Formula | C4:C12, C7:C15 | dAsn¹-Glu²-Glu³-Cys⁴-Xaa⁵-Xaa⁶-Cys⁷-Xaa⁸-Asn⁹-Xaa¹⁰-Xaa¹¹-Cys¹²-Xaa¹³-Xaa¹⁴-Cys¹⁵-d-Xaa¹⁶ | 50 |
| VI | | | |
| Formula | C4:C12, C7:C15 | dAsn¹-dGlu²-Asp³-Cys⁴-Xaa⁵-Xaa⁶-Cys⁷-Xaa⁸-Asn⁹-Xaa¹⁰-Xaa¹¹-Cys¹²-Xaa¹³-Xaa¹⁴-Cys¹⁵-d-Xaa¹⁶ | 51 |
| VII-a | | | |
| Formula | C4:C12, C7:C15 | dAsn¹-dAsp²-Glu³-Cys⁴-Xaa⁵-Xaa⁶-Cys⁷-Xaa⁸-Asn⁹-Xaa¹⁰-Xaa¹¹-Cys¹²-Xaa¹³-Xaa¹⁴-Cys¹⁵-d-Xaa¹⁶ | 52 |
| VII-b | | | |
| Formula | C4:C12, C7:C15 | dAsn¹-dAsp²-dGlu³-Cys⁴-Xaa⁵-Xaa⁶-Cys⁷-Xaa⁸-Tyr⁹-Xaa¹⁰-Xaa¹¹-Cys¹²-Xaa¹³-Xaa¹⁴-Cys¹⁵-d-Xaa¹⁶ | 53 |
| VIII | | | |
| Formula | C4:C12, C7:C15 | dAsn¹-dGlu²-dGlu³-Cys⁴-Xaa⁵-Xaa⁶-Cys⁷-Xaa⁸-Tyr⁹-Xaa¹⁰-Xaa¹¹-Cys¹²-Xaa¹³-Xaa¹⁴-Cys¹⁵-d-Xaa¹⁶ | 54 |
| IX | | | |
| Formula | C4:C12, C7:C15 | Xaaₙ₁-Cys⁴-Xaa⁵-Xaa⁶-Xaa⁷-Xaa⁸-Xaa⁹-Xaa¹⁰-Xaa¹¹-Cys¹²-Xaa¹³-Xaa¹⁴-Xaa¹⁵-Xaaₙ₂¹⁶ | 250 |
| XXI | | | |

TABLE 2
Linaclotide and Derivatives

| | Position of | | SEQ ID |
|---|---|---|---|
| Name | Disulfide Bonds | Structure | NO: |
| SP-339 | C1:C6, C2:C10, C5:C13 | Cys¹-Cys²-Glu³-Tyr⁴-Cys⁵-Cys⁶-Asn⁷-Pro⁸-Ala⁹-Cys¹⁰-Thr¹¹-Gly¹²-Cys¹³-Tyr¹⁴ | 55 |
| (linaclotide) | | | |
| SP-340 | C1:C6, C2:C10, C5:C13 | Cys¹-Cys²-Glu³-Tyr⁴-Cys⁵-Cys⁶-Asn⁷-Pro⁸-Ala⁹-Cys¹⁰-Thr¹¹-Gly¹²-Cys¹³ | 56 |
| SP-349 | C1:C6, C2:C10, C5:C13 | PEG3-Cys¹-Cys²-Glu³-Tyr⁴-Cys⁵-Cys⁶-Asn⁷-Pro⁸-Ala⁹-Cys¹⁰-Thr¹¹-Gly¹²-Cys¹³-Tyr¹⁴- | 57 |
| | | PEG3 | |
| SP-353 | C3:C8, C4:C12, C7:C15 | Asn¹-Phe²-Cys³-Cys⁴-Glu⁵-Ser⁶-Cys⁷-Cys⁸-Asn⁹-Pro¹⁰-Ala¹¹-Cys¹²-Thr¹³-Gly¹⁴-Cys¹⁵- | 58 |
| | | Tyr¹⁶ | |
| SP-354 | C3:C8, C4:C12, C7:C15 | Asn¹-Phe²-Cys³-Cys⁴-Glu⁵-Phe⁶-Cys⁷-Cys⁸-Asn⁹-Pro¹⁰-Ala¹¹-Cys¹²-Thr¹³-Gly¹⁴-Cys¹⁵- | 59 |
| | | Tyr¹⁶ | |
| SP-355 | C1:C6, C2:C10, C5:C13 | Cys¹-Cys²-Glu³-Tyr⁴-Cys⁵-Cys⁶-Asn⁷-Pro⁸-Ala⁹-Cys¹⁰-Thr¹¹-Gly¹²-Cys¹³-dTyr¹⁴ | 60 |
| SP-357 | C1:C6, C2:C10, C5:C13 | PEG3-Cys¹-Cys²-Glu³-Tyr⁴-Cys⁵-Cys⁶-Asn⁷-Pro⁸-Ala⁹-Cys¹⁰-Thr¹¹-Gly¹²-Cys¹³-Tyr¹⁴ | 61 |
| SP-374 | C3:C8, C4:C12, C7:C15 | Asn¹-Phe²-Cys³-Cys⁴-Glu⁵-Thr⁶-Cys⁷-Cys⁸-Asn⁹-Pro¹⁰-Ala¹¹-Cys¹²-Thr¹³-Gly¹⁴-Cys¹⁵- | 62 |
| | | Tyr¹⁶ | |
| SP-375 | C3:C8, C4:C12, C7:C15 | Asn¹-Phe²-Cys³-Cys⁴-Glu⁵-Ser⁶-Cys⁷-Cys⁸-Asn⁹-Pro¹⁰-Ala¹¹-Cys¹²-Thr¹³-Gly¹⁴-Cys¹⁵- | 63 |
| | | dTyr¹⁶ | |
| SP-376 | C3:C8, C4:C12, C7:C15 | dAsn¹-Phe²-Cys³-Cys⁴-Glu⁵-Ser⁶-Cys⁷-Cys⁸-Asn⁹-Pro¹⁰-Ala¹¹-Cys¹²-Thr¹³-Gly¹⁴-Cys¹⁵- | 64 |
| | | Tyr¹⁶ | |
| SP-377 | C3:C8, C4:C12, C7:C15 | dAsn¹-Phe²-Cys³-Cys⁴-Glu⁵-Ser⁶-Cys⁷-Cys⁸-Asn⁹-Pro¹⁰-Ala¹¹-Cys¹²-Thr¹³-Gly¹⁴-Cys¹⁵- | 65 |
| | | dTyr¹⁶ | |
| SP-378 | C3:C8, C4:C12, C7:C15 | Asn¹-Phe²-Cys³-Cys⁴-Glu⁵-Thr⁶-Cys⁷-Cys⁸-Asn⁹-Pro¹⁰-Ala¹¹-Cys¹²-Thr¹³-Gly¹⁴-Cys¹⁵- | 66 |
| | | dTyr¹⁶ | |
| SP-379 | C3:C8, C4:C12, C7:C15 | dAsn¹-Phe²-Cys³-Cys⁴-Glu⁵-Thr⁶-Cys⁷-Cys⁸-Asn⁹-Pro¹⁰-Ala¹¹-Cys¹²-Thr¹³-Gly¹⁴-Cys¹⁵- | 67 |
| | | Tyr¹⁶ | |
| SP-380 | C3:C8, C4:C12, C7:C15 | dAsn¹-Phe²-Cys³-Cys⁴-Glu⁵-Thr⁶-Cys⁷-Cys⁸-Asn⁹-Pro¹⁰-Ala¹¹-Cys¹²-Thr¹³-Gly¹⁴-Cys¹⁵- | 68 |
| | | dTyr¹⁶ | |
| SP-381 | C3:C8, C4:C12, C7:15 | Asn¹-Phe²-Cys³-Cys⁴-Glu⁵-Phe⁶-Cys⁷-Cys⁸-Asn⁹-Pro¹⁰-Ala¹¹-Cys¹²-Thr¹³-Gly¹⁴-Cys¹⁵- | 69 |
| | | dTyr¹⁶ | |
| SP-382 | C3:C8, C4:C12, C7:15 | dAsn¹-Phe²-Cys³-Cys⁴-Glu⁵-Phe⁶-Cys⁷-Cys⁸-Asn⁹-Pro¹⁰-Ala¹¹-Cys¹²-Thr¹³-Gly¹⁴-Cys¹⁵- | 70 |
| | | Tyr¹⁶ | |
| SP-383 | C3:C8, C4:C12, C7:15 | dAsn¹-Phe²-Cys³-Cys⁴-Glu⁵-Phe⁶-Cys⁷-Cys⁸-Asn⁹-Pro¹⁰-Ala¹¹-Cys¹²-Thr¹³-Gly¹⁴-Cys¹⁵- | 71 |
| | | dTyr¹⁶ | |
| SP384 | C1:C6, C2:C10, C5:C13 | Cys¹-Cys²-Glu³-Tyr⁴-Cys⁵-Cys⁶-Asn⁷-Pro⁸-Ala⁹-Cys¹⁰-Thr¹¹-Gly¹²-Cys¹³-Tyr¹⁴-PEG3 | 72 |
| N14 | C1:C6, C2:C10, C5:C13 | PEG3-Cys¹-Cys²-Glu³-Tyr⁴-Cys⁵-Cys⁶-Asn⁷-Pro⁸-Ala⁹-Cys¹⁰-Thr¹¹-Gly¹²-Cys¹³-PEG3 | 73 |
| N15 | C1:C6, C2:C10, C5:C13 | PEG3-Cys¹-Cys²-Glu³-Tyr⁴-Cys⁵-Cys⁶-Asn⁷-Pro⁸-Ala⁹-Cys¹⁰-Thr¹¹-Gly¹²-Cys¹³ | 74 |
| N16 | C1:C6, C2:C10, C5:C13 | Cys¹-Cys²-Glu³-Tyr⁴-Cys⁵-Cys⁶-Asn⁷-Pro⁸-Ala⁹-Cys¹⁰-Thr¹¹-Gly¹²-Cys¹³-PEG3 | 75 |
| N17 | C3:C8, C4:C12, C7:C15 | PEG3-Asn¹-Phe²-Cys³-Cys⁴-Glu⁵-Ser⁶-Cys⁷-Cys⁸-Asn⁹-Pro¹⁰-Ala¹¹-Cys¹²-Thr¹³-Gly¹⁴- | 76 |
| | | Cys¹⁵-Tyr¹⁶-PEG3 | |
| N18 | C3:C8, C4:C12, C7:C15 | PEG3-Asn¹-Phe²-Cys³-Cys⁴-Glu⁵-Ser⁶-Cys⁷-Cys⁸-Asn⁹-Pro¹⁰-Ala¹¹-Cys¹²-Thr¹³-Gly¹⁴- | 77 |
| | | Cys¹⁵-Tyr¹⁶ | |
| N19 | C3:C8, C4:C12, C7:C15 | Asn¹-Phe²-Cys³-Cys⁴-Glu⁵-Ser⁶-Cys⁷-Cys⁸-Asn⁹-Pro¹⁰-Ala¹¹-Cys¹²-Thr¹³-Gly¹⁴-Cys¹⁵- | 78 |
| | | Tyr¹⁶-PEG3 | |
| N20 | C3:C8, C4:C12, C7:C15 | PEG3-Asn¹-Phe²-Cys³-Cys⁴-Glu⁵-Phe⁶-Cys⁷-Cys⁸-Asn⁹-Pro¹⁰-Ala¹¹-Cys¹²-Thr¹³-Gly¹⁴- | 79 |
| | | Cys¹⁵-Tyr¹⁶-PEG3 | |
| N21 | C3:C8, C4:C12, C7:C15 | PEG3-Asn¹-Phe²-Cys³-Cys⁴-Glu⁵-Phe⁶-Cys⁷-Cys⁸-Asn⁹-Pro¹⁰-Ala¹¹-Cys¹²-Thr¹³-Gly¹⁴- | 80 |
| | | Cys¹⁵-Tyr¹⁶ | |
| N22 | C3:C8, C4:C12, C7:C15 | Asn¹-Phe²-Cys³-Cys⁴-Glu⁵-Phe⁶-Cys⁷-Cys⁸-Asn⁹-Pro¹⁰-Ala¹¹-Cys¹²-Thr¹³-Gly¹⁴-Cys¹⁵- | 81 |
| | | Tyr¹⁶-PEG3 | |
| N23 | C3:C8, C4:C12, C7:C15 | PEG3-Asn¹-Phe²-Cys³-Cys⁴-Glu⁵-Tyr⁶-Cys⁷-Cys⁸-Asn⁹-Pro¹⁰-Ala¹¹-Cys¹²-Thr¹³-Gly¹⁴- | 82 |
| | | Cys¹⁵-Tyr¹⁶-PEG3 | |
| N24 | C3:C8, C4:C12, C7:C15 | PEG3-Asn¹-Phe²-Cys³-Cys⁴-Glu⁵-Tyr⁶-Cys⁷-Cys⁸-Asn⁹-Pro¹⁰-Ala¹¹-Cys¹²-Thr¹³-Gly¹⁴- | 83 |
| | | Cys¹⁵-Tyr¹⁶ | |
| N25 | C3:C8, C4:C12, C7:C15 | Asn¹-Phe²-Cys³-Cys⁴-Glu⁵-Tyr⁶-Cys⁷-Cys⁸-Asn⁹-Pro¹⁰-Ala¹¹-Cys¹²-Thr¹³-Gly¹⁴-Cys¹⁵- | 84 |
| | | Tyr¹⁶-PEG3 | |
| N26 | C1:C6, C2:C10, C5:C13 | Cys¹-Cys²-Glu³-Ser⁴-Cys⁵-Cys⁶-Asn⁷-Pro⁸-Ala⁹-Cys¹⁰-Thr¹¹-Gly¹²-Cys¹³-Tyr¹⁴ | 85 |
| N27 | C1:C6, C2:C10, C5:C13 | Cys¹-Cys²-Glu³-Phe⁴-Cys⁵-Cys⁶-Asn⁷-Pro⁸-Ala⁹-Cys¹⁰-Thr¹¹-Gly¹²-Cys¹³-Tyr¹⁴ | 86 |
| N28 | C1:C6, C2:C10, C5:C13 | Cys¹-Cys²-Glu³-Ser⁴-Cys⁵-Cys⁶-Asn⁷-Pro⁸-Ala⁹-Cys¹⁰-Thr¹¹-Gly¹²-Cys¹³- | 87 |
| N29 | C1:C6, C2:C10, C5:C13 | Cys¹-Cys²-Glu³-Phe⁴-Cys⁵-Cys⁶-Asn⁷-Pro⁸-Ala⁹-Cys¹⁰-Thr¹¹-Gly¹²-Cys¹³ | 88 |
| N30 | 1:6, 2:10, 5:13 | Pen¹-Pen²-Glu³-Tyr⁴-Pen⁵-Pen⁶-Asn⁷-Pro⁸-Ala⁹-Pen¹⁰-Thr¹¹-Gly¹²-Pen¹³-Tyr¹⁴ | 89 |
| N31 | 1:6, 2:10, 5:13 | Pen¹-Pen²-Glu³-Tyr⁴-Pen⁵-Pen⁶-Asn⁷-Pro⁸-Ala⁹-Pen¹⁰-Thr¹¹-Gly¹²-Pen¹³ | 90 |
| Formula X | C9:C14, C10:C18, | Xaa¹-Xaa²-Xaa³-Xaa⁴-Xaa⁵-Xaa⁶-Asn⁷-Tyr⁸-Cys⁹-Cys¹⁰-Xaa¹¹-Tyr¹²-Cys¹³-Cys¹⁴-Xaa¹⁵- | 91 |
| | C13:C21 | Xaa¹⁶-Xaa¹⁷-Cys¹⁸-Xaa¹⁹-Xaa²⁰-Cys²¹-Xaa²² | |
| Formula XI | C9:C14, C10:C18, | Xaa¹-Xaa²-Xaa³-Xaa⁴-Xaa⁵-Xaa⁶-Asn⁷-Phe⁸-Cys⁹-Cys¹⁰-Xaa¹¹-Phe¹²-Cys¹³-Cys¹⁴-Xaa¹⁵- | 92 |
| | C13:C21 | Xaa¹⁶-Xaa¹⁷-Cys¹⁸-Xaa¹⁹-Xaa²⁰-Cys²¹-Xaa²² | |
| Formula XII | C3:C8, C4:C12, C7:C15 | Asn¹-Phe²-Cys³-Cys⁴-Xaa⁵-Phe⁶-Cys⁷-Cys⁸-Xaa⁹-Xaa¹⁰-Xaa¹¹-Cys¹²-Xaa¹³-Xaa¹⁴-Cys¹⁵- | 93 |
| | | Xaa¹⁶ | |
| Formula XIII | 3:8, 4:12, 7:15 | Asn¹-Phe²-Pen³-Cys⁴-Xaa⁵-Phe⁶-Cys⁷-Pen⁸-Xaa⁹-Xaa¹⁰-Xaa¹¹-Cys¹²-Xaa¹³-Xaa¹⁴-Cys¹⁵- | 94 |
| | | Xaa¹⁶ | |
| Formula XIV | 3:8, 4:12, 7:15 | Asn¹-Phe²-Maa³-Maa⁴-Xaa⁵-Xaa⁶-Maa⁷-Maa⁸-Xaa⁹-Xaa¹⁰-Xaa¹¹-Maa¹²-Xaa¹³-Xaa¹⁴- | 95 |
| | | Maa¹⁵-Xaa¹⁶ | |
| Formula XV | 1:6, 2:10, 5:13 | Maa¹-Maa²-Glu³-Xaa⁴-Maa⁵-Maa⁶-Asn⁷-Pro⁸-Ala⁹-Maa¹⁰-Thr¹¹-Gly¹²-Maa¹³-Tyr¹⁴ | 96 |
| Formula XVI | 1:6, 2:10, 5:13 | Maa¹-Maa²-Glu³-Xaa⁴-Maa⁵-Maa⁶-Asn⁷-Pro⁸-Ala⁹-Maa¹⁰-Thr¹¹-Gly¹²-Maa¹³ | 97 |
| Formula XVII | 1:6, 2:10, 5:13 | Xaaₙ₃-Maa¹-Maa²-Xaa³-Xaa⁴-Maa⁵-Maa⁶-Xaa⁷-Xaa⁸-Xaa⁹-Maa¹⁰-Xaa¹¹-Xaa¹²-Maa¹³- | 98 |
| | | Xaaₙ₂ | |

TABLE 3
GCRA Peptides

| | Position of | | SEQ ID |
|---|---|---|---|
| Name | Disulfide bonds | Structure | NO: |
| SP-363 | C4:C12, C7:C15 | dAsn¹-Asp²-Glu³-Cys⁴-Glu⁵-Leu⁶-Cys⁷-Val⁸-Asn⁹-Val¹⁰-Ala¹¹-Cys¹²-Thr¹³- | 99 |
| | | Gly¹⁴-Cys¹⁵-dLeu-AMIDE¹⁶ | |
| SP-364 | C4:C12, C7:C15 | dAsn¹-Asp²-Glu³-Cys⁴-Glu⁵-Leu⁶-Cys⁷-Val⁸-Asn⁹-Val¹⁰-Ala¹¹-Cys¹²-Thr¹³- | 100 |
| | | Gly¹⁴-Cys¹⁵-dSer¹⁶ | |
| SP-365 | C4:C12, C7:C15 | dAsn¹-Asp²-Glu³-Cys⁴-Glu⁵-Leu⁶-Cys⁷-Val⁸-Asn⁹-Val¹⁰-Ala¹¹-Cys¹²-Thr¹³- | 101 |
| | | Gly¹⁴-Cys¹⁵-dSer-AMIDE¹⁶ | |
| SP-366 | C4:C12, C7:C15 | dAsn¹-Asp²-Glu³-Cys⁴-Glu⁵-Leu⁶-Cys⁷-Val⁸-Asn⁹-Val¹⁰-Ala¹¹-Cys¹²-Thr¹³- | 102 |
| | | Gly¹⁴-Cys¹⁵-dTyr¹⁶ | |
| SP-367 | C4:C12, C7:C15 | dAsn¹-Asp²-Glu³-Cys⁴-Glu⁵-Leu⁶-Cys⁷-Val⁸-Asn⁹-Val¹⁰-Ala¹¹-Cys¹²-Thr¹³- | 103 |
| | | Gly¹⁴-Cys¹⁵-dTyr-AMIDE¹⁶ | |
| SP-373 | C4:C12, C7:C15 | Pyglu¹-Asp²-Glu³-Cys⁴-Glu⁵-Leu⁶-Cys⁷-Val⁸-Asn⁹-Val¹⁰-Ala¹¹-Cys¹²-Thr¹³- | 104 |
| | | Gly¹⁴-Cys¹⁵-dLeu-AMIDE¹⁶ | |
| / | C4:C12, C7:C15 | Pyglu¹-Asp²-Glu³-Cys⁴-Glu⁵-Leu⁶-Cys⁷-Val⁸-Asn⁹-Val¹⁰-Ala¹¹-Cys¹²-Thr¹³- | 251 |
| | | Gly¹⁴-Cys¹⁵-Leu¹⁶ | |
| SP- | C4:C12, C7:C15 | PEG3-Asn¹-Asp²-Glu³-Cys⁴-Glu⁵-Leu⁶-Cys⁷-Val⁸-Asn⁹-Val¹⁰-Ala¹¹-Cys¹²-Thr¹³- | 105 |
| 304diPEG | | Gly¹⁴-Cys¹⁵-Leu¹⁶-PEG3 | |
| SP-304N- | C4:C12, C7:C15 | PEG3-Asn¹-Asp²-Glu³-Cys⁴-Glu⁵-Leu⁶-Cys⁷-Val⁸-Asn⁹-Val¹⁰-Ala¹¹-Cys¹²-Thr¹³- | 106 |
| PEG | | Gly¹⁴-Cys¹⁵-Leu¹⁶ | |
| SP-304C- | C4:C12, C7:C15 | Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴- | 107 |
| PEG | | Cys¹⁵-Leu¹⁶-PEG3 | |

TABLE 4
SP-304 Analogs, Uroguanylin, and Uroguanylin Analogs

| | Position of | | SEQ ID |
|---|---|---|---|
| Name | Disulfide bonds | Structure | NO |
| Formula | C4:C12, | Xaa¹-Xaa²-Xaa³-Maa⁴-Xaa⁵-Xaa⁶-Maa⁷-Xaa⁸-Xaa⁹-Xaa¹⁰-Xaa¹¹-Maa¹²-Xaa¹³- | 108 |
| XVIII | C7:C15 | Xaa¹⁴-Maa¹⁵-Xaa¹⁶ | |
| Uro- | C4:C12, | Asn¹-Asp²-Asp³-Cys⁴-Glu⁵-Leu⁶-Cys⁷-Val⁸-Asn⁹-Val¹⁰-Ala¹¹-Cys¹²-Thr¹³- | 109 |
| guanylin | C7:C15 | Gly¹⁴-Cys¹⁵-Leu¹⁶ | |
| N32 | C4:C12, C7:C15 | Glu¹-Asp²-Asp³-Cys⁴-Glu⁵-Leu⁶-Cys⁷-Val⁸-Asn⁹-Val¹⁰-Ala¹¹-Cys¹²-Thr¹³- | 110 |
| | | Gly¹⁴-Cys¹⁵-Leu¹⁶ | |
| N33 | C4:C12, C7:C15 | Glu¹-Asp²-Glu³-Cys⁴-Glu⁵-Leu⁶-Cys⁷-Val⁸-Asn⁹-Val¹⁰-Ala¹¹-Cys¹²-Thr¹³- | 111 |
| | | Gly¹⁴-Cys¹⁵-Leu¹⁶ | |
| N34 | C4:C12, C7:C15 | Glu¹-Glu²-Asp³-Cys⁴-Glu⁵-Leu⁶-Cys⁷-Val⁸-Asn⁹-Val¹⁰-Ala¹¹-Cys¹²-Thr¹³- | 112 |
| | | Gly¹⁴-Cys¹⁵-Leu¹⁶ | |
| N35 | C4:C12, C7:C15 | Glu¹-Glu²-Glu³-Cys⁴-Glu⁵-Leu⁶-Cys⁷-Val⁸-Asn⁹-Val¹⁰-Ala¹¹-Cys¹²-Thr¹³- | 113 |
| | | Gly¹⁴-Cys¹⁵-Leu¹⁶ | |
| N36 | C4:C12, C7:C15 | Asp¹-Asp²-Asp³-Cys⁴-Glu⁵-Leu⁶-Cys⁷-Val⁸-Asn⁹-Val¹⁰-Ala¹¹-Cys¹²-Thr¹³- | 114 |
| | | Gly¹⁴-Cys¹⁵-Leu¹⁶ | |
| N37 | C4:C12, C7:C15 | Asp¹-Asp²-Glu³-Cys⁴-Glu⁵-Leu⁶-Cys⁷-Val⁸-Asn⁹-Val¹⁰-Ala¹¹-Cys¹²-Thr¹³- | 115 |
| | | Gly¹⁴-Cys¹⁵-Leu¹⁶ | |
| N38 | C4:C12, C7:C15 | Asp¹-Glu²-Asp³-Cys⁴-Glu⁵-Leu⁶-Cys⁷-Val⁸-Asn⁹-Val¹⁰-Ala¹¹-Cys¹²-Thr¹³- | 116 |
| | | Gly¹⁴-Cys¹⁵-Leu¹⁶ | |
| N39 | C4:C12, C7:C15 | Asp¹-Glu²-Glu³-Cys⁴-Glu⁵-Leu⁶-Cys⁷-Val⁸-Asn⁹-Val¹⁰-Ala¹¹-Cys¹²-Thr¹³- | 117 |
| | | Gly¹⁴-Cys¹⁵-Leu¹⁶ | |
| N40 | C4:C12, C7:C15 | Gln¹-Asp²-Asp³-Cys⁴-Glu⁵-Leu⁶-Cys⁷-Val⁸-Asn⁹-Val¹⁰-Ala¹¹-Cys¹²-Thr¹³- | 118 |
| | | Gly¹⁴-Cys¹⁵-Leu¹⁶ | |
| N41 | C4:C12, C7:C15 | Gln¹-Asp²-Glu³-Cys⁴-Glu⁵-Leu⁶-Cys⁷-Val⁸-Asn⁹-Val¹⁰-Ala¹¹-Cys¹²-Thr¹³- | 119 |
| | | Gly¹⁴-Cys¹⁵-Leu¹⁶ | |
| N42 | C4:C12, C7:C15 | Gln¹-Glu²-Asp³-Cys⁴-Glu⁵-Leu⁶-Cys⁷-Val⁸-Asn⁹-Val¹⁰-Ala¹¹-Cys¹²-Thr¹³- | 120 |
| | | Gly¹⁴-Cys¹⁵-Leu¹⁶ | |
| N43 | C4:C12, C7:C15 | Gln¹-Glu²-Glu³-Cys⁴-Glu⁵-Leu⁶-Cys⁷-Val⁸-Asn⁹-Val¹⁰-Ala¹¹-Cys¹²-Thr¹³- | 121 |
| | | Gly¹⁴-Cys¹⁵-Leu¹⁶ | |
| N44 | C4:C12, C7:C15 | Lys¹-Asp²-Asp³-Cys⁴-Glu⁵-Leu⁶-Cys⁷-Val⁸-Asn⁹-Val¹⁰-Ala¹¹-Cys¹²-Thr¹³- | 122 |
| | | Gly¹⁴-Cys¹⁵-Leu¹⁶ | |
| N45 | C4:C12, C7:C15 | Lys¹-Asp²-Glu³-Cys⁴-Glu⁵-Leu⁶-Cys⁷-Val⁸-Asn⁹-Val¹⁰-Ala¹¹-Cys¹²-Thr¹³- | 123 |
| | | Gly¹⁴-Cys¹⁵-Leu¹⁶ | |
| N46 | C4:C12, C7:C15 | Lys¹-Glu²-Asp³-Cys⁴-Glu⁵-Leu⁶-Cys⁷-Val⁸-Asn⁹-Val¹⁰-Ala¹¹-Cys¹²-Thr¹³- | 124 |
| | | Gly¹⁴-Cys¹⁵-Leu¹⁶ | |
| N47 | C4:C12, C7:C15 | Lys¹-Glu²-Glu³-Cys⁴-Glu⁵-Leu⁶-Cys⁷-Val⁸-Asn⁹-Val¹⁰-Ala¹¹-Cys¹²-Thr¹³- | 125 |
| | | Gly¹⁴-Cys¹⁵-Leu¹⁶ | |
| N48 | C4:C12, C7:C15 | Glu¹-Asp²-Asp³-Cys⁴-Glu⁵-Leu⁶-Cys⁷-Val⁸-Asn⁹-Val¹⁰-Ala¹¹-Cys¹²-Thr¹³- | 126 |
| | | Gly¹⁴-Cys¹⁵-Leu¹⁶ | |
| N49 | C4:C12, C7:C15 | Glu¹-Asp²-Glu³-Cys⁴-Glu⁵-Leu⁶-Cys⁷-Val⁸-Asn⁹-Val¹⁰-Ala¹¹-Cys¹²-Thr¹³- | 127 |
| | | Gly¹⁴-Cys¹⁵-Leu¹⁶ | |
| N50 | C4:C12, C7:C15 | Glu¹-Glu²-Asp³-Cys⁴-Glu⁵-Leu⁶-Cys⁷-Val⁸-Asn⁹-Val¹⁰-Ala¹¹-Cys¹²-Thr¹³- | 128 |
| | | Gly¹⁴-Cys¹⁵-Leu¹⁶ | |
| N51 | C4:C12, C7:C15 | Glu¹-Glu²-Glu³-Cys⁴-Glu⁵-Leu⁶-Cys⁷-Val⁸-Asn⁹-Val¹⁰-Ala¹¹-Cys¹²-Thr¹³- | 129 |
| | | Gly¹⁴-Cys¹⁵-Leu¹⁶ | |
| N52 | C4:C12, C7:C15 | Asp¹-Asp²-Asp³-Cys⁴-Glu⁵-Leu⁶-Cys⁷-Val⁸-Asn⁹-Val¹⁰-Ala¹¹-Cys¹²-Thr¹³-⁶ | 130 |
| | | Gly¹⁴-Cys¹⁵-Leu¹ | |
| N53 | C4:C12, C7:C15 | Asp¹-Asp²-Glu³-Cys⁴-Glu⁵-Leu⁶-Cys⁷-Val⁸-Asn⁹-Val¹⁰-Ala¹¹-Cys¹²-Thr¹³- | 131 |
| | | Gly¹⁴-Cys¹⁵-Leu¹⁶ | |
| N54 | C4:C12, C7:C15 | Asp¹-Glu²-Asp³-Cys⁴-Glu⁵-Leu⁶-Cys⁷-Val⁸-Asn⁹-Val¹⁰-Ala¹¹-Cys¹²-Thr¹³- | 132 |
| | | Gly¹⁴-Cys¹⁵-Leu¹⁶ | |
| N55 | C4:C12, C7:C15 | Asp¹-Glu²-Glu³-Cys⁴-Glu⁵-Leu⁶-Cys⁷-Val⁸-Asn⁹-Val¹⁰-Ala¹¹-Cys¹²-Thr¹³- | 133 |
| | | Gly¹⁴-Cys¹⁵-Leu¹⁶ | |
| N56 | C4:C12, C7:C15 | Gln¹-Asp²-Asp³-Cys⁴-Glu⁵-Leu⁶-Cys⁷-Val⁸-Asn⁹-Val¹⁰-Ala¹¹-Cys¹²-Thr¹³- | 134 |
| | | Gly¹⁴-Cys¹⁵-Leu¹⁶ | |
| N57 | C4:C12, C7:C15 | Gln¹-Asp²-Glu³-Cys⁴-Glu⁵-Leu⁶-Cys⁷-Val⁸-Asn⁹-Val¹⁰-Ala¹¹-Cys¹²-Thr¹³- | 135 |
| | | Gly¹⁴-Cys¹⁵-Leu¹⁶ | |
| N58 | C4:C12, C7:C15 | Gln¹-Glu²-Asp³-Cys⁴-Glu⁵-Leu⁶-Cys⁷-Val⁸-Asn⁹-Val¹⁰-Ala¹¹-Cys¹²-Thr¹³- | 136 |
| | | Gly¹⁴-Cys¹⁵-Leu¹⁶ | |
| N59 | C4:C12, C7:C15 | Gln¹-Glu²-Glu³-Cys⁴-Glu⁵-Leu⁶-Cys⁷-Val⁸-Asn⁹-Val¹⁰-Ala¹¹-Cys¹²-Thr¹³- | 137 |
| | | Gly¹⁴-Cys¹⁵-Leu¹⁶ | |
| N60 | C4:C12, C7:C15 | Lys¹-Asp²-Asp³-Cys⁴-Glu⁵-Leu⁶-Cys⁷-Val⁸-Asn⁹-Val¹⁰-Ala¹¹-Cys¹²-Thr¹³- | 138 |
| | | Gly¹⁴-Cys¹⁵-Leu¹⁶ | |
| N61 | C4:C12, C7:C15 | Lys¹-Asp²-Glu³-Cys⁴-Glu⁵-Leu⁶-Cys⁷-Val⁸-Asn⁹-Val¹⁰-Ala¹¹-Cys¹²-Thr¹³- | 139 |
| | | Gly¹⁴-Cys¹⁵-Leu¹⁶ | |
| N62 | C4:C12, C7:C15 | Lys¹-Glu²-Asp³-Cys⁴-Glu⁵-Leu⁶-Cys⁷-Val⁸-Asn⁹-Val¹⁰-Ala¹¹-Cys¹²-Thr¹³- | 140 |
| | | Gly¹⁴-Cys¹⁵-Leu¹⁶ | |
| N63 | C4:C12, C7:C15 | Lys¹-Glu²-Glu³-Cys⁴-Glu⁵-Leu⁶-Cys⁷-Val⁸-Asn⁹-Val¹⁰-Ala¹¹-Cys¹²-Thr¹³- | 141 |
| | | Gly¹⁴-Cys¹⁵-Leu¹⁶ | |
| N65 | C4:C12, C7:C15 | Glu¹-Asp²-Asp³-Cys⁴-Glu⁵-Leu⁶-Cys⁷-Ile⁸-Asn⁹-Met¹⁰-Ala¹¹-Cys¹²-Thr¹³- | 142 |
| | | Gly¹⁴-Cys¹⁵-Leu¹⁶ | |
| N66 | C4:C12, C7:C15 | Glu¹-Asp²-Glu³-Cys⁴-Glu⁵-Leu⁶-Cys⁷-Ile⁸-Asn⁹-Met¹⁰-Ala¹¹-Cys¹²-Thr¹³- | 143 |
| | | Gly¹⁴-Cys¹⁵-Leu¹⁶ | |
| N67 | C4:C12, C7:C15 | Glu¹-Glu²-Asp³-Cys⁴-Glu⁵-Leu⁶-Cys⁷-Ile⁸-Asn⁹-Met¹⁰-Ala¹¹-Cys¹²-Thr¹³- | 144 |
| | | Gly¹⁴-Cys¹⁵-Leu¹⁶ | |
| N68 | C4:C12, C7:C15 | Glu¹-Glu²-Glu³-Cys⁴-Glu⁵-Leu⁶-Cys⁷-Ile⁸-Asn⁹-Met¹⁰-Ala¹¹-Cys¹²-Thr¹³- | 145 |
| | | Gly¹⁴-Cys¹⁵-Leu¹⁶ | |
| N69 | C4:C12, C7:C15 | Asp¹-Asp²-Asp³-Cys⁴-Glu⁵-Leu⁶-Cys⁷-Ile⁸-Asn⁹-Met¹⁰-Ala¹¹-Cys¹²-Thr¹³- | 146 |
| | | Gly¹⁴-Cys¹⁵-Leu¹⁶ | |
| N70 | C4:C12, C7:C15 | Asp¹-Asp²-Glu³-Cys⁴-Glu⁵-Leu⁶-Cys⁷-Ile⁸-Asn⁹-Met¹⁰-Ala¹¹-Cys¹²-Thr¹³- | 147 |
| | | Gly¹⁴-Cys¹⁵-Leu¹⁶ | |
| N71 | C4:C12, C7:C15 | Asp¹-Glu²-Asp³-Cys⁴-Glu⁵-Leu⁶-Cys⁷-Ile⁸-Asn⁹-Met¹⁰-Ala¹¹-Cys¹²-Thr¹³- | 148 |
| | | Gly¹⁴-Cys¹⁵-Leu¹⁶ | |
| N72 | C4:C12, C7:C15 | Asp¹-Glu²-Glu³-Cys⁴-Glu⁵-Leu⁶-Cys⁷-Ile⁸-Asn⁹-Met¹⁰-Ala¹¹-Cys¹²-Thr¹³- | 149 |
| | | Gly¹⁴-Cys¹⁵-Leu¹⁶ | |
| N73 | C4:C12, C7:C15 | Gln¹-Asp²-Asp³-Cys⁴-Glu⁵-Leu⁶-Cys⁷-Ile⁸-Asn⁹-Met¹⁰-Ala¹¹-Cys¹²-Thr¹³- | 150 |
| | | Gly¹⁴-Cys¹⁵-Leu¹⁶ | |
| N74 | C4:C12, C7:C15 | Gln¹-Asp²-Glu³-Cys⁴-Glu⁵-Leu⁶-Cys⁷-Ile⁸-Asn⁹-Met¹⁰-Ala¹¹-Cys¹²-Thr¹³- | 151 |
| | | Gly¹⁴-Cys¹⁵-Leu¹⁶ | |
| N75 | C4:C12, C7:C15 | Gln¹-Glu²-Asp³-Cys⁴-Glu⁵-Leu⁶-Cys⁷-Ile⁸-Asn⁹-Met¹⁰-Ala¹¹-Cys¹²-Thr¹³- | 152 |
| | | Gly¹⁴-Cys¹⁵-Leu¹⁶ | |
| N76 | C4:C12, C7:C15 | Gln¹-Glu²-Glu³-Cys⁴-Glu⁵-Leu⁶-Cys⁷-Ile⁸-Asn⁹-Met¹⁰-Ala¹¹-Cys¹²-Thr¹³- | 153 |
| | | Gly¹⁴-Cys¹⁵-Leu¹⁶ | |
| N77 | C4:C12, C7:C15 | Lys¹-Asp²-Asp³-Cys⁴-Glu⁵-Leu⁶-Cys⁷-Ile⁸-Asn⁹-Met¹⁰-Ala¹¹-Cys¹²-Thr¹³- | 154 |
| | | Gly¹⁴-Cys¹⁵-Leu¹⁶ | |
| N78 | C4:C12, C7:C15 | Lys¹-Asp²-Glu³-Cys⁴-Glu⁵-Leu⁶-Cys⁷-Ile⁸-Asn⁹-Met¹⁰-Ala¹¹-Cys¹²-Thr¹³- | 155 |
| | | Gly¹⁴-Cys¹⁵-Leu¹⁶ | |
| N79 | C4:C12, C7:C15 | Lys¹-Glu²-Asp³-Cys⁴-Glu⁵-Leu⁶-Cys⁷-Ile⁸-Asn⁹-Met¹⁰-Ala¹¹-Cys¹²-Thr¹³- | 156 |
| | | Gly¹⁴-Cys¹⁵-Leu¹⁶ | |
| N80 | C4:C12, C7:C15 | Lys¹-Glu²-Glu³-Cys⁴-Glu⁵-Leu⁶-Cys⁷-Ile⁸-Asn⁹-Met¹⁰-Ala¹¹-Cys¹²-Thr¹³- | 157 |
| | | Gly¹⁴-Cys¹⁵-Leu¹⁶ | |
| N81 | C4:C12, C7:C15 | Glu¹-Asp²-Asp³-Cys⁴-Glu⁵-Leu⁶-Cys⁷-Ile⁸-Asn⁹-Met¹⁰-Ala¹¹-Cys¹²-Thr¹³- | 158 |
| | | Gly¹⁴-Cys¹⁵-Leu¹⁶ | |
| N82 | C4:C12, C7:C15 | Glu¹-Asp²-Glu³-Cys⁴-Glu⁵-Leu⁶-Cys⁷-Ile⁸-Asn⁹-Met¹⁰-Ala¹¹-Cys¹²-Thr¹³- | 159 |
| | | Gly¹⁴-Cys¹⁵-Leu¹⁶ | |
| N83 | C4:C12, C7:C15 | Glu¹-Glu²-Asp³-Cys⁴-Glu⁵-Leu⁶-Cys⁷-Ile⁸-Asn⁹-Met¹⁰-Ala¹¹-Cys¹²-Thr¹³- | 160 |
| | | Gly¹⁴-Cys¹⁵-Leu¹⁶ | |
| N84 | C4:C12, C7:C15 | Glu¹-Glu²-Glu³-Cys⁴-Glu⁵-Leu⁶-Cys⁷-Ile⁸-Asn⁹-Met¹⁰-Ala¹¹-Cys¹²-Thr¹³- | 161 |
| | | Gly¹⁴-Cys¹⁵-Leu¹⁶ | |
| N85 | C4:C12, C7:C15 | Asp¹-Asp²-Asp³-Cys⁴-Glu⁵-Leu⁶-Cys⁷-Ile⁸-Asn⁹-Met¹⁰-Ala¹¹-Cys¹²-Thr¹³- | 162 |
| | | Gly¹⁴-Cys¹⁵-Leu¹⁶ | |
| N86 | C4:C12, C7:C15 | Asp¹-Asp²-Glu³-Cys⁴-Glu⁵-Leu⁶-Cys⁷-Ile⁸-Asn⁹-Met¹⁰-Ala¹¹-Cys¹²-Thr¹³- | 163 |
| | | Gly¹⁴-Cys¹⁵-Leu¹⁶ | |
| N87 | C4:C12, C7:C15 | Asp¹-Glu²-Asp³-Cys⁴-Glu⁵-Leu⁶-Cys⁷-Ile⁸-Asn⁹-Met¹⁰-Ala¹¹-Cys¹²-Thr¹³- | 164 |
| | | Gly¹⁴-Cys¹⁵-Leu¹⁶ | |
| N88 | C4:C12, C7:C15 | Asp¹-Glu²-Glu³-Cys⁴-Glu⁵-Leu⁶-Cys⁷-Ile⁸-Asn⁹-Met¹⁰-Ala¹¹-Cys¹²-Thr¹³- | 165 |
| | | Gly¹⁴-Cys¹⁵-Leu¹⁶ | |
| N89 | C4:C12, C7:C15 | Gln¹-Asp²-Asp³-Cys⁴-Glu⁵-Leu⁶-Cys⁷-Ile⁸-Asn⁹-Met¹⁰-Ala¹¹-Cys¹²-Thr¹³- | 166 |
| | | Gly¹⁴-Cys¹⁵-Leu¹⁶ | |
| N90 | C4:C12, C7:C15 | Gln¹-Asp²-Glu³-Cys⁴-Glu⁵-Leu⁶-Cys⁷-Ile⁸-Asn⁹-Met¹⁰-Ala¹¹-Cys¹²-Thr¹³- | 167 |
| | | Gly¹⁴-Cys¹⁵-Leu¹⁶ | |
| N91 | C4:C12, C7:C15 | Gln¹-Glu²-Asp³-Cys⁴-Glu⁵-Leu⁶-Cys⁷-Ile⁸-Asn⁹-Met¹⁰-Ala¹¹-Cys¹²-Thr¹³- | 168 |
| | | Gly¹⁴-Cys¹⁵-Leu¹⁶ | |
| N92 | C4:C12, C7:C15 | Gln¹-Glu²-Glu³-Cys⁴-Glu⁵-Leu⁶-Cys⁷-Ile⁸-Asn⁹-Met¹⁰-Ala¹¹-Cys¹²-Thr¹³- | 169 |
| | | Gly¹⁴-Cys¹⁵-Leu¹⁶ | |
| N93 | C4:C12, C7:C15 | Lys¹-Asp²-Asp³-Cys⁴-Glu⁵-Leu⁶-Cys⁷-Ile⁸-Asn⁹-Met¹⁰-Ala¹¹-Cys¹²-Thr¹³- | 170 |
| | | Gly¹⁴-Cys¹⁵-Leu¹⁶ | |
| N94 | C4:C12, C7:C15 | Lys¹-Asp²-Glu³-Cys⁴-Glu⁵-Leu⁶-Cys⁷-Ile⁸-Asn⁹-Met¹⁰-Ala¹¹-Cys¹²-Thr¹³- | 171 |
| | | Gly¹⁴-Cys¹⁵-Leu¹⁶ | |
| N95 | C4:C12, C7:C15 | Lys¹-Glu²-Asp³-Cys⁴-Glu⁵-Leu⁶-Cys⁷-Ile⁸-Asn⁹-Met¹⁰-Ala¹¹-Cys¹²-Thr¹³- | 172 |
| | | Gly¹⁴-Cys¹⁵-Leu¹⁶ | |
| N96 | C4:C12, C7:C15 | Lys¹-Glu²-Glu³-Cys⁴-Glu⁵-Leu⁶-Cys⁷-Ile⁸-Asn⁹-Met¹⁰-Ala¹¹-Cys¹²-Thr¹³- | 173 |
| | | Gly¹⁴-Cys¹⁵-Leu¹⁶ | |

TABLE 5
Guanylin and Analogs

| | Position of | | SEQ |
|---|---|---|---|
| | Disulfide | | ID |
| Name | bonds | Structure | NO |
| Formula | 4:12, 7:15 | Xaa¹-Xaa²-Xaa³-Maa⁴-Xaa⁵-Xaaa⁶-Maa⁷-Xaa⁸-Xaa⁹-Xaa¹⁰-Xaa¹¹-Maa¹²-Xaa¹³-Xaa¹⁴- | 174 |
| XIX | | Maa¹⁵ | |
| Guanylin | C4:C12, C7:C15 | Ser¹-His²-Thr³-Cys⁴-Glu⁵-Ile⁶-Cys⁷-Ala⁸-Phe⁹-Ala¹⁰-Ala¹¹-Cys¹²-Ala¹³-Gly¹⁴- | 175 |
| | | Cys¹⁵ | |
| Human | C4:C12, C7:C15 | Pro¹-Gly²-Thr³-Cys⁴-Glu⁵-Ile⁶-Cys⁷-Ala⁸-Tyr⁹-Ala¹⁰-Ala¹¹-Cys¹²-Thr¹³-Gly¹⁴- | 252 |
| Guanylin | | Cys¹⁵ | |
| N97 | C4:C12, C7:C15 | Ser¹-His²-Thr³-Cys⁴-Glu⁵-Ile⁶-Cys⁷-Ala⁸-Asn⁹-Ala¹⁰-Ala¹¹-Cys¹²-Ala¹³-Gly¹⁴-Cys¹⁵ | 176 |
| N98 | C4:C12, C7:C15 | Ser¹-His²-Thr³-Cys⁴-Glu⁵-Leu⁶-Cys⁷-Ala⁸-Asn⁹-Ala¹⁰-Ala¹¹-Cys¹²-Ala¹³-Gly¹⁴-Cys¹⁵ | 177 |
| N99 | C4:C12, C7:C15 | Ser¹-His²-Thr³-Cys⁴-Glu⁵-Val⁶-Cys⁷-Ala⁸-Asn⁹-Ala¹⁰-Ala¹¹-Cys¹²-Ala¹³-Gly¹⁴-Cys¹⁵ | 178 |
| N100 | C4:C12, C7:C15 | Ser¹-His²-Thr³-Cys⁴-Glu⁵-Tyr⁶-Cys⁷-Ala⁸-Asn⁹-Ala¹⁰-Ala¹¹-Cys¹²-Ala¹³-Gly¹⁴-Cys¹⁵ | 179 |
| N101 | C4:C12, C7:C15 | Ser¹-His²-Thr³-Cys⁴-Glu⁵-Ile⁶-Cys⁷-Ala⁸-Asn⁹-Ala¹⁰-Ala¹¹-Cys¹²-Ala¹³-Gly¹⁴-Cys¹⁵ | 180 |
| N102 | C4:C12, C7:C15 | Ser¹-His²-Thr³-Cys⁴-Glu⁵-Leu⁶-Cys⁷-Ala⁸-Asn⁹-Ala¹⁰-Ala¹¹-Cys¹²-Ala¹³-Gly¹⁴-Cys¹⁵ | 181 |
| N103 | C4:C12, C7:C15 | Ser¹-His²-Thr³-Cys⁴-Glu⁵-Val⁶-Cys⁷-Ala⁸-Asn⁹-Ala¹⁰-Ala¹¹-Cys¹²-Ala¹³-Gly¹⁴-Cys¹⁵ | 182 |
| N104 | C4:C12, C7:C15 | Ser¹-His²-Thr³-Cys⁴-Glu⁵-Tyr⁶-Cys⁷-Ala⁸-Asn⁹-Ala¹⁰-Ala¹¹-Cys¹²-Ala¹³-Gly¹⁴-Cys¹⁵ | 183 |
| N105 | C4:C12, C7:C15 | Ser¹-His²-Thr³-Cys⁴-Glu⁵-Ile⁶-Cys⁷-Ala⁸-Asn⁹-Ala¹⁰-Ala¹¹-Cys¹²-Ala¹³-Gly¹⁴-Cys¹⁵ | 184 |
| N106 | C4:C12, C7:C15 | Ser¹-His²-Thr³-Cys⁴-Glu⁵-Leu⁶-Cys⁷-Ala⁸-Asn⁹-Ala¹⁰-Ala¹¹-Cys¹²-Ala¹³-Gly¹⁴-Cys¹⁵ | 185 |
| N107 | C4:C12, C7:C15 | Ser¹-His²-Thr³-Cys⁴-Glu⁵-Val⁶-Cys⁷-Ala⁸-Asn⁹-Ala¹⁰-Ala¹¹-Cys¹²-Ala¹³-Gly¹⁴-Cys¹⁵ | 186 |
| N108 | C4:C12, C7:C15 | Ser¹-His²-Thr³-Cys⁴-Glu⁵-Tyr⁶-Cys⁷-Ala⁸-Asn⁹-Ala¹⁰-Ala¹¹-Cys¹²-Ala¹³-Gly¹⁴-Cys¹⁵ | 187 |
| N109 | C4:C12, C7:C15 | Ser¹-His²-Thr³-Cys⁴-Glu⁵-Ile⁶-Cys⁷-Ala⁸-Asn⁹-Ala¹⁰-Ala¹¹-Cys¹²-Ala¹³-Gly¹⁴-Cys¹⁵ | 188 |
| N110 | C4:C12, C7:C15 | Ser¹-His²-Thr³-Cys⁴-Glu⁵-Leu⁶-Cys⁷-Ala⁸-Asn⁹-Ala¹⁰-Ala¹¹-Cys¹²-Ala¹³-Gly¹⁴-Cys¹⁵ | 189 |
| N111 | C4:C12, C7:C15 | Ser¹-His²-Thr³-Cys⁴-Glu⁵-Val⁶-Cys⁷-Ala⁸-Asn⁹-Ala¹⁰-Ala¹¹-Cys¹²-Ala¹³-Gly¹⁴-Cys¹⁵ | 190 |
| N112 | C4:C12, C7:C15 | Ser¹-His²-Thr³-Cys⁴-Glu⁵-Tyr⁶-Cys⁷-Ala⁸-Asn⁹-Ala¹⁰-Ala¹¹-Cys¹²-Ala¹³-Gly¹⁴-Cys¹⁵ | 191 |
| N113 | C4:C12, C7:C15 | Asn¹-Asp²-Glu³-Cys⁴-Glu⁵-Ile⁶-Cys⁷-Ala⁸-Asn⁹-Ala¹⁰-Ala¹¹-Cys¹²-Ala¹³-Gly¹⁴-Cys¹⁵ | 192 |
| N114 | C4:C12, C7:C15 | Asn¹-Asp²-Glu³-Cys⁴-Glu⁵-Leu⁶-Cys⁷-Ala⁸-Asn⁹-Ala¹⁰-Ala¹¹-Cys¹²-Ala¹³-Gly¹⁴-Cys¹⁵ | 193 |
| N115 | C4:C12, C7:C15 | Asn¹-Asp²-Glu³-Cys⁴-Glu⁵-Val⁶-Cys⁷-Ala⁸-Asn⁹-Ala¹⁰-Ala¹¹-Cys¹²-Ala¹³-Gly¹⁴-Cys¹⁵ | 194 |
| N116 | C4:C12, C7:C15 | Asn¹-Asp²-Glu³-Cys⁴-Glu⁵-Tyr⁶-Cys⁷-Ala⁸-Asn⁹-Ala¹⁰-Ala¹¹-Cys¹²-Ala¹³-Gly¹⁴-Cys¹⁵ | 195 |
| N117 | C4:C12, C7:C15 | Asn¹-Asp²-Glu³-Cys⁴-Glu⁵-Ile⁶-Cys⁷-Ala⁸-Asn⁹-Ala¹⁰-Ala¹¹-Cys¹²-Ala¹³-Gly¹⁴-Cys¹⁵ | 196 |
| N118 | C4:C12, C7:C15 | Asn¹-Asp²-Glu³-Cys⁴-Glu⁵-Leu⁶-Cys⁷-Ala⁸-Asn⁹-Ala¹⁰-Ala¹¹-Cys¹²-Ala¹³-Gly¹⁴-Cys¹⁵ | 197 |
| N119 | C4:C12, C7:C15 | Asn¹-Asp²-Glu³-Cys⁴-Glu⁵-Val⁶-Cys⁷-Ala⁸-Asn⁹-Ala¹⁰-Ala¹¹-Cys¹²-Ala¹³-Gly¹⁴-Cys¹⁵ | 198 |
| N120 | C4:C12, C7:C15 | Asn¹-Asp²-Glu³-Cys⁴-Glu⁵-Tyr⁶-Cys⁷-Ala⁸-Asn⁹-Ala¹⁰-Ala¹¹-Cys¹²-Ala¹³-Gly¹⁴-Cys¹⁵ | 199 |
| N121 | C4:C12, C7:C15 | Asn¹-Asp²-Glu³-Cys⁴-Glu⁵-Ile⁶-Cys⁷-Ala⁸-Asn⁹-Ala¹⁰-Ala¹¹-Cys¹²-Ala¹³-Gly¹⁴-Cys¹⁵ | 200 |
| N122 | C4:C12, C7:C15 | Asn¹-Asp²-Glu³-Cys⁴-Glu⁵-Leu⁶-Cys⁷-Ala⁸-Asn⁹-Ala¹⁰-Ala¹¹-Cys¹²-Ala¹³-Gly¹⁴-Cys¹⁵ | 201 |
| N123 | C4:C12, C7:C15 | Asn¹-Asp²-Glu³-Cys⁴-Glu⁵-Val⁶-Cys⁷-Ala⁸-Asn⁹-Ala¹⁰-Ala¹¹-Cys¹²-Ala¹³-Gly¹⁴-Cys¹⁵ | 202 |
| N124 | C4:C12, C7:C15 | Asn¹-Asp²-Glu³-Cys⁴-Glu⁵-Tyr⁶-Cys⁷-Ala⁸-Asn⁹-Ala¹⁰-Ala¹¹-Cys¹²-Ala¹³-Gly¹⁴-Cys¹⁵ | 203 |
| N125 | C4:C12, C7:C15 | Asn¹-Asp²-Glu³-Cys⁴-Glu⁵-Ile⁶-Cys⁷-Ala⁸-Asn⁹-Ala¹⁰-Ala¹¹-Cys¹²-Ala¹³-Gly¹⁴-Cys¹⁵ | 204 |
| N126 | C4:C12, C7:C15 | Asn¹-Asp²-Glu³-Cys⁴-Glu⁵-Leu⁶-Cys⁷-Ala⁸-Asn⁹-Ala¹⁰-Ala¹¹-Cys¹²-Ala¹³-Gly¹⁴-Cys¹⁵ | 205 |
| N127 | C4:C12, C7:C15 | Asn¹-Asp²-Glu³-Cys⁴-Glu⁵-Val⁶-Cys⁷-Ala⁸-Asn⁹-Ala¹⁰-Ala¹¹-Cys¹²-Ala¹³-Gly¹⁴-Cys¹⁵ | 206 |
| N128 | C4:C12, C7:C15 | Asn¹-Asp²-Glu³-Cys⁴-Glu⁵-Tyr⁶-Cys⁷-Ala⁸-Asn⁹-Ala¹⁰-Ala¹¹-Cys¹²-Ala¹³-Gly¹⁴-Cys¹⁵ | 207 |

TABLE 6
Lymphoguanylin and Analogs

| | Position of | | SEQ |
|---|---|---|---|
| | Disulfide | | ID |
| Name | bonds | Structure | NO |
| FormulaXX | 4:12 | Xaa¹-Xaa²-Xaa³-Maa⁴-Xaa⁵-Xaa⁶-Maa⁷-Xaa⁸-Xaa⁹-xaa¹⁰-Xaa¹¹-Maa¹²-Xaa¹³-Xaa¹⁴-Xaaₙ₁¹⁵ | 208 |
| Lympho- | C4:C12 | Gln¹-Glu²-Glu³-Cys⁴-Glu⁵-Leu⁶-Cys⁷-Ile⁸-Asn⁹-Met¹⁰-Ala¹¹-Cys¹²-Thr¹³-Gly¹⁴-Tyr¹⁵ | 209 |
| guanylin | | | |
| N129 | C4:C12 | Gln¹-Glu²-Glu³-Cys⁴-Glu⁵-Thr⁶-Cys⁷-Ile⁸-Asn⁹-Met¹⁰-Ala¹¹-Cys¹²-Thr¹³-Gly¹⁴-Tyr¹⁵ | 210 |
| N130 | C4:C12 | Gln¹-Asp²-Glu³-Cys⁴-Glu⁵-Thr⁶-Cys⁷-Ile⁸-Asn⁹-Met¹⁰-Ala¹¹-Cys¹²-Thr¹³-Gly¹⁴-Tyr¹⁵ | 211 |
| N131 | C4:C12 | Gln¹-Asp²-Asp³-Cys⁴-Glu⁵-Thr⁶-Cys⁷-Ile⁸-Asn⁹-Met¹⁰-Ala¹¹-Cys¹²-Thr¹³-Gly¹⁴-Tyr¹⁵ | 212 |
| N132 | C4:C12 | Gln¹-Glu²-Asp³-Cys⁴-Glu⁵-Thr⁶-Cys⁷-Ile⁸-Asn⁹-Met¹⁰-Ala¹¹-Cys¹²-Thr¹³-Gly¹⁴-Tyr¹⁵ | 213 |
| N133 | C4:C12 | Gln¹-Glu²-Glu³-Cys⁴-Glu⁵-Glu⁶-Cys⁷-Ile⁸-Asn⁹-Met¹⁰-Ala¹¹-Cys¹²-Thr¹³-Gly¹⁴-Tyr¹⁵ | 214 |
| N134 | C4:C12 | Gln¹-Asp²-Glu³-Cys⁴-Glu⁵-Glu⁶-Cys⁷-Ile⁸-Asn⁹-Met¹⁰-Ala¹¹-Cys¹²-Thr¹³-Gly¹⁴-Tyr¹⁵ | 215 |
| N135 | C4:C12 | Glni-Asp²-Asp³-Cys⁴-Glu⁵-Glu⁶-Cys⁷-Ile⁸-Asn⁹-Met¹⁰-Ala¹¹-Cys¹²-Thr¹³-Gly¹⁴-Tyr¹⁵ | 216 |
| N136 | C4:C12 | Gln¹-Glu²-Asp³-Cys⁴-Glu⁵-Glu⁶-Cys⁷-Ile⁸-Asn⁹-Met¹⁰-Ala¹¹-Cys¹²-Thr¹³-Gly¹⁴-Tyr¹⁵ | 217 |
| N137 | C4:C12 | Gln¹-Glu²-Glu³-Cys⁴-Glu⁵-Tyr⁶-Cys⁷-Ile⁸-Asn⁹-Met¹⁰-Ala¹¹-Cys¹²-Thr¹³-Gly¹⁴-Tyr¹⁵ | 218 |
| N138 | C4:C12 | Gln¹-Asp²-Glu³-Cys⁴-Glu⁵-Tyr⁶-Cys⁷-Ile⁸-Asn⁹-Met¹⁰-Ala¹¹-Cys¹²-Thr¹³-Gly¹⁴-Tyr¹⁵ | 219 |
| N139 | C4:C12 | Gln¹-Asp²-Asp³-Cys⁴-Glu⁵-Tyr⁶-Cys⁷-Ile⁸-Asn⁹-Met¹⁰-Ala¹¹-Cys¹²-Thr¹³-Gly¹⁴-Tyr¹⁵ | 220 |
| N140 | C4:C12 | Gln¹-Glu²-Asp³-Cys⁴-Glu⁵-Tyr⁶-Cys⁷-Ile⁸-Asn⁹-Met¹⁰-Ala¹¹-Cys¹²-Thr¹³-Gly¹⁴-Tyr¹⁵ | 221 |
| N141 | C4:C12 | Gln¹-Glu²-Glu³-Cys⁴-Glu⁵-Ile⁶-Cys⁷-Ile⁸-Asn⁹-Met¹⁰-Ala¹¹-Cys¹²-Thr¹³-Gly¹⁴-Tyr¹⁵ | 222 |
| N142 | C4:C12 | Gln¹-Asp²-Glu³-Cys⁴-Glu⁵-Ile⁶-Cys⁷-Ile⁸-Asn⁹-Met¹⁰-Ala¹¹-Cys¹²-Thr¹³-Gly¹⁴-Tyr¹⁵ | 223 |
| N143 | C4:C12 | Gln¹-Asp²-Asp³-Cys⁴-Glu⁵-Ile⁶-Cys⁷-Ile⁸-Asn⁹-Met¹⁰-Ala¹¹-Cys¹²-Thr¹³-Gly¹⁴-Tyr¹⁵ | 224 |
| N144 | C4:C12 | Gln¹-Glu²-Asp³-Cys⁴-Glu⁵-Ile⁶-Cys⁷-Ile⁸-Asn⁹-Met¹⁰-Ala¹¹-Cys¹²-Thr¹³-Gly¹⁴-Tyr¹⁵ | 225 |
| N145 | C4:C12, | Gln¹-Glu²-Glu³-Cys⁴-Glu⁵-Thr⁶-Cys⁷-Ile⁸-Asn⁹-Met¹⁰-Ala¹¹-Cys¹²-Thr¹³-Gly¹⁴-Cys¹⁵- | 226 |
| | C7:C15 | Ser¹⁶ | |
| N146 | C4:C12, | Gln¹-Asp²-Glu³-Cys⁴-Glu⁵-Thr⁶-Cys⁷-Ile⁸-Asn⁹-Met¹⁰-Ala¹¹-Cys¹²-Thr¹³-Gly¹⁴-Cys¹⁵- | 227 |
| | C7:C15 | Ser¹⁶ | |
| N147 | C4:C12, | Gln¹-Asp²-Asp³-Cys⁴-Glu⁵-Thr⁶-Cys⁷-Ile⁸-Asn⁹-Met¹⁰-Ala¹¹-Cys¹²-Thr¹³-Gly¹⁴-Cys¹⁵- | 228 |
| | C7:C15 | Ser¹⁶ | |
| N148 | C4:C12, | Gln¹-Glu²-Asp³-Cys⁴-Glu⁵-Thr⁶-Cys⁷-Ile⁸-Asn⁹-Met¹⁰-Ala¹¹-Cys¹²-Thr¹³-Gly¹⁴-Cys¹⁵- | 229 |
| | C7:C15 | Ser¹⁶ | |
| N149 | C4:C12, | Gln¹-Glu²-Glu³-Cys⁴-Glu⁵-Glu⁶-Cys⁷-Ile⁸-Asn⁹-Met¹⁰-Ala¹¹-Cys¹²-Thr¹³-Gly¹⁴-Cys¹⁵- | 230 |
| | C7:C15 | Ser¹⁶ | |
| N150 | C4:C12, | Gln¹-Asp²-Glu³-Cys⁴-Glu⁵-Glu⁶-Cys⁷-Ile⁸-Asn⁹-Met¹⁰-Ala¹¹-Cys¹²-Thr¹³-Gly¹⁴-Cys¹⁵- | 231 |
| | C7:C15 | Ser¹⁶ | |
| N151 | C4:C12, | Gln¹-Asp²-Asp³-Cys⁴-Glu⁵-Glu⁶-Cys⁷-Ile⁸-Asn⁹-Met¹⁰-Ala¹¹-Cys¹²-Thr¹³-Gly¹⁴-Cys¹⁵- | 232 |
| | C7:C15 | Ser¹⁶ | |
| N152 | C4:C12, | Gln¹-Glu²-Asp³-Cys⁴-Glu⁵-Glu⁶-Cys⁷-Ile⁸-Asn⁹-Met¹⁰-Ala¹¹-Cys¹²-Thr¹³-Gly¹⁴-Cys¹⁵- | 233 |
| | C7:C15 | Ser¹⁶ | |
| N153 | C4:C12, | Gln¹-Glu²-Glu³-Cys⁴-Glu⁵-Tyr⁶-Cys⁷-Ile⁸-Asn⁹-Met¹⁰-Ala¹¹-Cys¹²-Thr¹³-Gly¹⁴-Cys¹⁵- | 234 |
| | C7:C15 | Ser¹⁶ | |
| N154 | C4:C12, | Gln¹-Asp²-Glu³-Cys⁴-Glu⁵-Tyr⁶-Cys⁷-Ile⁸-Asn⁹-Met¹⁰-Ala¹¹-Cys¹²-Thr¹³-Gly¹⁴-Cys¹⁵- | 235 |
| | C7:C15 | Ser¹⁶ | |
| N155 | C4:C12, | Gln¹-Asp²-Asp³-Cys⁴-Glu⁵-Tyr⁶-Cys⁷-Ile⁸-Asn⁹-Met¹⁰-Ala¹¹-Cys¹²-Thr¹³-Gly¹⁴-Cys¹⁵- | 236 |
| | C7:C15 | Ser¹⁶ | |
| N156 | C4:C12, | Gln¹-Glu²-Asp³-Cys⁴-Glu⁵-Tyr⁶-Cys⁷-Ile⁸-Asn⁹-Met¹⁰-Ala¹¹-Cys¹²-Thr¹³-Gly¹⁴-Cys¹⁵- | 237 |
| | C7:C15 | Ser¹⁶ | |
| N157 | C4:C12, | Gln¹-Glu²-Glu³-Cys⁴-Glu⁵-Ile⁶-Cys⁷-Ile⁸-Asn⁹-Met¹⁰-Ala¹¹-Cys¹²-Thr¹³-Gly¹⁴-Cys¹⁵- | 238 |
| | C7:C15 | Ser¹⁶ | |
| N158 | C4:C12, | Gln¹-Asp²-Glu³-Cys⁴-Glu⁵-Ile⁶-Cys⁷-Ile⁸-Asn⁹-Met¹⁰-Ala¹¹-Cys¹²-Thr¹³-Gly¹⁴-Cys¹⁵- | 239 |
| | C7:C15 | Ser¹⁶ | |
| N159 | C4:C12, | Gln¹-Asp²-Asp³-Cys⁴-Glu⁵-Ile⁶-Cys⁷-Ile⁸-Asn⁹-Met¹⁰-Ala¹¹-Cys¹²-Thr¹³-Gly¹⁴-Cys¹⁵- | 240 |
| | C7:C15 | Ser¹⁶ | |
| N160 | C4:C12, | Gln¹-Glu²-Asp³-Cys⁴-Glu⁵-Ile⁶-Cys⁷-Ile⁸-Asn⁹-Met¹⁰-Ala¹¹-Cys¹²-Thr¹³-Gly¹⁴-Cys¹⁵- | 241 |
| | C7:C15 | Ser¹⁶ | |

TABLE 7
ST Peptide and Analogues

| | Position of | | SEQ ID |
|---|---|---|---|
| Name | Disulfide bonds | Structure | NO |
| STPeptide | C9:C14, C10:C18, | Asn¹-Ser²-Ser³-Asn⁴-Ser⁵-Ser⁶-Asn⁷-Tyr⁸-Cys⁹-Cys¹⁰-Glu¹¹-Lys¹²-Cys¹³- | 242 |
| | C13:C21 | Cys¹⁴-Asn¹⁵-Pro¹⁶-Ala¹⁷-Cys¹⁸-Thr¹⁹-Gly²⁰-Cys²¹-Tyr²² | |
| N161 | C3:C8, C4:C12, | PEG3-Asn¹-Phe²-Cys³-Cys⁴-Glu⁵-Thr⁶-Cys⁷-Cys⁸-Asn⁹-Pro¹⁰-Ala¹¹-Cys¹²- | 243 |
| | C7:C15 | Thr¹³-Gly¹⁴-Cys¹⁵-Tyr¹⁶-PEG3 | |
| N162 | C3:C8, C4:C12, | PEG3-Asn¹-Phe²-Cys³-Cys⁴-Glu⁵-Thr⁶-Cys⁷-Cys⁸-Asn⁹-Pro¹⁰-Ala¹¹-Cys¹²- | 244 |
| | C7:C15 | Thr¹³-Gly¹⁴-Cys¹⁵-Tyr¹⁶ | |
| N163 | C3:C8, C4:C12, | Asn¹-Phe²-Cys³-Cys⁴-Glu⁵-Thr⁶-Cys⁷-Cys⁸-Asn⁹-Pro¹⁰-Ala¹¹-Cys¹²-Thr¹³- | 245 |
| | C7:C15 | Gly¹⁴-Cys¹⁵-Tyr¹⁶-PEG3 | |
| N164 | C3:C8, C4:C12, | Asn¹-Phe²-Cys³-Cys⁴-Glu⁵-Tyr⁶-Cys⁷-Cys⁸-Asn⁹-Pro¹⁰-Ala¹¹-Cys¹²-Thr¹³- | 246 |
| | C7:C15 | Gly¹⁴-Cys¹⁵-Tyr¹⁶ | |
| N165 | C3:C8, C4:C12, | dAsn¹-Phe²-Cys³-Cys⁴-Glu⁵-Tyr⁶-Cys⁷-Cys⁸-Asn⁹-Pro¹⁰-Ala¹¹-Cys¹²-Thr¹³- | 247 |
| | C7:C15 | Gly¹⁴-Cys¹⁵-dTyr¹⁶ | |
| N166 | C3:C8, C4:C12, | Asn¹-Phe²-Cys³-Cys⁴-Glu⁵-Tyr⁶-Cys⁷-Cys⁸-Asn⁹-Pro¹⁰-Ala¹¹-Cys¹²-Thr¹³- | 248 |
| | C7:C15 | Gly¹⁴-Cys¹⁵-dTyr¹⁶ | |
| N167 | C3:C8, C4:C12, | dAsn¹-Phe²-Cys³-Cys⁴-Glu⁵-Tyr⁶-Cys⁷-Cys⁸-Asn⁹-Pro¹⁰-Ala¹¹-Cys¹²-Thr¹³- | 249 |
| | C7:C15 | Gly¹⁴-Cys¹⁵-Tyr¹⁶ | |

Preparation of GCRA Peptides

GCRA peptides are easily prepared using modern cloning techniques, or may be synthesized by solid state methods or by site-directed mutagenesis. A GCRA peptide may include dominant negative forms of a polypeptide.

Chemical synthesis may generally be performed using standard solution phase or solid phase peptide synthesis techniques, in which a peptide linkage occurs through the direct condensation of the amino group of one amino acid with the carboxy group of the other amino acid with the elimination of a water molecule. Peptide bond synthesis by direct condensation, as formulated above, requires suppression of the reactive character of the amino group of the first and of the carboxyl group of the second amino acid. The masking substituents must permit their ready removal, without inducing breakdown of the labile peptide molecule.

In solution phase synthesis, a wide variety of coupling methods and protecting groups may be used (See, Gross and Meienhofer, eds., “The Peptides: Analysis, Synthesis, Biology,” Vol. 1-4 (Academic Press, 1979); Bodansky and Bodansky, “The Practice of Peptide Synthesis,” 2d ed. (Springer Verlag, 1994)). In addition, intermediate purification and linear scale up are possible. Those of ordinary skill in the art will appreciate that solution synthesis requires consideration of main chain and side chain protecting groups and activation method. In addition, careful segment selection is necessary to minimize racemization during segment condensation. Solubility considerations are also a factor. Solid phase peptide synthesis uses an insoluble polymer for support during organic synthesis. The polymer-supported peptide chain permits the use of simple washing and filtration steps instead of laborious purifications at intermediate steps. Solid-phase peptide synthesis may generally be performed according to the method of Merrifield et al., J. Am. Chem. Soc., 1963, 85:2149, which involves assembling a linear peptide chain on a resin support using protected amino acids. Solid phase peptide synthesis typically utilizes either the Boc or Fmoc strategy, which is well known in the art.

Those of ordinary skill in the art will recognize that, in solid phase synthesis, deprotection and coupling reactions must go to completion and the side-chain blocking groups must be stable throughout the synthesis. In addition, solid phase synthesis is generally most suitable when peptides are to be made on a small scale.

Acetylation of the N-terminal can be accomplished by reacting the final peptide with acetic anhydride before cleavage from the resin. C-amidation is accomplished using an appropriate resin such as methylbenzhydrylamine resin using the Boc technology.

Alternatively the GCRA peptides are produced by modern cloning techniques. For example, the GCRA peptides are produced either in bacteria including, without limitation, E. coli, or in other existing systems for polypeptide or protein production (e.g., Bacillus subtilis, baculovirus expression systems using Drosophila Sf9 cells, yeast or filamentous fungal expression systems, mammalian cell expression systems), or they can be chemically synthesized. If the GCRA peptide or variant peptide is to be produced in bacteria, e.g., E. coli, the nucleic acid molecule encoding the polypeptide may also encode a leader sequence that permits the secretion of the mature polypeptide from the cell. Thus, the sequence encoding the polypeptide can include the pre sequence and the pro sequence of, for example, a naturally-occurring bacterial ST polypeptide. The secreted, mature polypeptide can be purified from the culture medium.

The sequence encoding a GCRA peptide described herein can be inserted into a vector capable of delivering and maintaining the nucleic acid molecule in a bacterial cell. The DNA molecule may be inserted into an autonomously replicating vector (suitable vectors include, for example, pGEM3Z and pcDNA3, and derivatives thereof). The vector nucleic acid may be a bacterial or bacteriophage DNA such as bacteriophage lambda or M13 and derivatives thereof. Construction of a vector containing a nucleic acid described herein can be followed by transformation of a host cell such as a bacterium. Suitable bacterial hosts include but are not limited to, E. coli, B subtilis, Pseudomonas, Salmonella. The genetic construct also includes, in addition to the encoding nucleic acid molecule, elements that allow expression, such as a promoter and regulatory sequences. The expression vectors may contain transcriptional control sequences that control transcriptional initiation, such as promoter, enhancer, operator, and repressor sequences.

A variety of transcriptional control sequences are well known to those in the art. The expression vector can also include a translation regulatory sequence (e.g., an untranslated 5′ sequence, an untranslated 3′ sequence, or an internal ribosome entry site). The vector can be capable of autonomous replication or it can integrate into host DNA to ensure stability during polypeptide production.

The protein coding sequence that includes a GCRA peptide described herein can also be fused to a nucleic acid encoding a polypeptide affinity tag, e.g., glutathione S-transferase (GST), maltose E binding protein, protein A, FLAG tag, hexa-histidine, myc tag or the influenza HA tag, in order to facilitate purification. The affinity tag or reporter fusion joins the reading frame of the polypeptide of interest to the reading frame of the gene encoding the affinity tag such that a translational fusion is generated. Expression of the fusion gene results in translation of a single polypeptide that includes both the polypeptide of interest and the affinity tag. In some instances where affinity tags are utilized, DNA sequence encoding a protease recognition site will be fused between the reading frames for the affinity tag and the polypeptide of interest.

Genetic constructs and methods suitable for production of immature and mature forms of the GCRA peptides and variants described herein in protein expression systems other than bacteria, and well known to those skilled in the art, can also be used to produce polypeptides in a biological system.

The peptides disclosed herein may be modified by attachment of a second molecule that confers a desired property upon the peptide, such as increased half-life in the body, for example, pegylation. Such modifications also fall within the scope of the term “variant” as used herein.

Compositions

The present invention provides a composition including at least one GC-C peptide (i.e., GCRA peptide). The composition may further include other therapeutic agents, including, but not limited to, a NF-κB inhibitor, a c-Src inhibitor, c-Myc inhibitors, Ikk inhibitors, an anti-inflammatory agent, an analgesic, a chemotherapeutic, or a combination thereof.

Exemplary NF-κB inhibitors include, but are not limited to, small molecules, chemical compounds and nucleic acid molecules which function to down regulate expression of target genes and inhibit the function of direct and indirect NF-κB signaling pathway, proteasome inhibitors, inhibitors of ubiquitin conjugation, inhibitors of proteasome peptidases, and protease inhibitors. Additionally, the use of antisense oligonucleotides to control the expression of cellular components is known in the art, and may be utilized in the present invention to reduce the expression of NFκB or its subunits. (Antisense oligonucleotides that hybridize to NF-κB mRNA, and their therapeutic use to suppress processes that depend on activation of NF-κB, are described in WO95/35032). Exemplary NF-κB inhibitors include, but are not limited to, inhibitors of chymotrypsin-like and trypsin-like proteases, and inhibitors of thiol (or cysteine) and serine proteases; natural and chemical protease inhibitors (such as peptides containing an α-diketone or an α-keto ester, peptide chloromethyl ketones, isocoumarins, peptide sulfonyl fluorides, peptidyl boronates, peptide epoxides, and peptidyl diazomethanes); pyrrolidine dithiocarbamate (PTDC); glucocorticoids, predonsone, prednisolone, methyl prednisolone, dexamethasone, prednisone, deoxycorticosterone, cortisone, hydrocortisone, nonglucocorticoid lazaroids, novel amides that are inhibitors of NF-κB DNA binding (WO 97/23457), antisense oligonucleotides that hybridize to NF-κB mRNA (WO95/35032). In a preferred embodiment, a NF-κB inhibitor is PTDC.

Exemplary src inhibitors include, without limitation, small molecules, chemical compounds and nucleic acid molecules which function to down regulate expression of target genes and inhibit the function of direct and indirect c-Src substrates, such as the focal adhesion kinase, signal transducer and activator of transcription 3 (STAT3), vascular endothelial growth factor (VEGF), paxillin, Cas, p190RhoGAP, RRas, E-cadherin, c-Jun amino-terminal kinase, NEDD9, and others. Exemplary agents include dasatinib, SU6656, and AZD05530. Src inhibitors are also available from Wyeth and include for example, 4-[(2,4-Dichloro-5-methoxyphenyl)amino]-7-[3-(4-ethyl-1-piperazinyl)propo-xy]-6-methoxy-3-quinolinecarbonitrile; 4-[(2,4-Dichloro-5-methoxyphenyl)amino]-6-methoxy-7-[2-(4-methyl-1-pipera-zinyl)ethoxy]-3-quinolinecarbonitrile; 4-[(2,4-Dichloro-5-methoxyphenyl)amino]-7-[2-(4-ethyl-1-piperazinyl)ethox-y]-6-methoxy-3-quinolinecarbonitrile; 4-[(2,4-Dichloro-5-methoxyphenyl)amino]-6-methoxy-7-[(1-methylpiperidin-4-yl)methoxy]-3-quinolinecarbonitrile; 4-[(2,4-Dichloro-5-methoxyphenyl)amino]-6-methoxy-7-[2-(1-methylpiperidin-4-yl)ethoxy]-3-quinolinecarbonitrile; 4-[(2,4-Dichloro-5-methoxyphenyl)amino]-6-methoxy-7-[3-(1-methylpiperidin-4-yl)propoxy]quinoline-3-carbonitrile; 4-[(2,4-Dichloro-5-methoxyphenyl)amino]-7-[(1-ethylpiperidin-4-yl)methoxy-]-6-methoxyquinoline-3-carbonitrile; 4-[(2,4-Dichloro-5-methoxyphenyl)amino]-6-ethoxy-7-[3-(4-methylpiperazin-1-yl)propoxy]quinoline-3-carbonitrile; 4-[(2,4-Dichloro-5-methoxyphenyl)amino]-6-ethoxy-7-[(1-methylpiperidin-4-yl)methoxy]quinoline-3-carbonitrile; 4-[(2,4-Dichloro-5-methoxyphenyl)amino]-6-ethoxy-7-[3-(4-ethylpiperazin-1-yl)propoxy]quinoline-3-carbonitrile; 4-[(2,4-Dichloro-5-methoxyphenyl)amino]-6-ethoxy-7-[3-(1-methylpiperidin-4-yl)propoxy]quinoline-3-carbonitrile; 4-[(2,4-Dichloro-5-methoxyphenyl)amino]-6-ethoxy-7-[2-(4-methyl-1-piperaz-inyl)ethoxy]quinoline-3-carbonitrile; 4-[(2,4-Dichloro-5-methoxyphenyl)amino]-6-ethoxy-7-[2-(1-methylpiperidin-4-yl)ethoxy]quinoline-3-carbonitrile; or 4-[(2,4-Dichloro-5-methoxyphenyl)amino]-6-methoxy-7-[3-(4-propyl-1-pipera-zinyl)propoxy]-3-quinolinecarbonitrile; and pharmaceutically acceptable salts thereof.

Suitable compounds possessing inhibitory activity against the Src family of non-receptor tyrosine kinases include the quinazoline derivatives disclosed in International Patent Applications WO 01/94341, WO 02/16352, WO 02/30924, WO 02/30926, WO 02/34744, WO 02/085895, WO 02/092577 (arising from PCT/GB 02/02117), WO 02/092578 (arising from PCT/GB 02/02124) and WO 02/092579 (arising from PCT/GB 02/02128), the quinoline derivatives described in WO 03/008409 (arising from PCT/GB 02/03177), WO 03/047584 and WO 03/048159 and the quinazoline derivatives described in European Patent Applications 02292736.2 (filed 4 Nov. 2002) and 03290900.4 (filed 10 Apr. 2003).

It is disclosed in Journal Medicinal Chemistry, 2001, 44, 822-833 and 3965-3977 that certain 4-anilino-3-cyanoquinoline derivatives are useful for the inhibition of Src-dependent cell proliferation. The 4-anilino-3-cyanoquinoline Src inhibitor known as SKI 606 is described in Cancer Research, 2003, 63, 375.

Other compounds which possess Src kinase inhibitory properties are described in, for example, International Patent Applications WO 96/10028, WO 97/07131, WO 97/08193, WO 97/16452, WO 97/28161, WO 97/32879 and WO 97/49706.

Other compounds which possess Src kinase inhibitory properties are described in, for example, J Bone Mineral Research, 1999, 14 (Suppl. 1), 5487, Molecular Cell, 1999, 3, 639-647, Journal Medicinal Chemistry, 1997, 40, 2296-2303, Journal Medicinal Chemistry, 1998, 41, 3276-3292 and Bioorganic & Medicinal Chemistry Letters, 2002, 12, 1361 and 3153.

Particular Src kinase inhibitors include the following:

(i) 4-amino-5-(3-methoxyphenyl)-7-{(4-[2-(2-methoxyethylamino)ethox-y]phenyl)-}-pyrrolo[2,3-d]pyrimidine and 4-amino-5-(3-methoxyphenyl)-7-(4-{(2-[di-(2-methoxyethyl)amino]ethoxy}phe-nyl)pyrrolo[2,3-d]pyrimidine which are obtainable by methods described in International Patent Application WO 96/10028;

(ii) 4-amino-7-tert-butyl-5-(4-tolyl)pyrazolo[3,4-d]pyrimidine which is also known as PP1 and is described in Molecular Cell, 1999, 3, 639-648;

(iii) 2-(2,6-dichloroanilino)-6,7-dimethyl-1,8-dihydroimidazo[4,5-h]isoquinolin-9-one and 2-(2,6-dichloroanilino)-7-[(E)-3-diethylaminoprop-1-enyl]-6-met-hyl-1,8-dihydroimidazo[4,5-h]isoquinolin-9-one which are obtainable by methods described in Journal Medicinal Chemistry, 2002, 45, 3394;

(iv) 1-[6-(2,6-dichlorophenyl)-2-(4-diethylaminobutyl)pyrido[2,3-d]pyrimidin-7-yl]-3-ethylurea which is obtainable by methods described in Journal Medicinal Chemistry, 1997, 40, 2296-2303 and Journal Medicinal Chemistry, 2001, 44, 1915;

(v) 6-(2,6-dichlorophenyl)-2-[4-(2-diethylaminoethoxy)anilino]-8-me-thyl-8H-pyrido[2,3-d]pyrimidin-7-one which is also known as PD166285 and is described in J. Pharmacol. Exp. Ther., 1997, 283, 1433-1444;

(vi) the compound known as PD 162531 which is described in Mol. Biol. Cell, 2000, 11, 51-64;

(vii) the compound known as PD166326 which is described in Biochem Pharmacol., 2000, 60, 885-898; and

(viii) the compound known as PD173955 which is described in Cancer Research, 1999, 59, 6145-6152.

Other compounds which may possess Src kinase inhibitory properties are described in, for example, International Patent Applications WO 02/079192, WO 03/000188, WO 03/000266, WO 03/000705, WO 02/083668, WO 02/092573, WO 03/004492, WO 00/49018, WO 03/013541, WO 01/00207, WO 01/00213 and WO 01/00214. Particular Src inhibitors include those provided in International Patent Application WO 01/94341. Further particular Src inhibitors include the following compounds from International Patent Application WO 02/16352, WO 02/30924, WO 02/30926 and WO 02/34744.

In a preferred embodiment, a c-Src tyrosine kinase inhibitor is: N-benzyl-2-(5-(4-(2-morpholinoethoxy)phenyl)pyridin-2-yl)acetamide (also called KX2-391) or PP2 (protein phosphatase 2).

Exemplary c-Myc inhibitors include, but are not limited to Myc inhibitiors, include, but are not limited to, Omomyc transgene, Small-molecule disruptors of MYC:MAX heterodimerization, small molecules (e.g. 10058-F4 (Huang et al. 2006)), BET bromodomain inhibitors (e.g. JQ1), alkylating agents (e.g. Mitomycin C), DHFR inhibitors (e.g. Methotrexate), histone deacetylase inhibitors (e.g. Trichostatin-A), protein synthesis inhibitors (e.g. anisomycin, cycloheximide), kinase inhibitors (e.g. staaurosporine), 20 S-proteasome chymotrypsin inhibitors (e.g. Gliotoxin), topoisomerase I inhibitors (e.g. Camptothecin, 10-Hydroxycamptothecin), Topoisomerase II inhibitors (e.g. Etoposide), tubulin inhibitors (e.g. podophyllotoxin, Vinblastine), RNA synthesis inhibitors (e.g. Actinomycin D), HSP-90 inhibitors (e.g. 17-allylamino-geldanamycin), and DNA polymerase inhibitors (e.g. Aphicicolin).

Exemplary Ikk inhibitors include, but are not limited to, NEMO Binding Domain Peptide, anti-inflammatory agents (e.g. aspirin, sodium salicylate, sulindac), thalidomide, cyclopentenone prostaglandins, Arsenic trioxide, quinazoline analogues (e.g. SPC 839), ATP analogs, β-carbolines (e.g. PS-1145, ML120B), aminothiophenecarboximide (e.g. SC514, BMS-34541), ureidocarboximide thiophenes, pyridooxazin derivatives, and small molecule inhibitors.

In some embodiments, the compositions described herein are formulated in a pH dependent release form. Alternatively, such compositions are formulated in a form that releases the peptides at a specific region of the gastrointestinal (GI) tract (e.g., duodenum, jejunum, ileum, terminal ileum, or ascending colon). The formulation may contain an inert carrier coated with a composition and an enteric coating which releases the peptides at a specific pH (such as pH5 or pH7). Preferred pH for duodenum or jejunum release is pH 4.5-5.5 or pH 5.5-6.5. Preferred pH for ileum, terminal ileum, or ascending colon release is pH 5.5-6.5 or pH 6.5-7.5. Preferably, the inert carrier is a selected from mannitol, lactose, a microcrystalline cellulose, or starch.

Methods

The invention relates in part to the use of GC-C agonists to inhibit activation of NF-κB, to reduce production of pro-inflammatory cytokines/chemokines, and/or to increase secretion of anti-inflammatory cytokines.

Accoridngly, the present invention provides methods of treating a disorder that is mediated by guanylate cyclase receptor agonists by administering to a subject in need thereof an effective amount of a GCRA peptide or pharmaceutical composition thereof or a composition described herein, where the effective amount is sufficient to inhibit NF-κB activation. Disorders mediated by the guanylate cyclase receptor agonists include lipid metabolism disorders, biliary disorders, gastrointestinal disorders, inflammatory disorders, lung disorders, cancer, cardiac disorders including cardiovascular disorders, eye disorders, oral disorders, blood disorders, liver disorders, skin disorders, prostate disorders, endocrine disorders, increasing gastrointestinal motility and obesity. Lipid metabolism disorders include, but not limited to, dyslipidemia, hyperlipidemia, hypercholesterolemia, hypertriglyceridemia, sitosterolemia, familial hypercholesterolemia, xanthoma, combined hyperlipidemia, lecithin cholesterol acyltransferase deficiency, tangier disease, abetalipoproteinemia, erectile dysfunction, fatty liver disease, and hepatitis. Billary disorders include gallbladder disorders such as for example, gallstones, gall bladder cancer cholangitis, or primary sclerosing cholangitis; or bile duct disorders such as for example,cholecystitis, bile duct cancer or fascioliasis. Gastointestinal disorders include for example, irritable bowel syndrome (IBS), non-ulcer dyspepsia, chronic intestinal pseudo-obstruction, functional dyspepsia, colonic pseudo-obstruction, duodenogastric reflux, gastroesophageal reflux disease (GERD), ileus inflammation (e.g., post-operative ileus), gastroparesis, heartburn (high acidity in the GI tract), constipation (e.g., IBS-associated constipation, constipation associated with use of medications such as opioids, osteoarthritis drugs, osteoporosis drugs; post surigical constipation, constipation associated with neuropathic disorders). Inflammatory disorders include tissue and organ inflammation such as kidney inflammation (e.g., nephritis), gastrointestinal system inflammation (e.g., chronic inflammatory bowel disease, Crohn's disease, colitis, and ulcerative colitis); necrotizing enterocolitis (NEC); pancreatic inflammation (e.g., pancreatis), lung inflammation (e.g., bronchitis or asthma) or skin inflammation (e.g., psoriasis, eczema). Lung disorders include for example chronic obstructive pulmonary disease (COPD), and fibrosis. Cancer includes tissue and organ carcinogenesis including metatases such as for example gastrointestinal cancer (e.g., gastric cancer, esophageal cancer, pancreatic cancer, colorectal cancer including colorectal metastasis, intestinal cancer, anal cancer, liver cancer, gallbladder cancer, or colon cancer); lung cancer; thyroid cancer; skin cancer (e.g., melanoma); oral cancer; urinary tract cancer (e.g. bladder cancer or kidney cancer); blood cancer (e.g. myeloma or leukemia) or prostate cancer. Cardiac disorders include for example, congestive heart failure, trachea cardia hypertension, high cholesterol, or high tryglycerides. Cardiovascular disorders include for example aneurysm, angina, atherosclerosis, cerebrovascular accident (stroke), cerebrovasculardisease, congestive heart failure, coronary artery disease, myocardial infarction (heart attack), or peripheral vascular disease. Liver disorders include for example cirrhosis and fibrosis. In addition, GC-C agonist may also be useful to facilitate liver regeneration in liver transplant patients. Eye disorders include for example increased intra-ocular pressure, glaucoma, dry eyes retinal degeneration, disorders of tear glands or eye inflammation. Skin disorders include for example xerosis. Oral disorders include for example dry mouth (xerostomia), Sjogren's syndrome, gum diseases (e.g., periodontal disease), or salivary gland duct blockage or malfunction. Prostate disorders include for example benign prostatic hyperplasia (BPH). Endocrine disorders include for example diabetes mellitus, hyperthyroidism, hypothyroidism, and cystic fibrosis.

In a preferred embodiment, the disorder mediated by the guanylate cyclase receptor agonists is a GI inflammatory disorder, such as chronic inflammatory bowel disease, Crohn's disease, colitis, ulcerative colitis, DSS- and TNBS-induced colitis, inflammation-induced colonic cancer, or colorectal metastasis.

The present invention also provides a method of modulating NF-κB induction in a cell by contacting the cell with an effective amount of a GCRA peptide or pharmaceutical composition thereof, or a composition described herein.

The present invention also provides a method of modulating NF-κB-dependent target gene expression in a cell by contacting the cell with an effective amount of a GCRA peptide or pharmaceutical composition thereof or a composition described herein, where the GCRA peptide or the composition inhibits NF-κB activation. NF-κB-dependent target gene includes, for example, TNF.

NF-κB may include one or more transcription factor of the NF-κB family, for example without being limited to the list herein, NF-κB1 (p50), NF-κB2 (p52), p65 (RelA), c-Rel, and RelB, or any protein that share a common structural motif called the Rel homology domain.

Pro-inflammatory cytokines include, but are not limited to, IL-1, IL-2, TNF, IL-12p40, IL-17, and IL-23. Chemokines include, but are not limited to, IL-8, RANTES and MIP-1α. Anti-inflammatory cytokines include, but are not limited to, IL-10.

In some embodiments, GCRA peptides inhibit the nuclear localization of NF-κB.

In some embodiments, GCRA peptides mediate inhibition of NF-κB activating factors. NF-κB activating factors are, without being limited to the examples herein, are cytokines such as tumor necrosis factor (TNF) and interleukin (IL)-1, lipopolysaccharides, bacterial and viral infections, activators of protein kinase C, and oxidants. The inhibition of NF-κB may result in reduced production of cytokines (such as, without being limited to the examples herein, TNF, IL-1, IL-2, IL-6, IL-8, IL-12p40, IL-17, and IL-23), adhesion molecules (such as ICAM-1, VCAM-1, E-selectin, and MAdCAM-1), and enzymes that are involved in inflammation, such as inducible nitric oxide synthase and cyclooxygenase-2. In some embodiments, the invention may provide inhibition of proteins whose genes are switched on by NF-κB, such as, without being limited to the examples herein, TNF and IL-1.

In some embodiments, the inhibition of NF-κB activation with GC-C agonists may prevent IkB degradation and may attenuate chronic inflammation associated with Crohn's disease. In another embodiment, inhibition of p65 subunit of NF-κB may effectively abrogate colonic inflammation associated with colitis.

In another embodiment of the current invention, inhibition of NF-κB with GC-C agonists may prevent mucosal NF-κB activation in ulcerative colitis patients. In particular the GC-C agonists of the current invention may inhibit NF-κB activation in macrophages.

In some embodiments, GC-C agonists mediated enhancement or stimulation of cGMP signaling pathway results in the inhibition of NF-κB. According to some embodiments, the GC-C agonist mediated enhancement or stimulation of cGMP may result in the activation of the cyclic dependent protein kinase (PKG). The cyclic GMP-dependent kinase (PKG) is an important mediator of signal transduction that may induce gene expression through cAMP response element binding protein (CREB).

By “inhibiting” or “inhibition” or “reduce”, it means the GCRA peptide decreases the activity and/or production of a protein by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, or more relative to the activity and/or production of the protein without the GCRA peptide.

By “induce”, it means the GCRA peptide increases the activity and/or production of a protein by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, or more relative to the activity and/or production of the protein without the GCRA peptide.

The term “treatment” or “treating” refers to reducing or alleviating symptoms in a subject, preventing symptoms from worsening or progressing, and/or preventing disease in a subject who is free therefrom. For a given subject, improvement, worsening, regression, or progression of a symptom may be determined by any objective or subjective measure. Efficacy of the treatment may be measured as an improvement in morbidity or mortality (e.g., lengthening of survival curve for a selected population). Thus, effective treatment would include therapy of existing disease, control of disease by slowing or stopping its progression, prevention of disease occurrence, reduction in the number or severity of symptoms, or a combination thereof. The effect may be shown in a controlled study using one or more statistically significant criteria.

The term “prevention” in relation to a given disease or disorder means: preventing the onset of disease development if none had occurred, preventing the disease or disorder from occurring in a subject that may be predisposed to the disorder or disease but has not yet been diagnosed as having the disorder or disease, and/or preventing further disease/disorder development if already present.

Intracellular cGMP produced by exposing, e.g., contacting a tissue (e.g., gastrointestinals tissue) or cell with GCRA agonists. By inducing is meant an increase in cGMP production compared to a tissue or cell that has not been in contact with GCRA peptide or variant. Tissues or cells are directly contacted with a GCRA peptide or variant. Alternatively, the GCRA peptide or variant is administered systemically. GCRA peptide or variant are administered in an amount sufficient to increase intracellular cGMP concentration. cGMP production is measured by a cell-based assay known in the art (25).

Disorders are treated, prevented or alleviated by administering to a subject, e.g., a mammal such as a human in need thereof, a therapeutically effective dose of a GCRA peptide. The GCRA peptides may be in a pharmaceutical composition in unit dose form, together with one or more pharmaceutically acceptable excipients. The term “unit dose form” refers to a single drug delivery entity, e.g., a tablet, capsule, solution or inhalation formulation. The amount of peptide present should be sufficient to have a positive therapeutic effect when administered to a patient (typically, between 10 μg and 3 g). What constitutes a “positive therapeutic effect” will depend upon the particular condition being treated and will include any significant improvement in a condition readily recognized by one of skill in the art.

The GCRA peptides can be administered alone or in combination with other agents. For example the GCRA peptides can be administered in combination with a NF-κB inhibitor, a src inhibitor, 5-ASA, or inhibitors of cGMP dependent phosphodiesterase, such as, for example, suldinac sulfone, zaprinast, motapizone, vardenafil or sildenifil; one or more other chemotherapeutic agents; or anti-inflammatory drugs such as, for example, steroids or non-steroidal anti-inflammatory drugs (NSAIDS), such as aspirin.

Exemplary NF-κB inhibitors include, but are not limited to, inhibitors of chymotrypsin-like and trypsin-like proteases, and inhibitors of thiol (or cysteine) and serine proteases; natural and chemical protease inhibitors (such as peptides containing an α-diketone or an α-keto ester, peptide chloromethyl ketones, isocoumarins, peptide sulfonyl fluorides, peptidyl boronates, peptide epoxides, and peptidyl diazomethanes); pyrrolidine dithiocarbamate (PTDC); glucocorticoids, predonsone, prednisolone, methyl prednisolone, dexamethasone, prednisone, deoxycorticosterone, cortisone, hydrocortisone, nonglucocorticoid lazaroids, novel amides that are inhibitors of NF-κB DNA binding (WO 97/23457), antisense oligonucleotides that hybridize to NF-κB mRNA (WO95/35032). In a preferred embodiment, a NF-κB inhibitor is PTDC.

Exemplary src inhibitors include, without limitation, small molecules, chemical compounds and nucleic acid molecules which function to down regulate expression of target genes and inhibit the function of direct and indirect c-Src substrates, such as the focal adhesion kinase, signal transducer and activator of transcription 3 (STAT3), vascular endothelial growth factor (VEGF), paxillin, Cas, p190RhoGAP, RRas, E-cadherin, c-Jun amino-terminal kinase, NEDD9, and others. Exemplary agents include dasatinib, SU6656, and AZD05530. Src inhibitors are also available from Wyeth and include for example, 4-[(2,4-Dichloro-5-methoxyphenyl)amino]-7-[3-(4-ethyl-1-piperazinyl)propo-xy]-6-methoxy-3-quinolinecarbonitrile; 4-[(2,4-Dichloro-5-methoxyphenyl)amino]-6-methoxy-7-[2-(4-methyl-1-pipera-zinyl)ethoxy]-3-quinolinecarbonitrile; 4-[(2,4-Dichloro-5-methoxyphenyl)amino]-7-[2-(4-ethyl-1-piperazinyl)ethox-y]-6-methoxy-3-quinolinecarbonitrile; 4-[(2,4-Dichloro-5-methoxyphenyl)amino]-6-methoxy-7-[(1-methylpiperidin-4-yl)methoxy]-3-quinolinecarbonitrile; 4-[(2,4-Dichloro-5-methoxyphenyl)amino]-6-methoxy-7-[2-(1-methylpiperidin-4-yl)ethoxy]-3-quinolinecarbonitrile; 4-[(2,4-Dichloro-5-methoxyphenyl)amino]-6-methoxy-7-[3-(1-methylpiperidin-4-yl)propoxy]quinoline-3-carbonitrile; 4-[(2,4-Dichloro-5-methoxyphenyl)amino]-7-[(1-ethylpiperidin-4-yl)methoxy-]-6-methoxyquinoline-3-carbonitrile; 4-[(2,4-Dichloro-5-methoxyphenyl)amino]-6-ethoxy-7-[3-(4-methylpiperazin-1-yl)propoxy]quinoline-3-carbonitrile; 4-[(2,4-Dichloro-5-methoxyphenyl)amino]-6-ethoxy-7-[(1-methylpiperidin-4-yl)methoxy]quinoline-3-carbonitrile; 4-[(2,4-Dichloro-5-methoxyphenyl)amino]-6-ethoxy-7-[3-(4-ethylpiperazin-1-yl)propoxy]quinoline-3-carbonitrile; 4-[(2,4-Dichloro-5-methoxyphenyl)amino]-6-ethoxy-7-[3-(1-methylpiperidin-4-yl)propoxy]quinoline-3-carbonitrile; 4-[(2,4-Dichloro-5-methoxyphenyl)amino]-6-ethoxy-7-[2-(4-methyl-1-piperaz-inyl)ethoxy]quinoline-3-carbonitrile; 4-[(2,4-Dichloro-5-methoxyphenyl)amino]-6-ethoxy-7-[2-(1-methylpiperidin-4-yl)ethoxy]quinoline-3-carbonitrile; or 4-[(2,4-Dichloro-5-methoxyphenyl)amino]-6-methoxy-7-[3-(4-propyl-1-pipera-zinyl)propoxy]-3-quinolinecarbonitrile; and pharmaceutically acceptable salts thereof.

Suitable compounds possessing inhibitory activity against the Src family of non-receptor tyrosine kinases include the quinazoline derivatives disclosed in International Patent Applications WO 01/94341, WO 02/16352, WO 02/30924, WO 02/30926, WO 02/34744, WO 02/085895, WO 02/092577 (arising from PCT/GB 02/02117), WO 02/092578 (arising from PCT/GB 02/02124) and WO 02/092579 (arising from PCT/GB 02/02128), the quinoline derivatives described in WO 03/008409 (arising from PCT/GB 02/03177), WO 03/047584 and WO 03/048159 and the quinazoline derivatives described in European Patent Applications 02292736.2 (filed 4 Nov. 2002) and 03290900.4 (filed 10 Apr. 2003).

It is disclosed in Journal Medicinal Chemistry, 2001, 44, 822-833 and 3965-3977 that certain 4-anilino-3-cyanoquinoline derivatives are useful for the inhibition of Src-dependent cell proliferation. The 4-anilino-3-cyanoquinoline Src inhibitor known as SKI 606 is described in Cancer Research, 2003, 63, 375.

Other compounds which possess Src kinase inhibitory properties are described in, for example, International Patent Applications WO 96/10028, WO 97/07131, WO 97/08193, WO 97/16452, WO 97/28161, WO 97/32879 and WO 97/49706.

Other compounds which possess Src kinase inhibitory properties are described in, for example, J Bone Mineral Research, 1999, 14 (Suppl. 1), S487, Molecular Cell, 1999, 3, 639-647, Journal Medicinal Chemistry, 1997, 40, 2296-2303, Journal Medicinal Chemistry, 1998, 41, 3276-3292 and Bioorganic & Medicinal Chemistry Letters, 2002, 12, 1361 and 3153.

Particular Src kinase inhibitors include the following:

(i) 4-amino-5-(3-methoxyphenyl)-7-{(4-[2-(2-methoxyethylamino)ethox-y]phenyl)-}-pyrrolo[2,3-d]pyrimidine and 4-amino-5-(3-methoxyphenyl)-7-(4-{(2-[di-(2-methoxyethyl)amino]ethoxy}phe-nyl)pyrrolo[2,3-d]pyrimidine which are obtainable by methods described in International Patent Application WO 96/10028;

(ii) 4-amino-7-tert-butyl-5-(4-tolyl)pyrazolo[3,4-d]pyrimidine which is also known as PP1 and is described in Molecular Cell, 1999, 3, 639-648;

(iii) 2-(2,6-dichloroanilino)-6,7-dimethyl-1,8-dihydroimidazo[4,5-h]isoquinolin-9-one and 2-(2,6-dichloroanilino)-7-[(E)-3-diethylaminoprop-1-enyl]-6-met-hyl-1,8-dihydroimidazo[4,5-h]isoquinolin-9-one which are obtainable by methods described in Journal Medicinal Chemistry, 2002, 45, 3394;

(iv) 1-[6-(2,6-dichlorophenyl)-2-(4-diethylaminobutyl)pyrido[2,3-d]pyrimidin-7-yl]-3-ethylurea which is obtainable by methods described in Journal Medicinal Chemistry, 1997, 40, 2296-2303 and Journal Medicinal Chemistry, 2001, 44, 1915;

(v) 6-(2,6-dichlorophenyl)-2-[4-(2-diethylaminoethoxy)anilino]-8-me-thyl-8H-pyrido[2,3-d]pyrimidin-7-one which is also known as PD166285 and is described in J. Pharmacol. Exp. Ther., 1997, 283, 1433-1444;

(vi) the compound known as PD 162531 which is described in Mol. Biol. Cell, 2000, 11, 51-64;

(vii) the compound known as PD166326 which is described in Biochem Pharmacol., 2000, 60, 885-898; and

(viii) the compound known as PD173955 which is described in Cancer Research, 1999, 59, 6145-6152.

Other compounds which may possess Src kinase inhibitory properties are described in, for example, International Patent Applications WO 02/079192, WO 03/000188, WO 03/000266, WO 03/000705, WO 02/083668, WO 02/092573, WO 03/004492, WO 00/49018, WO 03/013541, WO 01/00207, WO 01/00213 and WO 01/00214. Particular Src inhibitors include those provided in International Patent Application WO 01/94341. Further particular Src inhibitors include the following compounds from International Patent Application WO 02/16352, WO 02/30924, WO 02/30926 and WO 02/34744.

In a preferred embodiment, a c-Src tyrosine kinase inhibitor is: N-benzyl-2-(5-(4-(2-morpholinoethoxy)phenyl)pyridin-2-yl)acetamide (also called KX2-391) or PP2 (protein phosphatase 2).

The term “combination therapy” means administering two or more active agents concurrently or sequentially. Concurrent administration may be achieved with a formulation in which two or more active agents are mixed, or with simultaneous administration of two or more active agents formulated independently. Sequential administration of two or more active agents may be achieved with two or more active agents, formulated independently, administered in sequence with one agent administered first followed by the second agent administered seconds, minutes, hours, or days after the first agent.

Combination therapy can be achieved by administering two or more agents, e.g., a GCRA peptide described herein or a composition described herein and another compound, each of which is formulated and administered separately, or by administering two or more agents in a single formulation. Other combinations are also encompassed by combination therapy. For example, two agents can be formulated together and administered in conjunction with a separate formulation containing a third agent. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within 1, 2, 3, 6, 9, 12, 15, 18, or 24 hours of each other or within 1, 2, 3, 4, 5, 6, 7, 8, 9, 10, 12, or 14 days of each other or within 2, 3, 4, 5, 6, 7, 8, 9, or 10 weeks of each other. In some cases even longer intervals are possible. While in many cases it is desirable that the two or more agents used in a combination therapy be present in within the patient's body at the same time, this need not be so.

The combination therapies featured in the present invention can result in a synergistic effect in the treatment of a disease or cancer. A “synergistic effect” is defined as where the efficacy of a combination of therapeutic agents is greater than the sum of the effects of any of the agents given alone. A synergistic effect may also be an effect that cannot be achieved by administration of any of the compounds or other therapeutic agents as single agents.

In some embodiments, the term “synergistic effect” means the combination of a GCRA peptide and a selected compound described herein reduces about 5%, about 10%, about 20%, about 30%, about 40%, about 50%, about 75%, about 90% or more inflammation compared to a GCRA peptide alone or a selected compound alone.

In some embodiments, the term “synergistic effect” means the combination of a GCRA peptide and a selected compound described herein down regulates about 5%, about 10%, about 20%, about 30%, about 40%, about 50%, about 75%, about 90% or more NF-κB signaling compared to a GCRA peptide alone or a selected compound alone.

In some embodiments, the term “synergistic effect” means the combination of a GCRA peptide and a selected compound described herein induces about 5%, about 10%, about 20%, about 30%, about 40%, about 50%, about 75%, about 90% or more secretion of anti-inflammatory cytokines compared to a GCRA peptide alone or a selected compound alone.

In some embodiments, the term “synergistic effect” means the combination of a GCRA peptide and a selected compound described herein reducesabout 5%, about 10%, about 20%, about 30%, about 40%, about 50%, about 75%, about 90% or more production of pro-inflammatory ctyokines compared to a GCRA peptide alone or a selected compound alone.

In some embodiments, the term “synergistic effect” means the combination of a GCRA peptide and a selected compound described herein induces about 5%, about 10%, about 20%, about 30%, about 40%, about 50%, about 75%, about 90% or more apoptosis compared to a GCRA peptide alone or a selected compound alone.

Thus, GC-C agonists may be used either alone or in combination with other anti-inflammatory drugs, for example, Sulfasalazine (Azulfidine) Mesalamine (Asacol, Lialda), balsalazide (Colazal), olsalazine (Dipentum), Corticosteroids, immune system suppressors, for example, Azathioprine (Azasan, Imuran) and mercaptopurine (Purinethol), Cyclosporine (Gengraf, Neoral, Sandimmune), Infliximab (Remicade), and/or immunomodulatory agents (such as 6-mercaptopurine and methotrexate).

The GCRA peptides described herein may be combined with phosphodiesterase inhibitors, e.g., sulindae sulfone, Zaprinast, sildenafil, vardenafil or tadalafil to further enhance levels of cGMP in the target tissues or organs.

Combination therapy can also include two or more administrations of one or more of the agents used in the combination. For example, if agent X and agent Y are used in a combination, one could administer them sequentially in any combination one or more times, e.g., in the order X-Y-X, X-X-Y, Y-X-Y,Y-Y-X,X-X-Y-Y, etc.

Combination therapy can also include the administration of one of the GC-C agonist with azothioprine and/or other immunomodulating agents. The immunomodulating agents may include small molecule drugs and biologics such as Remicade, Humaira, Cimzia etc.

Combination therapy can also include the administration of two or more agents via different routes or locations. For example, (a) one agent is administered orally and another agents is administered intravenously or (b) one agent is administered orally and another is administered locally. In each case, the agents can either simultaneously or sequentially. Approximated dosages for some of the combination therapy agents described herein are found in the “BNF Recommended Dose” column of tables on pages 11-17 of WO01/76632 (the data in the tables being attributed to the March 2000 British National Formulary) and can also be found in other standard formularies and other drug prescribing directories. For some drugs, the customary prescribed dose for an indication will vary somewhat from country to country.

The GCRA peptides, alone or in combination, can be combined with any pharmaceutically acceptable carrier or medium. Thus, they can be combined with materials that do not produce an adverse, allergic or otherwise unwanted reaction when administered to a patient. The carriers or mediums used can include solvents, dispersants, coatings, absorption promoting agents, controlled release agents, and one or more inert excipients (which include starches, polyols, granulating agents, microcrystalline cellulose (e.g. celphere, Celphere beads®), diluents, lubricants, binders, disintegrating agents, and the like), etc. If desired, tablet dosages of the disclosed compositions may be coated by standard aqueous or nonaqueous techniques.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include: sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL□ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a GCRA agonist) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. Such as mannitol, fructooligosaccharides, polyethylene glycol and other excepients. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811, incorporated fully herein by reference.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved.

The pharmaceutical compositions can be included in a container, pack, or dispenser together with instructions for administration.

Compositions of the present invention may also optionally include other therapeutic ingredients, anti-caking agents, preservatives, sweetening agents, colorants, flavors, desiccants, plasticizers, dyes, glidants, anti-adherents, anti-static agents, surfactants (wetting agents), anti-oxidants, film-coating agents, and the like. Any such optional ingredient must be compatible with the compound described herein to insure the stability of the formulation.

The composition may contain other additives as needed, including for exanple lactose, glucose, fructose, galactose, trehalose, sucrose, maltose, raffnose, maltitol, melezitose, stachyose, lactitol, palatinite, starch, xylitol, mannitol, myoinositol, and the like, and hydrates thereof, and amino acids, for example alanine, glycine and betaine, and polypeptides and proteins, for example albumen.

Examples of excipients for use as the pharmaceutically acceptable carriers and the pharmaceutically acceptable inert carriers and the aforementioned additional ingredients include, but are not limited to binders, fillers, disintegrants, lubricants, anti-microbial agents, and coating agents such as: BINDERS: corn starch, potato starch, other starches, gelatin, natural and synthetic gums such as acacia, xanthan, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone (e.g., povidone, crospovidone, copovidone, etc), methyl cellulose, Methocel, pre-gelatinized starch (e.g., STARCH 1500® and STARCH 1500 LM®, sold by Colorcon, Ltd.), hydroxypropyl methyl cellulose, microcrystalline cellulose (FMC Corporation, Marcus Hook, Pa., USA), or mixtures thereof, FILLERS: talc, calcium carbonate (e.g., granules or powder), dibasic calcium phosphate, tribasic calcium phosphate, calcium sulfate (e.g., granules or powder), microcrystalline cellulose, powdered cellulose, dextrates, kaolin, mannitol, silicic acid, sorbitol, starch, pre-gelatinized starch, dextrose, fructose, honey, lactose anhydrate, lactose monohydrate, lactose and aspartame, lactose and cellulose, lactose and microcrystalline cellulose, maltodextrin, maltose, mannitol, microcrystalline cellulose &amp; guar gum, molasses, sucrose,or mixtures thereof, DISINTEGRANTS: agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, clays, other algins, other celluloses, gums (like gellan), low-substituted hydroxypropyl cellulose, or mixtures thereof, LUBRICANTS: calcium stearate, magnesium stearate, mineral oil, light mineral oil, glycerin, sorbitol, mannitol, polyethylene glycol, other glycols, stearic acid, sodium lauryl sulfate, sodium stearyl fumarate, vegetable based fatty acids lubricant, talc, hydrogenated vegetable oil (e.g., peanut oil, cottonseed oil, sunflower oil, sesame oil, olive oil, corn oil and soybean oil), zinc stearate, ethyl oleate, ethyl laurate, agar, syloid silica gel (AEROSIL 200, W.R. Grace Co., Baltimore, Md. USA), a coagulated aerosol of synthetic silica (Deaussa Co., Piano, Tex. USA), a pyrogenic silicon dioxide (CAB-O-SIL, Cabot Co., Boston, Mass. USA), or mixtures thereof, ANTI-CAKING AGENTS: calcium silicate, magnesium silicate, silicon dioxide, colloidal silicon dioxide, talc, or mixtures thereof, ANTIMICROBIAL AGENTS: benzalkonium chloride, benzethonium chloride, benzoic acid, benzyl alcohol, butyl paraben, cetylpyridinium chloride, cresol, chlorobutanol, dehydroacetic acid, ethylparaben, methylparaben, phenol, phenylethyl alcohol, phenoxyethanol, phenylmercuric acetate, phenylmercuric nitrate, potassium sorbate, propylparaben, sodium benzoate, sodium dehydroacetate, sodium propionate, sorbic acid, thimersol, thymo, or mixtures thereof, and COATING AGENTS: sodium carboxymethyl cellulose, cellulose acetate phthalate, ethylcellulose, gelatin, pharmaceutical glaze, hydroxypropyl cellulose, hydroxypropyl methylcellulose (hypromellose), hydroxypropyl methyl cellulose phthalate, methylcellulose, polyethylene glycol, polyvinyl acetate phthalate, shellac, sucrose, titanium dioxide, carnauba wax, microcrystalline wax, gellan gum, maltodextrin, methacrylates, microcrystalline cellulose and carrageenan or mixtures thereof.

The formulation can also include other excipients and categories thereof including but not limited to L-histidine, Pluronic®, Poloxamers (such as Lutrol® and Poloxamer 188), ascorbic acid, glutathione, permeability enhancers (e.g. lipids, sodium cholate, acylcarnitine, salicylates, mixed bile salts, fatty acid micelles, chelators, fatty acid, surfactants, medium chain glycerides), protease inhibitors (e.g. soybean trypsin inhibitor, organic acids), pH lowering agents and absorption enhancers effective to promote bioavailability (including but not limited to those described in U.S. Pat. No. 6,086,918 and U.S. 5,912,014), creams and lotions (like maltodextrin and carrageenans); materials for chewable tablets (like dextrose, fructose, lactose monohydrate, lactose and aspartame, lactose and cellulose, maltodextrin, maltose, mannitol, microcrystalline cellulose and guar gum, sorbitol crystalline); parenterals (like mannitol and povidone); plasticizers (like dibutyl sebacate, plasticizers for coatings, polyvinylacetate phthalate); powder lubricants (like glyceryl behenate); soft gelatin capsules (like sorbitol special solution); spheres for coating (like sugar spheres); spheronization agents (like glyceryl behenate and microcrystalline cellulose); suspending/gelling agents (like carrageenan, gellan gum, mannitol, microcrystalline cellulose, povidone, sodium starch glycolate, xanthan gum); sweeteners (like aspartame, aspartame and lactose, dextrose, fructose, honey, maltodextrin, maltose, mannitol, molasses, sorbitol crystalline, sorbitol special solution, sucrose); wet granulation agents (like calcium carbonate, lactose anhydrous, lactose monohydrate, maltodextrin, mannitol, microcrystalline cellulose, povidone, starch), caramel, carboxymethylcellulose sodium, cherry cream flavor and cherry flavor, citric acid anhydrous, citric acid, confectioner's sugar, D&C Red No. 33, D&C Yellow #10 Aluminum Lake, disodium edetate, ethyl alcohol 15%, FD&C Yellow No. 6 aluminum lake, FD&C Blue #1 Aluminum Lake, FD&C Blue No. 1, FD&C blue no. 2 aluminum lake, FD&C Green No. 3, FD&C Red No. 40, FD&C Yellow No. 6 Aluminum Lake, FD&C Yellow No. 6, FD&C Yellow No.10, glycerol palmitostearate, glyceryl monostearate, indigo carmine, lecithin, manitol, methyl and propyl parabens, mono ammonium glycyrrhizinate, natural and artificial orange flavor, pharmaceutical glaze, poloxamer 188, Polydextrose, polysorbate 20, polysorbate 80, polyvidone, pregelatinized corn starch, pregelatinized starch, red iron oxide, saccharin sodium, sodium carboxymethyl ether, sodium chloride, sodium citrate, sodium phosphate, strawberry flavor, synthetic black iron oxide, synthetic red iron oxide, titanium dioxide, and white wax.

Solid oral dosage forms may optionally be treated with coating systems (e.g. Opadry® fx film coating system, for example Opadry® blue (OY-LS-20921), Opadry® white (YS-2-7063), Opadry® white (YS-1-7040), and black ink (S-1-8 106).

The agents either in their free form or as a salt can be combined with a polymer such as polylactic-glycoloic acid (PLGA), poly-(I)-lactic-glycolic-tartaric acid (P(I)LGT) (WO 01/12233), polyglycolic acid (U.S. Pat. No. 3,773,919), polylactic acid (U.S. Pat. No. 4,767,628), poly(ε-caprolactone) and poly(alkylene oxide) (U.S. 20030068384) to create a sustained release formulation. Such formulations can be used to implants that release a polypeptide or another agent over a period of a few days, a few weeks or several months depending on the polymer, the particle size of the polymer, and the size of the implant (See, e.g., U.S. Pat. No. 6,620,422). Other sustained release formulations and polymers for use in are described in EP 0 467 389 A2, WO 93/24150, U.S. Pat. No. 5,612,052, WO 97/40085, WO 03/075887, WO 01/01964A2, U.S. Pat. No. 5,922,356, WO 94/155587, WO 02/074247A2, WO 98/25642, U.S. Pat. Nos. 5,968,895, 6,180,608, U.S. 20030171296, U.S. 20020176841, U.S. Pat. Nos. 5,672,659, 5,893,985, 5,134,122, 5,192,741, 5,192,741, 4,668,506, 4,713,244, 5,445,832 4,931,279, 5,980,945, WO 02/058672, WO 97/26015, WO 97/04744, and US20020019446. In such sustained release formulations microparticles (Delie and Blanco-Prieto 2005 Molecule 10:65-80) of polypeptide are combined with microparticles of polymer. One or more sustained release implants can be placed in the large intestine, the small intestine or both. U.S. Pat. No. 6,011,011 and WO 94/06452 describe a sustained release formulation providing either polyethylene glycols (i.e. PEG 300 and PEG 400) or triacetin. WO 03/053401 describes a formulation which may both enhance bioavailability and provide controlled releaseof the agent within the GI tract. Additional controlled release formulations are described in WO 02/38129, EP 326151, U.S. Pat. No. 5,236,704, WO 02/30398, WO 98/13029; U.S. 20030064105, U.S. 20030138488A1, U.S. 20030216307A1, U.S. Pat. No. 6,667,060, WO 01/49249, WO 01/49311, WO 01/49249, WO 01/49311, and U.S. Pat. No. 5,877,224 materials which may include those described in WO04041195 (including the seal and enteric coating described therein) and pH-sensitive coatings that achieve delivery in the colon including those described in U.S. Pat. No. 4,910,021 and WO9001329. U.S. Pat. No. 4,910,021 describes using a pH-sensitive material to coat a capsule. WO9001329 describes using pH-sensitive coatings on beads containing acid, where the acid in the bead core prolongs dissolution of the pH-sensitive coating. U.S. Pat. No. 5,175,003 discloses a dual mechanism polymer mixture composed of pH-sensitive enteric materials and film-forming plasticizers capable of conferring permeability to the enteric material, for use in drug-delivery systems; a matrix pellet composed of a dual mechanism polymer mixture permeated with a drug and sometimes covering a pharmaceutically neutral nucleus; a membrane-coated pellet comprising a matrix pellet coated with a dual mechanism polymer mixture envelope of the same or different composition; and a pharmaceutical dosage form containing matrix pellets. The matrix pellet releases acid-soluble drugs by diffusion in acid pH and by disintegration at pH levels of nominally about 5.0 or higher.

The GCRA peptideds described herein may be formulated in the pH triggered targeted control release systems described in WO04052339. The agents described herein may be formulated according to the methodology described in any of WO03105812 (extruded hydratable polymers); WO0243767 (enzyme cleavable membrane translocators); WO03007913 and WO03086297 (mucoadhesive systems); WO02072075 (bilayer laminated formulation comprising pH lowering agent and absorption enhancer); WO04064769 (amidated polypeptides); WO05063156 (solid lipid suspension with pseudotropic and/or thixotropic properties upon melting); WO03035029 and WO03035041 (erodible, gastric retentive dosage forms); U.S. Pat. Nos. 5,007,790 and 5,972,389 (sustained release dosage forms); WO041 1271 1 (oral extended release compositions); WO05027878, WO02072033, and WO02072034 (delayed release compositions with natural or synthetic gum); WO05030182 (controlled release formulations with an ascending rate of release); WO05048998 (microencapsulation system); U.S. Pat. No. 5,952,314 (biopolymer); U.S. Pat. No. 5,108,758 (glassy amylose matrix delivery); U.S. Pat. No. 5,840,860 (modified starch based delivery). JP10324642 (delivery system comprising chitosan and gastric resistant material such as wheat gliadin or zein); U.S. Pat. Nos. 5,866,619 and 6,368,629 (saccharide containing polymer); U.S. Pat. No. 6,531,152 (describes a drug delivery system containing a water soluble core (Ca pectinate or other water-insoluble polymers) and outer coat which bursts (e.g. hydrophobic polymer-Eudragrit)); U.S. Pat. Nos. 6,234,464; 6,403,130 (coating with polymer containing casein and high methoxy pectin; WO0174 175 (Maillard reaction product); WO05063206 (solubility increasing formulation); WO040 19872 (transferring fusion proteins).

The GCRA peptides described herein may be formulated using gastrointestinal retention system technology (GIRES; Merrion Pharmaceuticals). GIRES comprises a controlled-release dosage form inside an inflatable pouch, which is placed in a drug capsule for oral administration. Upon dissolution of the capsule, a gas-generating system inflates the pouch in the stomach where it is retained for 16-24 hours, all the time releasing agents described herein.

The GCRA peptides described herein can be formulated in an osmotic device including the ones disclosed in U.S. Pat. Nos. 4,503,030, 5,609,590 and 5,358,502. U.S. Pat. No. 4,503,030 discloses an osmotic device for dispensing a drug to certain pH regions of the gastrointestinal tract. More particularly, the invention relates to an osmotic device comprising a wall formed of a semi-permeable pH sensitive composition that surrounds a compartment containing a drug, with a passageway through the wall connecting the exterior of the device with the compartment. The device delivers the drug at a controlled rate in the region of the gastrointestinal tract having a pH of less than 3.5, and the device self-destructs and releases all its drug in the region of the gastrointestinal tract having a pH greater than 3.5, thereby providing total availability for drug absorption. U.S. Pat. Nos. 5,609,590 and 5,358,502 disclose an osmotic bursting device for dispensing a beneficial agent to an aqueous environment. The device comprises a beneficial agent and osmagent surrounded at least in part by a semi-permeable membrane. The beneficial agent may also function as the osmagent. The semi-permeable membrane is permeable to water and substantially impermeable to the beneficial agent and osmagent. A trigger means is attached to the semi-permeable membrane (e.g., joins two capsule halves). The trigger means is activated by a pH of from 3 to 9 and triggers the eventual, but sudden, delivery of the beneficial agent. These devices enable the pH-triggered release of the beneficial agent core as a bolus by osmotic bursting.

### Exemplary Additional Agents for Combination Therapy

### Analgesic Agents

The GCRA peptides described herein can be used in combination therapy with an analgesic agent, e.g., an analgesic compound or an analgesic polypeptide. These polypeptides and compounds can be administered with the GCRA peptides described herein (simultaneously or sequentially). They can also be optionally covalently linked or attached to an agent described herein to create therapeutic conjugates. Among the useful analgesic agents are: Calcium channel blockers, 5HT receptor antagonists (for example 5HT3, 5HT4 and 5HT1 receptor antagonists), opioid receptor agonists (loperamide, fedotozine, and fentanyl), NK1 receptor antagonists, CCK receptor agonists (e.g., loxiglumide), NK1 receptor antagonists, NK3 receptor antagonists, norepinephrine-serotonin reuptake inhibitors (NSRI), vanilloid and cannabanoid receptor agonists, and sialorphin. Analgesics agents in the various classes are described in the literature.

Among the useful analgesic polypeptides are sialorphin-related polypeptides, including those comprising the amino acid sequence QHNPR (SEQ ID NO: 250), including: VQHNPR (SEQ ID NO: 251); VRQHNPR (SEQ ID NO: 252); VRGQHNPR (SEQ ID NO: 253); VRGPQHNPR (SEQ ID NO: 254); VRGPRQHNPR (SEQ ID NO: 255); VRGPRRQHNPR (SEQ ID NO: 256); and RQHNPR (SEQ ID NO: 257). Sialorphin-related polypeptides bind to neprilysin and inhibit neprilysin-mediated breakdown of substance P and Met-enkephalin. Thus, compounds or polypeptides that are inhibitors of neprilysin are useful analgesic agents which can be administered with the polypeptides described herein in a co-therapy or linked to the polypeptides described herein, e.g., by a covalent bond. Sialophin and related polypeptides are described in U.S. Pat. No. 6,589,750; U.S. 20030078200 A1; and WO 02/051435 A2.

Opioid receptor antagonists and agonists can be administered with the GCRA peptides described herein in co-therapy or linked to the agent described herein, e.g., by a covalent bond. For example, opioid receptor antagonists such as naloxone, naltrexone, methyl nalozone, nalmefene, cypridime, beta funaltrexamine, naloxonazine, naltrindole, and nor-binaltorphimine are thought to be useful in the treatment of IBS. It can be useful to formulate opioid antagonists of this type is a delayed and sustained release formulation such that initial release of the antagonist is in the mid to distal small intestine and/or ascending colon. Such antagonists are described in WO 01/32180 A2. Enkephalin pentapeptide (HOE825; Tyr-D-Lys-Gly-Phe-L-homoserine) is an agonist of the mu and delta opioid receptors and is thought to be useful for increasing intestinal motility {Eur. J. Pharm. 219:445, 1992), and this polypeptide can be used in conjunction with the polypeptides described herein. Also useful is trimebutine which is thought to bind to mu/delta/kappa opioid receptors and activate release of motilin and modulate the release of gastrin, vasoactive intestinal polypeptide, gastrin and glucagons. Kappa opioid receptor agonists such as fedotozine, asimadoline, and ketocyclazocine, and compounds described in WO03/097051 and WO05/007626 can be used with or linked to the polypeptides described herein. In addition, mu opioid receptor agonists such as morphine, diphenyloxylate, frakefamide (H-Tyr-D-Ala-Phe(F)-Phe-NH 2; WO 01/019849 A1) and loperamide can be used.

Tyr-Arg (kyotorphin) is a dipeptide that acts by stimulating the release of met-enkephalins to elicit an analgesic effect (J. Biol. Chem 262:8165, 1987). Kyotorphin can be used with or linked to the GCRA peptides described herein.

Chromogranin-derived polypeptide (CgA 47-66; See, e.g., Ghia et al. 2004 Regulatory polypeptides 119:199) can be used with or linked to the GCRA peptides described herein.

CCK receptor agonists such as caerulein from amphibians and other species are useful analgesic agents that can be used with or linked to the GCRA peptides described herein.

Conotoxin polypeptides represent a large class of analgesic polypeptides that act at voltage gated calcium channels, NMDA receptors or nicotinic receptors. These polypeptides can be used with or linked to the polypeptides described herein.

Peptide analogs of thymulin (FR Application 2830451) can have analgesic activity and can be used with or linked to the polypeptides described herein.

CCK (CCKa or CCKb) receptor antagonists, including loxiglumide and dexloxiglumide (the R-isomer of loxiglumide) (WO 88/05774) can have analgesic activity and can be used with or linked to the polypeptides described herein.

Other useful analgesic agents include 5-HT4 agonists such as tegaserod (Zelnorm®), mosapride, metoclopramide, zacopride, cisapride, renzapride, benzimidazolone derivatives such as BIMU 1 and BIMU 8, and lirexapride. Such agonists are described in: EP1321 142 A1, WO 03/053432A1, EP 505322 A1, EP 505322 B1, U.S. Pat. No. 5,510,353, EP 507672 A1, EP 507672 B1, and U.S. Pat. No. 5,273,983.

Calcium channel blockers such as ziconotide and related compounds described in, for example, EP625162B1, U.S. Pat. Nos. 5,364,842, 5,587,454, 5,824,645, 5,859,186, 5,994,305, 6,087,091, 6,136,786, WO 93/13128 A1, EP 1336409 A1, EP 835126 A1, EP 835126 B1, U.S. Pat. No. 5,795,864, 5,891,849, 6,054,429, WO 97/01351 A1, can be used with or linked to the polypeptides described herein.

Various antagonists of the NK-I, NK-2, and NK-3 receptors (for a review see Giardina et al. 2003.Drugs 6:758) can be can be used with or linked to the polypeptides described herein.

NK1 receptor antagonists such as: aprepitant (Merck & Co Inc), vofopitant, ezlopitant (Pfizer, Inc.), R-673 (Hoffmann-La Roche Ltd), SR-48968 (Sanofi Synthelabo), CP-122,721 (Pfizer, Inc.), GW679769 (Glaxo Smith Kline), TAK-637 (Takeda/Abbot), SR-14033, and related compounds described in, for example, EP 873753 A1, US 20010006972 A1, US 20030109417 A1, WO 01/52844 A1, can be used with or linked to the polypeptides described herein.

NK-2 receptor antagonists such as nepadutant (Menarini Ricerche SpA), saredutant (Sanofi-Synthelabo), GW597599 (Glaxo Smith Kline), SR-144190 (Sanofi-Synthelabo) and UK-290795 (Pfizer Inc) can be used with or linked to the polypeptides described herein.

NK3 receptor antagonists such as osanetant (SR-142801; Sanoft-Synthelabo), SSR-241586, talnetant and related compounds described in, for example, WO 02/094187 A2, EP 876347 A1, WO 97/21680 A1, U.S. Pat. No. 6,277,862, WO 98/1 1090, WO 95/28418, WO 97/19927, and Boden et al. (J Med Chem. 39:1664-75, 1996) can be used with or linked to the polypeptides described herein.

Norepinephrine-serotonin reuptake inhibitors (NSRI) such as milnacipran and related compounds described in WO 03/077897 A1 can be used with or linked to the polypeptides described herein.

Vanilloid receptor antagonists such as arvanil and related compounds described in WO 01/64212 A1 can be used with or linked to the polypeptides described herein.

The analgesic polypeptides and compounds can be administered with the polypeptides and agonists described herein (simultaneously or sequentially). The analgesic agents can also be covalently linked to the polypeptides and agonists described herein to create therapeutic conjugates. Where the analgesic is a polypeptide and is covalently linked to an agent described herein the resulting polypeptide may also include at least one trypsin cleavage site. When present within the polypeptide, the analgesic polypeptide may be preceded by (if it is at the carboxy terminus) or followed by (if it is at the amino terminus) a trypsin cleavage site that allows release of the analgesic polypeptide.

In addition to sialorphin-related polypeptides, analgesic polypeptides include: AspPhe, endomorphin-1, endomorphin-2, nocistatin, dalargin, lupron, ziconotide, and substance P.

### Agents to Treat Gastrointestinal Disorders

Examples of additional therapeutic agents to treat gastrointestinal and other disorders include agents to treat constipation (e.g., a chloride channel activator such as the bicylic fatty acid, Lubiprostone (formerly known as SPI-0211; Sucampo Pharmaceuticals, Inc.; Bethesda, Md.), a laxative (e.g. a bulk-forming laxative (e.g. nonstarch polysaccharides, Colonel Tablet (polycarbophil calcium), Plantago Ovata®, Equalactin® (Calcium Polycarbophil)), fiber (e.g. FIBERCON® (Calcium Polycarbophil), an osmotic laxative, a stimulant laxative (such as diphenylmethanes (e.g. bisacodyl), anthraquinones (e.g. cascara, senna), and surfactant laxatives (e.g. castor oil, docusates), an emollient/lubricating agent (such as mineral oil, glycerine, and docusates), MiraLax (Braintree Laboratories, Braintree Mass.), dexloxiglumide (Forest Laboratories, also known as CR 2017 Rottapharm (Rotta Research Laboratorium SpA)), saline laxatives, enemas, suppositories, and CR 3700 (Rottapharm (Rotta Research Laboratorium SpA); acid reducing agents such as proton pump inhibitors (e.g., omeprazole (Prilosec®), esomeprazole (Nexium®), lansoprazole (Prevacid®), pantoprazole (Protonix®) and rabeprazole (Aciphex®)) and Histamine H2-receptor antagonist (also known as H2 receptor blockers including cimetidine, ranitidine, famotidine and nizatidine); prokinetic agents including itopride, octreotide, bethanechol, metoclopramide (Reglan®), domperidone (Motilium®), erythromycin (and derivatives thereof) or cisapride (Propulsid®); Prokineticin polypeptides homologs, variants and chimeras thereof including those described in U.S. Pat. No. 7,052,674 which can be used with or linked to the polypeptides described herein; pro-motility agents such as the vasostatin-derived polypeptide, chromogranin A (4-16) (See, e.g., Ghia et al. 2004 Regulatory polypeptides 121:31) or motilin agonists (e.g., GM-611 or mitemcinal fumarate) or nociceptin/Orphanin FQ receptor modulators (US20050169917); other peptides which can bind to and/or activate GC-C including those described in US20050287067; complete or partial 5HT (e.g. 5HT1, 5HT2, 5HT3, 5HT4) receptor agonists or antagonists (including 5HT1A antagonists (e.g. AGI-OO1 (AGI therapeutics), 5HT2B antagonists (e.g. PGN 1091 and PGNl 164 (Pharmagene Laboratories Limited), and 5HT4 receptor agonists (such as tegaserod (ZELNORM®), prucalopride, mosapride, metoclopramide, zacopride, cisapride, renzapride, benzimidazolone derivatives such as BIMU 1 and BIMU 8, and lirexapride). Such agonists/modulators are described in: EP1321142 A1, WO 03/053432A1, EP 505322 A1, EP 505322 B1, U.S. Pat. No. 5,510,353, EP 507672 A1, EP 507672 B1, U.S. Pat. Nos. 5,273,983, and 6,951,867); 5HT3 receptor agonists such as MKC-733; and 5HT3 receptor antagonists such as DDP-225 (MCI-225; Dynogen Pharmaceuticals, Inc.), cilansetron (Calmactin®), alosetron (Lotronex®), Ondansetron HCl (Zofran®), Dolasetron (ANZEMET®), palonosetron (Aloxi®), Granisetron (Kytril®), YM060 (ramosetron; Astellas Pharma Inc.; ramosetron may be given as a daily dose of 0.002 to 0.02 mg as described in EP01588707) and ATI-7000 (Aryx Therapeutics, Santa Clara Calif.); muscarinic receptor agonists; anti-inflammatory agents; antispasmodics including but not limited to anticholinergic drugs (like dicyclomine (e.g. Colimex®, Formulex®, Lomine®, Protylol®, Visceral®, Spasmoban®, Bentyl®, Bentylol®), hyoscyamine (e.g. IB-Stat®, Nulev®, Levsin®, Levbid®, Levsinex Timecaps®, Levsin/SL®, Anaspaz®, A-Spas S/L®, Cystospaz®, Cystospaz-M®, Donnamar®, Colidrops Liquid Pediatric®, Gastrosed®, Hyco Elixir®, Hyosol®, Hyospaz®, Hyosyne®, Losamine®, Medispaz®, Neosol®, Spacol®, Spasdel®, Symax®, Symax SL®), Donnatal (e.g. Donnatal Extentabs®), clidinium (e.g. Quarzan, in combination with Librium=Librax), methantheline (e.g. Banthine), Mepenzolate (e.g. Cantil), homatropine (e.g. hycodan, Homapin), Propantheline bromide (e.g. Pro-Banthine), Glycopyrrolate (e.g. Robinul®, Robinul Forte®), scopolamine (e.g. Transderm-Scop®, Transderm-V®), hyosine-N-butylbromide (e.g. Buscopan®), Pirenzepine (e.g. Gastrozepin®) Propantheline Bromide (e.g. Propanthel®), dicycloverine (e.g. Merbentyl®), glycopyrronium bromide (e.g. Glycopyrrolate®), hyoscine hydrobromide, hyoscine methobromide, methanthelinium, and octatropine); peppermint oil; and direct smooth muscle relaxants like cimetropium bromide, mebeverine (DUSPATAL®, DUSPATALIN®, COLOFAC MR®, COLOTAL®), otilonium bromide (octilonium), pinaverium (e.g. Dicetel® (pinaverium bromide; Solvay S. A.)), Spasfon® (hydrated phloroglucinol and trimethylphloroglucinol)and trimebutine (including trimebutine maleate (Modulon®); antidepressants, including but not limited to those listed herein, as well as tricyclic antidepressants like amitriptyline (Elavil®), desipramine (Norpramin®), imipramine (Tofranil®), amoxapine (Asendin®), nortriptyline; the selective serotonin reuptake inhibitors (SSRTs) like paroxetine (Paxil®), fluoxetine (Prozac®), sertraline (Zoloft®), and citralopram (Celexa®); and others like doxepin (Sinequan®) and trazodone (Desyrel®); centrally-acting analgesic agents such as opioid receptor agonists, opioid receptor antagonists (e.g., naltrexone); agents for the treatment of Inflammatory bowel disease; agents for the treatment of Crohn's disease and/or ulcerative colitis (e.g., alequel (Enzo Biochem, Inc.; Farmingsale, N.Y.), the anti-inflammatory polypeptide RDP58 (Genzyme, Inc.; Cambridge, Mass.), and TRAFICET-EN™ (ChemoCentryx, Inc.; San Carlos, Calif.); agents that treat gastrointestinal or visceral pain; agents that increase cGMP levels (as described in US20040121994) like adrenergic receptor antagonists, dopamine receptor agonists and PDE (phosphodiesterase) inhibitors including but not limited to those disclosed herein; purgatives that draw fluids to the intestine (e.g., VISICOL®, a combination of sodium phosphate monobasic monohydrate and sodium phosphate dibasic anhydrate); Corticotropin Releasing Factor (CRF) receptor antagonists (including NBI-34041 (Neurocrine Biosciences, San Diego, Calif.), CRH9-41, astressin, R121919 (Janssen Pharmaceutica), CP154,526, NBI-27914, Antalarmin, DMP696 (Bristol-Myers Squibb) CP-316,311 (Pfizer, Inc.), SB723620 (GSK), GW876008 (Neurocrine/Glaxo Smith Kline), ONO-2333Ms (Ono Pharmaceuticals), TS-041 (Janssen), AAG561 (Novartis) and those disclosed in U.S. Pat. Nos. 5,063,245, 5,861,398, US20040224964, US20040198726, US20040176400, US20040171607, US20040110815, US20040006066, and US20050209253); glucagon-like polypeptides (glp-1) and analogues thereof (including exendin-4 and GTP-010 (Gastrotech Pharma A)) and inhibitors of DPP-IV (DPP-IV mediates the inactivation of glp-1); tofisopam, enantiomerically-pure R-tofisopam, and pharmaceutically-acceptable salts thereof (US 20040229867); tricyclic anti-depressants of the dibenzothiazepine type including but not limited to Dextofisopam® (Vela Pharmaceuticals), tianeptine (Stablon®) and other agents described in U.S. Pat. No. 6,683,072; (E)-4 (1,3bis(cyclohexylmethyl)-1,2,34,-tetrahydro-2,6-diono-9H-purin-8-yl)cinnamic acid nonaethylene glycol methyl ether ester and related compounds described in WO 02/067942; the probiotic PROBACTRIX® (The BioBalance Corporation; New York, N.Y.) which contains microorganisms useful in the treatment of gastrointestinal disorders; antidiarrheal drugs including but not limited to loperamide (Imodium, Pepto Diarrhea), diphenoxylate with atropine (Lomotil, Lomocot), cholestyramine (Questran, Cholybar), atropine (Co-Phenotrope, Diarsed, Diphenoxylate, Lofene, Logen, Lonox, Vi-Atro, atropine sulfate injection) and Xifaxan® (rifaximin; Salix Pharmaceuticals Ltd), TZP-201(Tranzyme Pharma Inc.), the neuronal acetylcholine receptor (nAChR) blocker AGI-004 (AGI therapeutics), and bismuth subsalicylate (Pepto-bismol); anxiolytic drugs including but not limited toAtivan (lorazepam), alprazolam (Xanax®), chlordiazepoxide/clidinium (Librium®, Librax®), clonazepam (Klonopin®), clorazepate (Tranxene®), diazepam (Valium®), estazolam (ProSom®), flurazepam (Dalmane®), oxazepam (Serax®), prazepam (Centrax®), temazepam (Restoril®), triazolam (Halcion®; Bedelix® (Montmorillonite beidellitic; Ipsen Ltd), Solvay SLV332 (ArQuIe Inc), YKP (SK Pharma), Asimadoline (Tioga Pharmaceuticals/Merck), AGI-003 (AGI Therapeutics); neurokinin antagonists including those described in US20060040950; potassium channel modulators including those described in U.S. Pat. No. 7,002,015; the serotonin modulator AZD7371 (AstraZeneca Plc); M3 muscarinic receptor antagonists such as darifenacin (Enablex; Novartis AG and zamifenacin (Pfizer); herbal and natural therapies including but not limited to acidophilus, chamomile tea, evening primrose oil, fennel seeds,wormwood, comfrey, and compounds of Bao-Ji-Wan (magnolol, honokiol, imperatorin, and isoimperatorin) as in U.S. Pat. No. 6,923,992; and compositions comprising lysine and an anti-stress agent for the treatment of irritable bowel syndrome as described in EPO 1550443.

### Agents to Treat Gastrointestinal Cancers

The GCRA peptides described herein can be used in combination with one or more antitumor agents including but not limited to alkylating agents, epipodophyllotoxins, nitrosoureas, anti-metabolites, vinca alkaloids, anthracycline antibiotics, nitrogen mustard agents, and the like. Particular antitumor agents include tamoxifen, taxol, etoposide, and 5-fluorouracil. In one embodiment, the GCRA peptides are used in combination with an antiviral agent or a monoclonal antibody.

Non-limiting examples of antitumor agents that can be used in combination with the GCRA peptides of the invention for the treatment of colon cancer include anti-proliferative agents, agents for DNA modification or repair, DNA synthesis inhibitors, DNA/RNA transcription regulators, RNA processing inhibitors, agents that affect protein expression, synthesis and stability, agents that affect protein localization or their ability to exert their physiological action, agents that interfere with protein-protein or protein-nucleic acid interactions, agents that act by RNA interference, receptor binding molecules of any chemical nature (including small molecules and antibodies), targeted toxins, enzyme activators, enzyme inhibitors, gene regulators, HSP-90 inhibitors, molecules interfering with microtubules or other cytoskeletal components or cell adhesion and motility, agents for phototherapy, and therapy adjuncts.

Representative anti-proliferative agents include N-acetyl-D-sphingosine (C.sub.2 ceramide), apigenin, berberine chloride, dichloromethylenediphosphonic acid disodium salt, loe-emodine, emodin, HA 14-1, N-hexanoyl-D-sphingosine (C.sub.6 ceramide), 7b-hydroxycholesterol, 25-hydroxycholesterol, hyperforin, parthenolide, and rapamycin. Representative agents for DNA modification and repair include aphidicolin, bleomycin sulfate, carboplatin, carmustine, chlorambucil, cyclophosphamide monohydrate, cyclophosphamide monohydrate ISOPAC.®, cis-diammineplatinum(II) dichloride (Cisplatin), esculetin, melphalan, methoxyamine hydrochloride, mitomycin C, mitoxantrone dihydrochloride, oxaliplatin, and streptozocin.

Representative DNA synthesis inhibitors include (.+−.)amethopterin (methotrexate), 3-amino-1,2,4-benzotriazine 1,4-dioxide, aminopterin, cytosine b-D-arabinofurdnoside (Ara-C), cytosine b-D-arabinofuranoside (Ara-C) hydrochloride, 2-fluoroadenine-9-b-D-arabinofuranoside (Fludarabine des-phosphate; F-ara-A), 5-fluoro-5′-deoxyuridinc, 5-fluorouracil, ganciclovir, hydroxyurea, 6-mercaptopurine, and 6-thioguanine.

Representative DNA/RNA transcription regulators include actinomycin D, daunorubicin hydrochloride, 5,6-dichlorobenzimidazole 1-b-D-ribofuranoside, doxorubicin hydrochloride, homoharringtonine, and idarubicin hydrochloride.

Representative enzyme activators and inhibitors include forskolin, DL-aminoglutethimide, apicidin, Bowman-Birk Inhibitor, butein, (S)-(+)-camptothecin, curcumin, (−)-deguelin, (−)-depudecin, doxycycline hyclate, etoposide, formestane, fostriecin sodium salt, hispidin, 2-imino-1-imidazolidineacetic acid (Cyclocreatine), oxamflatin, 4-phenylbutyric acid, roscovitine, sodium valproate, trichostatin A, tyrphostin AG 34, tyrphostin AG 879, urinary trypsin inhibitor fragment, valproic acid (2-propylpentanoic acid), and XK469.

Representative gene regulators include 5-aza-2′-deoxycytidine, 5-azacytidine, cholecalciferol (Vitamin D3), ciglitizone, cyproterone acetate, 15-deoxy-D.sup.12,14-prostaglandin J.sub.2, epitestosterone, flutamide, glycyrrhizic acid ammonium salt (glycyrrhizin), 4-hydroxytamoxifen, mifepristone, procainamide hydrochloride, raloxifene hydrochloride, all trans-retinal (vitamin A aldehyde), retinoic acid (vitamin A acid), 9-cis-retinoic acid, 13-cis-retinoic acid, retinoic acid p-hydroxyanilide, retinol (Vitamin A), tamoxifen, tamoxifen citrate salt, tetradecylthioacetic acid, and troglitazone.

Representative HSP-90 inhibitors include 17-(allylamino)-17-demethoxygeldanamycin and geldanamycin.

Representative microtubule inhibitors include colchicines, dolastatin 15, nocodazole, taxanes and in particular paclitaxel, podophyllotoxin, rhizoxin, vinblastine sulfate salt, vincristine sulfate salt, and vindesine sulfate salt and vinorelbine (Navelbine) ditartrate salt.

Representative agents for performing phototherapy include photoactive porphyrin rings, hypericin, 5-methoxypsoralen, 8-methoxypsoralen, psoralen and ursodeoxycholic acid.

Representative agents used as therapy adjuncts include amifostine, 4-amino-1,8-naphthalimide, brefeldin A, cimetidine, phosphomycin disodium salt, leuprolide (leuprorelin) acetate salt, luteinizing hormone-releasing hormone (LH-RH) acetate salt, lectin, papaverine hydrochloride, pifithrin-a, (−)-scopolamine hydrobromide, and thapsigargin.

The agents can also be anti-VEGF (vascular endothelial growth factor) agents, as such are known in the art. Several antibodies and small molecules are currently in clinical trials or have been approved that function by inhibiting VEGF, such as Avastin (Bevacizumab), SU5416, SU11248 and BAY 43-9006. The agents can also be directed against growth factor receptors such as those of the EGF/Erb-B family such as EGF Receptor (Iressa or Gefitinib, and Tarceva or Erlotinib), Erb-B2, receptor (Herceptin or Trastuzumab), other receptors (such as Rituximab or Rituxan/MabThera), tyrosine kinases, non-receptor tyrosine kinases, cellular serine/threonine kinases (including MAP kinases), and various other proteins whose deregulation contribute to oncogenesis (such as small/Ras family and large/heterotrimeric G proteins). Several antibodies and small molecules targeting those molecules are currently at various stages of development (including approved for treatment or in clinical trials).

In a preferred embodiment, the invention provides a method for treating colon cancer in a subject in need thereof by administering to the subject a GCRA peptide or a composition described herein in combination with one or more antitumor agent selected from the group consisting of paclitaxel, docetaxel, tamoxifen, vinorelbine, gemcitabine, cisplatin, etoposide, topotecan, irinotecan, anastrozole, rituximab, trastuzumab, fludarabine, cyclophosphamide, gentuzumab, carboplatin, interferons, and doxorubicin. In a particular embodiment the antitumor agent is paclitaxel. In a further embodiment, the method further comprises an antitumor agent selected from the group consisting of 5-FU, doxorubicin, vinorelbine, cytoxan, and cisplatin.

Agents that Treat Crohn's Disease

In one embodiment, a GCRA peptide of the invention is administered as part of a combination therapy with one or more additional therapeutic agents for the treatment of Crohn's disease. Non-limiting examples of the one or more additional therapeutic agents include sulfasalazine and other mesalamine-containing drugs, generally known as 5-ASA agents, such as Asacol, Dipentum, or Pentasa, Salofalk®, sulfasalazine, Salazopyrin®, Salazopyrin En-tabs®, or generics thereof or infliximab (REMICADE). In certain embodiments, the one or more additional agents is a corticosteroid or an immunosuppressive agent such as 6-mercaptopurine or azathioprine. In another embodiment, the one or more additional agents are antidiarrheal agents such as diphenoxylate, loperamide, or codeine.

Agents that Treat Ulcerative Colitis

In one embodiment, a GCRA peptide of the invention is administered as part of a combination therapy with one or more additional therapeutic agents for the treatment of ulcerative colitis. The agents that are used to treat ulcerative colitis overlap with those used to treat Crohn's Disease. Non-limiting examples of the one or more additional therapeutic agents that can be used in combination with a GCRA peptide of the invention include aminosalicylates (drugs that contain 5-aminosalicyclic acid (5-ASA)) such as sulfasalazine, olsalazine, mesalamine, and balsalazide. Other therapeutic agents that can be used include corticosteroids, such as prednisone and hydrocortisone, immunomodulators, such as azathioprine, 6-mercapto-purine (6-MP), cytokines, interleukins, and lymphokines, and anti-TNF-alpha agents, including the thiazolidinediones or glitazones such as rosiglitazone and pioglitazone. In one emobidment, the one or more additional therapeutic agents include both cyclosporine A and 6-MP or azathioprine for the treatment of active, severe ulcerative colitis.

Agents that Treat Constipation/Irritable Bowel Syndrome

In one embodiment, a GCRA peptide of the invention is administered as part of a combination therapy with one or more additional therapeutic agents for the treatment of constipation, such as that associated with irritable bowel syndrome. Non-limiting examples of the one or more additional therapeutic agents include laxatives such as SENNA, MIRALAX, LACTULOSE, PEG, or calcium polycarbophil), stool softeners (such as mineral oil or COLACE), bulking agents (such as METAMUCIL or bran), agents such as ZELNORM (also called tegaserod), and anticholinergic medications such as BENTYL and LEVSIN.

### Insulin and Insulin Modulating Agents

The GCRA peptides described herein can be used in combination therapy with insulin and related compounds including primate, rodent, or rabbit insulin including biologically active variants thereof including allelic variants, more preferably human insulin available in recombinant form. Sources of human insulin include pharmaceutically acceptable and sterile formulations such as those available from Eli Lilly (Indianapolis, Ind. 46285) as Humulin™ (human insulin rDNA origin). See, the THE PHYSICIAN′S DESK REFERENCE, 55.sup.th Ed. (2001) Medical Economics, Thomson Healthcare (disclosing other suitable human insulins).

The GCRA peptides described herein can also be used in combination therapy with agents that can boost insulin effects or levels of a subject upon administration, e.g. glipizide and/or rosiglitazone. The polypeptides and agonistsdescribed herein can be used in combitherapy with SYMLIN® (pramlintide acetate) and Exenatide® (synthetic exendin-4; a 39 aa polypeptide).

### Agents for the Treatment of Postoperative Ileus

The GCRA peptides described herein can also be used in combination therapy with agents (e.g., Entereg™ (alvimopan; formerly called ado lor/ADL 8-2698), conivaptan and related agents describe in U.S. Pat. No. 6,645,959) used for the treatment of postoperative ileus and other disorders.

### Anti Hypertensive Agents

The GCRA peptides described herein can be used in combination therapy with an anti-hypertensive agent including but not limited to: (1) diuretics, such as thiazides, including chlorthalidone, chlorthiazide, dichlorophenamide, hydroflumethiazide, indapamide, polythiazide, and hydrochlorothiazide; loop diuretics, such as bumetanide, ethacrynic acid, furosemide, and torsemide; potassium sparing agents, such as amiloride, and triamterene; carbonic anhydrase inhibitors, osmotics(such as glycerin) and aldosterone antagonists, such as spironolactone, epirenone, and the like; (2) beta-adrenergic blockers such as acebutolol, atenolol, betaxolol, bevantolol, bisoprolol, bopindolol, carteolol, carvedilol, celiprolol, esmolol, indenolol, metaprolol, nadolol, nebivolol, penbutolol, pindolol, propanolol, sotalol, tertatolol, tilisolol, and timolol, and the like; (3) calcium channel blockers such as amlodipine, aranidipine, azelnidipine, barnidipine, benidipine, bepridil, cinaldipine, clevidipine, diltiazem, efonidipine, felodipine, gallopamil, isradipine, lacidipine, lemildipine, lercanidipine, nicardipine, nifedipine, nilvadipine, nimodepine, nisoldipine, nitrendipine, manidipine, pranidipine, and verapamil, and the like; (4) angiotensin converting enzyme (ACE) inhibitors such as benazepril; captopril; ceranapril; cilazapril; delapril; enalapril; enalopril; fosinopril; imidapril; lisinopril; losinopril; moexipril; quinapril; quinaprilat; ramipril; perindopril; perindropril; quanipril; spirapril; tenocapril; trandolapril, and zofenopril, and the like; (5) neutral endopeptidase inhibitors such as omapatrilat, cadoxatril and ecadotril, fosidotril, sampatrilat, AVE7688, ER4030, and the like; (6) endothelin antagonists such as tezosentan, A308165, and YM62899, and the like; (7) vasodilators such as hydralazine, clonidine, minoxidil, and nicotinyl alcohol, and the like; (8) angiotensin II receptor antagonists such as aprosartan, candesartan, eprosartan, irbesartan, losartan, olmesartan, pratosartan, tasosartan, telmisartan, valsartan, and EXP-3137, FI6828K, and RNH6270, and the like; (9) α/β adrenergic blockers such as nipradilol, arotinolol and amosulalol, and the like; (10) alpha 1 blockers, such as terazosin, urapidil, prazosin, tamsulosin, bunazosin, trimazosin, doxazosin, naftopidil, indoramin, WHP 164, and XENOlO, and the like; (11) alpha 2 agonists such as lofexidine, tiamenidine, moxonidine, rilmenidine and guanobenz, and the like; (12) aldosterone inhibitors, and the like; and (13) angiopoietin-2-binding agents such as those disclosed in WO03/030833. Specific anti-hypertensive agents that can be used in combination with polypeptides and agonists described herein include, but are not limited to: diuretics, such as thiazides (e.g., chlorthalidone, cyclothiazide (CAS RN 2259-96-3), chlorothiazide (CAS RN 72956-09-3, which may be prepared as disclosed in U.S. Pat. No. 2,809,194), dichlorophenamide, hydroflumethiazide, indapamide, polythiazide, bendroflumethazide, methyclothazide, polythiazide, trichlormethazide, chlorthalidone, indapamide, metolazone, quinethazone, althiazide (CAS RN 5588-16-9, which may be prepared as disclosed in British Patent No. 902,658), benzthiazide (CAS RN 91-33-8, which may be prepared as disclosed in U.S. Pat. No. 3,108,097), buthiazide (which may be prepared as disclosed in British Patent Nos. 861,367), and hydrochlorothiazide), loop diuretics (e.g. bumetanide, ethacrynic acid, furosemide, and torasemide), potassium sparing agents (e.g. amiloride, and triamterene (CAS Number 396-01-O)), and aldosterone antagonists (e.g. spironolactone (CAS Number 52-01-7), epirenone, and the like); β-adrenergic blockers such as Amiodarone (Cordarone, Pacerone), bunolol hydrochloride (CAS RN 31969-05-8, Parke-Davis), acebutolol (±N-[3-Acetyl-4-[2-hydroxy-3-[(1 methylethyl)amino]propoxy]phenyl]-butanamide, or (±)-3′-Acetyl-4′-[2-hydroxy-3-(isopropylamino) propoxy] butyranilide), acebutolol hydrochloride (e.g. Sectral®, Wyeth-Ayerst), alprenolol hydrochloride (CAS RN 13707-88-5 see Netherlands Patent Application No. 6,605,692), atenolol (e.g. Tenormin®, AstraZeneca), carteolol hydrochloride (e.g. Cartrol® Filmtab®, Abbott), Celiprolol hydrochloride (CAS RN 57470-78-7, also see in U.S. Pat. No. 4,034,009), cetamolol hydrochloride (CAS RN 77590-95-5, see also U.S. Pat. No. 4,059,622), labetalol hydrochloride (e.g. Normodyne Schering), esmolol hydrochloride (e.g. Brevibloc®, Baxter), levobetaxolol hydrochloride (e.g. Betaxon™ Ophthalmic Suspension, Alcon), levobunolol hydrochloride (e.g. Betagan® Liquifilm® with C CAP® Compliance Cap, Allergan), nadolol (e.g. Nadolol, Mylan), practolol (CAS RN 6673-35-4, see also U.S. Pat. No. 3,408,387), propranolol hydrochloride (CAS RN 318-98-9), sotalol hydrochloride (e.g. Betapace AF™, Berlex), timolol (2-Propanol,1-[(1,1-dimethylethyl)amino]-3-[[4-4(4-morpholinyl)-1,2,5-thiadiazol-3-yl]oxy]-, hemihydrate, (S)-, CAS RN 91524-16-2), timolol maleate (S)-I-[(1,1-dimethylethyl) amino]-3-[[4-(4-morpholinyl)-1,2,5-thiadiazol-3-yl]oxy]-2-propanol (Z)-2-butenedioate (1:1) salt, CAS RN 26921-17-5), bisoprolol (2-Propanol, 1-[4-[[2-(1-methylethoxy)ethoxy]-methyl]phenoxyl]-3-[(1-meth-ylethyl)amino]-, (±), CAS RN 66722-44-9), bisoprolol fumarate (such as (±)-1-[4-[[2-(1-Methylethoxy) ethoxy]methyl]phenoxy]-3-[(1-methylethyl)amino]-2-propanol (E)-2-butenedioate (2:1) (salt), e.g., Zebeta™, Lederle Consumer), nebivalol (2H-1-Benzopyran-2-methanol, αα′-[iminobis(methylene)]bis[6-fluoro-3,4-dihydro-, CAS RN 99200-09-6 see also U.S. Pat. No. 4,654,362), cicloprolol hydrochloride, such 2-Propanol, 1-[4-[2-(cyclopropylmethoxy)ethoxy]phenoxy]-3-[1-methylethyl)amino]-, hydrochloride, A.A.S. RN 63686-79-3), dexpropranolol hydrochloride (2-Propanol,1-[1-methylethy)-amino]-3-(1-naphthalenyloxy)-hydrochloride (CAS RN 13071-11-9), diacetolol hydrochloride (Acetamide, N-[3-acetyl-4-[2-hydroxy-3-[(1-methyl-ethyl)amino]propoxy] [phenyl]-, monohydrochloride CAS RN 69796-04-9), dilevalol hydrochloride (Benzamide, 2-hydroxy-5-[1-hydroxy-2-[1-methyl-3-phenylpropyl)amino]ethyl]-, monohydrochloride, CAS RN 75659-08-4), exaprolol hydrochloride (2-Propanol, 1-(2-cyclohexylphenoxy)-3-[(1-methylethyl)amino]-, hydrochloride CAS RN 59333-90-3), flestolol sulfate (Benzoic acid, 2-fluro-,3-[[2-[aminocarbonyl)amino]-dimethylethyl]amino]-2-hydroxypropyl ester, (+)-sulfate (1:1) (salt), CAS RN 88844-73-9; metalol hydrochloride (Methanesulfonamide, N-[4-[1-hydroxy-2-(methylamino)propyl]phenyl]-, monohydrochloride CAS RN 7701-65-7), metoprolol 2-Propanol, 1-[4-(2-methoxyethyl)phenoxy]-3-[1-methylethyl)amino]-; CAS RN 37350-58-6), metoprolol tartrate (such as 2-Propanol, 1-[4-(2-methoxyethyl)phenoxy]-3-[(1-methylethyl)amino]-, e.g., Lopressor®, Novartis), pamatolol sulfate (Carbamic acid, [2-[4-[2-hydroxy-3-[(1-methylethyl)amino]propoxyl]phenyl]-ethyl]-, methyl ester, (±) sulfate (salt) (2:1), CAS RN 59954-01-7), penbutolol sulfate (2-Propanol, 1-(2-cyclopentylphenoxy)-3-[11-dimethyle-thyl)amino] 1, (S)-, sulfate (2:1) (salt), CAS RN 38363-32-5), practolol (Acetamide, N-[4-[2-hydroxy-3-[(1-methylethyl)amino]-propoxy]phenyl]-, CAS RN 6673-35-4;) tiprenolol hydrochloride (Propanol, 1-[(1-methylethyl)amino]-3-[2-(methylthio)-phenoxy]-, hydrochloride, (±), CAS RN 39832-43-4), tolamolol (Benzamide, 4-[2-[[2-hydroxy-3-(2-methylphenoxy)-propyl] amino] ethoxyl]-, CAS RN 38103-61-6), bopindolol, indenolol, pindolol, propanolol, tertatolol, and tilisolol, and the like; calcium channel blockers such as besylate salt of amlodipine (such as 3-ethyl-5-methyl-2-(2-aminoethoxymethyl)-4-(2-chlorophenyl)-1,4-dihydro-6-methyl-3,5-pyridinedicarboxylate benzenesulphonate, e.g., Norvasc®, Pfizer), clentiazem maleate (1,5-Benzothiazepin-4(5H)-one, 3-(acetyloxy)-8-chloro-5-[2-(dimethylamino)ethyl]-2,3-dihydro-2-(4-methoxyphenyl)-(2S-cis)-, (Z)-2-butenedioate (1:1), see also U.S. Pat. No. 4,567,195), isradipine (3,5-Pyridinedicarboxylic acid, 4-(4-benzofurazanyl)-1,4-dihydro-2,6-dimethyl-, methyl 1-methylethyl ester, (±)-4(4-benzofurazanyl)-1,4-dihydro-2,6-dimethyl-3,5-pyridinedicarboxylate, see also U.S. Pat. No. 4,466,972); nimodipine (such as is isopropyl (2-methoxyethyl) 1,4-dihydro-2,6-dimethyl-4-(3-nitrophenyl)-3,5-pyridine-dicarboxylate, e.g. Nimotop®, Bayer), felodipine (such as ethyl methyl 4-(2,3-dichlorophenyl)-1,4-dihydro-2,6-dimethyl-3,5-pyridinedicarboxylate-, e.g. Plendil® Extended-Release, AstraZeneca LP), nilvadipine (3,5-Pyridinedicarboxylic acid, 2-cyano-1,4-dihydro-6-methyl-4-(3-nitrophenyl)-, 3-methyl 5-(1-methylethyl) ester, also see U.S. Pat. No. 3,799,934), nifedipine (such as 3,5-pyridinedicarboxylic acid,1,4-dihydro-2,6-dimethyl-4-(2-nitrophenyl)-, dimethyl ester, e.g., Procardia XL® Extended Release Tablets, Pfizer), diltiazem hydrochloride (such as 1,5-Benzothiazepin-4(5H)-one,3-(acetyloxy)-5[2-(dimethylamino)ethyl]-2,-3-dihydro-2(4-methoxyphenyl)-, monohydrochloride, (+)-cis., e.g., Tiazac®, Forest), verapamil hydrochloride (such as benzeneacetronitrile, (alpha)-[[3-[[2-(3,4-dimethoxyphenyl) ethyl]methylamino]propyl]-3,4-dimethoxy-(alpha)-(1-methylethyl) hydrochloride, e.g., Isoptin® SR, Knoll Labs), teludipine hydrochloride (3,5-Pyridinedicarboxylic acid, 2-[(dimethylamino)methyl]4-[2-[(1E)-3-(1,1-dimethylethoxy)-3-oxo-1-propenyl]phenyl]-1,4-dihydro-6-methyl-, diethyl ester, monohydrochloride) CAS RN 108700-03-4), belfosdil (Phosphonic acid, [2-(2-phenoxy ethyl)-1,3-propane-diyl]bis-, tetrabutyl ester CAS RN 103486-79-9), fostedil (Phosphonic acid, [[4-(2-benzothiazolyl)phenyl]methyl]-, diethyl ester CAS RN 75889-62-2), aranidipine, azelnidipine, barnidipine, benidipine, bepridil, cinaldipine, clevidipine, efonidipine, gallopamil, lacidipine, lemildipine, lercanidipine, monatepil maleate (1-Piperazinebutanamide, N-(6,11-dihydrodibenzo(b,e)thiepin-11-yl)₄-(4-fluorophenyl)-, (+)-, (Z)-2-butenedioate (1:1) (±)-N-(6,11-Dihydrodibenzo(b,e)thiep-in-11-yl)-4-(p-fluorophenyl)-1-piperazinebutyramide maleate (1:1) CAS RN 132046-06-1), nicardipine, nisoldipine, nitrendipine, manidipine, pranidipine, and the like; T-channel calcium antagonists such as mibefradil; angiotensin converting enzyme (ACE) inhibitors such as benazepril, benazepril hydrochloride (such as 3-[[1-(ethoxycarbonyl)-3-phenyl-(1S)-propyl]amino]-2,3,4,5-tetrahydro-2-oxo-1H -1-(3S)-benzazepine-1-acetic acid monohydrochloride, e.g., Lotrel®, Novartis), captopril (such as 1-[(2S)-3-mercapto-2-methylpropionyl]-L-proline, e.g., Captopril, Mylan, CAS RN 62571-86-2 and others disclosed in U.S. Pat. No. 4,046,889), ceranapril (and others disclosed in U.S. Pat. No. 4,452,790), cetapril (alacepril, Dainippon disclosed in Eur. Therap. Res. 39:671 (1986); 40:543 (1986)), cilazapril (Hoffman-LaRoche) disclosed in J. Cardiovasc. Pharmacol. 9:39 (1987), indalapril (delapril hydrochloride (2H-1,2,4-Benzothiadiazine-7-sulfonamide, 3-bicyclo[2.2.1]hept-5-en-2-yl-6-chloro-3,4-dihydro-, 1,1-dioxide CAS RN 2259-96-3); disclosed in U.S. Pat. No. 4,385,051), enalapril (and others disclosed in U.S. Pat. No. 4,374,829), enalopril, enaloprilat, fosinopril, ((such as L-proline, 4-cyclohexyl-1-[[[2-methyl-1-(1-oxopropoxy) propoxy](4-phenylbutyl) phosphinyl]acetyl]-, sodium salt, e.g., Monopril, Bristol-Myers Squibb and others disclosed in U.S. Pat. No. 4,168,267), fosinopril sodium (L-Proline, 4-cyclohexyl-1-[[(R)-[(1S)-2-methyl-1-(1-ox-opropoxy)propox), imidapril, indolapril (Schering, disclosed in J. Cardiovasc. Pharmacol. 5:643, 655 (1983)), lisinopril (Merck), losinopril, moexipril, moexipril hydrochloride (3-Isoquinolinecarboxylic acid, 2-[(2S)-2-[[(1S)-1-(ethoxycarbonyl)-3-phenylpropyl]amino]-1-oxopropyl]-1,-2,3,4-tetrahydro-6,7-dimethoxy-, monohydrochloride, (3S)-CAS RN 82586-52-5), quinapril, quinaprilat, ramipril (Hoechsst) disclosed in EP 79022 and Curr. Ther. Res. 40:74 (1986), perindopril erbumine (such as 2S,3aS,7aS-1-[(S)-N-[(S)-1-Carboxybutyljalanyljhexahydro\hat{ }-indolinecarboxylic acid, 1-ethyl ester, compound with tert-butylamine (1:1), e.g., Aceon®, Solvay), perindopril (Servier, disclosed in Eur. J. din. Pharmacol. 31:519 (1987)), quanipril (disclosed in U.S. Pat. No. 4,344,949), spirapril (Schering, disclosed in Acta. Pharmacol. Toxicol. 59 (Supp. 5): 173 (1986)), tenocapril, trandolapril, zofenopril (and others disclosed in U.S. Pat. No. 4,316,906), rentiapril (fentiapril, disclosed in Clin. Exp. Pharmacol. Physiol. 10:131 (1983)), pivopril, YS980, teprotide (Bradykinin potentiator BPP9a CAS RN 35115-60-7), BRL 36,378 (Smith Kline Beecham, see EP80822 and EP60668), MC-838 (Chugai, see CA. 102:72588v and Jap. J. Pharmacol. 40:373 (1986), CGS 14824 (Ciba-Geigy, 3-([1-ethoxycarbonyl-3-phenyl-(1S)-propyl]amino)-2,3,4,5-tetrahydro-2-ox-o-1-(3S)-benzazepine-1 acetic acid HCl, see U.K. Patent No. 2103614), CGS 16,617 (Ciba-Geigy, 3(S)-[[(1S)-5-amino-1-carboxypentyl]amino]-2,3,4,-5-tetrahydro-2-oxo-1H-1-benzazepine-1-ethanoic acid, see U.S. Pat. No. 4,473,575), Ru 44570 (Hoechst, see Arzneimittelforschung 34:1254 (1985)), R 31-2201 (Hoffman-LaRoche see FEBS Lett. 165:201 (1984)), CI925 (Pharmacologist 26:243, 266 (1984)), WY-44221 (Wyeth, see J. Med. Chem. 26:394 (1983)), and those disclosed in US2003006922 (paragraph 28), U.S. Pat. Nos. 4,337,201, 4,432,971 (phosphonamidates); neutral endopeptidase inhibitors such as omapatrilat (Vanlev®), CGS 30440, cadoxatril and ecadotril, fasidotril (also known as aladotril or alatriopril), sampatrilat, mixanpril, and gemopatrilat, AVE7688, ER4030, and those disclosed in U.S. Pat. Nos. 5,362,727, 5,366,973, 5,225,401, 4,722,810, 5,223,516, 4,749,688, 5,552,397, 5,504,080, 5,612,359, 5,525,723, EP0599444, EP0481522, EP0599444, EP0595610, EP0534363, EP534396, EP534492, EP0629627; endothelin antagonists such as tezosentan, A308165, and YM62899, and the like; vasodilators such as hydralazine (apresoline), clonidine (clonidine hydrochloride (1H-Imidazol-2-amine, N-(2,6-dichlorophenyl)4,5-dihydro-, monohydrochloride CAS RN 4205-91-8), catapres, minoxidil (loniten), nicotinyl alcohol (roniacol), diltiazem hydrochloride (such as 1,5-Benzothiazepin-4(5H)-one,3-(acetyloxy)-5[2-(dimethylamino)ethyl]-2,-3-dihydro-2(4-methoxyphenyl)-, monohydrochloride, (+)-cis, e.g., Tiazac®, Forest), isosorbide dinitrate (such as 1,4:3,6-dianhydro-D-glucitol 2,5-dinitrate e.g., Isordil® Titradose®, Wyeth-Ayerst), sosorbide mononitrate (such as 1,4:3,6-dianhydro-D-glucito-1,5-nitrate, an organic nitrate, e.g., Ismo®, Wyeth-Ayerst), nitroglycerin (such as 2,3 propanetriol trinitrate, e.g., Nitrostat® Parke-Davis), verapamil hydrochloride (such as benzeneacetonitrile, (±)-(alpha)[3-[[2-(3,4 dimethoxyphenyl)ethyl]methylamino]propyl]-3,4-dimethoxy-(alpha)-(1-methylethyl) hydrochloride, e.g., Covera HS® Extended-Release, Searle), chromonar (which may be prepared as disclosed in U.S. Pat. No. 3,282,938), clonitate (Annalen 1870 155), droprenilamine (which may be prepared as disclosed in DE2521113), lidoflazine (which may be prepared as disclosed in U.S. Pat. No. 3,267,104); prenylamine (which may be prepared as disclosed in U.S. Pat. No. 3,152,173), propatyl nitrate (which may be prepared as disclosed in French Patent No. 1,103,113), mioflazine hydrochloride (1-Piperazineacetamide, 3-(aminocarbonyl)₄-[4,4-bis(4-fluorophenyl)butyl]-N-(2,6-dichlorophenyl)-, dihydrochloride CAS RN 83898-67-3), mixidine (Benzeneethanamine, 3,4-dimethoxy-N-(1-methyl-2-pyrrolidinylidene)-Pyrrolidine, 2-[(3,4-dimethoxyphenethyl)imino]-1-methyl-1-Methyl-2-[(3,4-dimethoxyphenethyl)imino]pyrrolidine CAS RN 27737-38-8), molsidomine (1,2,3-Oxadiazolium, 5-[(ethoxycarbonyl)amino]-3-(4-morpholinyl)-, inner salt CAS RN 25717-80-0), isosorbide mononitrate (D-Glucitol, 1,4:3,6-dianhydro-, 5-nitrate CAS RN 16051-77-7), erythrityl tetranitrate (1,2,3,4-Butanetetrol, tetranitrate, (2R,3S)-rel-CAS RN 7297-25-8), clonitrate(1,2-Propanediol, 3-chloro-, dinitrate (7CI, 8CI, 9CI) CAS RN 2612-33-1), dipyridamole Ethanol, 2,2′,2″,2′″-[(4,8-di-1-piperidinylpyrimido[5,4-d]pyrimidine-2,6-diyl)dinitrilo]tetrakis-CAS RN 58-32-2), nicorandil (CAS RN 65141-46-0 3-), pyridinecarboxamide (N-[2-(nitrooxy)ethyl]-Nisoldipine3,5-Pyridinedicarboxylic acid, 1,4-dihydro-2,6-dimethyl-4-(2-nitrophenyl)-, methyl 2-methylpropyl ester CAS RN 63675-72-9), nifedipine3,5-Pyridinedicarboxylic acid, 1,4-dihydro-2,6-dimethyl-4-(2-nitrophenyl)-, dimethyl ester CAS RN 21829-25-4), perhexiline maleate (Piperidine, 2-(2,2-dicyclohexylethyl)-, (2Z)-2-butenedioate (1:1) CAS RN 6724-53-4), oxprenolol hydrochloride (2-Propanol, 1-[(1-methylethyl)amino]-3-[2-(2-propenyloxy)phenoxy]-, hydrochloride CAS RN 6452-73-9), pentrinitrol (1,3-Propanediol, 2,2-bis[(nitrooxy)methyl]-, mononitrate (ester) CAS RN 1607-17-6), verapamil (Benzeneacetonitrile, α-[3-[[2-(3,4-dimethoxyphenyl)ethyl]-methylamino]propyl]-3,4-dimethoxy-α-(1-methylethyl)-CAS RN 52-53-9) and the like; angiotensin II receptor antagonists such as, aprosartan, zolasartan, olmesartan, pratosartan, FI6828K, RNH6270, candesartan (1H-Benzimidazole-7-carboxylic acid, 2-ethoxy-1-[[2′-(1H-tetrazol-5-yl)[1,1′-biphenyl]4-yl]methyl]-CAS RN 139481-59-7), candesartan cilexetil ((+/−)-1-(cyclohexylcarbonyloxy)ethyl-2-ethoxy-1-[[2′-(1H-tetrazol-5-yl)biphenyl-4-yl]-1H-benzimidazole carboxylate, CAS RN 145040-37-5, U.S. Pat. Nos. 5,703,110 and 5,196,444), eprosartan (3[1-4-carboxyphenylmethyl)-2-n-butyl-imidazol-5-yl]-(2-thienylmethyl) propenoic acid, U.S. Pat. Nos. 5,185,351 and 5,650,650), irbesartan (2-n-butyl-3-[[2′-(1h-tetrazol-5-yl)biphenyl-4-yl]methyl]1,3-diazazspiro[4,4]non-1-en-4-one, U.S. Pat. Nos. 5,270,317 and 5,352,788), losartan (2-N-butyl-4-chloro-5-hydroxymethyl-1-[(2′-(1H-tetrazol-5-yl)biphenyl-4-yl)-methyl]imidazole, potassium salt, U.S. Pat Nos. 5,138,069, 5,153,197 and 5,128,355), tasosartan (5,8-dihydro-2,4-dimethyl-8-[(2′-(1H-tetrazol-5-yl)[1,r-biphenyl]4-yl)methyl]-pyrido[2,3-d]pyrimidin-7(6H)-one, U.S. Pat. No. 5,149,699), telmisartan (4′-[(1,4-dimethyl-2′-propyl-(2,6′-bi-1H-benzimidazol)-r-yl)]-[1,1′-biphenyl]-2-carboxylic acid, CAS RN 144701-48-4, U.S. Pat. No. 5,591,762), milfasartan, abitesartan, valsartan (Diovan® (Novartis), (S)-N-valeryl-N-[[2′-(1H-tetrazol-5-yl)biphenyl-4-yl)methyl]valine, U.S. Pat. No. 5,399,578), EXP-3137 (2-N-butyl-4-chloro-1-[(2′-(1H-tetrazol-5-yl)biphenyl-4-yl)-methyl]imidazole-5-carboxylic acid, U.S. Pat. Nos. 5,138,069, 5,153,197 and 5,128,355), 3-(2′-(tetrazol-5-yl)-1,r-biphen-4-yl)methyl-5,7-dimethyl-2-ethyl-3H-imidazo[4,5-b]pyridine, 4′[2-ethyl-4-methyl-6-(5,6,7,8-tetrahydroimidazo[1,2-a]pyridin-2-yl]-benzimidazol-1-yl]-methyl]-l,r-biphenyl]-2-carboxylic acid, 2-butyl-6-(1-methoxy-1-methylethyl)-2-[2′-)1H-tetrazol-5-yl)biphenyl-4-ylmethyl] guinazolin-4(3H)-one, 3-[2′-carboxybiphenyl-4-yl)methyl]-2-cyclopropyl-7-methyl-3H-imidazo[4,5-b]pyridine, 2-butyl-4-chloro-1-[(2′-tetrazol-5-yl)biphenyl-4-yl)methyl]imidazole-carboxylic acid, 2-butyl-4-chloro-1-[[2′-(1H-tetrazol-5-yl) [1,1′-biphenyl]-4-yl]methyl]-1H-imidazole-5-carboxylic acid-1-(ethoxycarbonyl-oxy)ethyl ester potassium salt, dipotassium 2-butyl-4-(methylthio)-1-[[2-[[[(propylamino)carbonyl]amino]-sulfonyl](1,1′-biphenyl)-4-yl]methyl]-1 H-imidazole-5-carboxylate, methyl-2-[[4-butyl-2-methyl-6-oxo-5-[[2′-(1H-tetrazol-5-yl)-[1,1′-biphenyl]-4-yl]methyl]-1-(6H)-pyrimidinyl]methyl]-3-thiophencarboxylate, 5-[(3,5-dibutyl-1H-1,2,4-triazol-1-yl)methyl]-2-[2-(1H-tetrazol-5-ylphenyl)]pyridine, 6-butyl-2-(2-phenylethyl)-5 [[2′-(I H-tetrazol-5-yl)[1,1′-biphenyl]-4-methyl]pyrimidin-4-(3H)-one D,L lysine salt, 5-methyl-7-n-propyl-8-[[2′-(1H-tetrazol-5-yl)biphenyl-4-yl]methyl]-[1,2,4]-triazolo[1,5-c]pyrimidin-2(3H)-one, 2,7-diethyl-5-[[2′-(5-tetrazoly)biphenyl-4-yl]methyl]-5H-pyrazolo[1,5-b][1,2,4]triazole potassium salt, 2-[2-butyl-4,5-dihydro-4-oxo-3-[2′-(1H-tetrazol-5-yl)-4-biphenylmethyl]-3H-imidazol [4,5-c]pyridine-5-ylmethyl]benzoic acid, ethyl ester, potassium salt, 3-methoxy-2,6-dimethyl-4-[[2′(1H-tetrazol-5-yl)-1,1′-biphenyl-4-yl]methoxy]pyridine, 2-ethoxy-1-[[2′-(5-oxo-2,5-dihydro-1,2,4-oxadiazol-3-yl)biphenyl-4-yl]methyl]-1 H-benzimidazole-7-carboxylic acid, 1-[N-(2′-(1H-tetrazol-5-yl)biphenyl-4-yl-methyl)-N-valerolylaminomethyl)cyclopentane-1-carboxylic acid, 7-methyl-2n-propyl-3-[[2′1H-tetrazol-5-yl)biphenyl-4-yl]methyl]-3H-imidazo[4,5-6]pyridine, 2-[5-[(2-ethyl-5,7-dimethyl-3H-imidazo[4,5-b]pyridine-3-yl)methyl]-2-quinolinyl]sodium benzoate, 2-butyl-6-chloro-4-hydroxymethyl-5-methyl-3-[[2′-(I H-tetrazol-5-yl)biphenyl-4-yl]methyl]pyridine, 2-[[[2-butyl-1-[(4-carboxyphenyl)methyl]-1H-imidazol-5-yl]methyl]amino]benzoic acid tetrazol-5-yl)biphenyl-4-yl]methyl]pyrimidin-6-one, 4(S)-[4-(carboxymethyl)phenoxy]-N-[2(R)-[4-(2-sulfobenzamido)imidazol-1-yl]octanoyl]-L-proline, 1-(2,6-dimethylphenyl)-4-butyl-1,3-dihydro-3-[[6-[2-(1H-tetrazol-5-yl)phenyl]-3-pyridinyl]methyl]-2H-imidazol-2-one, 5,8-ethano-5,8-dimethyl-2-n-propyl-5,6,7,8-tetrahydro-1-[[2′(1H-tetrazol-5-yl)biphenyl-4-yl]methyl]-1H,4H-1,3,4a,8a-tetrazacyclopentanaphthalene-9-one, 4[1-[2′-(1,2,3,4-tetrazol-5-yl)biphen-4-yl)methylamino]-5,6,7,8-tetrahydro-2-trifylquinazoline, 2-(2-chlorobenzoyl)imino-5-ethyl-3-[2′-(1H-tetrazole-5-yl)biphenyl-4-yl)methyl-1,3,4-thiadiazoline, 2-[5-ethyl-3-[2-(1H-tetrazole-5-yl)biphenyl-4-yl]methyl-1,3,4-thiazoline-2-ylidene]aminocarbonyl-1-cyclopentencarboxylic acid dipotassium salt, and 2-butyl-4-[N-methyl-N-(3-methylcrotonoyl)amino]-1-[[2′-(1H-tetrazol-5-yl)biphenyl-4-yl]methyl]-1H-imidzole-5-carboxylic acid 1-ethoxycarbonyloxyethyl ester, those disclosed in patent publications EP475206, EP497150, EP539086, EP539713, EP535463, EP535465, EP542059, EP497121, EP535420, EP407342, EP415886, EP424317, EP435827, EP433983, EP475898, EP490820, EP528762, EP324377, EP323841, EP420237, EP500297, EP426021, EP480204, EP429257, EP430709, EP434249, EP446062, EP505954, EP524217, EP514197, EP514198, EP514193, EP514192, EP450566, EP468372, EP485929, EP503162, EP533058, EP467207 EP399731, EP399732, EP412848, EP453210, EP456442, EP470794, EP470795, EP495626, EP495627, EP499414, EP499416, EP499415, EP511791, EP516392, EP520723, EP520724, EP539066, EP438869, EP505893, EP530702, EP400835, EP400974, EP401030, EP407102, EP411766, EP409332, EP412594, EP419048, EP480659, EP481614, EP490587, EP467715, EP479479, EP502725, EP503838, EP505098, EP505111 EP513,979 EP507594, EP510812, EP511767, EP512675, EP512676, EP512870, EP517357, EP537937, EP534706, EP527534, EP540356, EP461040, EP540039, EP465368, EP498723, EP498722, EP498721, EP515265, EP503785, EP501892, EP519831, EP532410, EP498361, EP432737, EP504888, EP508393, EP508445, EP403159, EP403158, EP425211, EP427463, EP437103, EP481448, EP488532, EP501269, EP500409, EP540400, EP005528, EP028834, EP028833, EP411507, EP425921, EP430300, EP434038, EP442473, EP443568, EP445811, EP459136, EP483683, EP518033, EP520423, EP531876, EP531874, EP392317, EP468470, EP470543, EP502314, EP529253, EP543263, EP540209, EP449699, EP465323, EP521768, EP415594, WO92/14468, WO93/08171, WO93/08169, WO91/00277, WO91/00281, WO91/14367, WO92/00067, WO92/00977, WO92/20342, WO93/04045, WO93/04046, WO91/15206, WO92/14714, WO92/09600, WO92/16552, WO93/05025, WO93/03018, WO91/07404, WO92/02508, WO92/13853, WO91/19697, WO91/11909, WO91/12001, WO91/11999, WO91/15209, WO91/15479, WO92/20687, WO92/20662, WO92/20661, WO93/01177, WO91/14679, WO91/13063, WO92/13564, WO91/17148, WO91/18888, WO91/19715, WO92/02257, WO92/04335, WO92/05161, WO92/07852, WO92/15577, WO93/03033, WO91/16313, WO92/00068, WO92/02510, WO92/09278, WO9210179, WO92/10180, WO92/10186, WO92/10181, WO92/10097, WO92/10183, WO92/10182, WO92/10187, WO92/10184, WO92/10188, WO92/10180, WO92/10185, WO92/20651, WO93/03722, WO93/06828, WO93/03040, WO92/19211, WO92/22533, WO92/06081, WO92/05784, WO93/00341, WO92/04343, WO92/04059, U.S. Pat. Nos. 5,104,877, 5,187,168, 5,149,699, 5,185,340, 4,880,804, 5,138,069, 4,916,129, 5,153,197, 5,173,494, 5,137,906, 5,155,126, 5,140,037, 5,137,902, 5,157,026, 5,053,329, 5,132,216, 5,057,522, 5,066,586, 5,089,626, 5,049,565, 5,087,702, 5,124,335, 5,102,880, 5,128,327, 5,151,435, 5,202,322, 5,187,159, 5,198,438, 5,182,288, 5,036,048, 5,140,036, 5,087,634, 5,196,537, 5,153,347, 5,191.086, 5,190.942, 5,177.097, 5,212,177, 5,208,234, 5,208,235, 5,212,195, 5,130,439, 5,045,540, 5,041,152, and 5,210,204, and pharmaceutically acceptable salts and esters thereof; α/β adrenergic blockers such as nipradilol, arotinolol, amosulalol, bretylium tosylate (CAS RN: 61-75-6), dihydroergtamine mesylate (such as ergotaman-3′,6′,18-trione,9,-10-dihydro-12′-hydroxy-2′-methyl-5′-(phenylmethyl)-,(5′(α))-, monomethanesulfonate, e.g., DHE 45® Injection, Novartis), carvedilol (such as (±)-1-(Carbazol-4-yloxy)-3-[[2-(o-methoxyphenoxy)ethyl]amino]-2-propanol, e.g., Coreg®, SmithKline Beecham), labetalol (such as 5-[1-hydroxy-2-[(1-methyl-3-phenylpropyl) amino] ethyljsalicylamide monohydrochloride, e.g., Normodyne®, Schering), bretylium tosylate (Benzenemethanaminium, 2-bromo-N-ethyl-N,N-dimethyl-, salt with 4-methylbenzenesulfonic acid (1:1) CAS RN 61-75-6), phentolamine mesylate (Phenol, 3-[[(4,5-dihydro-1H-imidazol-2-yl)methyl](4-methylphenyl)amino]-, monomethanesulfonate (salt) CAS RN 65-28-1), solypertine tartrate (5H-1,3-Dioxolo[4,5-f]indole, 7-[2-[4-(2-methoxyphenyl)-1-piperazinyl]ethyl]-, (2R,3R)-2,3-dihydroxybutanedioate (1:1) CAS RN 5591-43-5), zolertine hydrochloride (Piperazine, 1-phenyl4-[2-(1H-tetrazol-5-yl)ethyl]-, monohydrochloride (8Cl, 9Cl) CAS RN 7241-94-3) and the like; α adrenergic receptor blockers, such as alfuzosin (CAS RN: 81403-68-1), terazosin, urapidil, prazosin (Minipress®), tamsulosin, bunazosin, trimazosin, doxazosin, naftopidil, indoramin, WHP 164, XENOlO, fenspiride hydrochloride (which may be prepared as disclosed in U.S. Pat. No. 3,399,192), proroxan (CAS RN 33743-96-3), and labetalol hydrochloride and combinations thereof; α 2 agonists such as methyldopa, methyldopa HCL, lofexidine, tiamenidine, moxonidine, rilmenidine, guanobenz, and the like; aldosterone inhibitors, and the like; renin inhibitors including Aliskiren (SPPlOO; Novartis/Speedel); angiopoietin-2-binding agents such as those disclosed in WO03/030833; anti-angina agents such as ranolazine (hydrochloride 1-Piperazineacetamide, N-(2,6-dimethylphenyl)-4-[2-hydroxy-3-(2-methoxyphenoxy)propyl]-, dihydrochloride CAS RN 95635-56-6), b etaxolol hydrochloride (2-Propanol, 1-[4-[2(cyclopropylmethoxy)ethyl]phenoxy]-3-[(1-methylethyl)amino]-, hydrochloride CAS RN 63659-19-8), butoprozine hydrochloride (Methanone, [4-[3(dibutylamino)propoxy]phenyl](2-ethyl-3-indolizinyl)-, monohydrochloride CAS RN 62134-34-3), cinepazet maleatel-Piperazineacetic acid, 4-[1-oxo-3-(3,4,5-trimethoxyphenyl)-2-propenyl]-, ethyl ester, (2Z)-2-butenedioate (1:1) CAS RN 50679-07-7), tosifen (Benzenesulfonamide, 4-methyl-N-[[[(1S)-1-methyl -2-phenylethyl]amino]carbonyl]-CAS RN 32295-184), verapamilhydrochloride (Benzeneacetonitrile, α-[3-[[2-(3,4-dimethoxyphenyl)ethyl]methylamino]propyl]-3,4-dimethoxy-α-(1-methylethyl)-, monohydrochloride CAS RN 152-114), molsidomine (1,2,3-Oxadiazolium, 5-[(ethoxycarbonyl)amino]-3-(4-morpholinyl)-, inner salt CAS RN 25717-80-0), and ranolazine hydrochloride (1-Piperazineacetamide, N-(2,6-dimethylphenyl)₄-[2-hydroxy-3-(2-methoxyphenoxy)propyl]-, dihydrochloride CAS RN 95635-56-6); tosifen (Benzenesulfonamide, 4-methyl-N-[[[(1S)-1-methyl-2-phenylethyl]amino]carbonyl]-CAS RN 32295-184); adrenergic stimulants such as guanfacine hydrochloride (such as N-amidino-2-(2,6-dichlorophenyl) acetamide hydrochloride, e.g., Tenex® Tablets available from Robins); methyldopa-hydrochlorothiazide (such as levo-3-(3,4-dihydroxyphenyl)-2-methylalanine) combined with Hydrochlorothiazide (such as 6-chloro-3,4-dihydro-2H -1,2,4-benzothiadiazine-7-sulfonamide 1,1-dioxide, e.g., the combination as, e.g., Aldoril® Tablets available from Merck), methyldopa-chlorothiazide (such as 6-chloro-2H-1,2,4-benzothiadiazine-7-sulfonamide 1,1-dioxide and methyldopa as described above, e.g., Aldoclor®, Merck), clonidine hydrochloride (such as 2-(2,6-dichlorophenylamino)-2-imidazoline hydrochloride and chlorthalidone (such as 2-chloro-5-(1-hydroxy-3-oxo-1-isoindolinyl) benzenesulfonamide), e.g., Combipres®, Boehringer Ingelheim), clonidine hydrochloride (such as 2-(2,6-dichlorophenylamino)-2-imidazoline hydrochloride, e.g., Catapres®, Boehringer Ingelheim), clonidine (1H-Imidazol-2-amine, N-(2,6-dichlorophenyl)4,5-dihydro-CAS RN 4205-90-7), Hyzaar (Merck; a combination of losartan and hydrochlorothiazide), Co-Diovan (Novartis; a combination of valsartan and hydrochlorothiazide, Lotrel (Novartis; a combination of benazepril and amlodipine) and Caduet (Pfizer; a combination of amlodipine and atorvastatin), and those agents disclosed in US20030069221.

### Agents for the Treatment of Respiratory Disorders

The GCRA peptides described herein can be used in combination therapy with one or more of the following agents useful in the treatment of respiratory and other disorders including but not limited to: (1)β-agonists including but not limited to : albuterol (PRO VENTIL®, S ALBUT AMOI®, VENTOLIN®), bambuterol, bitoterol, clenbuterol, fenoterol, formoterol, isoetharine (BRONKOSOL®, BRONKOMETER®), metaproterenol (ALUPENT®, METAPREL®), pirbuterol (MAXAIR®), reproterol, rimiterol, salmeterol, terbutaline (BRETHAIRE®, BRETHINE®, BRICANYL®), adrenalin, isoproterenol (ISUPREL®), epinephrine bitartrate (PRIMATENE®), ephedrine, orciprenline, fenoterol and isoetharine; (2) steroids, including but not limited to beclomethasone, beclomethasone dipropionate, betamethasone, budesonide, bunedoside, butixocort, dexamethasone, flunisolide, fluocortin, fluticasone, hydrocortisone, methyl prednisone, mometasone, predonisolone, predonisone, tipredane, tixocortal, triamcinolone, and triamcinolone acetonide; (3) β2-agonist-corticosteroid combinations [e.g., salmeterol-fluticasone (AD V AIR®), formoterol-budesonid (SYMBICORT®)]; (4) leukotriene D4 receptor antagonists/leukotriene antagonists/LTD4 antagonists (i.e., any compound that is capable of blocking, inhibiting, reducing or otherwise interrupting the interaction between leukotrienes and the Cys LTI receptor) including but not limited to: zafhiukast, montelukast, montelukast sodium (SINGULAIR®), pranlukast, iralukast, pobilukast, SKB-106,203 and compounds described as having LTD4 antagonizing activity described in U.S. Pat. No. 5,565,473; (5) 5-lipoxygenase inhibitors and/or leukotriene biosynthesis inhibitors [e.g., zileuton and BAY1005 (CA registry 128253-31-6)]; (6) histamine Hl receptor antagonists/antihistamines (i.e., any compound that is capable of blocking, inhibiting, reducing or otherwise interrupting the interaction between histamine and its receptor) including but not limited to: astemizole, acrivastine, antazoline, azatadine, azelastine, astamizole, bromopheniramine, bromopheniramine maleate, carbinoxamine, carebastine, cetirizine, chlorpheniramine, chloropheniramine maleate, cimetidine clemastine, cyclizine, cyproheptadine, descarboethoxyloratadine, dexchlorpheniramine, dimethindene, diphenhydramine, diphenylpyraline, doxylamine succinate, doxylarnine, ebastine, efletirizine, epinastine, famotidine, fexofenadine, hydroxyzine, hydroxyzine, ketotifen, levocabastine, levocetirizine, levocetirizine, loratadine, meclizine, mepyramine, mequitazine, methdilazine, mianserin, mizolastine, noberastine, norasternizole, noraztemizole, phenindamine, pheniramine, picumast, promethazine, pynlamine, pyrilamine, ranitidine, temelastine, terfenadine, trimeprazine, tripelenamine, and triprolidine; (7) an anticholinergic including but not limited to: atropine, benztropine, biperiden, flutropium, hyoscyamine (e.g. Levsin®; Levbid®; Levsin/SL®, Anaspaz®, Levsinex timecaps®, NuLev®), ilutropium, ipratropium, ipratropium bromide, methscopolamine, oxybutinin, rispenzepine, scopolamine, and tiotropium; (8) an anti-tussive including but not limited to: dextromethorphan, codeine, and hydromorphone; (9) a decongestant including but not limited to: pseudoephedrine and phenylpropanolamine; (10) an expectorant including but not limited to: guafenesin, guaicolsulfate, terpin, ammonium chloride, glycerol guaicolate, and iodinated glycerol; (11) a bronchodilator including but not limited to: theophylline and aminophylline; (12) an anti-inflammatory including but not limited to: fluribiprofen, diclophenac, indomethacin, ketoprofen, S-ketroprophen, tenoxicam; (13) a PDE (phosphodiesterase) inhibitor including but not limited to those disclosed herein; (14) a recombinant humanized monoclonal antibody [e.g. xolair (also called omalizumab), rhuMab, and talizumab]; (15) a humanized lung surfactant including recombinant forms of surfactant proteins SP-B, SP-C or SP-D [e.g. SURFAXIN®, formerly known as dsc-104 (Discovery Laboratories)], (16) agents that inhibit epithelial sodium channels (ENaC) such as amiloride and related compounds; (17) antimicrobial agents used to treat pulmonary infections such as acyclovir, amikacin, amoxicillin, doxycycline, trimethoprin sulfamethoxazole, amphotericin B, azithromycin, clarithromycin, roxithromycin, clarithromycin, cephalosporins(ceffoxitin, cefmetazole etc), ciprofloxacin, ethambutol, gentimycin, ganciclovir, imipenem, isoniazid, itraconazole, penicillin, ribavirin, rifampin, rifabutin,amantadine, rimantidine, streptomycin, tobramycin, and vancomycin; (18) agents that activate chloride secretion through Ca++ dependent chloride channels (such as purinergic receptor (P2Y(2) agonists); (19) agents that decrease sputum viscosity, such as human recombinant DNase 1, (Pulmozyme®); (20) nonsteroidal anti-inflammatory agents (acemetacin, acetaminophen, acetyl salicylic acid, alclofenac, alminoprofen, apazone, aspirin, benoxaprofen, bezpiperylon, bucloxic acid, carprofen, clidanac, diclofenac, diclofenac, diflunisal, diflusinal, etodolac, fenbufen, fenbufen, fenclofenac, fenclozic acid, fenoprofen, fentiazac, feprazone, flufenamic acid, flufenisal, flufenisal, fluprofen, flurbiprofen, flurbiprofen, furofenac, ibufenac, ibuprofen, indomethacin, indomethacin, indoprofen, isoxepac, isoxicam, ketoprofen, ketoprofen, ketorolac, meclofenamic acid, meclofenamic acid, mefenamic acid, mefenamic acid, miroprofen, mofebutazone, nabumetone oxaprozin, naproxen, naproxen, niflumic acid, oxaprozin, oxpinac, oxyphenbutazone, phenacetin, phenylbutazone, phenylbutazone, piroxicam, piroxicam, pirprofen, pranoprofen, sudoxicam, tenoxican, sulfasalazine, sulindac, sulindac, suprofen, tiaprofenic acid, tiopinac, tioxaprofen, tolfenamic acid, tolmetin, tolmetin, zidometacin, zomepirac, and zomepirac); and (21) aerosolized antioxidant therapeutics such as S-Nitrosoglutathione.

### Anti-Obesity Agents

The GCRA peptides described herein can be used in combination therapy with an anti-obesity agent. Suitable such agents include, but are not limited to: 1 1β HSD-I (11-beta hydroxy steroid dehydrogenase type 1) inhibitors, such as BVT 3498, BVT 2733, 3-(1-adamantyl)-4-ethyl-5-(ethylthio)-4H-1,2,4-triazole, 3-(1-adamantyl)-5-(3,4,5-trimethoxyphenyl)-4-methyl-4H-1,2,4-triazole, 3-adamantanyl-4,5,6,7,8,9,10,11,12,3a-decahydro-1,2,4-triazolo[4,3-a][1 1]annulene, and those compounds disclosed in WO01/90091, WO01/90090, WO01/90092 and WO02/072084; 5HT antagonists such as those in WO03/037871, WO03/037887, and the like; 5HTIa modulators such as carbidopa, benserazide and those disclosed in U.S. Pat. No. 6,207,699, WO03/031439, and the like; 5HT2c (serotonin receptor 2c) agonists, such as BVT933, DPCA37215, IK264, PNU 22394, WAY161503, R-1065, SB 243213 (Glaxo Smith Kline) and YM 348 and those disclosed in U.S. Pat. No. 3,914,250, WO00/77010, WO02/36596, WO02/48124, WO02/10169, WO01/66548, WO02/44152, WO02/51844, WO02/40456, and WO02/40457; 5HT6 receptor modulators, such as those in WO03/030901, WO03/035061, WO03/039547, and the like; acyl-estrogens, such as oleoyl-estrone, disclosed in del Mar-Grasa, M. et al, Obesity Research, 9:202-9 (2001) and Japanese Patent Application No. JP 2000256190; anorectic bicyclic compounds such as 1426 (Aventis) and 1954 (Aventis), and the compounds disclosed in WO00/18749, WO01/32638, WO01/62746, WO01/62747, and WO03/015769; CB 1 (cannabinoid-1 receptor) antagonist/inverse agonists such as rimonabant (Acomplia; Sanofi), SR-147778 (Sanofi), SR-141716 (Sanofi), BAY 65-2520 (Bayer), and SLV 319 (Solvay), and those disclosed in patent publications U.S. Pat. Nos. 4,973,587, 5,013,837, 5,081,122, 5,112,820, 5,292,736, 5,532,237, 5,624,941, 6,028,084, 6,509,367, 6,509,367, WO96/33159, WO97/29079, WO98/31227, WO98/33765, WO98/37061, WO98/41519, WO98/43635, WO98/43636, WO99/02499, WO00/10967, WO00/10968, WO01/09120, WO01/58869, WO01/64632, WO01/64633, WO01/64634, WO01/70700, WO01/96330, WO02/076949, WO03/006007, WO03/007887, WO03/020217, WO03/026647, WO03/026648, WO03/027069, WO03/027076, WO03/027114, WO03/037332, WO03/040107, WO03/086940, WO03/084943 and EP658546; CCK-A (cholecystokinin-A) agonists, such as AR-R 15849, GI 181771 (GSK), JMV-180, A-71378, A-71623 and SR146131 (Sanofi), and those described in U.S. Pat. No. 5,739,106; CNTF (Ciliary neurotrophic factors), such as GI-181771 (Glaxo-SmithKline), SR1 46131 (Sanofi Synthelabo), butabindide, PD 170,292, and PD 149164 (Pfizer); CNTF derivatives, such as Axokine® (Regeneron), and those disclosed in WO94/09134, WO98/22128, and WO99/43813; dipeptidyl peptidase IV (DP-IV) inhibitors, such as isoleucine thiazolidide, valine pyrrolidide, NVP-DPP728, LAF237, P93/01, P 3298, TSL 225 (tryptophyl-1,2,3,4-tetrahydroisoquinoline-3-carboxylic acid; disclosed by Yamada et al, Bioorg. & Med. Chem. Lett. 8 (1998) 1537-1540), TMC-2A/2B/2C, CD26 inhibitors, FE 999011, P9310/K364, VIP 0177, SDZ 274-444, 2-cyanopyrrolidides and 4-cyanopyrrolidides as disclosed by Ashworth et al, Bioorg. & Med. Chem. Lett., Vol. 6, No. 22, pp 1163-1166 and 2745-2748 (1996) and the compounds disclosed patent publications. WO99/38501, WO99/46272, WO99/67279 (Probiodrug), WO99/67278 (Probiodrug), WO99/61431 (Probiodrug), WO02/083128, WO02/062764, WO03/000180, WO03/000181, WO03/000250, WO03/002530, WO03/002531, WO03/002553, WO03/002593, WO03/004498, WO03/004496,WO03/017936, WO03/024942, WO03/024965, WO03/033524, WO03/037327 and EP1258476; growth hormone secretagogue receptor agonists/antagonists, such as NN703, hexarelin, MK-0677 (Merck), SM-130686, CP-424391 (Pfizer), LY 444,711 (Eli Lilly), L-692,429 and L-163,255, and such as those disclosed in U.S. Ser. No. 09/662,448, U.S. provisional application 60/203,335, U.S. Pat. No. 6,358,951, US2002049196, US2002/022637, WO01/56592 and WO02/32888; H3 (histamine H3) antagonist/inverse agonists, such as thioperamide, 3-(1H-imidazol-4-yl)propyl N-(4-pentenyl)carbamate), clobenpropit, iodophenpropit, imoproxifan, GT2394 (Gliatech), and A331440, O-[3-1H-imidazol-4-yl)propanol]carbamates (Kiec-Kononowicz, K. et al., Pharmazie, 55:349-55 (2000)), piperidine-containing histamine H3-receptor antagonists (Lazewska, D. et al., Pharmazie, 56:927-32 (2001), benzophenone derivatives and related compounds (Sasse, A. et al., Arch. Pharm. (Weinheim) 334:45-52 (2001)), substituted N-phenylcarbamates (Reidemeister, S. et al., Pharmazie, 55:83-6 (2000)), and proxifan derivatives (Sasse, A. et al., J. Med. Chem. 43:3335-43 (2000)) and histamine H3 receptor modulators such as those disclosed in WO02/15905, WO03/024928 and WO03/024929; leptin derivatives, such as those disclosed in U.S. Pat. Nos. 5,552,524, 5,552,523, 5,552,522, 5,521,283, WO96/23513, WO96/23514, WO96/23515, WO96/23516, WO96/23517, WO96/23518, WO96/23519, and WO96/23520; leptin, including recombinant human leptin (PEG-OB, Hoffman La Roche) and recombinant methionyl human leptin (Amgen); lipase inhibitors, such as tetrahydrolipstatin (orlistat/Xenical®), Triton WRl 339, RHC80267, lipstatin, teasaponin, diethylumbelliferyl phosphate, FL-386, WAY-121898, Bay-N-3176, valilactone, esteracin, ebelactone A, ebelactone B, and RHC 80267, and those disclosed in patent publications WO01/77094, U.S. Pat. Nos. 4,598,089, 4,452,813, 5,512.565, 5,391,571, 5,602,151, 4,405,644, 4,189,438, and 4,242,453; lipid metabolism modulators such as maslinic acid, erythrodiol, ursolic acid uvaol, betulinic acid, betulin, and the like and compounds disclosed in WO03/011267; Mc4r (melanocortin 4 receptor) agonists, such as CHIR86036 (Chiron), ME-10142, ME-10145, and HS-131 (Melacure), and those disclosed in PCT publication Nos. WO99/64002, WO00/74679, WO01/991752, WO01/25192, WO01/52880, WO01/74844, WO01/70708, WO01/70337, WO01/91752, WO02/059095, WO02/059107, WO02/059108, WO02/059117, WO02/06276, WO02/12166, WO02/11715, WO02/12178, WO02/15909, WO02/38544, WO02/068387, WO02/068388, WO02/067869, WO02/081430, WO03/06604, WO03/007949, WO03/009847, WO03/009850, WO03/013509, and WO03/031410; Mc5r (melanocortin 5 receptor) modulators, such as those disclosed in WO97/19952, WO00/15826, WO00/15790, US20030092041; melanin-concentrating hormone 1 receptor (MCHR) antagonists, such as T-226296 (Takeda), SB 568849, SNP-7941 (Synaptic), and those disclosed in patent publications WO01/21169, WO01/82925, WO01/87834, WO02/051809, WO02/06245, WO02/076929, WO02/076947, WO02/04433, WO02/51809, WO02/083134, WO02/094799, WO03/004027, WO03/13574, WO03/15769, WO03/028641, WO03/035624, WO03/033476, WO03/033480, JP13226269, and JP1437059; mGluR5 modulators such as those disclosed in WO03/029210, WO03/047581, WO03/048137, WO03/051315, WO03/051833, WO03/053922, WO03/059904, and the like; serotoninergic agents, such as fenfluramine (such as Pondimin® (Benzeneethanamine, N-ethyl-alpha-methyl-3-(trifluoromethyl)-, hydrochloride), Robbins), dexfenfluramine (such as Redux® (Benzeneethanamine, N-ethyl-alpha-methyl-3-(trifluoromethyl)-, hydrochloride), Interneuron) and sibutramine ((Meridia®, Knoll/Reductil™) including racemic mixtures, as optically pure isomers (+) and (−), and pharmaceutically acceptable salts, solvents, hydrates, clathrates and prodrugs thereof including sibutramine hydrochloride monohydrate salts thereof, and those compounds disclosed in U.S. Pat. Nos. 4,746,680, 4,806,570, and 5,436,272, US20020006964, WO01/27068, and WO01/62341; NE (norepinephrine) transport inhibitors, such as GW 320659, despiramine, talsupram, and nomifensine; NPY 1 antagonists, such as BIBP3226, J-115814, BIBO 3304, LY-357897, CP-671906, GI-264879A, and those disclosed in U.S. Pat. No. 6,001,836, WO96/14307, WO01/23387, WO99/51600, WO01/85690, WO01/85098, WO01/85173, and WO01/89528; NPY5 (neuropeptide Y Y5) antagonists, such as 152,804, GW-569180A, GW-594884A, GW-587081X, GW-548118X, FR235208, FR226928, FR240662, FR252384, 1229U91, GI-264879A, CGP71683A, LY-377897, LY-366377, PD-160170, SR-120562A, SR-120819A, JCF-104, and H409/22 and those compounds disclosed in patent publications U.S. Pat. Nos. 6,140,354, 6,191,160, 6,218,408, 6,258,837, 6,313,298, 6,326,375, 6,329,395, 6,335,345, 6,337,332, 6,329,395, 6,340,683, EP01010691, EP-01044970, WO97/19682, WO97/20820, WO97/20821, WO97/20822, WO97/20823, WO98/27063, WO00/107409, WO00/185714, WO00/185730, WO00/64880, WO00/68197, WO00/69849, WO/0113917, WO01/09120, WO01/14376, WO01/85714, WO01/85730, WO01/07409, WO01/02379, WO01/23388, WO01/23389, WO01/44201, WO01/62737, WO01/62738, WO01/09120, WO02/20488, WO02/22592, WO02/48152, WO02/49648, WO02/051806, WO02/094789, WO03/009845, WO03/014083, WO03/022849, WO03/028726 and Norman et al, J. Med. Chem. 43:4288-4312 (2000); opioid antagonists, such as nalmefene (REVEX®), 3-methoxynaltrexone, methylnaltrexone, naloxone, and naltrexone (e.g. PT901; Pain Therapeutics, Inc.) and those disclosed in US20050004155 and WO00/21509; orexin antagonists, such as SB-334867-A and those disclosed in patent publications WO01/96302, WO01/68609, WO02/44172, WO02/51232, WO02/51838, WO02/089800, WO02/090355, WO03/023561, WO03/032991, and WO03/037847; PDE inhibitors (e.g. compounds which slow the degradation of cyclic AMP (cAMP) and/or cyclic GMP (cGMP) by inhibition of the phosphodiesterases, which can lead to a relative increase in the intracellular concentration of cAMP and cGMP; possible PDE inhibitors are primarily those substances which are to be numbered among the class consisting of the PDE3 inhibitors, the class consisting of the PDE4 inhibitors and/or the class consisting of the PDE5 inhibitors, in particular those substances which can be designated as mixed types of PDE3/4 inhibitors or as mixed types of PDE3/4/5 inhibitors) such as those disclosed in patent publications DE1470341, DE2108438, DE2123328, DE2305339, DE2305575, DE2315801, DE2402908, DE2413935, DE2451417, DE2459090, DE2646469, DE2727481, DE2825048, DE2837161, DE2845220, DE2847621, DE2934747, DE3021792, DE3038166, DE3044568, EP000718, EP0008408, EP0010759, EP0059948, EP0075436, EP0096517, EPOl 12987, EPOl 16948, EP0150937, EP0158380, EP0161632, EP0161918, EP0167121, EP0199127, EP0220044, EP0247725, EP0258191, EP0272910, EP0272914, EP0294647, EP0300726, EP0335386, EP0357788, EP0389282, EP0406958, EP0426180, EP0428302, EP0435811, EP0470805, EP0482208, EP0490823, EP0506194, EP0511865, EP0527117, EP0626939, EP0664289, EP0671389, EP0685474, EP0685475, EP0685479, JP92234389, JP94329652, JP95010875, U.S. Pat. Nos. 4,963,561, 5,141,931, WO9117991, WO9200968, WO9212961, WO9307146, WO9315044, WO9315045, WO9318024, WO9319068, WO9319720, WO9319747, WO9319749, WO9319751, WO9325517, WO9402465, WO9406423, WO9412461, WO9420455, WO9422852, WO9425437, WO9427947, WO9500516, WO9501980, WO9503794, WO9504045, WO9504046, WO9505386, WO9508534, WO9509623, WO9509624, WO9509627, WO9509836, WO9514667, WO9514680, WO9514681, WO9517392, WO9517399, WO9519362, WO9522520, WO9524381, WO9527692, WO9528926, WO9535281, WO9535282, WO9600218, WO9601825, WO9602541, WO9611917, DE3142982, DEl 116676, DE2162096, EP0293063, EP0463756, EP0482208, EP0579496, EP0667345 U.S. Pat. No. 6,331,543, US20050004222 (including those disclosed in formulas I-XIII and paragraphs 37-39, 85-0545 and 557-577), WO9307124, EP0163965, EP0393500, EP0510562, EP0553174, WO9501338 and WO9603399, as well as PDE5 inhibitors (such as RX-RA-69, SCH-51866, KT-734, vesnarinone, zaprinast, SKF-96231, ER-21355, BF/GP-385, NM-702 and sildenafil (Viagra™)), PDE4 inhibitors (such as etazolate, ICI63197, RP73401, imazolidinone (RO-20-1724), MEM 1414 (R1533/R1500; Pharmacia Roche), denbufylline, rolipram, oxagrelate, nitraquazone, Y-590, DH-6471, SKF-94120, motapizone, lixazinone, indolidan, olprinone, atizoram, KS-506-G, dipamfylline, BMY-43351, atizoram, arofylline, filaminast, PDB-093, UCB-29646, CDP-840, SKF-107806, piclamilast, RS-17597, RS-25344-000, SB-207499, TIBENELAST, SB-210667, SB-211572, SB-211600, SB-212066, SB-212179, GW-3600, CDP-840, mopidamol, anagrelide, ibudilast, amrinone, pimobendan, cilostazol, quazinone and N-(3,5-dichloropyrid-4-yl)-3-cyclopropylmethoxy4-difluoromethoxybenzamide, PDE3 inhibitors (such as ICI153, 100, bemorandane (RWJ 22867), MCI-154, UD-CG 212, sulmazole, ampizone, cilostamide, carbazeran, piroximone, imazodan, CI-930, siguazodan, adibendan, saterinone, SKF-95654, SDZ-MKS-492, 349-U-85, emoradan, EMD-53998, EMD-57033, NSP-306, NSP-307, revizinone, NM-702, WIN-62582 and WIN-63291, enoximone and milrinone, PDE3/4 inhibitors (such as benafentrine, trequinsin, ORG-30029, zardaverine, L-686398, SDZ-ISQ-844, ORG-20241, EMD-54622, and tolafentrine) and other PDE inhibitors (such as vinpocetin, papaverine, enprofylline, cilomilast, fenoximone, pentoxifylline, roflumilast, tadalafil (Cialis®), theophylline, and vardenafil (Levitra®); Neuropeptide Y2 (NPY2) agonists include but are not limited to: polypeptide YY and fragments and variants thereof (e.g. YY3-36 (PYY3-36) (N. Engl. J. Med. 349:941, 2003; IKPEAPGE DASPEELNRY YASLRHYLNL VTRQRY (SEQ ID NO: 2 of WO 2009080608, which is herein incorporated by reference in its entirety)) and PYY agonists such as those disclosed in WO02/47712, WO03/026591, WO03/057235, and WO03/027637; serotonin reuptake inhibitors, such as, paroxetine, fluoxetine (Prozac™), fluvoxamine, sertraline, citalopram, and imipramine, and those disclosed in U.S. Pat. Nos. 6,162,805, 6,365,633, WO03/00663, WO01/27060, and WO01/162341; thyroid hormone β agonists, such as KB-2611 (KaroBioBMS), and those disclosed in WO02/15845, WO97/21993, WO99/00353, GB98/284425, U.S. Provisional Application No. 60/183,223, and Japanese Patent Application No. JP 2000256190; UCP-I (uncoupling protein-1), 2, or 3 activators, such as phytanic acid, 4-[(E)-2-(5,6,7,8-tetrahydro-5,5,8,8-tetramethyl-2-napthalenyl)-1-propenyl]benzoic acid (TTNPB), retinoic acid, and those disclosed in WO99/00123; β3 (beta adrenergic receptor 3) agonists, such as AJ9677/TAK677 (Dainippon/Takeda), L750355 (Merck), CP331648 (Pfizer), CL-316,243, SB 418790, BRL-37344, L-796568, BMS-196085, BRL-35135A, CGP12177A, BTA-243, GW 427353, Trecadrine, Zeneca D7114, N-5984 (Nisshin Kyorin), LY-377604 (Lilly), SR 59119A, and those disclosed in U.S. Pat. Nos. 5,541,204, 5,770,615, 5,491,134, 5,776,983, 488,064, 5,705,515, 5,451,677, WO94/18161, WO95/29159, WO97/46556, WO98/04526 and WO98/32753, WO01/74782, WO02/32897, WO03/014113, WO03/016276, WO03/016307, WO03/024948, WO03/024953 and WO03/037881; noradrenergic agents including, but not limited to, diethylpropion (such as Tenuate® (1-propanone, 2-(diethylamino)-1-phenyl-, hydrochloride), Merrell), dextroamphetamine (also known as dextroamphetamine sulfate, dexamphetamine, dexedrine, Dexampex, Ferndex, Oxydess II, Robese, Spancap #1), mazindol ((or 5-(p-chlorophenyl)-2,5-dihydro-3H-imidazo[2,1-a]isoindol-5-ol) such as Sanorex®, Novartis or Mazanor®, Wyeth Ayerst), phenylpropanolamine (or Benzenemethanol, alpha-(1-aminoethyl)-, hydrochloride), phentermine ((or Phenol, 3-[[4,5-duhydro-1H-imidazol-2-yl)ethyl](4-methylpheny-1)amino], monohydrochloride) such as Adipex-P®, Lemmon, FASTIN®, Smith-Kline Beecham and Ionamin®, Medeva), phendimetrazine ((or (2S,3S)-3,4-Dimethyl-2phenylmorpholine L-(+)-tartrate (1:1)) such as Metra® (Forest), Plegine® (Wyeth-Ayerst), Prelu-2® (Boehringer Ingelheim), and Statobex® (Lemmon), phendamine tartrate (such as Thephorin® (2,3,4,9-Tetrahydro-2-methyl-9-phenyl-1H-indenol[2,1-c]pyridine L-(+)-tartrate (1:1)), Hoffmann-LaRoche), methamphetamine (such as Desoxyn®, Abbot ((S)-N, (alpha)-dimethylbenzeneethanamine hydrochloride)), and phendimetrazine tartrate (such as Bontril® Slow-Release Capsules, Amarin (-3,4-Dimethyl-2-phenylmorpholine Tartrate); fatty acid oxidation upregulator/inducers such as Famoxin® (Genset); monamine oxidase inhibitors including but not limited to befloxatone, moclobemide, brofaromine, phenoxathine, esuprone, befol, toloxatone, pirlindol, amiflamine, sercloremine, bazinaprine, lazabemide, milacemide, caroxazone and other certain compounds as disclosed by WO01/12176; and other anti-obesity agents such as 5HT-2 agonists, ACC (acetyl-CoA carboxylase) inhibitors such as those described in WO03/072197, alpha-lipoic acid (alpha-LA), AOD9604, appetite suppressants such as those in WO03/40107, ATL-962 (Alizyme PLC), benzocaine, benzphetamine hydrochloride (Didrex), bladderwrack (focus vesiculosus), BRS3 (bombesin receptor subtype 3) agonists, bupropion, caffeine, CCK agonists, chitosan, chromium, conjugated linoleic acid, corticotropin-releasing hormone agonists, dehydroepiandrosterone, DGAT1 (diacylglycerol acyltransferase 1) inhibitors, DGAT2 (diacylglycerol acyltransferase 2) inhibitors, dicarboxylate transporter inhibitors, ephedra, exendin-4 (an inhibitor of glp-1) FAS (fatty acid synthase) inhibitors (such as Cerulenin and C75), fat resorption inhibitors (such as those in WO03/053451, and the like), fatty acid transporter inhibitors, natural water soluble fibers (such as psyllium, plantago, guar, oat, pectin), galanin antagonists, galega (Goat's Rue, French Lilac), garcinia cambogia, germander (teucrium chamaedrys), ghrelin antibodies and ghrelin antagonists (such as those disclosed in WO01/87335, and WO02/08250), polypeptide hormones and variants thereof which affect the islet cell secretion, such as the hormones of the secretin/gastric inhibitory polypeptide (GIP)/vasoactive intestinal polypeptide (VIP)/pituitary adenylate cyclase activating polypeptide (PACAP)/glucagon-like polypeptide II (GLP-II)/glicentin/glucagon gene family and/or those of the adrenomedullin/amylin/calcitonin gene related polypeptide (CGRP) gene family includingGLP-1 (glucagon-like polypeptide 1) agonists (e.g. (1) exendin-4, (2) those GLP-I molecules described in US20050130891 including GLP-1(7-34), GLP-1(7-35), GLP-1(7-36) or GLP-1(7-37) in its C-terminally carboxylated or amidated form or as modified GLP-I polypeptides and modifications thereof including those described in paragraphs 17-44 of US20050130891, and derivatives derived from GLP-1-(7-34)COOH and the corresponding acid amide are employed which have the following general formula: R-NH-HAEGTFTSDVSYLEGQAAKEFIAWLVK-CONH₂ wherein R═H or an organic compound having from 1 to 10 carbon atoms. Preferably, R is the residue of a carboxylic acid. Particularly preferred are the following carboxylic acid residues: formyl, acetyl, propionyl, isopropionyl, methyl, ethyl, propyl, isopropyl, n-butyl, sec-butyl, tert-butyl.) and glp-1 (glucagon-like polypeptide-1), glucocorticoid antagonists, glucose transporter inhibitors, growth hormone secretagogues (such as those disclosed and specifically described in U.S. Pat. No. 5,536,716), interleukin-6 (IL-6) and modulators thereof (as in WO03/057237, and the like), L-carnitine, Mc3r (melanocortin 3 receptor) agonists, MCH2R (melanin concentrating hormone 2R) agonist/antagonists, melanin concentrating hormone antagonists, melanocortin agonists (such as Melanotan II or those described in WO 99/64002 and WO 00/74679), nomame herba, phosphate transporter inhibitors, phytopharm compound 57 (CP 644,673), pyruvate, SCD-I (stearoyl-CoA desaturase-1) inhibitors, T71 (Tularik, Inc., Boulder Colo.), Topiramate (Topimax®, indicated as an anti-convulsant which has been shown to increase weight loss), transcription factor modulators (such as those disclosed in WO03/026576), β-hydroxy steroid dehydrogenase-1 inhibitors (β-HSD-I), β-hydroxy-β-methylbutyrate, p57 (Pfizer), Zonisamide (Zonegran™, indicated as an anti-epileptic which has been shown to lead to weight loss), and the agents disclosed in US20030119428 paragraphs 20-26.

### Anti-Diabetic Agents

Patients with diabetic nephropathy may have higher NF-κB activity. Thus, to the GCRA peptides for inhibiting NF-κB activation described herein can be used in therapeutic combination with one or more anti-diabetic agents, including but not limited to: PPARγ agonists such as glitazones (e.g., WAY-120,744, AD 5075, balaglitazone, ciglitazone, darglitazone (CP-86325, Pfizer), englitazone (CP-68722, Pfizer), isaglitazone (MIT/J&J), MCC-555 (Mitsibishi disclosed in U.S. Pat. No. 5,594,016), pioglitazone (such as such as Actos™ pioglitazone; Takeda), rosiglitazone (Avandia™; Smith Kline Beecham), rosiglitazone maleate, troglitazone (Rezulin®, disclosed in U.S. Pat. No. 4,572,912), rivoglitazone (CS-Ol 1, Sankyo), GL-262570 (Glaxo Welcome), BRL49653 (disclosed in WO98/05331), CLX-0921, 5-BTZD, GW-0207, LG-100641, JJT-501 (JPNT/P&U), L-895645 (Merck), R-119702 (Sankyo/Pfizer), NN-2344 (Dr. Reddy/NN), YM-440 (Yamanouchi), LY-300512, LY-519818, R483 (Roche), T131 (Tularik), and the like and compounds disclosed in U.S. Pat. Nos. 4,687,777, 5,002,953, 5,741,803, 5,965,584, 6,150,383, 6,150,384, 6,166,042, 6,166,043, 6,172,090, 6,211,205, 6,271,243, 6,288,095, 6,303,640, 6,329,404, 5,994,554, WO97/10813, WO97/27857, WO97/28115, WO97/28137,WO97/27847, WO00/76488, WO03/000685,WO03/027112, WO03/035602, WO03/048130, WO03/055867, and pharmaceutically acceptable salts thereof; biguanides such as metformin hydrochloride (N,N-dimethylimidodicarbonimidic diamide hydrochloride, such as Glucophage™, Bristol-Myers Squibb); metformin hydrochloride with glyburide, such as Glucovance™, Bristol-Myers Squibb); buformin (Imidodicarbonimidic diamide, N-butyl-); etoformine (1-Butyl-2-ethylbiguanide, Schering A. G.); other metformin salt forms (including where the salt is chosen from the group of, acetate, benzoate, citrate, ftimarate, embonate, chlorophenoxyacetate, glycolate, palmoate, aspartate, methanesulphonate, maleate, parachlorophenoxyisobutyrate, formate, lactate, succinate, sulphate, tartrate, cyclohexanecarboxylate, hexanoate, octanoate, decanoate, hexadecanoate, octodecanoate, benzenesulphonate, trimethoxybenzoate, paratoluenesulphonate, adamantanecarboxylate, glycoxylate, glutamate, pyrrolidonecarboxylate, naphthalenesulphonate, 1-glucosephosphate, nitrate, sulphite, dithionate and phosphate), and phenformin; protein tyrosine phosphatase-IB (PTP-IB) inhibitors, such as A-401,674, KR 61639, OC-060062, OC-83839, OC-297962, MC52445, MC52453, ISIS 113715, and those disclosed in WO99/585521, WO99/58518, WO99/58522, WO99/61435, WO03/032916, WO03/032982, WO03/041729, WO03/055883, WO02/26707, WO02/26743, JP2002114768, and pharmaceutically acceptable salts and esters thereof; sulfonylureas such as acetohexamide (e.g. Dymelor, Eli Lilly), carbutamide, chlorpropamide (e.g. Diabinese®, Pfizer), gliamilide (Pfizer), gliclazide (e.g. Diamcron, Servier Canada Inc), glimepiride (e.g. disclosed in U.S. Pat. No. 4,379,785, such as Amaryl, Aventis), glipentide, glipizide (e.g. Glucotrol or Glucotrol XL Extended Release, Pfizer), gliquidone, glisolamide, glyburide/glibenclamide (e.g. Micronase or Glynase Prestab, Pharmacia & Upjohn and Diabeta, Aventis), tolazamide (e.g. Tolinase), and tolbutamide (e.g. Orinase), and pharmaceutically acceptable salts and esters thereof; meglitinides such as repaglinide (e.g. Pranidin®, Novo Nordisk), KAD1229 (PF/Kissei), and nateglinide (e.g. Starlix®, Novartis), and pharmaceutically acceptable salts and esters thereof; a glucoside hydrolase inhibitors (or glucoside inhibitors) such as acarbose (e.g. Precose™, Bayer disclosed in U.S. Pat. No. 4,904,769), miglitol (such as GLYSET™, Pharmacia & Upjohn disclosed in U.S. Pat. No. 4,639,436), camiglibose (Methyl 6-deoxy-6-[(2R,3R,4R,5S)-3,4,5-trihydroxy-2-(hydroxymethyl)piperidino]-alpha-D-glucopyranoside, Marion Merrell Dow), voglibose (Takeda), adiposine, emiglitate, pradimicin-Q, salbostatin, CKD-711, MDL-25,637, MDL-73,945, and MOR 14, and the compounds disclosed in U.S. Pat. Nos. 4,062,950, 4,174,439, 4,254,256, 4,701,559, 4,639,436, 5,192,772, 4,634,765, 5,157,116, 5,504,078, 5,091,418, 5,217,877 and WO01/47528 (polyamines); α-amylase inhibitors such as tendamistat, trestatin, and Al-3688, and the compounds disclosed in U.S. Pat. Nos. 4,451,455, 4,623,714, and 4,273,765; SGLT2 inhibitors including those disclosed in U.S. Pat. Nos. 6,414,126 and 6,515,117; an aP2 inhibitor such as disclosed in U.S. Pat. No. 6,548,529; insulin secreatagogues such as linogliride, A-4166, forskilin, dibutyrl cAMP, isobutylmethylxanthine (IBMX), and pharmaceutically acceptable salts and esters thereof; fatty acid oxidation inhibitors, such as clomoxir, and etomoxir, and pharmaceutically acceptable salts and esters thereof; A2 antagonists, such as midaglizole, isaglidole, deriglidole, idazoxan, earoxan, and fluparoxan, and pharmaceutically acceptable salts and esters thereof; insulin and related compounds (e.g. insulin mimetics) such as biota, LP-100, novarapid, insulin detemir, insulin lispro, insulin glargine, insulin zinc suspension (lente and ultralente), Lys-Pro insulin, GLP-I (1-36) amide, GLP-I (73-7) (insulintropin, disclosed in U.S. Pat. No. 5,614,492), LY-315902 (Lilly), GLP-I (7-36)-NH2), AL-401 (Autoimmune), certain compositions as disclosed in U.S. Pat Nos. 4,579,730, 4,849,405, 4,963,526, 5,642,868, 5,763,396, 5,824,638, 5,843,866, 6,153,632, 6,191,105, and WO 85/05029, and primate, rodent, or rabbit insulin including biologically active variants thereof including allelic variants, more preferably human insulin available in recombinant form (sources of human insulin include pharmaceutically acceptable and sterile formulations such as those available from Eli Lilly (Indianapolis, Ind. 46285) as Humulin™ (human insulin rDNA origin), also see the THE PHYSICIAN′S DESK REFERENCE, 55.sup.th Ed. (2001) Medical Economics, Thomson Healthcare (disclosing other suitable human insulins); non-thiazolidinediones such as JT-501 and farglitazar (GW-2570/GI-262579), and pharmaceutically acceptable salts and esters thereof; PPARα/γ dual agonists such as AR-HO39242 (Aztrazeneca), GW-409544 (Glaxo-Wellcome), BVT-142, CLX-0940, GW-1536, GW-1929, GW-2433, KRP-297 (Kyorin Merck; 5-[(2,4-Dioxo thiazolidinyl)methyl] methoxy-N-[[4-(trifluoromethyl)phenyl] methyljbenzamide), L-796449, LR-90, MK-0767 (Merck/Kyorin/Banyu), SB 219994, muraglitazar (BMS), tesaglitzar (Astrazeneca), reglitazar (JTT-501) and those disclosed in WO99/16758, WO99/19313, WO99/20614, WO99/38850, WO00/23415, WO00/23417, WO00/23445, WO00/50414, WO01/00579, WO01/79150, WO02/062799, WO03/004458, WO03/016265, WO03/018010, WO03/033481, WO03/033450, WO03/033453, WO03/043985, WO 031053976, U.S. application Ser. No. 09/664,598, filed Sep. 18, 2000, Murakami et al. Diabetes 47, 1841-1847 (1998), and pharmaceutically acceptable salts and esters thereof; other insulin sensitizing drugs; VPAC2 receptor agonists; GLK modulators, such as those disclosed in WO03/015774; retinoid modulators such as those disclosed in WO03/000249; GSK 3β/GSK 3 inhibitors such as 4-[2-(2-bromophenyl)-4-(4-fluorophenyl-1H-imidazol-5-yl]pyridine and those compounds disclosed in WO03/024447, WO03/037869, WO03/037877, WO03/037891, WO03/068773, EP1295884, EP1295885, and the like; glycogen phosphorylase (HGLPa) inhibitors such as CP-368,296, CP-316,819, BAYR3401, and compounds disclosed in WO01/94300, WO02/20530, WO03/037864, and pharmaceutically acceptable salts or esters thereof; ATP consumption promotors such as those disclosed in WO03/007990; TRB3 inhibitors; vanilloid receptor ligands such as those disclosed in WO03/049702; hypoglycemic agents such as those disclosed in WO03/015781 and WO03/040114; glycogen synthase kinase 3 inhibitors such as those disclosed in WO03/035663 agents such as those disclosed in WO99/51225, US20030134890, WO01/24786, and WO03/059870; insulin-responsive DNA binding protein-1 (IRDBP-I) as disclosed in WO03/057827, and the like; adenosine A2 antagonists such as those disclosed in WO03/035639, WO03/035640, and the like; PPARδ agonists such as GW 501516, GW 590735, and compounds disclosed in JP10237049 and WO02/14291; dipeptidyl peptidase IV (DP-IV) inhibitors, such as isoleucine thiazolidide, NVP-DPP728A (1-[[[2-[(5-cyanopyridin-2-yl)amino]ethyl]amino]acetyl]-2-cyano-(S)-pyrrolidine, disclosed by Hughes et al, Biochemistry, 38(36), 11597-11603, 1999), P32/98, NVP-LAF-237, P3298, TSL225 (tryptophyl-1,2,3,4-tetrahydro-isoquinoline-3-carboxylic acid, disclosed by Yamada et al, Bioorg. & Med. Chem. Lett. 8 (1998) 1537-1540), valine pyrrolidide, TMC-2A/2B/2C, CD-26 inhibitors, FE999011, P9310/1(364, VIP 0177, DPP4, SDZ 274-444, 2-cyanopyrrolidides and 4-cyanopyrrolidides as disclosed by Ashworth et al, Bioorg. & Med. Chem. Lett., Vol. 6, No. 22, pp 1163-1166 and 2745-2748 (1996), and the compounds disclosed in U.S. Pat. Nos. 6,395,767, 6,573,287, 6,395,767 (compounds disclosed include BMS-477118, BMS-471211 and BMS 538,305), WO99/38501, WO99/46272, WO99/67279, WO99/67278, WO99/61431WO03/004498, WO03/004496, EP1258476, WO02/083128, WO02/062764, WO03/000250, WO03/002530, WO03/002531, WO03/002553, WO03/002593, WO03/000180, and WO03/000181; GLP-I agonists such as exendin-3 and exendin-4 (including the 39 aa polypeptide synthetic exendin-4 called Exenatide®), and compounds disclosed in US2003087821 and NZ 504256, and pharmaceutically acceptable salts and esters thereof; peptides including amlintide and Symlin® (pramlintide acetate); and glycokinase activators such as those disclosed in US2002103199 (fused heteroaromatic compounds) and WO02/48106 (isoindolin-1-one-substituted propionamide compounds).

### Phosphodiesterase Inhibitors

The GCRA peptides described herein can be used in combination therapy with a phosphodiesterase inhibitor. PDE inhibitors are those compounds which slow the degradation of cyclic AMP (cAMP) and/or cyclic GMP (cGMP) by inhibition of the phosphodiesterases, which can lead to a relative increase in the intracellular concentration of c AMP and/or cGMP. Possible PDE inhibitors are primarily those substances which are to be numbered among the class consisting of the PDE3 inhibitors, the class consisting of the PDE4 inhibitors and/or the class consisting of the PDES inhibitors, in particular those substances which can be designated as mixed types of PDE3/4 inhibitors or as mixed types of PDE3/4/5 inhibitors. By way of example, those PDE inhibitors may be mentioned such as are described and/or claimed in the following patent applications and patents: DE1470341, DE2108438, DE2123328, DE2305339, DE2305575, DE2315801, DE2402908, DE2413935, DE2451417, DE2459090, DE2646469, DE2727481, DE2825048, DE2837161, DE2845220, DE2847621, DE2934747, DE3021792, DE3038166, DE3044568, EP000718, EP0008408, EP0010759, EP0059948, EP0075436, EP0096517, EPOl 12987, EPOl 16948, EP0150937, EP0158380, EP0161632, EP0161918, EP0167121, EP0199127, EP0220044, EP0247725, EP0258191, EP0272910, EP0272914, EP0294647, EP0300726, EP0335386, EP0357788, EP0389282, EP0406958, EP0426180, EP0428302, EP0435811, EP0470805, EP0482208, EP0490823, EP0506194, EP0511865, EP0527117, EP0626939, EP0664289, EP0671389, EP0685474, EP0685475, EP0685479, JP92234389, JP94329652, JP95010875, U.S. Pat. Nos. 4,963,561, 5,141,931, WO9117991, WO9200968, WO9212961, WO9307146, WO9315044, WO9315045, WO9318024, WO9319068, WO9319720, WO9319747, WO9319749, WO9319751, WO9325517, WO9402465, WO9406423, WO9412461, WO9420455, WO9422852, WO9425437, WO9427947, WO9500516, WO9501980, WO9503794, WO9504045, WO9504046, WO9505386, WO9508534, WO9509623, WO9509624, WO9509627, WO9509836, WO9514667, WO9514680, WO9514681, WO9517392, WO9517399, WO9519362, WO9522520, WO9524381, WO9527692, WO9528926, WO9535281, WO9535282, WO9600218, WO9601825, WO9602541, WO9611917, DE3142982, DEl 116676, DE2162096, EP0293063, EP0463756, EP0482208, EP0579496, EP0667345 U.S. Pat. No. 6,331,543, US20050004222 (including those disclosed in formulas I-XIII and paragraphs 37-39, 85-0545 and 557-577) and WO9307124, EP0163965, EP0393500, EP0510562, EP0553174, WO9501338 and WO9603399. PDE5 inhibitors which may be mentioned by way of example are RX-RA-69, SCH-51866, KT-734, vesnarinone, zaprinast, SKF-96231, ER-21355, BF/GP-385, NM-702 and sildenafil (Viagra®). PDE4 inhibitors which may be mentioned by way of example are RO-20-1724, MEM 1414 (R1533/R1500; Pharmacia Roche), DENBUFYLLINE, ROLIPRAM, OXAGRELATE, NITRAQUAZONE, Y-590, DH-6471, SKF-94120, MOTAPIZONE, LIXAZINONE, INDOLIDAN, OLPRINONE, ATIZORAM, KS-506-G, DIPAMFYLLINE, BMY-43351, ATIZORAM, AROFYLLINE, FILAMINAST, PDB-093, UCB-29646, CDP-840, SKF-107806, PICLAMILAST, RS-17597, RS-25344-000, SB-207499, TIBENELAST, SB-210667, SB-211572, SB-211600, SB-212066, SB-212179, GW-3600, CDP-840, MOPIDAMOL, ANAGRELIDE, IBUDILAST, AMRINONE, PIMOBENDAN, CILOSTAZOL, QUAZINONE and N-(3,5-dichloropyrid-4-yl)-3-cyclopropylmethoxy4-difluoromethoxybenzamide. PDE3 inhibitors which may be mentioned by way of example are SULMAZOLE, AMPIZONE, CILOSTAMIDE, CARBAZERAN, PIROXIMONE, IMAZODAN, CI-930, SIGUAZODAN, ADIBENDAN, SATERINONE, SKF-95654, SDZ-MKS-492, 349-U-85, EMORADAN, EMD-53998, EMD-57033, NSP-306, NSP-307, REVIZINONE, NM-702, WIN-62582 and WIN-63291, ENOXIMONE and MILRINONE. PDE3/4 inhibitors which may be mentioned by way of example are BENAFENTRINE, TREQUINSIN, ORG-30029, ZARDAVERINE, L-686398, SDZ-ISQ-844, ORG-20241, EMD-54622, and TOLAFENTRINE. Other PDE inhibitors include: cilomilast, pentoxifylline, roflumilast, tadalafil (Cialis®), theophylline, and vardenafil(Levitra®), zaprinast (PDE5 specific).

### Anti-Uterine Contractions Agents

The GCRA peptides described herein can be used in combination therapy (for example, in order to decrease or inhibit uterine contractions) with a tocolytic agent including but not limited to beta-adrenergic agents, magnesium sulfate, prostaglandin inhibitors, and calcium channel blockers.

### Anti-Neoplastic Agents

The GCRA peptides described herein can be used in combination therapy with an antineoplastic agents including but not limited to alkylating agents, epipodophyllotoxins, nitrosoureas, antimetabolites, vinca alkaloids, anthracycline antibiotics, nitrogen mustard agents, and the like. Particular anti-neoplastic agents may include tamoxifen, taxol, etoposide and 5-fluorouracil.

The GCRA peptides described herein can be used in combination therapy (for example as in a chemotherapeutic composition) with an antiviral and monoclonal antibody therapies.

### Agents to Treat Congestive Heart Failure

The GCRA peptides described herein can be used in combination therapy (for example, in prevention/treatment of congestive heart failure or another method described herein) with the partial agonist of the nociceptin receptor ORL1 described by Dooley et al. (The Journal of Pharmacology and Experimental Therapeutics, 283 (2): 735-741, 1997). The agonist is a hexapeptide having the amino acid sequence Ac-RYY (RK) (WI) (RK)-NH2 (“the Dooley polypeptide”), where the brackets show allowable variation of amino acid residue. Thus Dooley polypeptide can include but are not limited to KYYRWR, RYYRWR, KWRYYR, RYYRWK, RYYRWK (all-D amin acids), RYYRIK, RYYRIR, RYYKIK, RYYKIR, RYYKWR, RYYKWK, RYYRWR, RYYRWK, RYYRIK, RYYKWR, RYYKWK, RYYRWK and KYYRWK, wherein the amino acid residues are in the L-form unless otherwise specified. The GCRA peptides described herein can also be used in combination therapy with polypeptide conjugate modifications of the Dooley polypeptide described in WO0198324.

### Fibrate

The GCRA peptides described herein can be used in combination therapy with a fibrate. The term “fibrate” is also interchangeably used herein and in the art with the term “fibric acid derivative,” and means any of the fibric acid derivatives useful in the methods described herein, e.g., fenofibrate. Fenofibrate is a fibrate compound, other examples of which include, for example, bezafibrate, beclofibrate, benzafibrate, binifibrate, ciprofibrate, clinofibrate, clofibrate, etofibrate, gemcabene, gemfibrozil, lifibrol, nicofibrate, pirifibrate, ronifibrate, simfibrate, theofibrate, etc.

### Lipid Altering Agents

NF-κB has a coordinating role in inflammation and cellular proliferation and may be involved in early atherosclerosis. The GCRA peptides for inhibiting NF-κB activation described herein can be used in combination therapy with a lipid altering agent. As used herein the term “lipid altering agent” or “dyslipidemia agent” refers to compounds including, but not limited to, bile acid sequestrants such as cholestyramine (a styrene-divinylbenzene copolymer containing quaternary ammonium cationic groups capable of binding bile acids, such as QUESTRAN® or QUESTRAN LIGHT® cholestyramine which are available from Bristol-Myers Squibb), colesevelam hydrochloride (such as WELCHOL® Tablets (polyallylamine hydrochloride) cross-linked with epichlorohydrin and alkylated with 1-bromodecane and (6-bromohexyl)-trimethylammonium bromide) which are available from Sankyo), colestipol (a copolymer of diethylenetriamine and 1-chloro-2,3-epoxypropane, such as COLESTID® tablets which are available from Pharmacia), dialkylaminoalkyl derivatives of a cross-linked dextran, LOCHOLEST®, DEAE-Sephadex (SECHOLEX®, POLICEXIDE®), water soluble derivatives such as 3,3-ioene, N-(cycloalkyl)alkylamines and poliglusam, insoluble quaternized polystyrenes, saponins and mixtures thereof and those bile acid sequestrants disclosed in WO97/11345, WO98/57652, U.S. Pat. Nos. 3,692,895, and 5,703,188. Suitable inorganic cholesterol sequestrants include bismuth salicylate plus montmorillonite clay, aluminum hydroxide and calcium carbonate antacids.

### HMG-CoA Reductase Inhibitors

The 3-hydroxy-3-methylglutaryl coenzyme A (HMGCoA) reductase inhibitors, commonly referred to as statins, are potent inhibitors of cholesterol biosynthesis and widely prescribed for the treatment of hypercholesterolemia. For example, Pitavastatin is highly potent inhibitor of HMG-CoA reductase, the rate-limiting enzyme in cholesterol biosynthesis. Pitavastatin shows protective action on vascular endothelial cells (ECs) by inducing the activation of eNOS, thereby protecting the vascular ECs from injury due to the inflammatory reaction induced by TNF-ϵ, and increasing NO production, which is dependent on post-transcriptional regulation, and involves phosphoinositide 3-kinase and the Akt pathway. Pitavastatin also inhibits NF-kB activation and IL-6 protein production induced by TNF-α in hepatocarcinoma cells.

The GCRA peptides for inhibiting NF-κB activation described herein can be used in combination therapy with a HMG-CoA reductase inhibitor. HMG-CoA reductase inhibitors are dyslipidemic agents that can be used in therapeutic combinations with compounds described herein. Suitable HMG-CoA reductase inhibitors for use in therapeutic combination with a compounds described herein include: atorvastatin (LIPITOR®; disclosed in U.S. Pat. Nos. 4,681,893, 5,385,929 and 5,686,104), atorvastatin calcium (disclosed in U.S. Pat. No. 5,273,995), dihydrocompactin, (disclosed in U.S. Pat. No. 4,450,171), bervastatin (disclosed in U.S. Pat. No. 5,082,859), carvastatin, cerivastatin (BAYCOL®; disclosed in U.S. Pat. Nos. 5,006,530, 5,502,199, and 5,177,080), crilvastatin, dalvastatin (disclosed in EP738510A2), fluvastatin (LESCOL®; disclosed in U.S. Pat. Nos. 4,739,073 and 534,772), glenvastatin, fluindostatin (disclosed in EP363934A1), velostatin (visinolin; disclosed in U.S. Pat. Nos. 4,448,784 and 4,450,171), lovastatin (mevinolin; MEVACOR® (Merck and Co.) and related compounds disclosed in U.S. Pat. No. 4,231,938), mevastatin (and related compound disclosed in U.S. Pat. No. 3,983,140), compactin (and related compounds disclosed in U.S. Pat. No. 4,804,770), pravastatin (also known as NK-104, itavastatin, nisvastatin, nisbastatin disclosed in U.S. Pat. No. 5,102,888), pravastatin (PRAVACHOL® (Bristol Myers Squibb) and related compounds disclosed in U.S. Pat. No. 4,346,227), rivastatin (sodium 7-(4-fluorophenyl)-2,6-diisopropyl-5-methoxymethylpyridin-3-yl)-3,5-dihydroxy-6-heptanoate), rosuvastatin (CRESTOR®; also known as ZD-4522 disclosed in U.S. Pat. No. 5,260,440), atavastatin, visastatin, simvastatin (ZOCOR® (Merck and Co.) and related compounds as disclosed in U.S. Pat. Nos. 4,448,784 and 4,450,171), simvastatin, CI-981, compounds disclosed in WO03/033481, U.S. Pat. Nos. 4,231,938, 4,444,784, 4,647,576, 4,686,237, 4,499,289, 4,346,227, 5,753,675, 4,613,610, EP0221025, and EP491226, and optical or geometric isomers thereof and nontoxic pharmaceutically acceptable salts, N-oxides, esters, quaternary ammonium salts, and prodrugs thereof. In HMG-CoA reductase inhibitors where an open-acid form can exist, salt and ester forms may preferably be formed from the open-acid, and all such forms are included within the meaning of the term “HMG-CoA reductase inhibitor” as used herein. Pharmaceutically acceptable salts with respect to the HMG-CoA reductase inhibitor includes non-toxic salts of the compounds which are generally prepared by reacting the free acid with a suitable organic or inorganic base, particularly those formed from cations such as sodium, potassium, aluminum, calcium, lithium, magnesium, zinc and tetramethylammonium, as well as those salts formed from amines such as ammonia, ethylenediamine, N-methylglucamine, lysine, arginine, ornithine, choline, N,N′-dibenzyl ethyl enediamine, chloroprocaine, diethanolamine, procaine, N-b enzylphenethylamine, 1-p-chlorobenzyl-2-pyrrolidine-1′-yl-methylbenzim-idazole, diethylamine, piperazine, and tris(hydroxymethyl) aminomethane. Further examples of salt forms of HMG-CoA reductase inhibitors may include, but are not limited to, acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynapthoate, iodide, isothionate, lactate, lactobionate, laurate, malate, maleate, mandelate, mesylate, methylsulfate, mucate, napsylate, nitrate, oleate, oxalate, pamaote, palmitate, pantothenate, phosphate/diphosphate, polygalacturonate, salicylate, stearate, subacetate, succinate, tannate, tartrate, teoclate, tosylate, triethiodide, and valerate.

Other dyslipidemic agents which can be used in therapeutic combination with a compound described herein include: HMG-CoA synthase inhibitors such as L-659,699 ((E E)-I1-[3′R-(hydroxy-methyl)-4′-oxo-2′R-oxetanyl]-3,5,7R-trimethyl-2,4-undecadienoic acid) and those disclosed in U.S. Pat Nos. 5,120,729, 5,064,856, and 4,847,271; cholesterol absorption inhibitors such as plant sterols, plant stanols and/or fatty acid estesrs of plant stanols such as sitostanol ester used in BENECOL® margarine, stanol esters, beta-sitosterol, and sterol glycosides such as tiqueside. Other cholesterol absorption inhibitors include 1,4-Diphenylazetidin-2-ones; 4-biarylyl-1-phenylazetidin-2-ones; 4-(hydroxyphenyl)azetidin-2-ones; 1,4-diphenyl-3-hydroxyalkyl-2-azetidinones; 4-biphenyl-1-phenylazetidin-2-ones; 4-biarylyl-1-phenylazetidin-2-ones; and 4-biphenylylazetidinones.acyl coenzyme A-cholesterol acyl transferase (ACAT) inhibitors such as avasimibe (Current Opinion in Investigational Drugs. 3(9):291-297 (2003)), eflucimibe, HL-004, lecimibe, DuP-128, KY505, SMP 797, CL-277,082 (Clin Pharmacol Ther. 48(2): 189-94 (1990)) and the like; and those disclosed in U.S. Pat. No. 5,510,379, WO96/26948 and WO96/10559; CETP inhibitors such as JTT 705 identified as in Nature 406, (6792):203-7 (2000), torcetrapib (CP-529,414 described in US20030186952 and WO00/017164), CP 532,632, BAY63-2149, SC 591, SC 795, and the like including those described in Current Opinion in Investigational Drugs. 4(3):291-297 (2003) and those disclosed in J. Antibiot, 49(8): 815-816 (1996), and Bioorg. Med. Chem. Lett, 6:1951-1954 (1996) and patent publications U.S. Pat. Nos. 5,512,548, 6,147,090, WO99/20302, WO99/14204, WO99/41237, WO95/04755, WO96/15141, WO96/05227,WO038721, EP796846, EP818197, EP818448, DE19704244, DE19741051,DE19741399, DE197042437, DE19709125, DE19627430, DE19832159,DE19741400, JP 11049743, and JP 09059155; squalene synthetase inhibitors such as squalestatin-1, TAK-475, and those disclosed in U.S. Pat. Nos. 4,871,721, 4,924,024, 5,712,396 (α-phosphono-sulfonates), Biller et al (1988) J. Med. Chem., 31:1869 (e.g. isoprenoid (phosphinyl-methyl)phosphonates), Biller et al (1996) Current Pharmaceutical Design, 2:1, P. Ortiz de Montellano et al (1977) J. Med. Chem. 20:243 (terpenoid pyrophosphates), Corey and Volante (1976) J. Am. Chem. Soc, 98:1291 (farnesyl diphosphate analog A and presqualene pyrophosphate (PSQ-PP) analogs), McClard et al (1987) J.A.C.S., 109:5544 (phosphinylphosphonates), Capson, T. L., PhD dissertation, June, 1987, Dept. Med. Chem. U of Utah, Abstract, Table of Contents, pp 16, 17, 40-43, 48-51, Summary, (cyclopropanes), Curr. Op. Ther. Patents (1993) 861, and patent publications EP0567026A1, EP0645378A1, EP0645377A1, EP0611749A1, EP0705607A2, EP0701725A1, and WO96/09827; antioxidants such as probucol (and related compounds disclosed in U.S. Pat. Nos. 3,674,836), probucol derivatives such as AGI-1067 (and other derivatives disclosed in U.S. Pat. Nos. 6,121,319 and 6,147,250), tocopherol, ascorbic acid, β-carotene, selenium and vitamins such as vitamin B6 or vitamin B12 and pharmaceutically acceptable salts and esters thereof; PPARα agonists such as those disclosed in U.S. Pat. No. 6,028,109 (fluorophenyl compounds), WO00/75103 (substituted phenylpropionic compounds), WO98/43081 and fibric acid derivatives (fibrates) such as beclofibrate, benzafibrate, bezafibrate (C.A.S. Registry No. 41859-67-0, see U.S. Pat. No. 3,781,328), binifibrate (C.A.S. Registry No. 69047-39-8, see BE884722), ciprofibrate (C.A.S. Registry No. 52214-84-3, see U.S. Pat. No. 3,948,973), clinofibrate (C.A.S. Registry No. 30299-08-2, see U.S. Pat. No. 3,716,583), clofibrate (such as ethyl 2-(p-chlorophenoxy)-2-methyl-propionate, e.g. Atromid-S® capsules (Wyeth-Ayerst), etofibrate, fenofibrate (such as Tricor® micronized fenofibrate ((2-[4-(4-chlorobenzoyl)phenoxy]-2-methyl-propanoic acid, 1-methylethyl ester; Abbott Laboratories) or Lipanthyl® micronized fenofibrate (Labortoire Founier, France)), gemcabene, gemfibrozil (such as 5-(2,5-dimethylphenoxy)-2,2-dimethylpentanoic acid, e.g. Lopid® tablets (Parke Davis)), lifibrol, GW 7647, BM 170744, LYS 18674 and those fibrate and fibrate acid derivatives disclosed in WO03/033456, WO03/033481, WO03/043997, WO03/048116, WO03/053974, WO03/059864, and WO03/05875; FXR receptor modulators such as GW 4064, SR 103912, and the like; LXR receptor modulators such as GW 3965, T9013137, and XTC0179628, and those disclosed in US20030125357, WO03/045382, WO03/053352, WO03/059874, and the like; HM74 and HM74A (human HM74A is Genbank Accession No. AY148884 and rat HM74A is EMM_patAR09 8624) receptor agonists such as nicotinic acid (niacin) and derivatives thereof (e.g. compounds comprising a pyridine-3-carboxylate structure or a pyrazine-2-carboxylate structure, including acid forms, salts, esters, zwitterions and tautomers, where available) including but not limited to those disclosed in Wise et al (2003) J. Biol. Chem. 278: 9869 (e.g. 5-methylpyrazole-3-carboxylic acid and acifran (4,5-dihydro-5-methyl-4-oxo-5-phenyl-2-furan carboxylic acid pyradine-3-acetic acid)), as well as 5-methyl nicotinic acid, nicotinuric acid, niceritrol, nicofuranose, acipimox (5-methylpyrazine-2-carboxylic acid 4-oxide), Niaspan® (niacin extended-release tablets; Kos) and those which can be easily identified by one skilled in the art which bind to and agonize the HM74A or HM74 receptor (for example using the assays disclosed in Wise et al (2003) J. Biol. Chem 278:9869 (nicotine binding and [35S]-GTPyS binding assays), Soga et al (2003) Biochem. Biophys. Res. Comm. 303:364 (radiolabel binding assay using the HM74 receptor which could be adapted to the HM74A receptor), Tunaru et al (2003) Nature Medicine 9:352 (calcium mobilization assay using the HM74 receptor which could be adapted to the HM74A receptor) and U.S. Pat. No. 6,420,183 (FLIPR assays are described generally in and may be adapted to the HM74A or HM74 receptor); renin angiotensin system inhibitors; bile acid reabsorption inhibitors (bile acid reuptake inhibitors), such as BARI 1453, SC435, PHA384640, S8921, AZD7706, and the like; PPARδ agonists (including partial agonists) such as GW 501516, and GW 590735, and those disclosed in U.S. Pat. No. 5,859,051 (acetophenols), WO03/024395, WO97/28149, WO01/79197, WO02/14291, WO02/46154, WO02/46176, WO02/076957, WO03/0 16291, WO03/033493, WO99/20275 (quinoline phenyl compounds), WO99/38845 (aryl compounds), WO00/63161 (1,4-disubstituted phenyl compounds), WO01/00579 (aryl compounds), WO01/12612 & WO01/12187 (benzoic acid compounds), and WO97/31907 (substituted 4-hydroxy-phenylalconic acid compound); sterol biosynthesis inhibitors such as DMP-565; triglyceride synthesis inhibitors; microsomal triglyceride transport (MTTP) inhibitors, such as inplitapide, LAB687, and CP346086, AEGR 733, implitapide and the like; HMG-CoA reductase gene expression inhibitors (e.g. compounds that decrease HMG-CoA reductase expression by affecting (e.g. blocking) transcription or translation of HMG-CoA reductase into protein or compounds that maybe biotransformed into compounds that have the aforementioned attributes by one or more enzymes in the cholesterol biosynthetic cascade or may lead to the accumulation of an isoprene metabolite that has the aforementioned activities (such regulation is readily determined by those skilled in the art according to standard assays (Methods of Enzymology, 110:9-19 1985))) such as those disclosed in U.S. Pat. No. 5,041,432 (certain 15-substituted lanosterol derivatives) and E. I. Mercer (1993) Prog. Lip. Res. 32:357 (oxygenated sterols that suppress the biosynthesis of HMG-CoA reductase); squalene epoxidase inhibitors such as NB-598 ((E)-N-ethyl-N-(6,6-dimethyl-2-hepten-4-y-nyl)-3-[(3,3′-bithiophen-5-yl)methoxy]benzene-methanamine hydrochloride); low density lipoprotein (LDL) receptor inducers such as HOE-402 (an imidazolidinyl-pyrimidine derivative that directly stimulates LDL receptor activity, see Huettinger et al (1993) Arterioscler. Thromb. 13:1005); platelet aggregation inhibitors; 5-LO or FLAP inhibitors; PPAR modulators (including compounds that may have multiple functionality for activating various combinations of PPARα, PPARγ, and PPARδ) such as those disclosed in U.S. Pat. Nos. 6,008,237, 6,248,781, 6,166,049, WO00/12491, WO00/218355, WO00/23415, WO00/23416, WO00/23425, WO00/23442, WO00/23445, WO00/23451, WO00/236331, WO00/236332, WO00/238553, WO00/50392, WO00/53563, WO00/63153, WO00/63190, WO00/63196, WO00/63209, WO00/78312, WO00/78313, WO01/04351, WO01/14349, WO01/14350, WO01/16120, WO01/17994, WO01/21181, WO01/21578, WO01/25 181, WO01/25225, WO01/25226, WO01/40192, WO01/79150, WO02/081428, WO02/100403, WO02/102780, WO02/79162, WO03/016265, WO03/033453, WO03/042194, WO03/043997, WO03/066581, WO97/25042, WO99/07357, WO99/11255, WO99/12534, WO99/15520, WO99/46232, and WO98/05331 (including GW233 1 or (2-(4-[difluorophenyl]-1 heptylureido)ethyl]phenoxy)-2-methylbutyric)); niacin-bound chromium, as disclosed in WO03/039535; substituted acid derivatives disclosed in WO03/040114; apolipoprotein B inhibitors such as those disclosed in WO02/090347, WO02/28835, WO03/045921, WO03/047575; Factor Xa modulators such as those disclosed in WO03/047517, WO03/047520, WO03/048081; ileal bile acid transport (“IBAT”) inhibitors (or apical sodium co-dependent bile acid transport (“ASBT”) inhibitors) such as benzothiepines (including 1,2-benzothiazepines; 1,4-benzodiazepines; 1,5-benzothiazepines; 1,2,5-benzothiadiazepines); PPARδ activators such as disclosed in WO01/00603 (thiazole and oxazole derivates (e.g. C.A.S. Registry No. 317318-32-4), WO97/28149 (fluoro, chloro and thio phenoxy phenylacetic), U.S. Pat. No. 5,093,365 (non-1-oxidizable fatty acid analogues), and WO99/04815. Tests showing the efficacy of the therapy and the rationale for the combination therapy with a dyslipidemic agent are presented in US2003 0069221 (where the dyslipidemic agents are called “cardiovascular agents”)

Dosage

Dosage levels of active ingredients in a pharmaceutical composition can also be varied so as to achieve a transient or sustained concentration of the compound in a subject, especially in and around the site of inflammation or disease area, and to result in the desired response. It is well within the skill of the art to start doses of the compound at levels lower than required to achieve the desired effect and to gradually increase the dosage until the desired effect is achieved. It will be understood that the specific dose level for any particular subject will depend on a variety of factors, including body weight, general health, diet, natural history of disease, route and scheduling of administration, combination with one or more other drugs, and severity of disease.

An effective dosage of the composition will typically be between about 1 μg and about 10 mg per kilogram body weight, preferably between about 10 μg to 5 mg of the compound per kilogram body weight. Adjustments in dosage will be made using methods that are routine in the art and will be based upon the particular composition being used and clinical considerations.

The guanylate cyclase receptor agonists used in the methods described above may be administered orally, systemically or locally. Dosage forms include preparations for inhalation or injection, solutions, suspensions, emulsions, tablets, capsules, topical salves and lotions, transdermal compositions, other known peptide formulations and pegylated peptide analogs. Agonists may be administered as either the sole active agent or in combination with other drugs, e.g., an inhibitor of cGMP-dependent phosphodiesterase and anti-inflammatory agent. In all cases, additional drugs should be administered at a dosage that is therapeutically effective using the existing art as a guide. Drugs may be administered in a single composition or sequentially.

Dosage levels of the GCR agonist for use in methods of this invention typically are from about 0.001 mg to about 10,000 mg daily, preferably from about 0.005 mg to about 1,000 mg daily. For example, an effective dosage of the GCRA peptide for use in methods of this invention is 0.1, 0.2. 0.3, 0.4. 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 18, 19, or 20 mg per day, or optionally twice a day. Preferably the GCRA peptide is given after a meal (i.e, 30 minutes). In some embodiments a second agent useful for treating a lipid metabolism disorder, a binary disorder, a cardiovascular disease, obesity or an an endocrine disorder is administered. Suitable second agents are described herein. In some aspects the second agent is administered at less than the standard does for treating the particular disorder because the GCRA peptide acts synergistically with the second agent. For example, 2.5, 5. 7.5 or 10 mg of Liptor is given twice a day after a meal (i.e, 30 minutes). On the basis of mg/kg daily dose, either given in single or divided doses, dosages typically range from about 0.001/75 mg/kg to about 10,000/75 mg/kg, preferably from about 0.005/75 mg/kg to about 1,000/75 mg/kg.

The total daily dose of each inhibitor can be administered to the patient in a single dose, or in multiple subdoses. Typically, subdoses can be administered two to six times per day, preferably two to four times per day, and even more preferably two to three times per day. Doses can be in immediate release form or sustained release form sufficiently effective to obtain the desired control over the medical condition.

The dosage regimen to prevent, treat, give relief from, or ameliorate a medical condition or disorder, or to otherwise protect against or treat a medical condition with the combinations and compositions of the present invention is selected in accordance with a variety of factors. These factors include, but are not limited to, the type, age, weight, sex, diet, and medical condition of the subject, the severity of the disease, the route of administration, pharmacological considerations such as the activity, efficacy, pharmacokinetics and toxicology profiles of the particular inhibitors employed, whether a drug delivery system is utilized, and whether the inhibitors are administered with other active ingredients. Thus, the dosage regimen actually employed may vary widely and therefore deviate from the preferred dosage regimen set forth above.

### EXAMPLES

### Example 1

### GC-C Agonists Ameliorate Colitis in Mice Via a Cyclic GMP Mediated Mechanism to Downregulate NF-κB and Pro-Inflammatory Cytokines

### Materials and Methods

Materials: T84 cells were obtained from Leonard Forte, University of Missouri, Columbia, Mo. UG and its analogs were chemically synthesized using the procedure as described and purified by BACHEM BioSciences, PA. Plecanatide, SP-333 and UG were chemically synthesized by procedures as described previously (Shailubhai and Jacob, 2009). All other chemicals, cytokines ELISA kits, and antibodies were obtained from commercially available vendors.

Cyclic GMP stimulation assay: The potency of test peptides to stimulate cGMP synthesis in T84 cells was assayed by a published procedure (Shailubhai et al. 2000). Briefly, confluent monolayers of T-84 cells in 24-well plates were washed twice with 250 μl of DMEM containing 50 mM HEPES (pH 7.4), pre-incubated at 37° C. for 10 min with 250 μl of DMEM containing 50 mM HEPES (pH 7.4) and 1 mM isobutylmethylxanthine (IBMX), followed by incubation with test peptide for 30 min. The medium was aspirated, and the reaction was terminated by the addition of 3% perchloric acid. Following centrifugation, and neutralization with 0.1 N NaOH, the resulting supernatant was used directly for measurements of intracellular cGMP using an ELISA kit (Caymen Chemical, Ann Arbor, Mich.). Results are expressed as pmol of cGMP/mg of protein in the cell extracts.

Inhibition of NF-κB signaling by SP-333: Approximately 1.5×10⁶ T84 cells were seeded into 100 mm dishes and cultured essentially as described (Shailubhai et al. 2000). At 85-90% confluence, cells were washed and treated with 10 μg/ml lipopolysaccharides (LPS) from Escherichia coli (Sigma-Aldrich, St. Louis, MO) for 4 h. Subsequent to LPS treatment, cells were washed and replenished with fresh media containing 8-Bromo-cyclic GMP (8-Br-cGMP) at 0, 0.1, 1, 10, 100 and 1000 μM (Enzo Life Science, Farmingdale, N.Y.), or SP-333 at 0, 0.01, 0.1, 1 and 10 μM together with 500 μM of phosphodiesterase inhibitor Zaprinast (Sigma-Aldrich, St. Louis, Mo.) for 16 h before harvest. For analyzing phosphorylation status of IKK-Δ/β, T84 cells were stimulated with LPS (10 μg/ml) for 4 h followed by treatment with 0.1 and 1.0 μM of SP-333 in the presence of 500 μM Zaprinast, for 2 h. The control cells (without LPS treatment) were washed and refilled with fresh media and harvested simultaneously with treated cells. Nuclear and cytosolic extracts were prepared by the previously described method (Aiamkitsumrit et al. 2007). Protein concentration was measured with a Bio-Rad protein assay kit (Bio-Rad, Hercules, Calif.). The extracts were stored at −80° C. until use.

Inhibition of NF-κB signaling by SP-333: T84 cells at 85-90% confluence were washed and treated with 10 μg/ml Escherichia coli LPS (Sigma-Aldrich, St. Louis, Mo.) for 4 h. Subsequently, cells were washed, replenished with fresh media containing either SP-333 (0 to 10 μM) or 8-Bromo-cyclic GMP (8-Br-cGMP (0-1 mM) for 16 h. Zaprinast (500 μM), a cGMP-specific PDE inhibitor, was used in all cell culture experiments to inhibit cGMP degradation. Treatment of T84 cells with zaprinast (500 μM) alone did not increase cGMP levels and it did not inhibit NF-κB activation. The control cells (without LPS) were processed using identical procedures.

Western Blot analysis: This was performed essentially as described²¹ employing either 50 μg of cytoplasmic or 25 μg proteins of the nuclear fractions. The blots were probed overnight with primary antibodies of interest (anti-p65 antibody, 1:200 dilution; anti-IκB antibody 1:500 dilution; anti-phosphorylated p65 antibody, 1:250 dilution; anti-phosphorylated IκB antibody 1:250 dilution; all were from Santa Cruz Biotechnology (Santa Cruz, Calif.). Anti-IKK-β and phosphorylated IKK-α/β (Ser 176/180) were purchased from Cell Signaling Technology, Boston, Mass. and used at 1:500 dilution; anti-mouse or anti-rabbit antibodies conjugated to horseradish peroxide were used for chemiluminescence detection as recommended by manufacturer (GE Healthcare, Piscataway, N.J.).

Quantitative RT-PCR for Transcripts: T-84 cells (˜10⁷ cells) were treated with 1 μM SP-333 for 5 h. Subsequently, cDNA was prepared from total RNA using High Capacity RNA-to-cDNA Kit (Applied Biosystems, Carlsbad, Calif.) according to manufacturer's instructions. Quantitative Real-Time PCR amplification and analysis were carried out using Lightcycler 480 (Roche, Basel Switzerland), p65 and IKK-β specific Taqman gene expression assays and Lightcycler 480 Probe master. All amplification assays were performed in duplicate employing 20 ng cDNA (based on input RNA). The total reaction volume was adjusted to 20 μL with appropriate amounts of TaqMan gene expression assays, Lightcycler 480 probe master and distilled water, then subjected to 45 PCR cycles using default cycling parameters. GAPDH transcript was used as endogenous control. The data generated were analyzed and expressed as target gene expression relative to endogenous control using comparative Cp method and 2^{-ΔΔ}Cp formula (comparative Cp values and advanced quantitative relative quantification method). TaqMan reagents were obtained from Applied Biosystems (Carlsbad, Calif.).

Studies in experimental models of murine colitis: BALB/c and TCRα−/− mice were obtained from Jackson Laboratories and handled as per the IACUC-approved protocols of the University of Pittsburgh School of Medicine, PA. BDF1 mice used for dextran sulfate sodium (DSS)- and 2,4,6 trinitrobenzenesulfonic acid (TNBS)-induced colitis studies conducted by Epistem Co. (Manchester, UK) were obtained from Harlan Laboratories, UK. All animals were handled and euthanized in compliance with the institutional and national animal welfare regulations.

Cytokines expression analysis: To evaluate the effect of different concentrations of SP-333 on secretion of cytokines (IL-8 and TNF), 12-well plates seeded with 10⁵ cells/well were used. At 85-90% confluence, cells were washed with DPBS and treated with or without LPS (10 μg/ml) for 4 h, followed by washing with phosphate buffered saline (PBS). SP-333 at 0, 0.01, 0.1, 1 and 10 μM) together with 500 μM of Zaprinast was added to the treated cells and incubated further for 16 h. The levels of IL-8 and TNF were quantified using human IL-8 and TNF ELISA development kits (Peprotech, Rocky Hill, N.J.) Each ELISA was performed in triplicate with cell-free supernatants from two independent experiments. The protein concentration of each well was assessed by Bio-Rad protein dye detection kit.

TNBS-induced colitis in Mice: Mouse strains BDF1 and BALB/c were used to examine the efficacy of plecanatide in TNBS induced colitis. The procedures used were as previously described (Hegazi et al., 2005; Dave et al., 2009). Briefly, to induce colitis in specific pathogen-free 2-4 months old female BALB/c mice, 0.5 mg of TNBS in 50% ethanol was slowly administered into the lumen of the colon via a 3.5 F catheter fitted onto a 1-ml syringe (total injection volume 100 μl). Plecanatide formulated in PBS was administered by oral gavage for a total of 7 days, with the first dose given one day prior to TNBS challenge. Mice were observed daily for body weight, food consumption and for signs of any apparent toxicity, rectal bleeding or prolapse. After 7 days of treatment, GI tissue collection, fixation and histopathological scoring were performed essentially as described previously (Sheikh et al., 2011). GI tissues were also collected and immediately processed for explant culture and cytokine expression as described below.

TNBS-induced colitis in BDF-1 mice: To induce colitis in BDF1 mice, a total of 90, 10-12 week old, H. pylori free male mice were used. Animals were randomized into 9 groups. TNBS was rectally administered as described above in a total injection volume of 100 μl. Oral administration of PBS formulated plecanatide (0.0005-50 mg/kg), animal monitoring and collection of GI tissues was performed as described above. Daily administration of plecanatide was continued through day 6, when the mice were euthanized. Colon tissues were removed and weighed. Distal sections were fixed, stained with H&E, and evaluated for histopathology and visual severity scores (Hegazi et al., 2005; Dave et al., 2009). Scoring criteria were similar to those described for DSS-induced colitis studies.

TCRα knockout mice: TCRα^{-/-} mice were matched for age and sex in all experiments. 16 week old TCRα_{-/-} mice were treated with plecanatide at 0.5 or 2.5 mg/kg/day or PBS vehicle control by oral gavage for 14 days (6 mice per group). Mice were sacrificed 12 h after the final dose, and GI tissues were collected for experimental analyses. Colitis severity in TCRα^{-/-} model was scored as described (Berg et al., 1996).

DSS-induced colitis in mice: To evaluate the efficacy of plecanatide in DSS induced colitis model, 48 BDF-1 mice were divided into 8 treatment groups (6 mice/group). One group was not exposed to DSS (untreated control) and groups 2-8 were treated with 5% DSS in the drinking water on day 0. Plecanatide or SP-333 solutions formulated in 0.1M phosphate buffer (pH 7) was administered by oral gavage, once a day at 0.005, 0.05, 0.5, 2.5 and 5 mg/kg, from study day −1 (i.e. prior to initiation of DSS treatment) through day 6 for SP-333 or day 7 for plecanatide. DSS treatment was initiated on day 0 for groups 2-8 until the end of the study. Sulfasalazine (80 mg/kg) or 5-ASA (100 mg/kg) was used as positive control and was administered daily. Mice were sacrificed on day 7. The large intestine was removed and weighed. Distal section of the large intestine was collected and fixed as described above.

Evaluation of colitis: Disease activity index (DAI) was calculated by scoring in-life parameters starting from day −1 until the end of the study. Once daily, all mice were weighed and assessed for stool consistency, and the presence of overt blood in the stool or around the anus essentially similar to that reported earlier. Colitis severity was assessed by histopathological analyses of H&E stained tissue sections (Hegazi et al., 2005; Dave et al., 2009) employing the following criteria: (score 0) normal appearing crypts; (score 1) abnormal crypt pathology without ulceration; (score 2) depleted crypts with ulceration; (score 3) 20-50% depleted crypts and increased ulceration; (score 4)>50% depleted crypts with substantial ulceration and (score 5) totally ulcerated/inflamed colon with no crypts remaining. All slides were scored in a blinded manner. Colitis severity in TCRα_{-/-} model was scored as previously described²⁵. All slides were scored in a blinded manner. Disease activity index (DAI) was calculated by assessing body weight, stool consistency, and the presence of overt blood in stools or around the anus, essentially similar to that reported earlier (Hamamoto et al., 1999).

Explant cultures to measure cytokine expression: Colonic tissue fragments (0.5 g dry weight) from the TNBS-induced colitis mice and TCRα^{-/-} knockout mice were processed as previously described (Hegazi et al., 2005). Tissue fragment supernatants were collected after 24 h for cytokine ELISAs. Murine immunoassay kits (R&D System) were used according to the manufacturer's instructions for IL-12p40, IL-23 and TNF. Linco Cytokine-16 plex Mouse ELISA was performed for MIP-1α, RANTES, IL-10 and IL-17 (Millipore) as per manufacturer's instructions.

Cytokine analysis: Murine IL-12 p40, IL-10, TNF, IL-1β, IFN-γ, immunoassay kits (R&D System, MN, USA) were used according to the manufacturer's instructions. Linco Cytokine-16 plex Mouse ELISA was performed for MIP-1α, RANTES, IL-10, TNF and IL-17 (Millipore, Billerica, Mass.) as per manufacturer's instructions.

Selection of samples for Ki-67 labeling and Myeloperoxidase (MPO) assay: A total of 40 samples were chosen for analysis of MPO activity and Ki-67 labeling from the treatment groups that received SP-333, its vehicle control (0.1M phosphate buffer, pH 7) and the reference compound (5-ASA). A random number generator (www.random.org) was employed to select samples from these groups (7 from each of the SP-333-recipient groups and 6 from each of the vehicle and 5-ASA groups).

Ki-67 labeling in large bowel cross sections: Employing standard procedures, two non-serial sections from each animal, were de-waxed in xylene and rehydrated in graded alcohols to PBS (pH 7.4). Endogenous peroxidase was blocked by incubation in 0.3% hydrogen peroxidase for 30 min. Antigen retrieval was performed by placing the slides in 1 L of citrate buffer (pH 6) and microwaving at high power for 20 minutes. After cooling for 15 minutes, sections were incubated with 10% normal goat serum for 30 minutes to block non-specific binding. Primary antibody (mouse monoclonal IgG, clone MIB-1, Dako) at 1/50 dilution was added and incubated for 1 hour at room temperature. Sections were washed and incubated with biotinylated secondary IgG antibody (1/200 dilution; Vector Labs Inc.), for 45 minutes at room temperature. Subsequently, slides were washed and incubated with ABC Elite reagents (Vector Labs Inc.), for 30 minutes at room temperature. Presence of bound antibody was demonstrated with 3,3′-diaminobenzidine (DAB: 0.5 mg/ml in PBS). Sections were counterstained with thionine, dehydrated and mounted.

MPO activity in lysates prepared from snap-frozen, mid-colon tissues: Myeloperoxidase activity in colonic tissue samples was performed according to the method described (Krawisc et al., 1984). Briefly, the rate of change in absorbance at 450 nm was recorded when 20 μl of tissue extract was incubated with 150 μl of reaction buffer containing 0.26 mg/ml O-dianisidine and 0.6 μ/ml H₂O₂. Each reaction was performed in triplicate. The rate of reaction was determined (initial slope) and normalized to protein concentration of sample. Statistical analysis: Where mentioned, statistical comparisons of group data were performed using 2-way, unpaired T tests, assuming unequal variance, using Microsoft Excel. Statistically significant differences (p<0.05) and borderline non-significant differences (p<0.1) are indicated on the figures.

### Results

### Plecanatide and SP-333 are Potent Analogs of Human Natriuretic Peptide UG

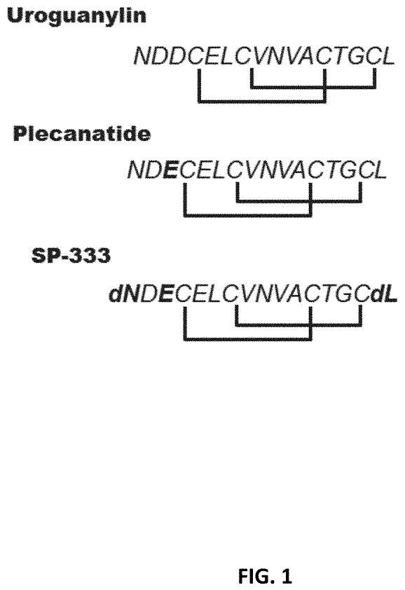

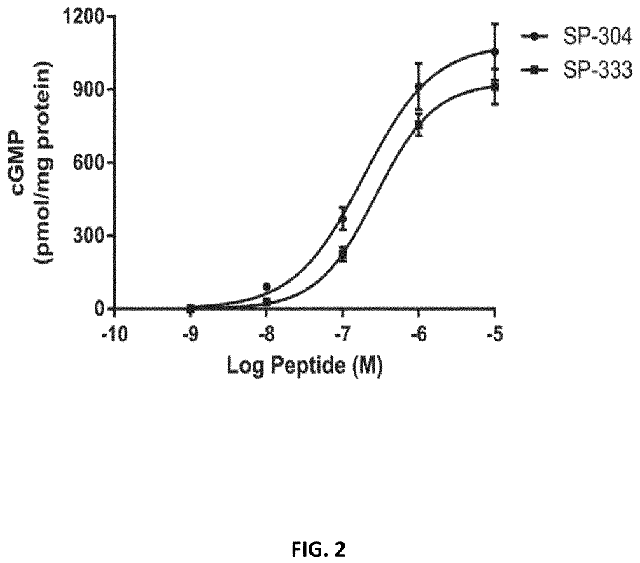

Plecanatide is structurally similar to UG except for the substitution of aspartic acid with glutamate at the 3rd position from N-terminal. SP-333 is similar to plecanatide in structure except that L-stereoisomer amino acids at the N- and C-termini are replaced with the corresponding D stereoisomers Based on three-dimensional structures and energy calculations of UG isomers, substitution of the third amino acid from the N-terminus (D->E) in the parent UG peptide was predicted to stabilize the peptide structure predominantly in an active form. Plecanatide and SP-333 represent two potent synthetic analogs of human UG (FIG. 1). These analogs were stable during solid-phase synthesis and folding, and produced negligible amounts of the inactive isomer during purification. SP-333 was designed with D-stereoisomer amino acids at the N- and C-terminus (FIG. 1) to make the peptide more resistant to proteolytic degradation. Both analogs possess disulfide-linked bridges between amino acid position 4-12 and 7-15 as observed in UG peptide (schematically shown FIG. 1). Potencies of plecanatide and SP-333 were evaluated in a bioassay employing the human T84 colon cell line that exhibits robust cGMP responses. The peptides are equipotent causing a dose dependent increase in cGMP (FIG. 2) with an EC₅₀ of 1.889×10⁻⁷ M and 2.826×10⁻⁷ M, respectively.

SP-333 inhibits NF-κB Activation

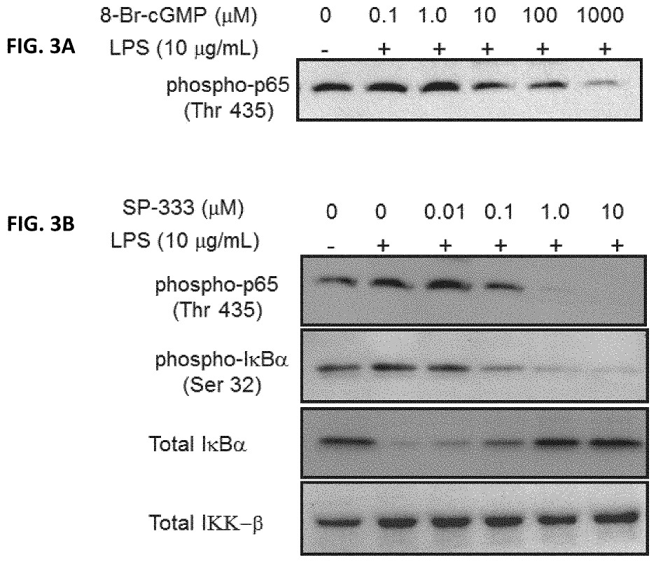

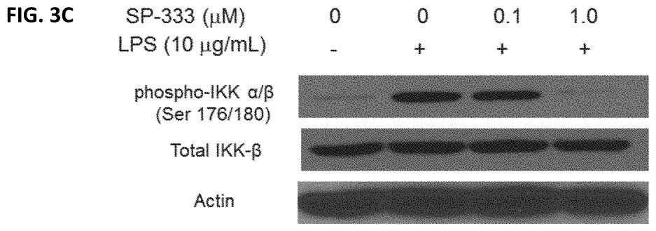

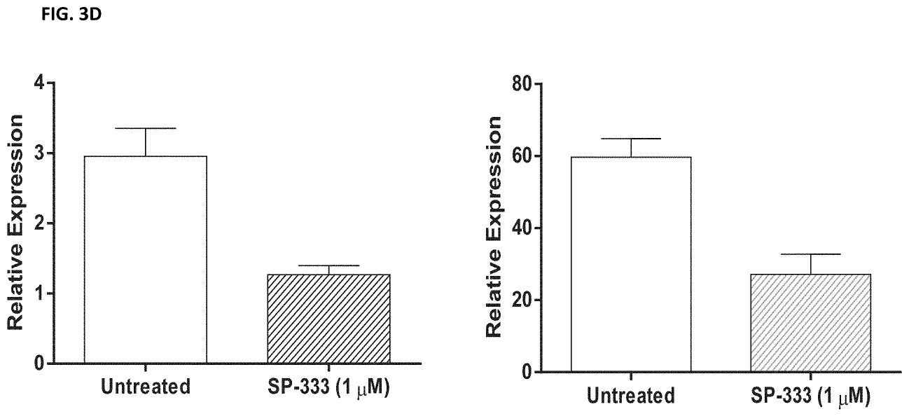

Phosphorylated-p65 (phospho-p65) protein expression, a measure of NF-κB activation, was markedly enhanced in the cytosolic fraction of T84 cells following 4 h treatment with LPS (10 μg/mL) (FIG. 2A). Treatment of LPS-activated T84 cells, either with 8-Br-cGMP (1 mM) or with SP-333 (1.0 and 10 μM), reduced levels of phospho-p65 in a dose-dependent manner (FIGS. 3A&B). In addition, SP-333 also reduced levels of phospho-IκBα with concomitant increase in levels of IκBα, an endogenous inhibitor of NF-κB, which is phosphorylated primarily by I-κ-kinase (IKKβ). In FIG. 3C, the immunoblot, developed using an anti-IKKβ antibody recognizing both phosphorylated and unphosphorylated IKKβ, showed that the level of total IKKβ remained unchanged following treatment with SP-333. However, when the immunoblot was developed using an antibody specific for phosphorylated IKKβ, low levels of phosphorylated IKKβ (phospho-IKKβ were detected in untreated control T84 cells (lane 1, FIG. 3C), but stimulation of cells with LPS resulted in a significant increase in the levels of phosphorylated IKKβ (lane 2, FIG. 3C). Treatment with SP-333 (1 μM) completely reversed the LPS-mediated increase in phosphorylation of IKKβ (lane 4, FIG. 3C). The total levels of IKK-β and actin remained unchanged, indicating the same level of protein loading in each lane (FIG. 3C). Taken together, these results suggested that SP-333-mediated inhibition of NF-κB activation might occur through a cGMP-mediated mechanism.

It was also of interest to examine if SP-333 treatment had any on expression of IKKβ and p65 transcripts. Treatment with SP-333 reduced transcript levels of IKK-β and p65 by approximately 50% as compared to those in the unstimulated T84 cells (FIG. 3D). These in vitro results prompted us to further examine the anti-inflammatory actions of plecanatide and SP-333 in experimental models of murine colitis.

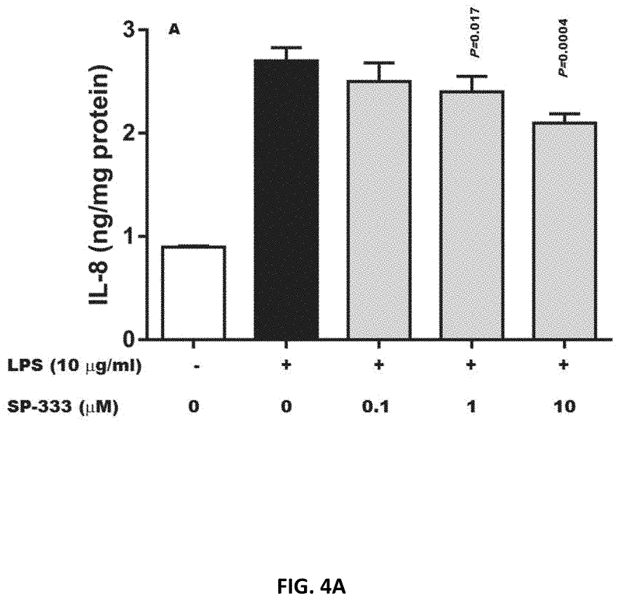

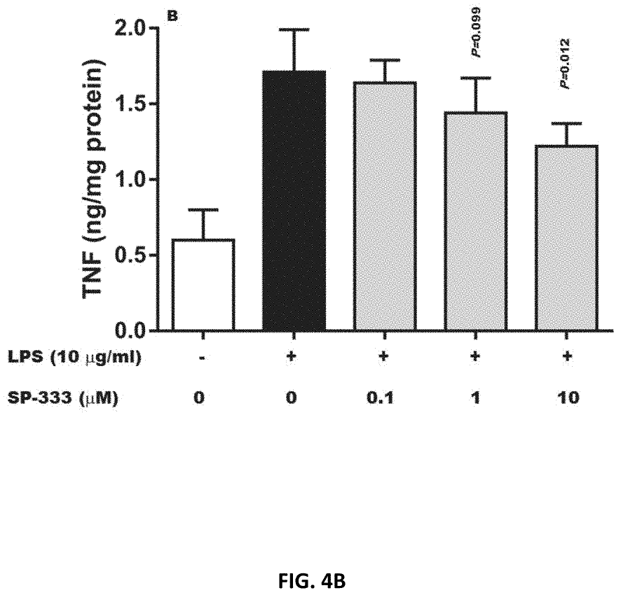

NF-κB is a central regulator of pro-inflammatory cytokine expression in multiple cell types. T84 cells were stimulated with LPS (10 μg/ml) for 4 h and subsequently treated with indicated amounts of SP-333 for 16 hr. Secretion of IL-8, and TNF in the extracellular medium were determined by ELISA. Results presented in FIGS. 4A and B demonstrated that LPS treatment caused a significant increase in the levels of IL-8 and TNF as compared to those in untreated controls. SP-333 treatment caused concentration dependent reversal of LPS stimulated production of IL-8 and TNF (FIGS. 4A and B).

### Plecanatide Ameliorates GI Inflammation in Several Murine Colitis Models

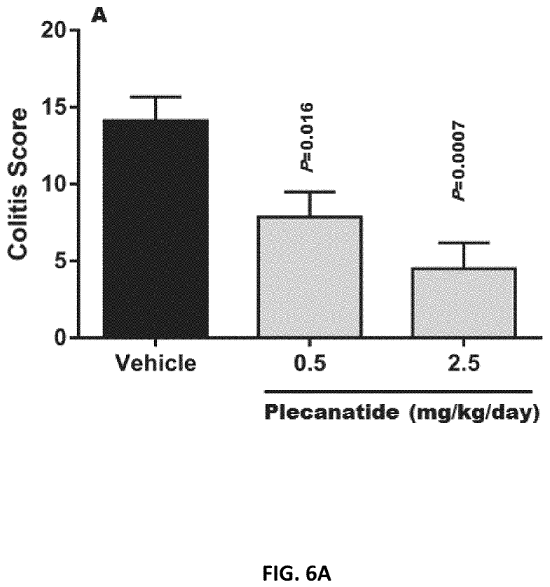

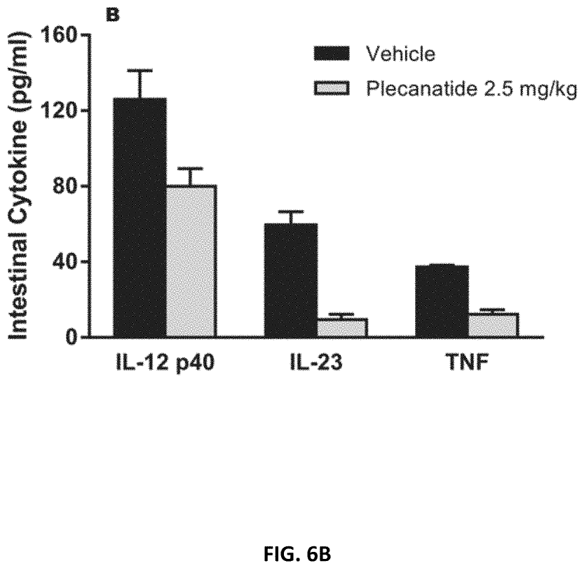

Plecanatide ameliorates DSS- and TNBS-induced colitis in mice: In a preliminary study, oral treatment with plecanatide at 0.5 and 2.5 mg/kg, given once daily dose for 7 days, effectively ameliorated TNBS-induced colitis in BALB/c mice (FIG. 6A). Colon tissues from these mice were used for explant cultures to examine secretion of certain pro-inflammatory cytokines. Plecanatide treatment suppressed secretion of IL-12p40 (37%), IL-23 (84%), and TNF (67%) in colonic explants as compared to the vehicle-treated explants (FIG. 6B).

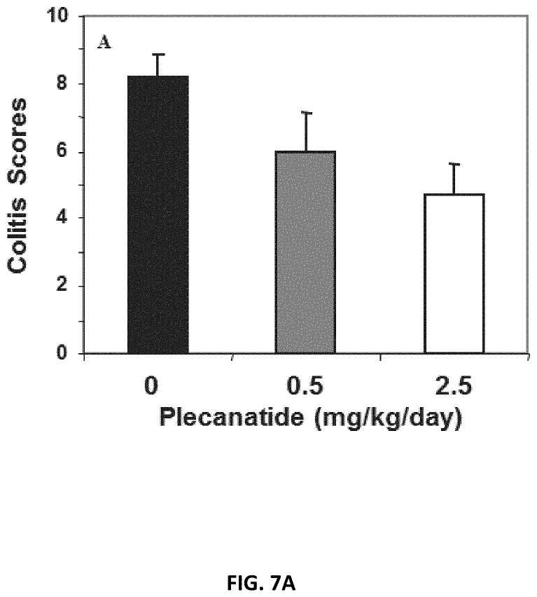

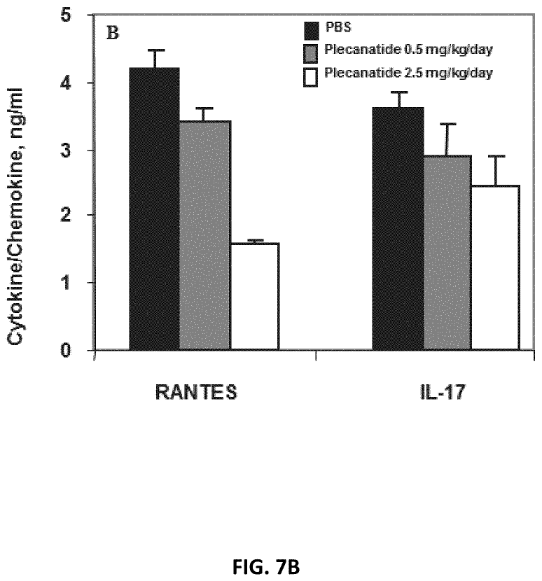

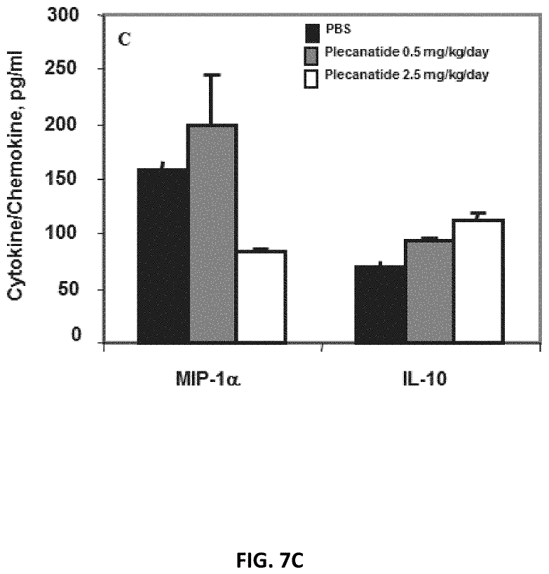

Effectiveness of oral treatment with plecanatide to ameliorate colitis was also examined in TCRα −/− mice, which are known to spontaneously develop chronic colitis. Daily treatment with plecanatide for two weeks reduced colitis scores as compared to those in the vehicle treated mice (FIG. 7A). Colon tissues from these mice were used in explant cultures to measure secretion of chemokines and cytokines in the culture media. Plecanatide treatment considerably suppressed production of RANTES and MIP-1α, and IL-17 (FIG. 7B). Interestingly, the treatment also increased levels of the secreted IL-10, a cytokine known to be reduced during GI inflammation (FIG. 7C). Taken together, these preliminary results prompted a further evaluation of plecanatide in other murine models of experimental colitis.

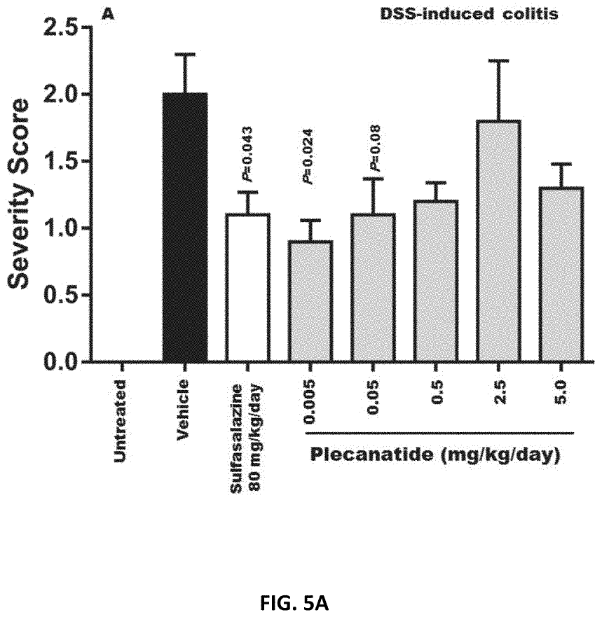

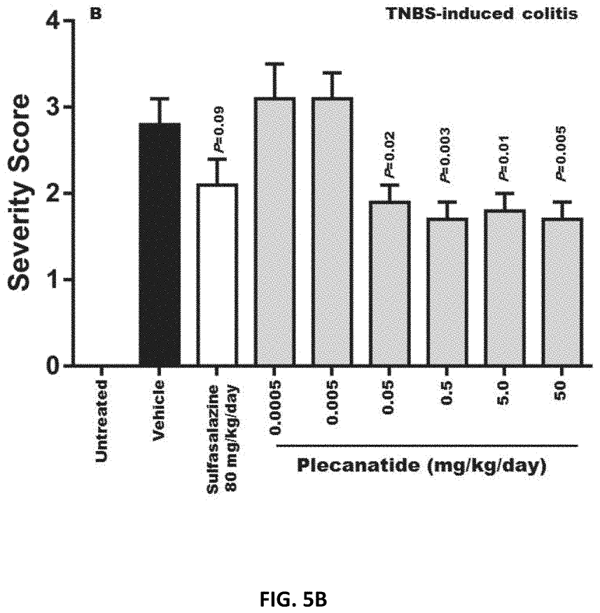

The effectiveness of plecanatide to ameliorate colitis was further evaluated in DSS- and TNBS-induced colitis in BDF-1 mice. Consistent with the results from the preliminary studies described above, oral treatment with plecanatide, even at a dose as low as 0.005 mg/kg, was as effective as sulfasalazine (80 mg/kg) in ameliorating DSS-induced colitis in BDF-1 mice (FIG. 5A). However, doses higher than 0.005 mg/day did not produce incremental effect on amelioration of colitis. Surprisingly, the colitis severity scores in the group of mice treated with 2.5 mg/kg showed an unusually high level of experimental variability. Nevertheless, the anti-inflammatory activity of plecanatide, at a broader range between 0.0005 to 50 mg/kg, was further examined in the TNBS-induced colitis in BDF1 mice. As shown in FIG. 5B, plecanatide treatment at doses under 0.005 mg/kg was ineffective but 0.05 mg/kg and higher doses produced statistically significant reduction in colitis severity. Again, there was no further reduction in colitis severity with incremental doses was observed, suggesting that the dose above 0.05 mg/kg might be saturating. The effective dose-range of plecanatide was found to be in range between 0.05-0.5 mg/kg in this model (FIG. 5B).

Oral Treatment with SP-333 Ameliorates DSS-Induced Colitis in Mice

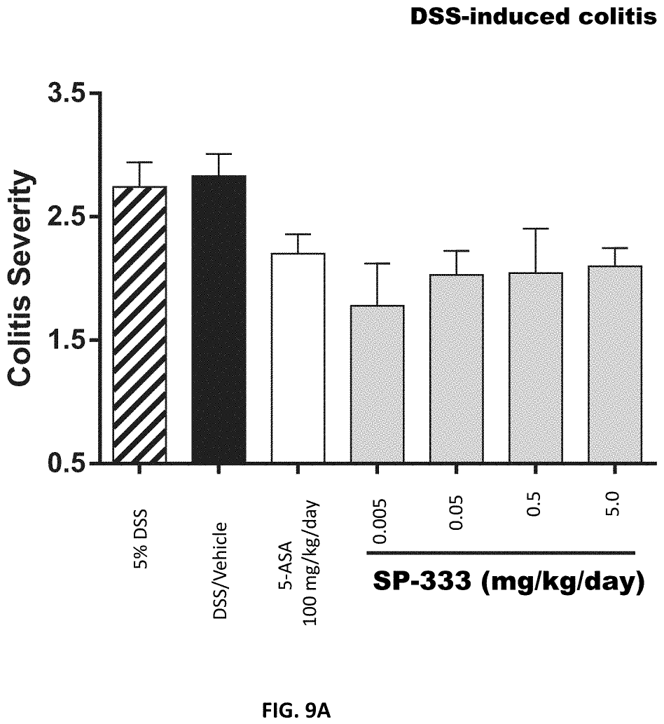

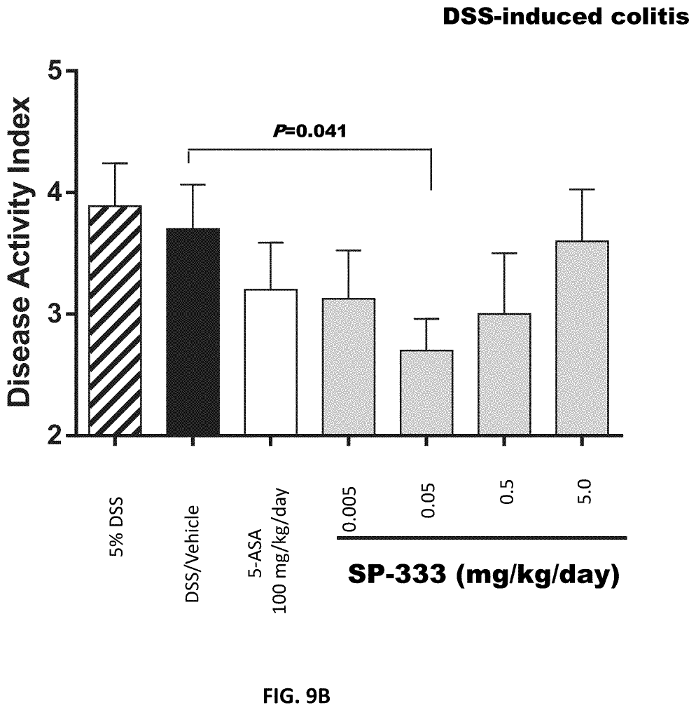

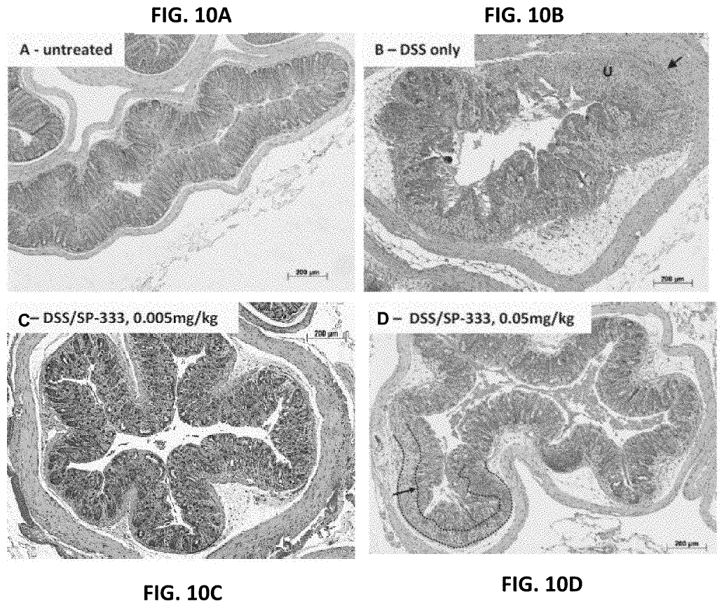

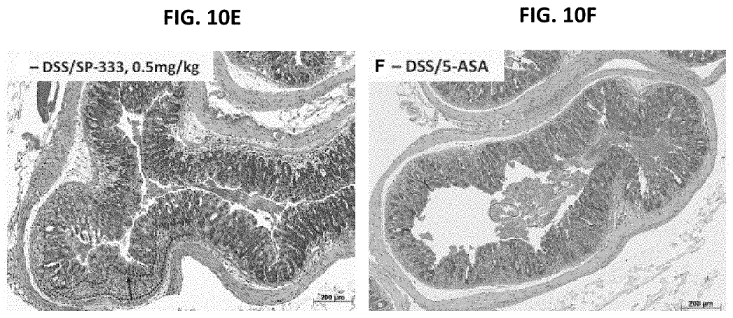

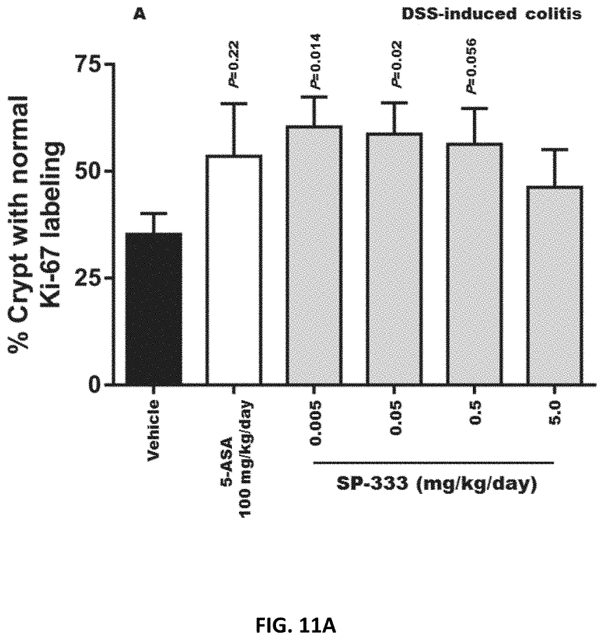

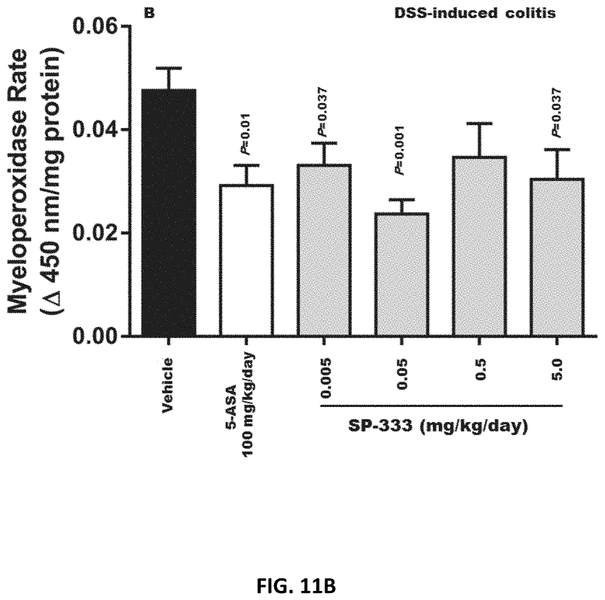

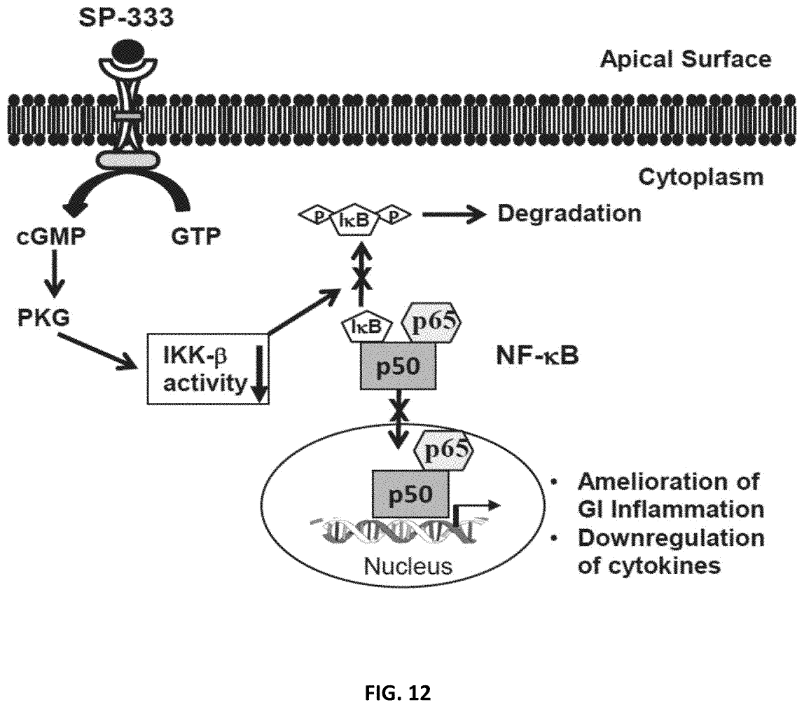

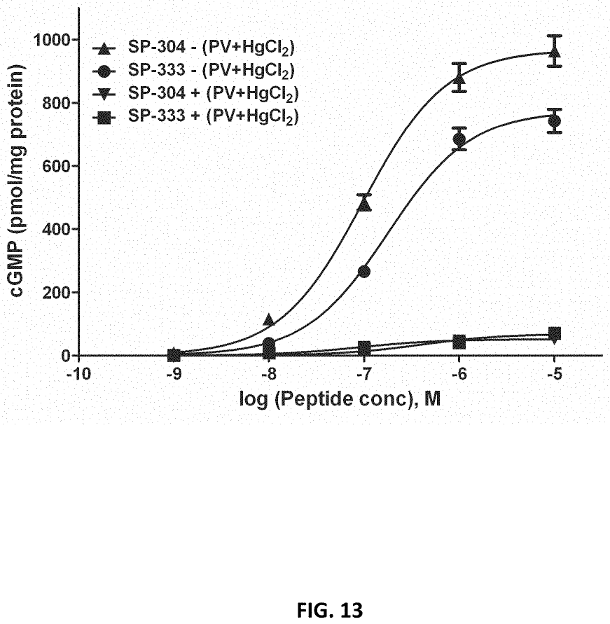

BDF-1 mice were administered SP-333 (0.005 mg/kg to 5 mg/kg) by oral gavage to determine efficacy in acute DSS-induced colitis. Mice that received either SP-333 or reference compound 5-ASA (100 mg/kg) had lower mean DAI scores than mice treated with vehicle control or DSS alone (FIG. 9A). Effect of oral treatment with SP-333 was evaluated on DSS-induced colitis in BDF-1 mice to further confirm anti-inflammatory activity of GC-C agonists. As shown in FIG. 9, a daily dose of SP-333 of between a 0.005 to 5 mg/kg dose range or of 5-ASA (100 mg/kg; as positive control) consistently showed lower mean scores of colitis severity (FIG. 9B) and DAI (FIG. 9A) as compared to those in DSS alone or DSS plus vehicle-treated mice. The effect of SP-333 treatment on levels of MPO activity in colon tissues was measured as an indirect way to assess severity of GI inflammation. As expected, the colon tissues from DSS-treated mice exhibited the highest levels of MPO (0.048±0.004 units/min). The MPO activity was considerably reduced following treatment either with 5-ASA (100 mg/kg) or with SP-333 at all doses (FIG. 11B). Mice administered 0.05 mg/kg of SP-333 exhibited the lowest (˜50% reduction) level of MPO activity (0.024±0.003 units/minute; p=0.001). Histological evaluation of colon tissues from DSS-treated mice showed substantial loss of crypts, changes in crypt architecture, ulceration, and localized infiltration of inflammatory cells (FIG. 10B; score=4) as compared to those in the naive tissues (FIG. 10A; score 0). Substantial loss of crypts and ulceration can be seen upon DSS treatment (FIGS. 10B and H score=/>3). As expected, colon tissues from the 5-ASA-treated mice group exhibited only patchy loss of crypts and the distortion of crypt morphology was limited (FIG. 10F; score=2). The colon tissues from mice in SP-333 treatment groups (0.005 mg/kg and 0.05 mg/kg) exhibited with minimal loss of crypts with very low distortions in crypt morphology (FIGS. 10C, D and E; scores 1, 2 and 2, respectively). Stability of plecanatide and SP-333 against proteolysis

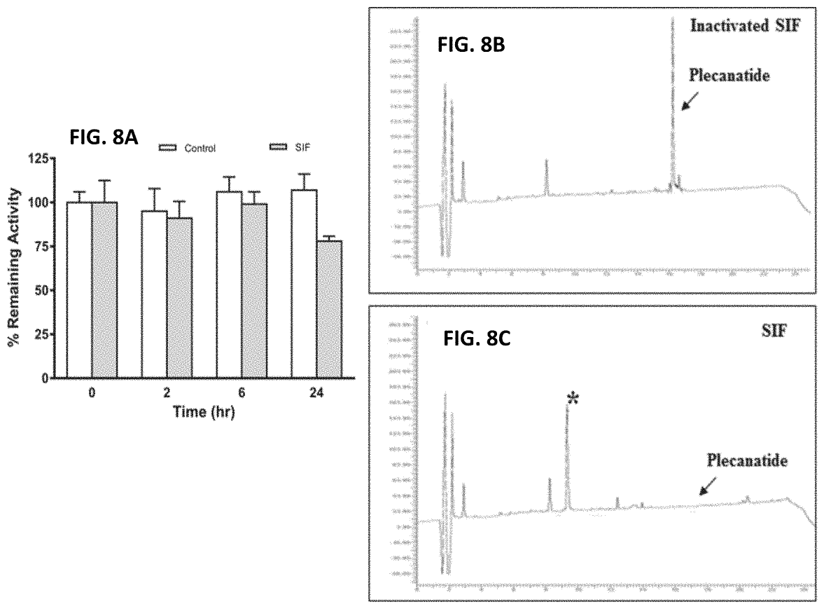

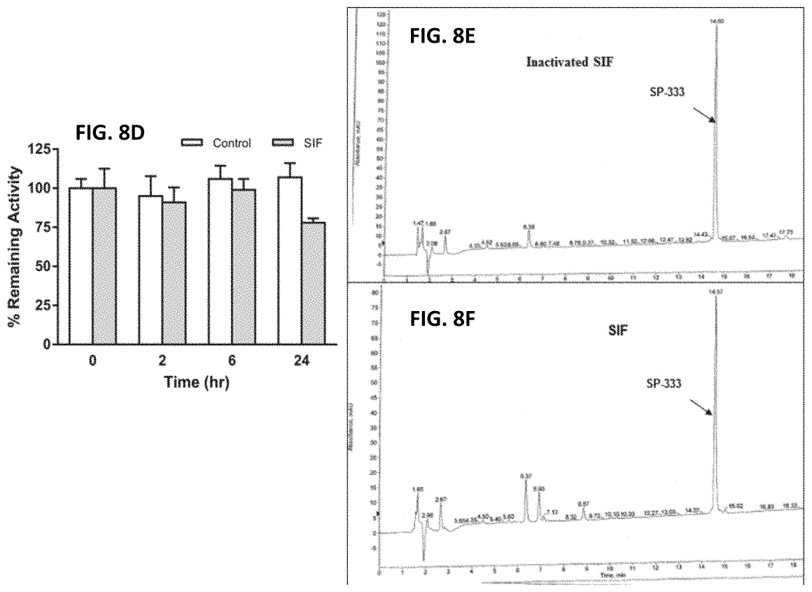

Plecanatide and SP-333 were incubated in simulated intestinal fluid (SIF) for 6-24 h. Inactivated SIF was used as a control. Following the treatment, samples were analyzed by HPLC by cGMP production in T84 cells. HPLC analysis revealed that intact plecanatide incubated in heat inactivated SIF was stable up to 6 h, eluting at 13.8 min (FIG. 8B). Treatment with active SIF resulted in complete degradation of plecanatide within 2 h to form an active metabolite that eluted at 9.4 min (indicated by an asterisk in FIG. 8C). The active metabolite, SP-338 was characterized by MS and MS/MS and identified to be formed via removal of carboxyl terminal leucine possibly due to cleavage by carboxypeptidases. Although, plecanatide is degraded rapidly in SIF, the major metabolite appears to retain biological activity, as only ˜35% of biological activity was lost within 2.5 h (FIG. 8A). HPLC analyses reveled that as expected, SP-333 was relatively more stable in SIF (FIG. 8F) losing ˜20% biological activity after a 24 h incubation in SIF (FIG. 8D). These results suggested that plecanatide is sensitive to proteolysis, which is problematic especially when considering administration to species with far longer GI transit times than mice. Hence, we chose to advance SP-333 in further animal studies.

Mice, randomly selected from treatment groups, were used to determine the proportion of normal Ki-67-immune reactive colonic epithelial cells (as a marker of regenerative capability) in large bowel cross-sections (FIG. 11A) and levels of MPO activity in lysates prepared from large bowel samples (as a marker for neutrophil infiltration and acute inflammation severity) (FIG. 11B). Strong immune reactivity demonstrating nuclear localization was observed in large bowel cross-sections incubated with anti-Ki-67 IgG. As shown in FIG. 11A SP-333 treated sections exhibited larger proportions of crypts with Ki-67 labeling than in DSS (35.3±4.86%) treated control sections. The proportion of Ki-67 positive crypts observed in animals administered 0.005 and 0.05 mg/kg/day of SP-333 was 60.4±6.96% and 58.7±7.3% respectively. The corresponding value in 5-ASA treated mice was 53.5 ±12.33%. Spearman rank correlation coefficients showed a statistically significant inverse correlation between colitis severity and the percentage of epithelial crypts demonstrating normal Ki-67 labeling (correlation coefficient of −0.449, p=0.003). Thus, oral treatment with SP-333 ameliorated DSS-induced changes in the Ki-67 labeling of crypt epithelial cells.

Lysates prepared from snap-frozen colon were analyzed for myeloperoxidase (MPO) activity (FIG. 11B). The DSS treated group exhibited the highest MPO activity (0.048±0.004 units/minute). MPO activity was much lower in lysates derived from SP-333 treated groups compared to DSS treated animals. The 0.05 mg/kg SP-333 treatment group had the lowest initial MPO activity (0.024±0.003 units/minute; p=0.001 compared to DSS group). Statistical significance was achieved for all other SP-333 treated groups except the 0.5 mg/kg group. Treatment with the reference compound, 5-ASA, was also associated with a low initial MPO activity (0.029±0.004; p=0.010). Calculation of the Spearman rank correlation coefficient revealed a significant, positive relationship between MPO activity and colitis severity scores (rs=0.277, p=0.05).

### DISCUSSION

This is the first study reporting that plecanatide and SP-333, superior analogs of UG, ameliorated GI inflammation in acute as well as chronic models of murine colitis. Oral treatment, once daily, with plecanatide or SP-333 at a dose range between 0.005 to 2.5 mg/kg was observed to be as effective as once daily treatment with 5-ASA (100 mg/kg) or sulfasalazine (80 mg/kg). However, doses of either of the GC-C agonists higher than 2.5 mg/kg did not show incremental dose response in amelioration of colitis, which may be attributed to the saturation of the available GC-C receptors on the epithelial cells lining the GI mucosa. The lowest effective dose of plecanatide varied from 0.005 to 2.5 mg/day in different models used at three different sites, which may be due to external factors such as diet and animal husbandry conditions of contract research organizations in US and UK which are known to impact the composition of gut microflora influencing the severity of colitis in different species mice (Fri swell et al., 2010; Gill and Finlay, 2011).

The GI immune system is controlled primarily by a milieu of pro-inflammatory and anti-inflammatory cytokines and growth factors promoting mucosal defense and integrity of intestinal tissue. Following the loss of barrier function, this regulatory balance is disrupted by the massive recruitment of leucocytes and macrophages, producing increased amounts of destructive inflammatory cytokines (Neuman, 2007). Our results demonstrate that plecanatide treatment significantly reduced production of IL-12 p40, IL-23 and TNF in colon explants from TNBS-treated BALB/c mice. Similarly, plecanatide treatment reduced production of RANTES, IL-17 and MIP1α with a concomitant increase in IL-10 in colon explants from TCRα^{-/-} mice. Furthermore, our in vitro results further show that SP-333 inhibits NF-κB activation via a cGMP-mediated mechanism in T84 cells. We also recently reported that oral treatment with plecanatide reduced formation of colon dysplasia in DSS treated Apc^{Min/+-FCCC} through downregulation of a number of pro-inflammatory cytokines and growth factors (Chang et al., 2014). Taken together, these results suggest that orally-administered plecanatide or SP-333 ameliorate colitis, though inhibition of NF-κB and downstream signaling resulting in suppressed production of pro-inflammatory cytokines.

During the GI renewal process, the epithelium goes through a cycle of proliferation, migration, differentiation, apoptosis and eventual loss of epithelial cells into the lumen (Lipkin, 1972; Eastwood, 1992). This process is crucial for maintaining the integrity of the intestinal mucosa and barrier function. Any disruption in this renewal process, impairing epithelial cell homeostasis and causing loss of intestinal barrier function, could potentially lead to GI inflammation and colon carcinogenesis (Lipkin, 1972; Eastwood, 1992). in this regard, deletion of GC-C gene in mice disrupts barrier function, resulting in increased permeation of luminal antigens that promote inflammation, damaging the intestinal mucosal architecture (Li et al., 2007; Han et al., 2011; Harmel-Laws et al., 2013). Disruption in GC-C signaling, reflecting early down regulation of UG and GN in colon polyps, tumors and in inflamed tissues from UC and CD patients, could contribute to loss of barrier function and chromosomal instability underlying the colonic inflammation.

Oral treatment with analogs of the endogenous GC-C ligands has a unique mechanism of action, enabling restoration of homeostatic signaling responsible for maintenance of colonic mucosa integrity. This study has important implications for treatment and maintenance of IBD in humans. First, oral treatment with locally-acting GC-C agonists eliminates toxicity concerns associated with the existing systemic therapies of IBD. Second, the evolving paradigm demonstrates that the reduced production of UG and GN is associated with the pathologies of UC and CD in humans. Accordingly, oral therapy with homologues of UG may be a replacement therapy to overcome the UG deficiency underlying the etiology of IBD. Finally, it is now well established that patients suffering with chronic UC and CD are at higher risk of developing colon cancer (Ullman and Itzkowitz, 2011). Chronic treatment with orally safe drug candidates such plecanatide or SP-333 are useful as maintenance therapy to delay the onset of IBD in to colon carcinogenesis. In this regard, we recently reported that oral treatment with plecanatide considerably reduced colonic dysplasia in Apc^{min/+-FCCC} mice that were treated with DSS to induce GI inflammation (Shailubhai et al., 2012; Chang et al., 2014).

Although the potency of plecanatide to stimulate cGMP in T84 cells and to ameliorate colitis in animal studies was comparable to that of SP-333, the latter drug candidate was advanced further for clinical development because of its enhanced stability against proteolysis in SIF. The non-clinical safety and toxicology studies conducted in rodents and monkeys suggest that SP-333 is an orally-safe drug candidate.

### Example 2

### SP-333, a Guanylate Cyclase-C Agonist, Inhibits NF-κB Signaling and Modulates Related Genes and miRNAs Implicated in GI Inflammation and Carcinogenesis

MicroRNAs are small non-coding RNA that are post-transcriptional negative regulators of gene expression and play important roles in biological processes such as cell cycle differentiation, metabolic pathways, and immune responses. MiRs are known to regulate expression of genes involved in the processes leading to inflammation and carcinogenesis. Aberrant expression of miR-21, let-7 family, miR-155, and miR-133a is linked to several human diseases such as ulcerative colitis, Crohn's disease, and colitis-induced colorectal cancer. SP-333, a proteolytically resistant analog of uroguanylin, is currently under clinical development for the treatment of ulcerative colitis. Studies have shown that SP-333 activated guanylate cyclase-C (GC-C) expressed on the epithelial cells lining the gastrointestinal (GI) mucosa to stimulate production of cyclic GMP (cGMP), a second messenger known to mediate anti-inflammatory effects on NF-κB and downstream signaling. Previous studies have revealed that oral treatment with SP-333 ameliorates colitis in DSS- and TNBS-induced colitis animal models. In human T84 cells, SP-333 treatment results in down regulation of NF-κB signaling and production of pro-inflammatory cytokines. Previously, we reported that SP-333 ameliorated colitis through inhibition of NF-κB signaling in mice. The objective of this study was to examine the effect of SP-333 treatment on expression of genes and miRs that are implication in processes underlying GI inflammation and cancer.

FIG. 1 shows the relationship of Uroguanylin (UG) and SP-333. SP-333 is a 16-mer of the endogenous BC-C ligand UG. The aspartic acid (D) at position 3 from the NH2-terminus of UG is substituted with glutamic acid (E). D-stereoisomers of asparagine (N) and leucine (L) at position 1 and 16 respectively enhance proteolytic stability. This enhanced proteolytic stability results in SP-333 being stable in simulated gastric and intestinal fluids, making this a highly potent peptide that can be administered orally.

### Methods

The T84 cell line was used to evaluate the ability f SP-333 to stimulate cGMP production. Total RNA and miRs were isolated from SP-333 treated T84 cells and levels of gene transcripts were determined using the TaqMan Open Array platform. Extracted total miRs were analyzed by MiR array. Statistical analyses were performed using Student's t-test.

Cell Culture: Human colon carcinoma cells T84 (ATCC Number CCL-248) were provided by Dr. Leonard Forte, University of Missouri at Columbia, Mo. Cells were cultured in Dulbeco's Modified Eagle Medium (DMEM) and Ham's F-12 medium (1:1) containing 10% fetal bovine serum, 1% Glutamax, 1% penicillin, and 1% streptomycin at 37° C. in a 5% C)2 controlled incubator.

cGMP Stimulation Assay: Confluent monolayers of T84 cells in 24-well plates were pre-incubated with indicated amounts of SP-333 in the presence of zaprinast for 30 minutes at 37° C. Subsequently, the reaction was terminated by 3% perchloric acid and neutralized with 0.5N NaOH. Intracellular cGMP levels were determined in lysates using a ELISA kit (Cayman Chemical). All samples were analyzed in duplicates. Total protein was quantitated using Pierce BCA protein assay (Thermo Fisher Scientific).

MicroRNA Microarray Analysis: T84 cells were grown to confluence in 100 mm tissue culture dishes. Cells were treated with pre-determined concentration of SP-333 (1 μM) in the presence of phosphodiesterase inhibitor, zaprinast (500 μM; Sigma Aldrich) for 5 hours. Following treatment, cells were trypsinized and the sample was divided into 3 aliquots. One aliquot was used to determine total cGMP. Another was used for extracting total RNA. The last aliquot was used to extract and purify miRNAs using Norgen's Kit (Norgen BioteK). Briefly, cells were lysed using lysis buffer, passed through a column to remove large RNA, followed by capturing miRNA using microRNA enrichment column. Purified samples were stored at −20° C. MicroRNAs were profiled on a LC Sciences microarray chip and TaqMan Open Array Human miRNA panel.

Quantitative RT-PCR for Transcripts: T-84 cells were treated with 1 μM SP-333 for 5 hours. Subsequently, cDNA was prepared from total RNA and subjected to quantitative Real-Time PCR using TaqMan reagents from Applied Biosystems.

### Results

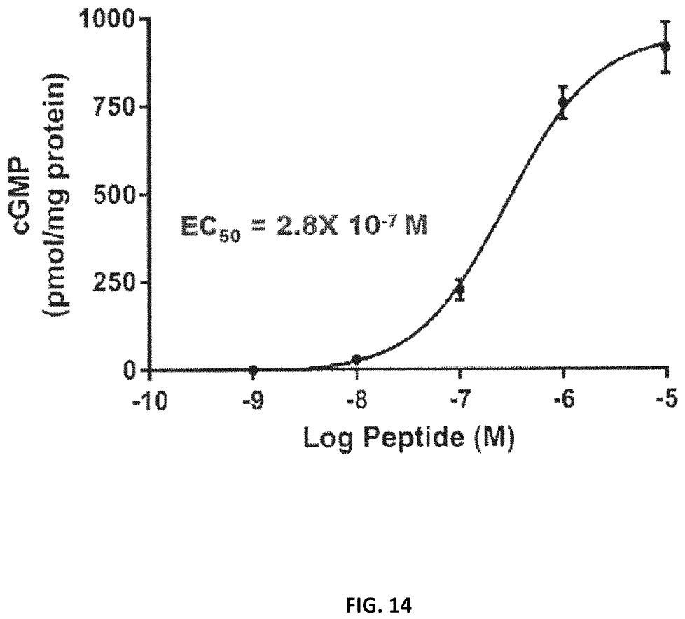

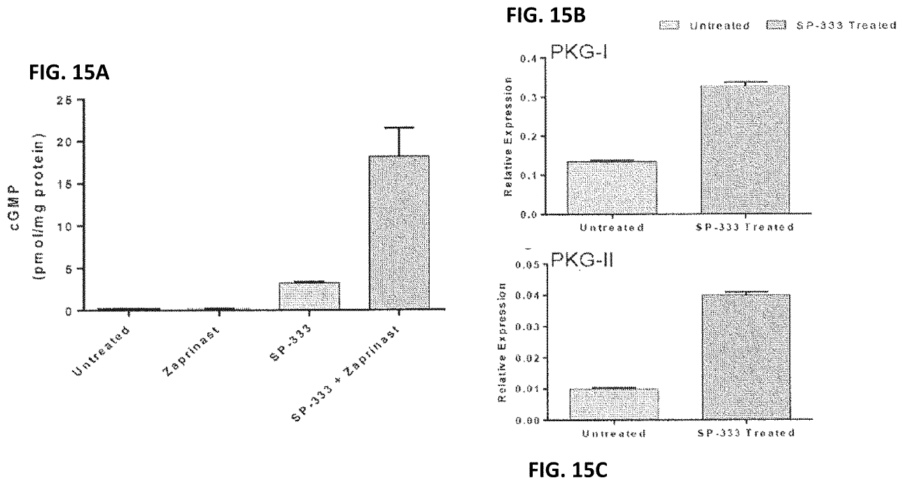

SP-333 stimulated cGMP synthesis in T84 cells with an EC₅₀ of 2.83×10⁻⁷ M. As shown in FIG. 14, SP-333 treatment stimulated cGMP synthesis in a dose-dependent manner in T84 cells, and approached a plateau at a concentration of 1 μM. Further, treatment with SP-333 enhanced cGMP production and Expression of Protein Kinase G I and II Transcripts (FIG. 15).

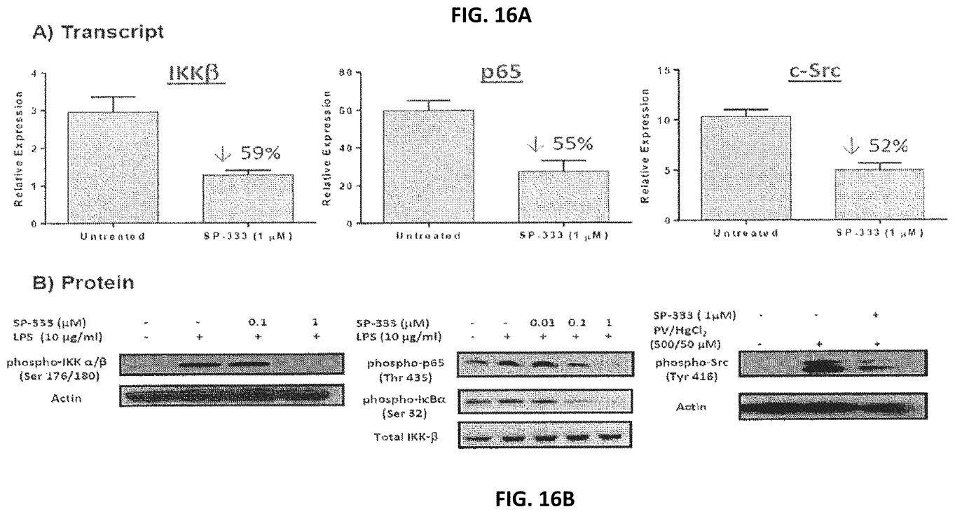

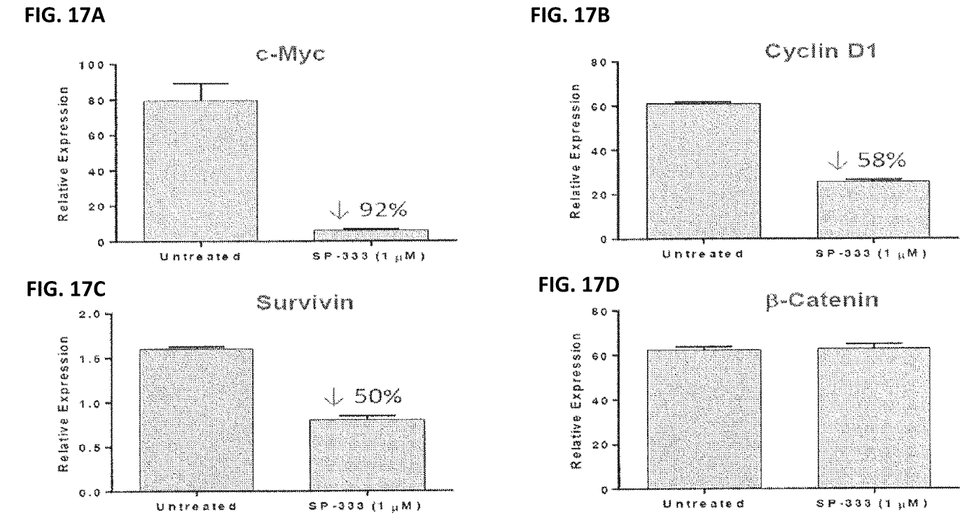

As shown in FIG. 16, as compared to vehicle, treatment with SP-333 downregulated NF-κB subunits, IKK-β, c-Src, and p65 as judged by reduction in their transcript and protein levels. After treatment with SP-333, a 59% decrease in IKKβ expression, a 55% decrease in p65 expression, and a 52% decrease in c-Src expression compared to untreated cells. FIG. 17 shows that SP-333 downregulates c-Myc and transcripts of genes related to cell-cycle in T84 cells. Treatment with SP-333 results in a 92% decrease in c-Myc expression a 58% decrease in Cyclin D1 expression, and a 50% decrease in Survivin expression. Treatment with SP-333 appears to have no effect on the expression of β-Catenin.

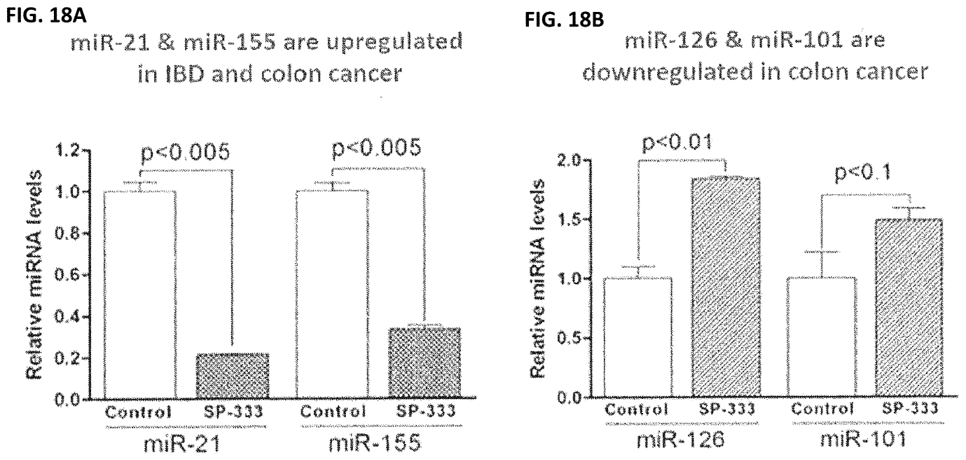

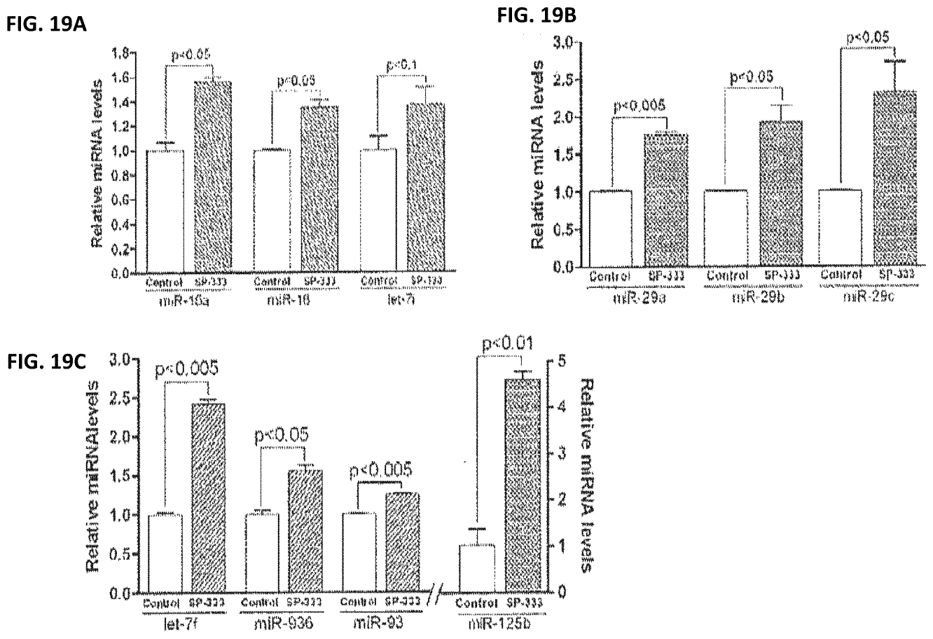

SP-333 treatment modulates miRNAs known to be dysregulated in inflammation and cancer. As shown in FIG. 18, shows that in IBD and colon cancer, treatment with SP-333 upregulates miR-21 and MiR-155 levels, while treatment with SP-333 downregulates levels of miR-126 and miR-101 in colon cancer. Further, FIG. 19 shows that SP-333 upregulates expression of miRNAs that are known to be expressed following NF-κB activation. NF-κB activation down-regulates miR-29 family and let-7i. MiR-15a/miR-16 down-regulate IKKα and inhibit proliferation, and induce apoptosis. Down regulation of miR-15b promotes production of TNFα. Let-7f and miR-936 levels inversely correlate with IL-23R and IL-17. Treatment with SP-333 upregulated miRs such as miR-15a (p<0.05), miR-16 (p<0.01), let-7i (p<0.005), miR-125b (p<0.001) and the family of miR-29 (p<0.05), all of which are negative regulators of NF-κB signaling, which is known to augment production of pro-inflammatory cytokines during GI inflammation.

SP-333 also significantly increased levels of miR-41-3p (p<0.01), which is a negative regulator of c-Src. Activation of c-Src is known to activate proto-oncogene c-Myc and suppress GC-C signaling. Activation of c-Myc transcriptionally suppresses miR-29b resulting in enhanced proliferation and resistance to apoptosis in malignant cells. In addition, Sp-333 treatment reduced transcripts of several putative c-Myc target genes such as cell division cycle 25a, cyclin D1, and p53. Consistent with these findings, SP-333 treatment increased levels of anti-carcinogenic miR-126 (p<0.01), miR-133a (p<0.01), miR-30c (p<0.05), and reduced levels of pro-carcinogenic miR-21 (p<0.005), miR-155 (p<0.005), miR-92a (p<0.05), miR-200c (p<0.01), and miR-494 (p<0.005). Table 8 shows that treatment with SP-333 modulates expression of miRNAs implicated in IBD and colon cancer.

TABLE 8

| miRNA | SP-333 | miRNA | SP-333 |
|---|---|---|---|
| expression | Treatment | Expression | Treatment |
| IBD | | NF-κB | |
| Upregulated | | Upregulated | |
| miR-21 | decrease | miR-21 | decrease |
| miR-362-3p | decrease | miR-27b | decrease |
| miR-532-3p | decrease | miR-30b | decrease |
| miR-28-5p | decrease | miR-10a | decrease |
| miR-99a | decrease | miR-199-3p | decrease |
| miR-203 | decrease | miR-301 | decrease |
| miR-26b | decrease | | |
| miR-98 | decrease | | |
| Downregulated | | Downregulated | |
| miR-192 | increase | miR-218 | increase |
| | | miR-223 | increase |
| | | miR-16 | increase |
| | | mir-29b | increase |
| | | miR-15a | increase |
| CRC | | cMyc | |
| Upregulated | | Upregulated | |
| miR-21 | decrease | miR-21 | decrease |
| miR-92 | decrease | miR-192 | decrease |
| miR-155 | decrease | miR-199-3p | decrease |
| | | miR-224 | decrease |
| Downregulated | | Downregulated | |
| miR-133a | increase | | |
| miR-126 | increase | | |
| miR-101 | increase | | |
| miR-133b | increase | | |
| miR-139-5p | increase | | |

### Conclusions

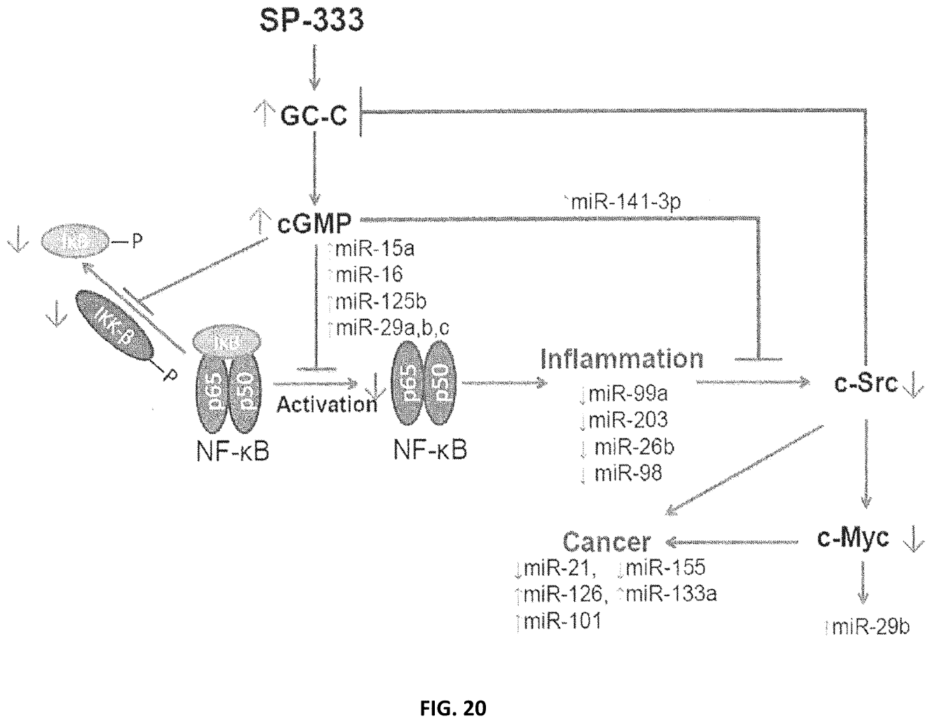

SP-333 binds and activates GC-C receptors expressed on T84 cells to stimulate cGMP production. SP-333 treatment attenuates LPS-mediated activation of NF-κB at transcriptional and post-translation levels in T-84 cells, and down-regulates transcripts of proto-oncogenes c-Src and c-Myc in T84 cells. Key miRNAs that are known to be dysregulated in inflammation and cancer are inversely modulated by SP-333 treatment. Further SP-333-mediated activation of GC-C signaling may contribute to its anti-inflammatory effects through modulation of miRNAs and genes implicated in activation of NF-κB, C-Src, and c-Myc signaling. This is the first report demonstrating that SP-333 mediated activation of GC-C signaling may contribute to its anti-inflammatory effects through suppression of c-Src, c-Myc, IKK-β, and NF-κB signaling, possibly through modulation of related miRs implicated in GI inflammation and cancer. FIG. 20 shows the putative mechanism by which SP-333 modulates expression of genes and miRNAs implicated in inflammation and cancer. These data will facilitate evaluation of the select miRNAs and corresponding target genes in IBD tissues.

### Other Embodiments

Those skilled in the art will recognize, or be able to ascertain using no more than routine experimentation, many equivalents to the specific embodiments described herein. Such equivalents are intended to be encompassed by the claims provided herein.

### INCORPORATION BY REFERENCE

This application incorporates by reference all publications or references disclosed herein for all purposes in their entireties.

### REFERENCES

Aiamkitsumrit B, Zhang X, Block T M, Norton P, Fraser N W and Su Y H (2007) Herpes simplex virus type 1 ICP4 deletion mutant virus d120 infection failed to induce apoptosis in nerve growth factor-differentiated PC12 cells. J Neurovirol 13:305-314.

Berg D J, Davidson N, Kuhn R, Muller W, Menon S, Holland G, Thompson-Snipes L, Leach M W and Rennick D (1996) Enterocolitis and colon cancer in interleukin-10-deficient mice are associated with aberrant cytokine production and CD4(+) TH1-like responses. J Clin Invest 98:1010-1020.

Blomain E S, Lin J E, Kraft C L, Trela U T, Rock J M, Aing A S, Snook A E and Waldman SA (2013) Translating colorectal cancer prevention through the guanylyl cyclase C signaling axis. Expert review of clinical pharmacology 6:557-564.

Chang W, Masih S, Cooper H, Palejwala V, Clapper M L and Shailubhai K (2014) Enhancement of Cyclic GMP Production through Activation of Guanylate Cyclase-C Inhibits Colorectal Adenomas in Apc+/Min-FCCC Mice with Dextran Sulfate Sodium-induced Inflammation. Mol Cancer Ther Communicated.

Choudhari S R, Khan M A, Harris G, Picker D, Jacob G S, Block T and Shailubhai K (2007) Deactivation of Akt and STAT3 signaling promotes apoptosis, inhibits proliferation, and enhances the sensitivity of hepatocellular carcinoma cells to an anticancer agent, atiprimod. Molecular Cancer Therapeutics 6:112-121.

Cohen M B, Hawkins J A and Witte D P (1998) Guanylin mRNA expression in human intestine and colorectal adenocarcinoma. Lab Invest 78:101-108.

Das S, Periyasamy R and Pandey K N (2012) Activation of IKK/NF-kappaB provokes renal inflammatory responses in guanylyl cyclase/natriuretic peptide receptor-A gene-knockout mice. Physiological genomics 44:430-442.

Dave S H, Tilstra J S, Matsuoka K, Li F, DeMarco R A, Beer-Stolz D, Sepulveda A R, Fink M P, Lotze M T and Plevy S E (2009) Ethyl pyruvate decreases HMGB1 release and ameliorates murine colitis. J Leukoc Biol 86:633-643.

Eastwood G L (1992) Epithelial renewal in premalignant conditions of the gastrointestinal tract: a review. J Clin Gastroenterol 14 Suppl 1:S29-33.

Forte L R, Eber S L, Fan X, London R M, Wang Y, Rowland L M, Chin D T, Freeman R H and Krause W J (1999) Lymphoguanylin: cloning and characterization of a unique member of the guanylin peptide family. Endocrinology 140:1800-1806.

Friswell M K, Gika H, Stratford U, Theodoridis G, Telfer B, Wilson I D and McBain A J (2010) Site and strain-specific variation in gut microbiota profiles and metabolism in experimental mice. PLoS One 5:e8584.

Gill N and Finlay B B (2011) The gut microbiota: challenging immunology. Nature reviews Immunology 11:636-637.

Hamamoto N, Maemura K, Hirata I, Murano M, Sasaki S and Katsu K (1999) Inhibition of dextran sulphate sodium (DSS)-induced colitis in mice by intracolonically administered antibodies against adhesion molecules (endothelial leucocyte adhesion molecule-1 (ELAM-1) or intercellular adhesion molecule-1 (ICAM-1)). Clin Exp Immunol 117:462-468.

Han X, Mann E, Gilbert S, Guan Y, Steinbrecher K A, Montrose M H and Cohen M B (2011) Loss of guanylyl cyclase C (GCC) signaling leads to dysfunctional intestinal barrier. PLoS One 6:e16139.

Harmel-Laws E, Mann E A, Cohen M B and Steinbrecher K A (2013) Guanylate cyclase C deficiency causes severe inflammation in a murine model of spontaneous colitis. PLoS One 8:e79180.

Hegazi R A, Rao K N, Mayle A, Sepulveda A R, Otterbein L E and Plevy S E (2005) Carbon monoxide ameliorates chronic murine colitis through a heme oxygenase 1-dependent pathway. J Exp Med 202:1703-1713.

Krawisz J E, Sharon P and Stenson W F (1984) Quantitative assay for acute intestinal inflammation based on myeloperoxidase activity. Assessment of inflammation in rat and hamster models. Gastroenterology 87:1344-1350.

Ladetzki-Baehs K, Keller M, Kiemer A K, Koch E, Zahler S, Wendel A and Vollmar A M (2007) Atrial natriuretic peptide, a regulator of nuclear factor-kappaB activation in vivo. Endocrinology 148:332-336.

Li P, Lin J E, Chervoneva I, Schulz S, Waldman S A and Pitari G M (2007) Homeostatic control of the crypt-villus axis by the bacterial enterotoxin receptor guanylyl cyclase C restricts the proliferating compartment in intestine. Am J Pathol 171:1847-1858.

Lin J E, Snook A E, Li P, Stoecker B A, Kim G W, Magee M S, Garcia A V, Valentino M A, Hyslop T, Schulz S and Waldman S A (2012) GUCY2C opposes systemic genotoxic tumorigenesis by regulating AKT-dependent intestinal barrier integrity. PLoS One 7:e31686.

Lipkin M (1972) Gastric cell regeneration. Arch Fr Mal App Dig 61:691-693.

Loftus E V, Jr. and Sandborn W J (2002) Epidemiology of inflammatory bowel disease. Gastroenterol Clin North Am 31:1-20.

London R M, Krause W J, Fan X, Eber S L and Forte L R (1997) Signal transduction pathways via guanylin and uroguanylin in stomach and intestine. Am J Physiol 273:G93-105.

Neuman M G (2007) Immune dysfunction in inflammatory bowel disease. Translational research : the journal of laboratory and clinical medicine 149:173-186.

Papachristou G I and Plevy S (2004) Novel biologics in inflammatory bowel disease. Gastroenterol Clin North Am 33:251-269, ix.

Rogler G, Brand K, Vogl D, Page S, Hofmeister R, Andus T, Knuechel R, Baeuerle P A, Scholmerich J and Gross V (1998) Nuclear factor kappaB is activated in macrophages and epithelial cells of inflamed intestinal mucosa. Gastroenterology 115:357-369.

Rutgeerts P, Sandborn W J, Feagan B G, Reinisch W, Olson A, Johanns J, Travers S, Rachmilewitz D, Hanauer S B, Lichtenstein G R, de Villiers W J, Present D, Sands B E and Colombel J F (2005) Infliximab for induction and maintenance therapy for ulcerative colitis. N Engl J Med 353:2462-2476.

Sandborn W J and Targan S R (2002) Biologic therapy of inflammatory bowel disease. Gastroenterology 122:1592-1608.

Schreiber S, Nikolaus S and Hampe J (1998) Activation of nuclear factor kappa B inflammatory bowel disease. Gut 42:477-484.

Shailubhai K (2002) Therapeutic applications of guanylate cyclase-C receptor agonists. Curr Opin Drug Discov Devel 5:261-268.

Shailubhai K, Chang W-C, Masih S, Cooper H S and Clapper M L (2012) Enhancement of cyclic GMP production by guanylate cyclase-C agonists delays progression of colitis into colon cancer though downregulation of pro-inflammatory cytokines. Cancer Res 72:Abstract nr 1636.

Shailubhai K, Comiskey S, Foss J A, Feng R, Barrow L, Comer G M and Jacob G S (2013) Plecanatide, an oral guanylate cyclase C agonist acting locally in the gastrointestinal tract, is safe and well-tolerated in single doses. Dig Dis Sci 58:2580-2586.

Shailubhai K and Jacob G (2009) Agonists of guanylate cyclase useful for the treatment of gastrointestinal disorders, in (Office USPaT ed), Synergy Pharmaceuticals Inc.

Shailubhai K, Yu H H, Karunanandaa K, Wang J Y, Eber S L, Wang Y, Joo N S, Kim H D, Miedema B W, Abbas S Z, Boddupalli S S, Currie M G and Forte L R (2000) Uroguanylin treatment suppresses polyp formation in the Apc(Min/+) mouse and induces apoptosis in human colon adenocarcinoma cells via cyclic GMP. Cancer Res 60:5151-5157.

Sheikh S Z, Hegazi R A, Kobayashi T, Onyiah J C, Russo S M, Matsuoka K, Sepulveda A R, Li F, Otterbein L E and Plevy S E (2011) An anti-inflammatory role for carbon monoxide and heme oxygenase-1 in chronic Th2-mediated murine colitis. J Immunol 186:5506-5513.

Tak P P and Firestein G S (2001) NF-kappaB: a key role in inflammatory diseases. J Clin Invest 107:7-11.

Targan S R, Hanauer S B, van Deventer S J, Mayer L, Present D H, Braakman T, DeWoody K L, Schaible T F and Rutgeerts P J (1997) A short-term study of chimeric monoclonal antibody cA2 to tumor necrosis factor alpha for Crohn's disease. Crohn's Disease cA2 Study Group. N Engl J Med 337:1029-1035.

Tsukagoshi H, Shimizu Y, Kawata T, Hisada T, Shimizu Y, Iwamae S, Ishizuka T, Iizuka K, Dobashi K and Mori M (2001) Atrial natriuretic peptide inhibits tumor necrosis factor-alpha production by interferon-gamma-activated macrophages via suppression of p38 mitogen-activated protein kinase and nuclear factor-kappa B activation. Regul Pept 99:21-29.

Ullman T A and Itzkowitz S H (2011) Intestinal inflammation and cancer. Gastroenterology 140:1807-1816.

Vaandrager A B, Bot A G, Ruth P, Pfeifer A, Hofmann F and De Jonge H R (2000) Differential role of cyclic GMP-dependent protein kinase II in ion transport in murine small intestine and colon. Gastroenterology 118:108-114.

Vaandrager A B and de Jonge H R (1996) Signalling by cGMP-dependent protein kinases. Mol Cell Biochem 157:23-30.

Vellaichamy E, Sommana N K and Pandey K N (2005) Reduced cGMP signaling activates NF-kappaB in hypertrophied hearts of mice lacking natriuretic peptide receptor-A. Biochem Biophys Res Commun 327:106-111.

Wu F, Dassopoulos T, Cope L, Maitra A, Brant S R, Harris M L, Bayless T M, Parmigiani G and Chakravarti S (2007) Genome-wide gene expression differences in Crohn's disease and ulcerative colitis from endoscopic pinch biopsies: insights into distinctive pathogenesis. Inflamm Bowel Dis 13:807-821.

Xavier R J and Podolsky D K (2007) Unravelling the pathogenesis of inflammatory bowel disease. Nature 448:427-434.

Zhang G, Arjunan K P, Foss J, Comiskey S and Shailubhai K (2012) SP-333, a Proteolysis-resistant Agonist of Guanylate Cyclase-C, Inhibits Activation of NF-kappa B and Suppresses Production of Inflammatory Cytokines to Ameliorate DSS-induced Colitis in Mice. Am J Gastroenterol 107:S626-S626.

We claim:

1. A method of treating celiac disease in a patient in need thereof, the method comprising administering a therapeutically effective amount of a guanylate receptor agonist (GCRA) peptide, wherein the GCRA peptide has the sequence of SEQ ID NO: 1 or SEQ ID NO: 9.

2. The method of claim 1, wherein the GCRA peptide has the sequence of SEQ ID NO: 1.

3. The method of claim 1, wherein the GCRA peptide has the sequence of SEQ ID NO: 9.

4. The method of claim 1, further comprising administering an additional therapeutic agent selected from the group consisting of an immune system suppressor and a steroid.

5. The method of claim 4, wherein the immune system suppressor comprises azathioprine.

6. The method of claim 4, wherein the steroid comprises budesonide.
